(19) United States
(12) Patent Application Publication
(10) Pub. No.: US 2025/0340506 A1
(43) Pub. Date: Nov. 6, 2025

(54) LIPID FORMULATIONS FOR GENE EDITING

(71) Applicant: Verve Therapeutics, Inc., Boston, MA (US)

(72) Inventors: Kallanthottathil G. Rajeev, Wayland, MA (US); Souvik Biswas, Woburn, MA (US); Padma Malyala, Burlington, MA (US); Lisa N. Kasiewicz, Cambridge, MA (US); Aaron Beach, Salem, MA (US); Thomas V. Colace, Cambridge, MA (US)

(21) Appl. No.: 19/208,278

(22) Filed: May 14, 2025

Related U.S. Application Data
(63) Continuation of application No. 18/654,660, filed on May 3, 2024, now Pat. No. 12,410,122.
Application No. 18/654,660 is a continuation of application No. 18/180,306, filed on Mar. 8, 2023, now Pat. No. 12,024,484.
Application No. 18/180,306 is a continuation of application No. PCT/US2021/050511, filed on Sep. 15, 2021.
(60) Provisional application No. 63/078,738, filed on Sep. 15, 2020.
(60) Provisional application No. 63/220,340, filed on Jul. 9, 2021.

(51) Int. Cl. C07C 229/12

(52) U.S. Cl. CPC C07C 229/12

(57) ABSTRACT

The present disclosure relates to PEG-lipids, cationic and/or ionizable lipids and nucleic acid-lipid particle compositions comprising the same. The present disclosure also relates to methods of making, using and delivering the described lipids and lipid-containing particles.

### CROSS-REFERENCE

This application is a continuation of U.S. patent application Ser. No. 18/654,660, filed May 3, 2024 which is a continuation of U.S. patent application Ser. No. 18/180,306, filed Mar. 8, 2023 which is a continuation of International Patent Application No. PCT/US2021/050511, filed Sep. 15, 2021 which claims the benefit of U.S. Provisional Application No. 63/220,340, filed Jul. 9, 2021, and U.S. Provisional Application No. 63/078,738, filed Sep. 15, 2020, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jul. 14, 2023, is named 53989-703_301_US_SL.xml and is 1,069,846 bytes in size.

### BACKGROUND

Lipid-containing particles have been used to encapsulate, and as transport vehicles for therapeutic agents such as nucleic acids, small molecules compounds, and proteins into cells and other intracellular compartments. There remains an ongoing need to develop new lipids to encapsulate therapeutic agents and improve the safety, efficacy, and specificity of such nanoparticle-based transport vehicles.

### SUMMARY

Described herein are novel lipids and lipid nanoparticles comprised thereof. In one aspect, novel amino lipids are described. In another aspect novel PEG-lipids are described. In yet another aspects, novel lipid nanoparticles are described comprising one or more of the novel amino lipids and/or novel PEG-lipids. The nanoparticles, as described herein, in one aspect, are comprised of one or more of the following: an amino lipid, a neutral lipid, a PEG-lipid, a sterol or a derivative thereof, and optionally one or more of a nucleic acid molecular entity, a nucleic acid stabilizer, a surfactant, and an antioxidant. Further described herein are methods of using and making the same.

In one aspect, disclosed herein is an amino lipid having a structure of Formula (I), or a pharmaceutically acceptable salt or solvate thereof,

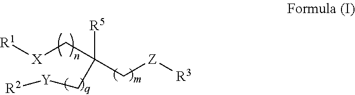

wherein
each of R¹ and R² is independently C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, or a bond;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴═N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloaryl is independently substituted or unsubstituted;
each of R⁴ is independently hydrogen or substituted or unsubstituted C₁-C₆ alkyl; R⁵ is hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl; and
each of n, m, and q is independently 0, 1, 2, 3, 4, or 5.

In one aspect, disclosed herein is an amino lipid having a structure of Formula (I*), or a pharmaceutically acceptable salt or solvate thereof,

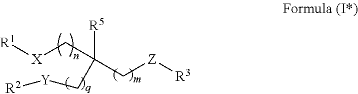

wherein
R¹ is hydrogen, C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
R² is hydrogen, C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —R¹¹C(═O)N(R⁴)R⁹—, —NR⁷C(═O)R⁹—, —C(═O)O—, —OC(═O)—, —R¹¹C(═O)OR⁹—, —R¹¹OC(═O)R⁹—, —R¹¹OC(═O)OR⁹—, —R¹¹N(R⁴)C(═O)OR⁹—, —R¹¹OC(═O)N(R⁴)R⁹—, —R¹¹(NR⁴)C(═O)N(R⁴)R⁹—, —R¹¹N(R⁴)C(═NR⁴)R⁹—, —R¹¹C(═S)N(R⁴)R⁹—, —R¹¹N(R⁴)C(═S)R⁹—, —R¹¹C(═S)OR⁹—, —R¹¹OC(═S)R⁹—, —R¹¹OC(═S)OR⁹—, —R¹¹N(R⁴)C(═S)OR⁹—, —R¹¹OC(═S)N(R⁴)R⁹—, —R¹¹N(R⁴)C(═S)N(R⁴)R⁹—, —R¹¹C(═O)SR⁹—, —R¹¹SC(═O)R⁹—, —R¹¹OC(═O)SR⁹—, —R¹¹N(R⁴)C(═O)SR⁹—, —R¹¹SC(═O)N(R⁴)R⁹—, —R¹¹C(═S)SR⁹—, —R¹¹SC(═S)R⁹—, —R¹¹SC(═S)OR⁹—, —R¹¹N(R⁴)C(═S)SR⁹—, —R¹¹SC(═S)N(R⁴)R⁹—, —R¹¹C(═S)SR⁹—, —R¹¹SC(═S)R⁹—, —R¹¹SC(═O)SR⁹—, —R¹¹SC(═S)SR⁹—, —R¹¹N(R⁴)C(═S)SR⁹—, —R¹¹SC(═S)N(R⁴)R⁹—, —R¹¹OR⁹—, —R¹¹SR⁹—, —O—, —S—, or a bond;
each of L is independently —C(═O)NR⁴—, —NR⁴C(═O)—, —C(═O)O—, —OC(═O)—, —C(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴═N—O—, —O—, —S—, —R¹¹C(═O)N(R⁴)R⁹—, —R¹¹N(R⁴)C(═O)R⁹—, —R¹¹C(═O)OR⁹—, —R¹¹OC(═O)R⁹—, —R¹¹OC(═O)OR⁹—, —R¹¹N(R⁴)C(═O)OR⁹—, —R¹¹OC(═O)N(R⁴)R⁹—, —R¹¹N(R⁴)C(═O)N(R⁴)R⁹—, —R¹¹N(R⁴)C(═NR⁴)N(R⁴)R⁹—, —R¹¹C(═S)N(R⁴)R⁹—, —R¹¹N(R⁴)C(═S)R⁹—, —R¹¹C(═S)OR⁹—, —R¹¹OC(═S)R⁹—, —R¹¹OC(═S)OR⁹—, —R¹¹N(R⁴)C(═S)OR⁹—, —R¹¹OC(═S)N(R⁴)R⁹—, —R¹¹N(R⁴)C(═S)N(R⁴)R⁹—, —R¹¹C(═O)SR⁹—, —R¹¹SC(═O)R⁹—, —R¹¹OC(═O)SR⁹—, —R¹¹N(R⁴)C(═O)SR⁹—, —R¹¹SC(═O)N(R⁴)R⁹—, —R¹¹C(═S)SR⁹—, —R¹¹SC(═S)R⁹—, —R¹¹SC(═S)OR⁹—, —R¹¹N(R⁴)C(═S)SR⁹—, —R¹¹SC(═S)N(R⁴)R⁹—, —R¹¹C(═S)SR⁹—, —R¹¹SC(═S)R⁹—, —R¹¹SC(═O)SR⁹—, —R¹¹SC(═S)SR⁹—, —R¹¹N(R⁴)C(═S)SR⁹—, —R¹¹SC(═S)N(R⁴)R⁹—, —R¹¹O—N═CR⁹—, —R¹¹C(R¹⁰)=N—OR⁹—, —R¹¹OR⁹—, —R¹¹SR⁹—, —C₁-C₁₀ alkylene-C(═O)O—, —C₁-C₁₀ alkylene-OC(═O)—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloaryl is independently substituted or unsubstituted;
each of R⁴ is independently hydrogen, substituted or unsubstituted C₁-C₁₆ alkyl or substituted or unsubstituted C₁-C₁₆ heteroalkyl;
R⁵ is hydrogen, substituted or unsubstituted —C₀-C₁₀ alkylene-L-R⁴;
each of R⁶ is independently hydrogen, C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₁₆ alkyl, substituted or unsubstituted C₁-C₁₆ heteroalkyl, or R⁷ and R⁸ taken together with the nitrogen to which they are attached form a substituted or unsubstituted C₂-C₆ heterocyclyl; and
each of R⁹ is independently hydrogen or substituted or unsubstituted C₁-C₁₆ alkylene, or unsubstituted C₁-C₁₆ heteroalkylene;
each of R¹⁰ is independently hydrogen or substituted or unsubstituted C₁-C₁₆ alkylene, or unsubstituted C₁-C₁₆ heteroalkylene; and each of R¹¹ is independently substituted or unsubstituted C₁-C₁₆ alkylene, or unsubstituted C₁-C₁₆ heteroalkylene.
each of n, m, and q is independently 0, 1, 2, 3, 4, or 5.

In some embodiments, the amino lipid of Formula (I) or (I*) has a structure of Formula (Ia), or a pharmaceutically acceptable salt or solvate thereof,

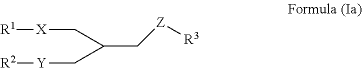

wherein
each of R¹ and R² is independently C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, —C₁-C₁₀ alkylene-O—, or a bond, wherein the alkylene is substituted or unsubstituted;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸ or —C₀-C₁₀ alkylene-heterocycloalkyl, wherein the alkylene and heterocycloalkyl are each independently substituted or unsubstituted;
each of R⁴ is independently hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl; and
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl.

In another aspect, disclosed herein is an amino lipid having structure of Formula (Ib), or a pharmaceutically acceptable salt thereof,

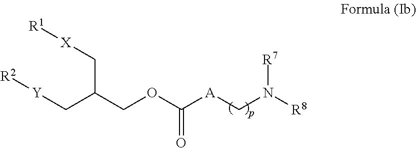

wherein
each of X and Y are the same and are selected from the group consisting of: —OC(═O)—, —OC(═O)O—, —OC(═O)NR⁴—, —C(═S)O—, —OC(═S)—, OC(═S)O—, —NR⁴C(═S)O—, —OC(═S)NR⁴—, SC(═O)—, —OC(═O)S—, —SC(═S)O—, —O—, —C₁-C₁₀ alkylene-O—, and a bond, wherein the alkylene is substituted or unsubstituted
R¹ and R² are the same and are selected from the group consisting of: hydrogen, C₁-C₃₀ alkyl, C₁-C₃₀ heteroalkyl, and Cy-C₃₋₃₀ alkyl, wherein the alkyl and heteroalkyl are each independently substituted or unsubstituted;
each of R⁴ is independently hydrogen or substituted or unsubstituted C₁-C₆ alkyl;
each of Cy is substituted or unsubstituted cycloalkyl, substituted or unsubstituted heterocycloalkyl, substituted or unsubstituted aryl, substituted or unsubstituted heteroaryl; and
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl.
A is —O—, —CH₂—, —S—, or —NR¹²—;
R¹² is hydrogen, C₁-C₁₀ alkyl, or C₁-C₁₀ heteroalkyl, wherein the alkyl and heteroalkyl are each independently substituted or unsubstituted; and
Cy is substituted or unsubstituted cycloalkyl, substituted or unsubstituted heterocycloalkyl, substituted or unsubstituted aryl, or substituted or unsubstituted heteroaryl.
p is 1, 2, 3, 4, 5 or 6

In some embodiments, R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;

In one aspect, disclosed herein is an amino lipid having a structure of Formula (II), or a pharmaceutically acceptable salt or solvate thereof,

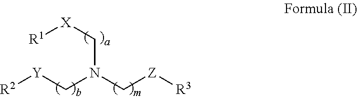

wherein
each of R¹ and R² is independently C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₁-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, or a bond;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloaryl is independently substituted or unsubstituted;
each of R⁴ is independently hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl;
each of a and b is independently 1, 2, 3, 4, 5, 6, 7, 8, 9, or 10; and
m is 0, 1, 2, 3, 4, or 5.

In one aspect, disclosed herein is an amino lipid having a structure of Formula (II*), or a pharmaceutically acceptable salt or solvate thereof,

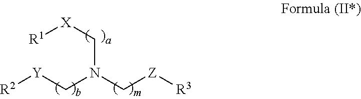

wherein
each of R¹ and R² is independently hydrogen, C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₁-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently —C(═O)NR⁴—, —C(═O)—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, or a bond;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is hydrogen, —C₁-C₆ alkyl, —C₀-C₁₀ alkylene-NR⁷R⁸, —C₀-C₁₀ alkylene-heterocycloalkyl, or —C₀-C₁₀ alkylene-heterocycloaryl, wherein the alkyl, alkylene, heterocycloalkyl and heterocycloaryl is independently substituted or unsubstituted;
each of R⁴ is independently hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl;
each of a and b is independently 1, 2, 3, 4, 5, 6, 7, 8, 9, or 10; and
m is 0, 1, 2, 3, 4, or 5.

In one aspect, disclosed herein is a nanoparticle composition that comprises an amino lipid described herein or a pharmaceutically acceptable salt or solvate thereof. In one aspect, disclosed herein is an amino lipid having a structure selected from Table 1A, or a pharmaceutically acceptable salt or solvate thereof. In one aspect, disclosed herein is a nanoparticle composition that comprises an amino lipid having a structure selected from Table 1A, or a pharmaceutically acceptable salt or solvate thereof. In some embodiments, said amino lipid or a pharmaceutically acceptable salt or solvate thereof, comprises from 20 mol % to 80 mol % of a total lipid content present in said nanoparticle composition. In some embodiments, said amino lipid or a pharmaceutically acceptable salt or solvate thereof, comprises from 40 mol % to 60 mol % of a total lipid content present in said nanoparticle composition. In some embodiments, said amino lipid or a pharmaceutically acceptable salt or solvate thereof, comprises from 50 mol % to 60 mol % of a total lipid content present in said nanoparticle composition. In some embodiments, said amino lipid or a salt or solvate thereof, comprises one or more ionizable nitrogen atoms. In some embodiments, the nanoparticle composition comprises one or more ionizable nitrogen atoms that are from the one or more amino lipids. In some embodiments, said nanoparticle composition comprises only one amino lipid or a salt or solvate thereof. In some embodiments, said nanoparticle composition comprises one or more nucleic acid molecular entities. In some embodiments, a molar ratio of said ionizable nitrogen atoms to phosphate groups present in said nucleic acid molecular entities (the N to P or N/P ratio) is from about 2 to about 20. In some embodiments, said N/P ratio is from about 2 to about 15, from about 2 to about 10, from about 2 to about 8, from about 2 to about 6, from about 3 to about 15, from about 3 to about 10, from about 3 to about 8, from about 3 to about 6, from about 4 to about 15, from about 4 to about 10, from about 4 to about 8, or from about 4 to about 6. In some embodiments, said N/P ratio is from about 3.5 to about 10. In some embodiments, said nanoparticle composition comprises a neutral lipid. In some embodiments, said neutral lipid comprises from about 1 mol % to about 20 mol % of a total lipid content present in said nanoparticle composition. In some embodiments, said neutral lipid comprises from about 2 mol % to about 25 mol % of a total lipid content present in said nanoparticle composition. In some embodiments, said neutral lipid comprises from about 5 mol % to about 10 mol % of a total lipid content present in said nanoparticle composition. In some embodiments, said neutral lipid is selected from 1,2-distearoyl-sn-glycero-3-phosphocholine (DSPC), 1,2-dipalmitoyl-sn-glycero-3-phosphocholine (DPPC), 1,2-dimyristoyl-sn-glycero-phosphocholine (DMPC), 2-oleoyl-1-palmitoyl-sn-glycero-3-phosphocholine (POPC), 1,2-dioleoyl-sn-glycero-3-phosphoethanolamine (DOPE), and sphingomyelin. In some embodiments, said neutral lipid is DSPC. In some embodiments, said nanoparticle composition comprises a structural lipid. In some embodiments, said structural lipid is sterol or a derivative thereof. In some embodiments, said sterol or said derivative thereof is cholesterol or cholesterol derivative. In some embodiments, said structural lipid comprises from about 15 mol % to about 65 mol % of a total lipid content present in the nanoparticle composition. In some embodiments, said structural lipid comprises from about 30 mol % to about 60 mol % of a total lipid content present in the nanoparticle composition. In some embodiments, said structural lipid comprises from about 30 mol % to about 40 mol % of a total lipid content present in the nanoparticle composition. In some embodiments, said nanoparticle composition comprises a PEG-lipid. In some embodiments, said PEG-lipid is a PEG-lipid of Table 2. In some embodiments, said PEG-lipid comprises from about 0.1 mol % to about 6 mol % of a total lipid content present in said nanoparticle composition. In some embodiments, said PEG-lipid comprises about 2.0 mol % to about 2.5 mol % of a total lipid content present in said nanoparticle composition. In some embodiments, a number average molecular weight of said PEG-lipid is from about 200 Da to about 5000 Da. In some embodiments, said one or more nucleic acid molecular entities comprise a guide RNA (gRNA) targeting a disease causing gene of interest. In some embodiments, the guide RNA is a single guide RNA (sgRNA). In some embodiments, the disease causing gene of interest is produced in hepatocytes. In some embodiments, said one or more nucleic acid molecular entities comprise an mRNA encoding SpCas9, CBE, and/or ABE proteins. In some embodiments, the composition comprises a nucleic acid stabilizer.

In one aspect, disclosed herein is a nanoparticle composition comprising: (a) one or more nucleic acid molecular entities; (b) an amino lipid described herein, or a salt or solvate thereof, wherein said amino lipid or a salt thereof, comprises from 20 mol % to 80 mol % of a total lipid content present in said nanoparticle composition, wherein said amino lipid or a salt thereof, comprises one or more ionizable nitrogen atoms, and wherein a molar ratio of said ionizable nitrogen atoms to phosphate groups present in said nucleic acid molecular entity is from 2 to 12; (c) a neutral lipid, comprising from 2 mol % to 25 mol % of said total lipid content present in said nanoparticle composition; (d) a structural lipid, comprising from 30 mol % to 60 mol % of said total lipid content present in said nanoparticle composition; and (e) a PEG-lipid, comprising from 0.1 mol % to 6 mol % of said total lipid content present in said nanoparticle composition. In some embodiments, the composition further comprises a nucleic acid stabilizer. In some embodiments, the nucleic acid stabilizer comprises polyethylene glycol, cetrimonium bromide, or chitosan. In some embodiments, the nucleic acid stabilizer comprises polyethylene glycol that has a number average molecular weight of about 120 to about 2000 Da.

In one aspect, disclosed herein is a nanoparticle composition comprising: (a) one or more nucleic acid molecular entities; (b) an amino lipid or a salt thereof, wherein said amino lipid or a salt thereof, comprises from 20 mol % to 80 mol % of a total lipid content present in said nanoparticle composition, wherein said amino lipid or a salt thereof, comprises one or more ionizable nitrogen atoms, and wherein a molar ratio of said ionizable nitrogen atoms to phosphate groups present in said nucleic acid molecular entity is from 2 to 12; (c) a neutral lipid, comprising from 2 mol % to 25 mol % of said total lipid content present in said nanoparticle composition; (d) a structural lipid, comprising from 30 mol % to 60 mol % of said total lipid content present in said nanoparticle composition; (e) a PEG-lipid, comprising from 0.1 mol % to 6 mol % of said total lipid content present in said nanoparticle composition; and (f) a nucleic acid stabilizer, wherein the nucleic acid stabilizer comprises chitosan, cetrimonium bromide, polyethylene glycol that has a number average molecular weight of about 120 to about 2000 Da, or a combination thereof. In some embodiments, the nucleic acid stabilizer comprises PEG 200, PEG 400, or PEG 600. In some embodiments, the nucleic acid stabilizer is PEG 400. In some embodiments, the nucleic acid stabilizer is present in the nanoparticle composition in an amount of about 0.01% to about 20% by total weight. In some embodiments, the nucleic acid stabilizer is present in the nanoparticle composition in an amount of about 0.5% to about 5% by total weight. In some embodiments, said one or more nucleic acid molecular entities comprise a PCSK9 gRNA. In some embodiments, said one or more nucleic acid molecular entities comprise an mRNA encoding a Cas nuclease. In some embodiments, said one or more nucleic acid molecular entities comprise an mRNA, a gRNA, a siRNA, an antisense oligonucleotide, a microRNA, an anti-microRNA, an RNA activator, an aptamer, or a combination thereof. In some embodiments, said nanoparticle composition comprises an antioxidant. In some embodiments, said antioxidant comprise EDTA. In some embodiments, the composition comprises a surfactant. In some embodiments, the surfactant is a fatty acid or a fatty alcohol. In some embodiments, the surfactant is a C₁₂-C₂₄ fatty alcohol. In some embodiments, the C₁₂-C₂₄ fatty alcohol is lauryl alcohol, tridecyl alcohol, myristyl alcohol, pentadecyl alcohol, cetyl alcohol, palmitoleyl alcohol, heptadecyl alcohol, stearyl alcohol, oleyl alcohol, nonadecyl alcohol, arachidyl alcohol, or a combination thereof. In some embodiments, the C₁₂-C₂₄ fatty alcohol is oleyl alcohol, stearyl alcohol, or a mixture thereof. In some embodiments, the surfactant comprises about 1.0 mol % to about 10 mol % of a total lipid content present in said nanoparticle composition. In some embodiments, a median diameter of the nanoparticle is from about 50 nm to about 150 nm. In some embodiments, a polydispersity index of the nanoparticle is from 0 to 0.15. In some embodiments, a polydispersity index of the nanoparticle is from 0 to 0.05. In some embodiments, a nucleic acid entrapment efficiency of the nanoparticle composition is at least 80%, at least 85%, at least 90%, at least 91%, at least 92%, at least 93%, at least 94%, at least 95%, at least 96%, at least 97%, at least 98%, or at least 99%.

In one aspect, disclosed herein is a PEG-lipid having the structure of Formula (III), or a pharmaceutically acceptable salt or solvate thereof,

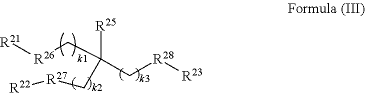

wherein
each of R²¹ and R²² is independently substituted or unsubstituted C₃-C₃₀ alkyl, substituted or unsubstituted C₃-C₃₀ alkenyl, or substituted or unsubstituted C₃-C₃₀ alkynyl;
each of R²⁶, R²⁷ and R²⁸ is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH—, —O—, —S—, or a bond;
each of R⁴ is independently hydrogen or substituted or unsubstituted C₁-C₆ alkyl;
R²³ is —C₀-C₁₀ alkylene-(CH₂—CH₂—O)ₖ₄—R²⁴ or —C₁-C₁₀ heteroalkylene-(CH₂—CH₂—O)ₖ₄—R²⁴, wherein the alkylene and heteroalkylene are each independently substituted or unsubstituted;
R²⁴ is independently hydrogen or substituted or unsubstituted C₁-C₃ alkyl;
R²⁵ is hydrogen or substituted or unsubstituted C₁-C₆ alkyl;
each of k1, k2, and k3 is independently 0, 1, 2, 3, 4, or 5; and
k4 is an integer selected from 1 to 100.

In one aspect, disclosed herein is a PEG-lipid having the structure of Formula (III*), or a pharmaceutically acceptable salt or solvate thereof,

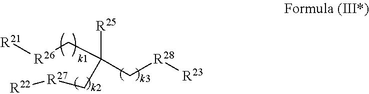

wherein
R²¹ is hydrogen, substituted or unsubstituted C₁-C₃₀ alkyl, substituted or unsubstituted C₃-C₃₀ alkenyl, or substituted or unsubstituted C₃-C₃₀ alkynyl;
R²² is hydrogen, substituted or unsubstituted C₁-C₃₀ alkyl, substituted or unsubstituted C₃-C₃₀ alkenyl, or substituted or unsubstituted C₃-C₃₀ alkynyl;
each of R²⁶, R²⁷ and R²⁸ is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —N(Ac)—, —OC(O)OCH₂C(O)O—, —OC(O)OCH₂C(O)NH— —OC(O)NHCH₂C(O)O—, —OC(O)NHCH₂C(O)NH—, —OC(O)O(CH₂)₂C(O)O—, —OC(O)O(CH₂)₂C(O)NH—, —OC(O)NH(CH₂)₂C(O)O—, —OC(O)NH(CH₂)₂C(O)NH—, —OC(O)O(CH₂)₃C(O)O—, —OC(O)O(CH₂)₃C(O)NH—, —OC(O)NH(CH₂)₃C(O)O—, —OC(O)NH(CH₂)₃C(O)NH—, —C(O)OCH₂C(O)NH— —C(O)NHCH₂C(O)O—, —C(O)NHCH₂C(O)NH—, —C(O)O(CH₂)₂C(O)O—, —C(O)O(CH₂)₂C(O)NH—, —C(O)NH(CH₂)₂C(O)O—, —C(O)NH(CH₂)₂C(O)NH—, —C(O)O(CH₂)₃C(O)O—, —C(O)O(CH₂)₃C(O)NH—, —C(O)NH(CH₂)₃C(O)O—, —C(O)NH(CH₂)₃C(O)NH—, —OC(═O)NR⁴(CH₂)₁₋₃C(═O)NH—, —OC(═O)NR⁴(CH₂)₁₋₃C(═O)O—, —C(═O)NR⁴(CH₂)₁₋₃C(═O)NH—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH—, —O—, —S—, or a bond;
each of R⁴ is independently hydrogen or substituted or unsubstituted C₁-C₆ alkyl;
R²³ is —C₀-C₁₀ alkylene-(CH₂—CH₂—O)ₖ₄—R²⁴, —C₁-C₁₀ heteroalkylene-(CH₂—CH₂—O)ₖ₄—R²⁴, —C₀-C₁₀ alkylene-(O—CH₂—CH₂)ₖ₄—R²⁴ or —C₁-C₁₀ heteroalkylene-(O—CH₂—CH₂)ₖ₄—R²⁴, wherein the alkylene and heteroalkylene are each independently substituted or unsubstituted;
R²⁴ is independently hydrogen, substituted or unsubstituted C₁-C₃ alkyl, —O—R⁴, —C(═O)OH, or —C(═O)R⁴;
R²⁵ is hydrogen or substituted or unsubstituted C₁-C₂₂ alkyl;
each of k1, k2, and k3 is independently 0, 1, 2, 3, 4, or 5; and
k4 is an integer selected from 1 to 100.

In some embodiments, the PEG-lipid of Formula (III) or (III*), or a pharmaceutically acceptable salt or solvate thereof, wherein
R²¹ is substituted or unsubstituted C₃-C₃₀ alkyl, substituted or unsubstituted C₃-C₃₀ alkenyl, or substituted or unsubstituted C₃-C₃₀ alkynyl;
R²² is hydrogen, substituted or unsubstituted C₃-C₃₀ alkyl, substituted or unsubstituted C₃-C₃₀ alkenyl, or substituted or unsubstituted C₃-C₃₀ alkynyl;
each of R²⁶, R²⁷ and R²⁸ is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —OC(═O)NR⁴(CH₂)₁₋₃C(═O)NH—, —OC(═O)NR⁴(CH₂)₁₋₃C(═O)O—, —C(═O)NR⁴(CH₂)₁₋₃C(═O)NH—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH—, —O—, —S—, or a bond;
each of R⁴ is independently hydrogen or substituted or unsubstituted C₁-C₆ alkyl;
R²³ is —C₀-C₁₀ alkylene-(CH₂—CH₂—O)ₖ₄—R²⁴ or —C₁-C₁₀ heteroalkylene-(CH₂—CH₂—O)ₖ₄—R²⁴, wherein the alkylene and heteroalkylene are each independently substituted or unsubstituted;
R²⁴ is independently hydrogen, substituted or unsubstituted C₁-C₃ alkyl, —O—R⁴, —C(═O)OR⁴, or —C(═O)R⁴;
R²⁵ is hydrogen or substituted or unsubstituted C₁-C₆ alkyl;
each of k1, k2, and k3 is independently 0, 1, 2, 3, 4, or 5; and
k4 is an integer selected from 1 to 100.

In one aspect, disclosed herein is a PEG-lipid having the structure selected from Table 2, or a pharmaceutically acceptable salt or solvate thereof. In one aspect, disclosed herein is a pharmaceutical composition comprising a described nanoparticle composition, and an excipient or carrier. In some embodiments, said pharmaceutical composition comprises an mRNA encoding a gene editor nuclease. In some embodiments, said pharmaceutical composition comprises one or more guide RNA molecules. In some embodiments, said pharmaceutical composition comprises a PCSK9 guide RNA. In some embodiments, said pharmaceutical composition comprises two or more guide RNA molecules. In some embodiments, said two or more guide RNA molecules target two or more genes of interest. In some embodiments, said mRNA encodes Cas9 nuclease. In some embodiments, said mRNA encodes a base editor nuclease. In some embodiments, said mRNA and said one or more guide RNA molecules are present in a same nanoparticle composition. In some embodiments, said mRNA and said one or more guide RNA molecules are present in different nanoparticle compositions. In some embodiments, a ratio of said gRNA molecules to said mRNA in said pharmaceutical composition is from about 0.01 to about 100 by weight or by mole. In some embodiments, a ratio of said gRNA molecules to said mRNA in said pharmaceutical composition is about 50:1, about 40:1, about 30:1, about 20:1, about 18:1, about 16:1, about 14:1, about 12:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10 by weight or by mole. In one aspect, disclosed herein is a pharmaceutical composition comprising: a first nanoparticle composition as described herein, and a second nanoparticle composition as described herein. In some embodiments, said first nanoparticle composition comprises a gene editor mRNA, and said second nanoparticle composition comprises one or more guide RNA molecules. In some embodiments, a ratio of guide RNA molecules to mRNA in said pharmaceutical composition is about 50:1, about 40:1, about 30:1, about 20:1, about 18:1, about 16:1, about 14:1, about 12:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10 by weight or by mole. In some embodiments, a ratio of guide RNA to mRNA in said pharmaceutical composition is about 1:1 by weight or by mole.

In one aspect, disclosed herein is a method of delivering a nucleic acid molecular entity to a cell, the method comprising contacting said cell with a nanoparticle composition or a pharmaceutical composition as described herein, whereby said nucleic acid molecular entity is delivered to said cell. In some embodiments, said cell is contacted in vivo, ex vivo, or in intro. In one aspect, disclosed herein is a method of producing a polypeptide of interest in a cell, the method comprising contacting said cell with a nanoparticle composition or a pharmaceutical composition described herein, wherein said nanoparticle composition or said pharmaceutical composition comprises a nucleic acid molecular entity, and wherein said nucleic acid molecular entity is translated in the cell thereby producing the polypeptide. In one aspect, disclosed herein is a method of making a pharmaceutical composition, comprising combining a first nanoparticle composition and a second nanoparticle composition described herein. In one aspect, disclosed herein is a method of treating a disease or condition in a mammal, the method comprising administering to a mammal a therapeutically effective amount of a pharmaceutical composition described herein. In one aspect, disclosed herein is a method of editing PCSK9 gene in a cell comprising contacting said cell with a nanoparticle composition or a pharmaceutical composition described herein, wherein said nanoparticle composition or said pharmaceutical composition comprises a PCSK9 guide RNA. In one aspect, disclosed herein is a method of producing a stabilized nanoparticle composition described herein, comprising combining a nucleic acid stabilizer with a nanoparticle composition that lacks the nucleic acid stabilizer. In some embodiments, the nucleic acid stabilizer is combined with the nanoparticle composition before freezing or storage. In some embodiments, the nucleic acid stabilizer is combined with the nanoparticle composition before, concurrently, or after the addition of the one or more nucleic acid entities. In some embodiments, the combining comprises mixing the nucleic acid stabilizer with the one or more nucleic acid entities in an aqueous buffer.

In one aspect, disclosed herein is a method of producing a stabilized nanoparticle composition, comprising (a) combining a nucleic acid stabilizer with one or more nucleic acid molecular entities thereby producing a solution comprising the stabilized one or more nucleic acid molecular entities; and (b) combining the solution of (a) with a nanoparticle composition that comprises one or more of an amino lipid, a neutral lipid, a structural lipid, and a PEG-lipid. In some embodiments, the combining in (a) comprises mixing the nucleic acid stabilizer with the one or more nucleic acid entities in an aqueous buffer. In some embodiments, the method comprising collecting and dialyzing the nanoparticle composition against a buffer with a pH of about 6.5 to about 8.0. In some embodiments, the nucleic acid stabilizer is polyethylene glycol that has a number average molecular weight of about 120 to about 1000.

In one aspect, disclosed herein is a method of preparing a formulation comprising lipid nanoparticles, wherein the nanoparticles comprise (i) one or more nucleic acid molecular entities, (ii) an amino lipid, and (iii) one or more lipids selected from a structural lipid, a neutral lipid, and a PEG-lipid, the method comprising, (a) combining a first faction of the amino lipid with the one or more nucleic acid molecular entities in a first solution, wherein the first fraction comprises 0.1 mol % to 99 mol % of the total amino lipid; (b) combining the remaining of the amino lipid with the one or more lipids selected from a structural lipid, a neutral lipid, and a PEG-lipid in a second solution; (c) mixing the first solution and the second solution, thereby producing the lipid nanoparticles. In some embodiments, the first fraction of the amino lipid is configured to neutralize between 0.1-99% of the phosphate (on an N:P basis) in the one or more nucleic acid molecular entities. In some embodiments, the first fraction of the amino lipid is configured to neutralize between 0.5-90% of the phosphate (on an N:P basis) in the one or more nucleic acid molecular entities. In some embodiments, the first fraction of the amino lipid is configured to neutralize about 10%, 15%, 25%, 50%, or 75% of the phosphate (on an N:P basis) in the one or more nucleic acid molecular entities. In some embodiments, the first solution is an aqueous buffer solution. In some embodiments, the first solution further comprises a nucleic acid stabilizer. In some embodiments, the first solution and the second solution are mixed in an inline mixer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates PCSK9 gene editing in wild type C57BL/6 female mice (n=5) at 0.5 mg/kg total RNA dose for LNPs except F1. Total RNA dose F1: 0.75 mg/kg. LNPs F1-F5 were constituting by using SpCas9 mRNA and PCSK9 gRNA.

FIGS. 2A and 2B illustrate the calibration curve and example chromatogram from the IP-RPLC-HPLC-ELSD method used to determine PEG400 (w/w) %. The calibration curve is fit to a cubic fit, as the ELSD detection has a non-linear response.

FIG. 3 illustrates the GFP mRNA loaded LNPs (identical in lipid composition and freezing buffer, but differing in the presence and location of PEG400) were applied to HUH7 human liver cell line in both never-frozen and post three freeze/thaw cycle aliquots. GFP fluorescence was measured 72 hours later. PEG400 containing formulations demonstrated no decrease in GFP expression and mRNA delivery efficacy after three freeze/thaw cycles, while the formulation without PEG400 experienced a loss of delivery efficacy.

FIG. 4 illustrates PCSK9 gene editing in mouse hepatocytes in wild type C57BL/6 female mice for LNPs F45-F51 (n=5) at doses given in Tables 9 and 10, and described in Example 14.

FIG. 5 illustrates PCSK9 gene editing in mouse hepatocytes in wild type C57BL/6 female mice (n=5) for LNPs F52-F60 at doses given in Tables 9 and 10, and described in Example 14. Doses and cargos can be found in those tables.

FIG. 6 illustrates PCSK9 gene editing in mouse hepatocytes in wild type C57BL/6 female mice (n=5) for LNPs F32, F61, F62, F63 at doses given in Tables 9 and 10, and described in Example 14. Doses and cargos can be found in those tables.

FIG. 7 illustrates PCSK9 gene editing in mouse hepatocytes in wild type C57BL/6 female mice (n=5) for LNPs F64-F68 at doses given in Tables 9 and 10, and described in Example 14. Doses and cargos can be found in those tables

FIG. 8 illustrates PCSK9 gene editing in mouse hepatocytes in wild type C57BL/6 female mice (n=5) for LNPs F69-F83 at doses given in Tables 9 and 10, and described in Example 14. Doses and cargos can be found in those tables. All LNPs were intravenously administered (RO) to wild type C57BL/6 female mice (n=5).

FIG. 9 illustrates PCSK9 gene editing in mouse hepatocytes in wild type C57BL/6 female mice (n=5) for LNPs F84-F91 at doses given in Tables 9 and 10, and described in Example 14.

FIG. 10 illustrates PCSK9 gene editing in mouse hepatocytes in wild type C57BL/6 female mice (n=3-5) for F110, F123, F124, and F143-F146 at doses given in Tables 9 and 10, and described in Example 14.

FIG. 11 illustrates PCSK9 gene editing in mouse hepatocytes in wild type C57BL/6 female mice (n=5) for LNPs F92 and F97-F100 at doses given in Table 11 and Example 15.

FIG. 12 illustrates PCSK9 gene editing in mouse hepatocytes in wild type C57BL/6 female mice (n=5) for LNPs F93, F101-F103 and F155 at doses given in Table 12 and as described in Example 16. All LNPs were intravenously administered (RO) to wild type C57BL/6 female mice (n=3-5).

FIG. 13 illustrates PCSK9 gene editing at hepatocytes in wild type C57BL/6 female mice (n=5) at 1 mg/kg total RNA dose for LNPs F106-109 as described in Table 15 and 16. All LNPs were intravenously administered (RO) to wild type C57BL/6 female mice (n=5).

FIG. 14 illustrates PCSK9 gene editing at mouse hepatocytes in wild type C57BL/6 female mice (n=5) at 0.5 mg/kg total RNA dose for LNPs F111-118 as described in Table 15 and 16. All LNPs were intravenously administered (RO) to wild type C57BL/6 female mice (n=5).

FIG. 15 illustrates PCSK9 gene editing at mouse hepatocytes in wild type C57BL/6 female mice (n=5) at 0.5 mg/kg total RNA dose for LNPs F110, F111, 119, 120, 121 and 122 as described in Table 9, 15 and 16. All LNPs were intravenously administered (RO) to wild type C57BL/6 female mice (n=5).

FIG. 16 illustrates PCSK9 liver editing % given for LNPs F104 and F105, as described in Examples 17, 18 and 23 respectively. Formulation composition and characterization data are given in Tables 13 and 14 for F104 and F105 respectively.

FIG. 17 illustrates PCSK9 editing for LNPs F94-F96 and F147-F150. These are referenced in Example 14 and Tables 9 and 10.

FIG. 18 illustrates PCSK9 gene editing at mouse hepatocytes in wild type C57BL/6 female mice (n=5) at 0.05 mg/kg total RNA dose for LNPs F128 and F129 as described in Table 15 and 16. All LNPs were intravenously administered (RO) to wild type C57BL/6 female mice (n=5).

FIG. 19 illustrates the % editing for F151-F154 as described in Example 22 and Table 17.

FIG. 20. illustrates the percentage of PCSK9 gene editing achieved in wild type C57BL/6 female mice liver after a single dose administration of LNPs F160-F165 comprising amino lipids VL454, VL459 and VL455 at N to P ratios 6 and 8 as shown in Table 18; (n=5) at 0.125 mg/kg dose, as described in Example 24. F151A is the reference LNP constituted using amino lipid 502.

FIG. 21. illustrates the percentage of PCSK9 gene editing in wild type C57BL/6 female mice liver after single dose administration of LNPs F172-F177 (n=5) at 0.1 mg/kg dose of total RNA, as described in Example 24, Table 19. All LNPs administered were constituted using the amino lipid VL422.

FIG. 22A illustrates the percentage of PCSK9 gene editing in wild type C57BL/6 female mice liver after single dose administration of LNPs F151A, 172 and 178-181 (n=5) at 0.1 mg/kg dose of total RNA, Table 20. F151A is the reference LNP constituted using amino lipid 502. The commercially available PEG-Lipid 507 was replaced with PEG-Lipid VP159 in LNPs F178, F180 and F181. F179-F181 comprising amino lipid VL422. FIG. 22B illustrates the percentage of PCSK9 gene editing in wild type C57BL/6 female mice liver after single dose administration of LNPs F151B, F193A-F193C (n=5) at 0.1 mg/kg dose of total RNA, Table 20. F151B is the reference LNP constituted using amino lipid 502. The commercially available PEG-Lipid 507 was replaced with PEG-Lipid VP177 in LNPs F19B and F193C and formulated with amino lipid VL422.

FIG. 23A illustrates the percentage of ANGPTL3 gene editing in the livers of cynomolgus monkeys (n=3) after single dose administration of LNPs F188, F190-F193 as described in Example 25, Table 23. F188 and F192 were dosed at 1.5 mg/kg total RNA dose. F190, F191 and F193 were dosed at 2 mg/kg total RNA dose. F188 is a reference LNP comprising amino lipid 502 and PEG-Lipid 507. The PEG-Lipid 507 of F188 is replaced with PEG-Lipid VP159 in F192. F190, 190A, F191 and F193 comprising of amino lipid VL422 and PEG-Lipid 507. FIG. 23B illustrates the percentage of ANGPTL3 gene editing in the livers of cynomolgus monkeys (n=3) after single dose administration of LNPs F190A and F190B as described in Example 25, Table 23. F190A and F190B were dosed at 2 mg/kg total RNA dose. The PEG-Lipid 507 of F190A is replaced with PEG-Lipid VP159 in F190B.

FIG. 24A illustrates the percentage of PCSK9 gene editing in wild type C57BL/6 female mice liver after single dose administration of LNPs F122A and F221-F226 (n=5) at 0.25 mg/kg total RNA dose and FIG. 24B illustrates the percentage of PCSK9 gene editing in wild type C57BL/6 female mice liver after single dose administration of LNPs F122A at 0.5 and 0.25 mg/kg, and F226A-F226C (n=5) at 0.25 mg/kg total RNA dose as described in Example 26, Table 24. Doping of fatty alcohol to LNP comprising amino lipid VL422 improve editing. F223 comprising about 6 mol % of fatty alcohol 504 at 0.25 mg/kg total RNA produced editing equivalent to the corresponding LNP F122A with no fatty alcohol at 0.5 mg/kg. 10 mol % of the alcohol 504 in the LNP composition further improved the editing efficiency. All LNPs evaluated comprise the amino lipid VL422. Formulations F226B and F226C comprising about 6 mol % and 10% of fatty alcohol 513 at 0.25 mg/kg total RNA dose improved the editing efficacy compare to the corresponding LNP F122A with no fatty alcohol at 0.25 mg/kg total RNA dose. All LNPs evaluated comprise the amino lipid VL422.

FIG. 25 illustrates the percentage of ANGPTL3 gene editing in the livers of cynomolgus monkeys (n=3) after single dose administration of LNPs F190A, F235-F237 as described in Example 40 (Table 26). LNP F190A was dosed at 2, 1.5 and 1 mg/kg total RNA. F236, F235 and F237 were dosed at 2 mg/kg total RNA.

FIG. 26 illustrates the percentage of ANGPTL3 protein knockdown from baseline at day 15 in serum of cynomolgus monkeys (n=3) after single dose administration of LNPs F190A, F235-F237 (Example 40). F190A was dosed at 2, 1.5 and 1 mg/kg RNA respectively. F235, F236 and F237 were dosed at 2 mg/kg RNA.

FIG. 27 illustrates the percentage of ANGPTL3 gene editing in the livers of cynomolgus monkeys (n=3) after single dose administration of LNPs F238 and F239 as described in Example 42 (Table 27).

FIG. 28 illustrates the degradation rate of amino lipids 502, VL422 and VL454 in human liver S9 at 0, 1, 6 and 24 hour.

FIG. 29 illustrates amino lipid VL422 exposure profiles in plasma from cynomolgus monkeys after LNP administration. LNPs F188 was dosed at 1.5 mg/kg total RNA dose wheres as F190, F191 and F193 was dosed 2 mg/kg total RNA dose as described in Example 25 and Table 23.

### DETAILED DESCRIPTION

The present disclosure relates to cationic and/or ionizable lipids and lipid-containing particles comprising the same. The disclosure also relates to methods of delivering a therapeutic agent (such as a nucleic acid) to a mammalian cell, methods of producing a polypeptide of interest in a mammalian cell, and methods of treating a disease or disorder in a mammal in need thereof. For example, a method of producing a polypeptide of interest in a cell can comprise a step of contacting a herein described lipid-containing particle with the cell, thereby delivering an mRNA that encodes the polypeptide of interest into the cell, and thereby the mRNA can be translated to produce the polypeptide of interest. For another example, a method of delivering a therapeutic agent to a mammalian cell or organ may involve administration of a herein described lipid-containing particle comprising the therapeutic agent to a subject, in which the administration comprises contacting the cell or organ with the lipid-containing particle, whereby the therapeutic agent is delivered to the cell or organ.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

The term “derivative” as used herein indicates a chemical or biological substance that is related structurally to a second substance and derivable from the second substance through a modification of the second substance. In particular, if a first compound is a derivative of a second compound and the second compound is associated with a chemical and/or biological activity, the first compound differs from the second compound for at least one structural feature, while retaining (at least to a certain extent) the chemical and/or biological activity of the second compound and at least one structural feature (e.g. a sequence, a fragment, a functional group and others) associated thereto. A skilled person will be able to identify, on a case by case basis and upon reading of the present disclosure, structural features of the second compound that have to be maintained in the first compound to retain the second compound chemical and/or biological activity as well as assays that can be used to prove retention of the chemical and/or biological activity. Exemplary “derivatives” can include a prodrug, a metabolite, an enantiomer, a diastereomer, esters (e.g. acyloxyalkyl esters, alkoxycarbonyloxyalkyl esters, alkyl esters, aryl esters, phosphate esters, sulfonate esters, sulfate esters and disulfide containing esters), ethers, amides, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts, sulfonate esters, and the like. In some cases, a derivative may include trivial substitutions (i.e. additional alkyl/akylene groups) to a parent compound that retains the chemical and/or biological activity of the parent compound.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like are meant to refer to reducing or ameliorating a disorder and/or symptoms associated therewith (e.g., a neoplasia or tumor). “Treating” may refer to administration of the LNP composition to a subject after the onset, or suspected onset, of a disease or condition. “Treating” includes the concepts of “alleviating”, which refers to lessening the frequency of occurrence or recurrence, or the severity, of any symptoms or other ill effects related to a disease or condition and/or the side effects associated with the disease or condition. The term “treating” also encompasses the concept of “managing” which refers to reducing the severity of a particular disease or disorder in a patient or delaying its recurrence, e.g., lengthening the period of remission in a patient who had suffered from the disease. The term “treating” further encompasses the concept of “prevent,” “preventing,” and “prevention,” as previously stated. It is appreciated that, although not precluded, treating a disorder or condition does not require that the disorder, condition, or symptoms associated therewith be completely eliminated.

The term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Exemplary substituents include amino, alkylamino, and the like.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “=0”) as the point of attachment may be described, shown or listed herein within a substituent group, wherein the structure may only show a single bond as the point of attachment to the core structure of Formula (I), Formula (I*), Formula (Ia), Formula (II), Formula (II*), Formula (III) or Formula (III*). A person of ordinary skill in the art would understand that, while only a single bond is shown, a double bond is intended for those substituents.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “aryl” refers to a radical derived from a hydrocarbon ring system comprising at least one aromatic ring. In some embodiments, an aryl comprises hydrogens and 6 to 30 carbon atoms. The aryl radical can be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl can be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

The term “alkenyl” refers to a type of alkyl group in which at least one carbon-carbon double bond is present. In one embodiment, an alkenyl group has the formula —C(R^{a})=CR^{a}₂, wherein R^{a} refers to the remaining portions of the alkenyl group, which may be the same or different. In some embodiments, R^{a} is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)=CH₂, —CH═CHCH₃, —C(CH₃)=CHCH₃, and —CH₂CH═CH₂. “Alkenylene” or “alkenylene chain” refers to a alkylene group in which at least one carbon-carbon double bond is present. In some embodiments, the alkenylene is —CH═CH—, —CH₂CH₂CH═CH—, or —CH═CHCH₂CH₂—. In some embodiments, the alkenylene is —CH═CH—. In some embodiments, the alkenylene is —CH₂CH₂CH═CH—. In some embodiments, the alkenylene is —CH═CHCH₂CH₂—.

The term “alkynyl” refers to a type of alkyl group in which at least one carbon-carbon triple bond is present. In one embodiment, an alkynyl group has the formula —C≡CR^{a}, wherein R^{a} refers to the remaining portions of the alkynyl group. In some embodiments, R^{a} is H or an alkyl. In some embodiments, an alkynyl is selected from ethynyl (i.e., acetylenyl), propynyl (i.e., propargyl), butynyl, pentynyl, and the like. Non-limiting examples of an alkynyl group include —C≡CH, —C≡CCH₃, and —CH₂C≡CH. “Alkynylene” or “alkynylene chain” refers to a alkylene group in which at least one carbon-carbon triple bond is present. In some embodiments, the alkynylene is —C≡C—, —CH₂CH₂C≡C—, or —C≡CCH₂CH₂—. In some embodiments, the alkynylene is —C═C—. In some embodiments, the alkynylene is —CH₂CH₂C≡C—. In some embodiments, the alkynylene is —C≡CCH₂CH₂—.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (=O) moieties, such as pyrrolidin-2-one. In some embodiments, at least one of the two rings of a bicyclic heterocycle is aromatic. In some embodiments, both rings of a bicyclic heterocycle are aromatic.

The term “heterocycloalkyl” refers to a cycloalkyl group that includes at least one ring heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted. As used herein, the term “heterocycloalkylene” can refer to a divalent heterocycloalkyl group.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

The term “heteroalkyl” refers to an alkyl group in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆ heteroalkyl. Representative heteroalkyl groups include, but are not limited to —OCH₂OMe, —OCH₂CH₂OH, —OCH₂CH₂OMe, or —OCH₂CH₂OCH₂CH₂NH₂. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted. Representative heteroalkylene groups include, but are not limited to —OCH₂CH₂O—, —OCH₂CH₂OCH₂CH₂O—, or —OCH₂CH₂OCH₂CH₂OCH₂CH₂O—.

The term “heteroalkenyl” refers to an alkenyl group in which one or more skeletal atoms of the alkenyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkenyl is attached to the rest of the molecule at a carbon atom of the heteroalkenyl. In some embodiments, a heteroalkenyl is attached to the rest of the molecule at a heteroatom of the heteroalkenyl. In some embodiments, a heteroalkyl is a C₁-C₆ heteroalkenyl.

The term “heteroalkynyl” refers to an alkynyl group in which one or more skeletal atoms of the alkynyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkynyl is attached to the rest of the molecule at a carbon atom of the heteroalkynyl. In some embodiments, a heteroalkynyl is attached to the rest of the molecule at a heteroatom of the heteroalkynyl. In some embodiments, a heteroalkyl is a C₁-C₆ heteroalkynyl.

As used herein, the “N/P ratio” is the molar ratio of ionizable (e.g., within a pH range close to the pKa of the lipid nanoparticle) nitrogen atoms in an amino lipid (or lipids) to phosphate groups in a nucleic acid molecular entity (or nucleic acid molecular entities), e.g., in a nanoparticle composition comprising a lipid component and an RNA. Ionizable nitrogen atoms can include, for example, nitrogen atoms that can be protonated at about pH 1, about pH 2, about pH 3, about pH 4, about pH5, about pH 6, about pH 7, about pH 7.5, or about pH 8 or higher. The physiological pH range can include, for example, the pH range of different cellular compartments (such as organs, tissues, and cells) and bodily fluids (such as blood, CSF, gastric juice, milk, bile, saliva, tears, and urine). In certain specific embodiments, the physiological pH range refers to the pH range of blood in a mammal, for example, from about 7.35 to about 7.45. In some embodiments, ionizable nitrogen atoms refer to those nitrogen atoms that are ionizable within a pH range between 5 and 14.

For the payload that does not contain a phosphate group, the N/P ratio can refer to a molar ratio of ionizable nitrogen atoms in a lipid to the total negative charge in the payload. For example, the N/P ratio of an LNP composition can refer to a molar ratio of the total ionizable nitrogen atoms in the LNP composition to the total negative charge in the payload that is present in the composition.

As used herein, amino lipids can contain at least one primary, secondary or tertiary amine moiety that is protonatable (or ionizable) between pH range 4 and 14. In some embodiments, and the amine moiety/moieties function as the hydrophilic headgroup of the amino lipids described in Tables 1A and 1B. When most of the amine moiety(ies) of an amino lipid (or amino lipids) in a nucleic acid-lipid nanoparticle formulation is protonated at physiological pH, then the nanoparticles can be termed as cationic lipid nanoparticle (cLNP). When most of the amine moiety(ies) of an amino lipid (or amino lipids) in a nucleic acid-lipid nanoparticle formulation is not protonated at physiological pH but can be protonated at acidic pH, endosomal pH for example, can be termed as ionizable lipid nanoparticle (iLNP). The amino lipids that constitute cLNPs can be generally called cationic amino lipids (cLipids). The amino lipids that constitute iLNPs can be called ionizable amino lipids (iLipids). The amino lipids described in Tables 1A and 1B can be an iLipid or a cLipid at physiological pH.

As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. For example, a nanoparticle composition may be a liposome having a lipid bilayer with a diameter of 500 nm or less. The LNPs described herein can have a mean diameter of from about 1 nm to about 2500 nm, from about 10 nm to about 1500 nm, from about 20 nm to about 1000 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, or from about 70 nm to about 80 nm. The LNPs described herein can have a mean diameter of about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or greater. The LNPs described herein can be substantially non-toxic.

As used herein, a “PEG-lipid” or “PEG-lipid” refers to a lipid comprising a polyethylene glycol component.

As used herein, a “phospholipid” can refer to a lipid that includes a phosphate moiety and one or more carbon chains, such as unsaturated fatty acid chains. A phospholipid may include one or more multiple (e.g., double or triple) bonds. In some embodiments, a phospholipid may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of an LNP to pass through the membrane, i.e., delivery of the one or more elements to a cell.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

The term “nucleic acid molecular entity” is used interchangeably with “nucleic acid.” The term “nucleic acid” as used herein generally refers to one or more nucleobases, nucleosides, or nucleotides, and the term includes polynucleobases, polynucleosides, and polynucleotides. A nucleic acid can include polynucleotides, mononucleotides, and oligonucleoitdes. A nucleic acid can include DNA, RNA, or a mixture thereof, and can be single stranded, double stranded, or partially single or double stranded, and can form secondary structures. In some embodiments, a nucleic acid has multiple double-stranded segments and single stranded segments. For example, a nucleic acid may comprise a polynucleotide, e.g. a mRNA, with multiple double stranded segments within it. DNA may be in the form of, e.g., antisense molecules, plasmid DNA, pre-condensed DNA., a PCR product, vectors, expression cassettes, chimeric sequences, chromosomalDNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), CRISPR RNA, base editor RNA and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′, 3′, 4′ and 5′ substituted ribonucleotide, 2′, 3′, 4′ and 5′ substituted 2′-ribonucleotide, substituted and unsubstituted carbocyclic nucleotides, substituted and unsubstituted acyclic nucleotides and peptide-nucleic acids (PNAs). Examples of nucleic acids also include acyclic and carbocyclic nucleotide, such as Glycol nucleic acid. Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J Biol. Chem., 260:2605-2608 (1985); Rossolini et al., Mal. Cell. Probes, 8:91-98 (1994)). “Nucleotides” contain a substituted and/or unsubstituted sugar deoxyribose (DNA), or a substituted and/or unsubstituted sugar ribose (RNA), or a substituted and/or unsubstituted carbocyclic, or a substituted and/or unsubstituted acyclic moiety (e.g., glycol nucleic), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. The term “gene” refers to a nucleic acid (e.g., DNA or RNA) sequence that comprises partial length or entire length coding sequences necessary for the production of a polypeptide or precursor polypeptide.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The present disclosure encompasses isolated or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ends of the nucleic acid) in the genomic DNA of the organism from which the nucleic acid is derived. For example, in some embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

### II. Lipid Nanoparticle (LNP) Composition

### Amino Lipid

Described herein are LNP compositions comprising an amino lipid, a phospholipid, a PEG-lipid, a cholesterol or a derivative thereof, a payload, or any combination thereof. In some embodiments, the LNP composition comprises an amino lipid. Exemplary amino lipids include, but are not limited to, the lipids in Table 1A. In some embodiments, the LNP composition comprises an amino lipid having the structure of Formula (I), Formula (I*), Formula (Ia), Formula (II), Formula (II*), or a pharmaceutically acceptable salt or solvate thereof. In some embodiments, the LNP composition comprises an amino lipid of having the structure of Formula (Ia), or a pharmaceutically acceptable salt or solvate thereof. In some embodiments, the LNP comprises a plurality of amino lipids. For example, the LNP composition can comprise 2, 3, 4, 5, 6, 7, 8, 9, 10, or more amino lipids. For another example, the LNP composition can comprise at least 2, at least 3, at least 4, at least 5, at least 6, at least 7, at least 9, at least 10, or at least 20 amino lipids. For yet another example, the LNP composition can comprise at most 2, at most 3, at most 4, at most 5, at most 6, at most 7, at most 9, at most 10, at most 20, or at most 30 amino lipids.

In some embodiments, the LNP composition comprises one or more amino lipids. In some embodiments, the one or more amino lipids comprise from about 40 mol % to about 65 mol % of the total lipid present in the particle. In some embodiments, the one or more amino lipids comprise about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, or about 65 mol % of the total lipid present in the particle.

In some embodiments, the amino lipid is an ionizable lipid. An ionizable lipid can comprise one or more ionizable nitrogen atoms. In some embodiments, at least one of the one or more ionizable nitrogen atoms is positively charged. In some embodiments, at least 10 mol %, 20 mol %, 30 mol %, 40 mol %, 50 mol %, 60 mol %, 70 mol %, 80 mol %, 90 mol %, 95 mol %, or 99 mol % of the ionizable nitrogen atoms in the LNP composition are positively charged. In some embodiments, the amino lipid comprises a primary amine, a secondary amine, a tertiary amine, an imine, an amide, a guanidine moiety, a histidine residue, a lysine residue, an arginine residue, or any combination thereof. In some embodiments, the amino lipid comprises a primary amine, a secondary amine, a tertiary amine, a guanidine moiety, or any combination thereof. In some embodiments, the amino lipid comprises a tertiary amine.

In some embodiments, the amino lipid is a cationic lipid. In some embodiments, the amino lipid is an ionizable lipid. In some embodiments, the amino lipid comprises one or more nitrogen atoms. In some embodiments, the amino lipid comprises one or more ionizable nitrogen atoms. Exemplary cationic and/or ionizable lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).

In some embodiments, the amino lipid is symmetric, which may result in fewer metabolites and make the molecule achiral. In some embodiments, the amino lipid contains more number of hydrolysable bonds, which results in a faster metabolic clearance of the nanoparticles comprising the lipid. In some embodiments the symmetric achiral amino lipid helps yield tighter and/or smaller lipid nanoparticle than unsymmetric amino lipid after LNP formation wherein small particles translate efficient delivery to livers of mammalian subject compared to LNP particle of larger size

In one aspect, disclosed herein is an amino lipid having the structure of Formula (I), or a pharmaceutically acceptable salt or solvate thereof,

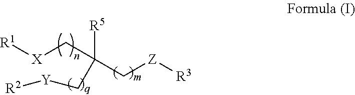

wherein
each of R¹ and R² is independently C₃-C₃₀ alkyl, C₃-C₃₀ alkenyl, C₃-C₃₀ alkynyl, C₃-C₁₀ cycloalkyl, C₃-C₃₀ heteroalkyl, C₃-C₃₀ heteroalkenyl, C₃-C₃₀ heteroalkynyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, or a bond;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloaryl is independently substituted or unsubstituted;
each of R⁴ is independently hydrogen or substituted or unsubstituted C₁-C₆ alkyl;
R⁵ is hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl; and
each of n, m, and q is independently 0, 1, 2, 3, 4, or 5.

In some embodiments of Formula (I),
each of X, Y, and Z is independently —R¹¹C(═O)N(R⁹)R⁴—, —NR⁷C(═O)R⁴—, —C(═O)O—, —OC(═O)—, —R¹¹C(═O)OR⁴—, —R¹¹OC(═O)R⁴—, —R¹¹OC(═O)OR⁴—, —R¹¹N(R⁹)C(═O)OR⁴—, —R¹¹OC(═O)N(R⁹)R⁴—, —R¹¹(NR⁹)C(═O)N(R⁹)R⁴—, —R¹¹N(R⁹)C(═NR⁹)R⁴—, —R¹¹C(═S)N(R⁹)R⁴—, —R¹¹N(R⁹)C(═S)R⁴—, —R¹¹C(═S)OR⁴—, —R¹¹OC(═S)R⁴—, —R¹¹OC(═S)OR⁴—, —R¹¹N(R⁹)C(═S)OR⁴—, —R¹¹OC(═S)N(R⁹)R⁴—, —R¹¹N(R⁹)C(═S)N(R⁹)R⁴—, —R¹¹C(═O)SR⁴—, —R¹¹SC(═O)R⁴—, —R¹¹OC(═O)SR⁴—, —R¹¹N(R⁹)C(═O)SR⁴—, —R¹¹SC(═O)N(R⁹)R⁴—, —R¹¹C(═S)SR⁴—, —R¹¹SC(═S)R⁴—, —R¹¹SC(═S)OR⁴—, —R¹¹N(R⁹)C(═S)SR⁴—, —R¹¹SC(═S)N(R⁹)R⁴—, —R¹¹C(═S)SR⁴—, —R¹¹SC(═S)R⁴—, —R¹¹SC(═O)SR⁴—, —R¹¹SC(═S)SR⁴—, —R¹¹N(R⁹)C(═S)SR⁴—, —R¹¹SC(═S)N(R⁹)R⁴—, —R¹¹OR⁴—, —R¹¹SR⁴—, or a bond;
each of L is independently —R¹¹C(═O)N(R⁹)R⁴—, —R¹¹N(R⁹)C(═O)R⁴—, —R¹¹C(═O)OR⁴—, —R¹¹OC(═O)R⁴—, —R¹¹OC(═O)OR⁴—, —R¹¹N(R⁹)C(═O)OR⁴—, —R¹¹OC(═O)N(R⁹)R⁴—, —R¹¹N(R⁹)C(═O)N(R⁹)R⁴—, —R¹¹N(R⁹)C(═NR⁹)N(R⁹)R⁴—, —R¹¹C(═S)N(R⁹)R⁴—, —R¹¹N(R⁹)C(═S)R⁴—, —R¹¹C(═S)OR⁴—, —R¹¹OC(═S)R⁴—, —R¹¹OC(═S)OR⁴—, —R¹¹N(R⁹)C(═S)OR⁴—, —R¹¹OC(═S)N(R⁹)R⁴—, —R¹¹N(R⁹)C(═S)N(R⁹)R⁴—, —R¹¹C(═O)SR⁴—, —R¹¹SC(═O)R⁴—, —R¹¹OC(═O)SR⁴—, —R¹¹N(R⁹)C(═O)SR⁴—, —R¹¹SC(═O)N(R⁹)R⁴—, —R¹¹C(═S)SR⁴—, —R¹¹SC(═S)R⁴—, —R¹¹SC(═S)OR⁴—, —R¹¹N(R⁹)C(═S)SR⁴—, —R¹¹SC(═S)N(R⁹)R⁴—, —R¹¹C(═S)SR⁴—, —R¹¹SC(═S)R⁴—, —R¹¹SC(═O)SR⁴—, —R¹¹SC(═S)SR⁴—, —R¹¹N(R⁹)C(═S)SR⁴—, —R¹¹SC(═S)N(R⁹)R⁴—, —R¹¹O—N═CR⁴—, —R¹¹C(R¹⁰)=N—OR⁴—, —R¹¹OR⁴—, —R¹¹SR⁴—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
each of R⁴ is independently substituted or unsubstituted C₁-C₁₆ alkyl, or unsubstituted C₁-C₁₆ heteroalkyl or absent;
R⁵ is hydrogen or substituted or unsubstituted C₁-C₁₆ alkyl;
each of R⁶ is independently hydrogen, C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each R⁷ is independently substituted or unsubstituted C₁-C₁₆ alkyl, substituted or unsubstituted C₁-C₁₆ heteroalkyl or absent;
each of R⁹ is independently hydrogen or substituted or unsubstituted C₁-C₁₆ alkyl, or unsubstituted C₁-C₁₆ heteroalkyl;
each of R¹⁰ is independently hydrogen or substituted or unsubstituted C₁-C₁₆ alkyl, or unsubstituted C₁-C₁₆ heteroalkyl; and
each of R¹¹ is independently substituted or unsubstituted C₁-C₁₆ alkyl, or unsubstituted C₁-C₁₆ heteroalkyl.

In one aspect, disclosed herein is an amino lipid having a structure of Formula (I*), or a pharmaceutically acceptable salt or solvate thereof,

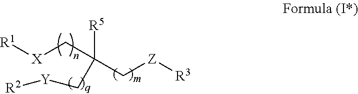

wherein
R¹ is hydrogen, C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
R² is hydrogen, C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —R¹¹C(═O)N(R⁴)R⁹—, —NR⁷C(═O)R⁹—, —C(═O)O—, —OC(═O)—, —R¹¹C(═O)OR⁹—, —R¹¹OC(═O)R⁹—, —R¹¹OC(═O)OR⁹—, —R¹¹N(R⁴)C(═O)OR⁹—, —R¹¹OC(═O)N(R⁴)R⁹—, —R¹¹(NR⁴)C(═O)N(R⁴)R⁹—, —R¹¹N(R⁴)C(═NR⁴)R⁹—, —R¹¹C(═S)N(R⁴)R⁹—, —R¹¹N(R⁴)C(═S)R⁹—, —R¹¹C(═S)OR⁹—, —R¹¹OC(═S)R⁹—, —R¹¹OC(═S)OR⁹—, —R¹¹N(R⁴)C(═S)OR⁹—, —R¹¹OC(═S)N(R⁴)R⁹—, —R¹¹N(R⁴)C(═S)N(R⁴)R⁹—, —R¹¹C(═O)SR⁹—, —R¹¹SC(═O)R⁹—, —R¹¹OC(═O)SR⁹—, —R¹¹N(R⁴)C(═O)SR⁹—, —R¹¹SC(═O)N(R⁴)R⁹—, —R¹¹C(═S)SR⁹—, —R¹¹SC(═S)R⁹—, —R¹¹SC(═S)OR⁹—, —R¹¹N(R⁴)C(═S)SR⁹—, —R¹¹SC(═S)N(R⁴)R⁹—, —R¹¹C(═S)SR⁹—, —R¹¹SC(═S)R⁹—, —R¹¹SC(═O)SR⁹—, —R¹¹SC(═S)SR⁹—, —R¹¹N(R⁴)C(═S)SR⁹—, —R¹¹SC(═S)N(R⁴)R⁹—, —R¹¹OR⁹—, —R¹¹SR⁹—, —O—, —S—, or a bond;
each of L is independently —C(═O)NR⁴—, —NR⁴C(═O)—, —C(═O)O—, —OC(═O)—, —C(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —R¹¹C(═O)N(R⁴)R⁹—, —R¹¹N(R⁴)C(═O)R⁹—, —R¹¹C(═O)OR⁹—, —R¹¹OC(═O)R⁹—, —R¹¹OC(═O)OR⁹—, —R¹¹N(R⁴)C(═O)OR⁹—, —R¹¹OC(═O)N(R⁴)R⁹—, —R¹¹N(R⁴)C(═O)N(R⁴)R⁹—, —R¹¹N(R⁴)C(═NR⁴)N(R⁴)R⁹—, —R¹¹C(═S)N(R⁴)R⁹—, —R¹¹N(R⁴)C(═S)R⁹—, —R¹¹C(═S)OR⁹—, —R¹¹OC(═S)R⁹—, —R¹¹OC(═S)OR⁹—, —R¹¹N(R⁴)C(═S)OR⁹—, —R¹¹OC(═S)N(R⁴)R⁹—, —R¹¹N(R⁴)C(═S)N(R⁴)R⁹—, —R¹¹C(═O)SR⁹—, —R¹¹SC(═O)R⁹—, —R¹¹OC(═O)SR⁹—, —R¹¹N(R⁴)C(═O)SR⁹—, —R¹¹SC(═O)N(R⁴)R⁹—, —R¹¹C(═S)SR⁹—, —R¹¹SC(═S)R⁹—, —R¹¹SC(═S)OR⁹—, —R¹¹N(R⁴)C(═S)SR⁹—, —R¹¹SC(═S)N(R⁴)R⁹—, —R¹¹C(═S)SR⁹—, —R¹¹SC(═S)R⁹—, —R¹¹SC(═O)SR⁹—, —R¹¹SC(═S)SR⁹—, —R¹¹N(R⁴)C(═S)SR⁹—, —R¹¹SC(═S)N(R⁴)R⁹—, —R¹¹O—N=CR⁹—, —R¹¹C(R¹⁰)=N—OR⁹—, —R¹¹OR⁹—, —R¹¹SR⁹—, —C₁-C₁₀ alkylene-C(═O)O—, —C₁-C₁₀ alkylene-OC(═O)—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloaryl is independently substituted or unsubstituted;
each of R⁴ is independently hydrogen, substituted or unsubstituted C₁-C₁₆ alkyl or substituted or unsubstituted C₁-C₁₆ heteroalkyl;
R⁵ is hydrogen or substituted or unsubstituted —C₀-C₁₀ alkylene-L-R⁴;
each of R⁶ is independently hydrogen, C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₁₆ alkyl, substituted or unsubstituted C₁-C₁₆ heteroalkyl, or R⁷ and R⁸ taken together with the nitrogen to which they are attached form a substituted or unsubstituted C₂-C₆ heterocyclyl; and
each of R⁹ is independently hydrogen or substituted or unsubstituted C₁-C₁₆ alkylene, or unsubstituted C₁-C₁₆ heteroalkylene;
each of R¹⁰ is independently hydrogen or substituted or unsubstituted C₁-C₁₆ alkylene, or unsubstituted C₁-C₁₆ heteroalkylene; and each of R¹¹ is independently substituted or unsubstituted C₁-C₁₆ alkylene, or unsubstituted C₁-C₁₆ heteroalkylene.
each of n, m, and q is independently 0, 1, 2, 3, 4, or 5.

In some embodiments, R⁵ of Formula (I*) is hydrogen or substituted or unsubstituted C₁-C₁₆ alkyl. In some embodiments, R⁵ of Formula (I*) is substituted or unsubstituted C₁-C₁₆ alkyl.

In some embodiments of Formula (I) or (I*), if the structure carries more than one asymmetric C-atom, each asymmetric C-atom independently represents racemic, chirally pure R and/or chirally pure S isomer, or a combination thereof.

In some embodiments, each of n, m, and q in Formula (I) or (I*) is independently 0, 1, 2, or 3. In some embodiments, each of n and m in Formula (I) or (I*) is 1. In some embodiments, n is 0. In some embodiments, n is 1. In some embodiments, n is 2. In some embodiments, n is 3. In some embodiments, m is 0. In some embodiments, m is 1. In some embodiments, m is 2. In some embodiments, m is 3. In some embodiments, q is 0. In some embodiments, q is 1. In some embodiments, q is 2. In some embodiments, q is 3.

In some embodiments, the compound of Formula (I) or (I*) has a structure of Formula (Ia), or a pharmaceutically acceptable salt or pharmaceutically acceptable solvate thereof:

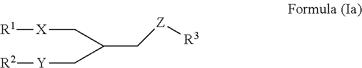

wherein
each of R¹ and R² is independently C₃-C₃₀ alkyl, C₃-C₃₀ alkenyl, C₃-C₃₀ alkynyl, C₃-C₁₀ cycloalkyl, C₃-C₃₀ heteroalkyl, C₃-C₃₀ heteroalkenyl, C₃-C₃₀ heteroalkynyl, —C₀-C₁₀ alkylene-L-R⁶, or —C₂-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, —C₁-C₁₀ alkylene-O—, or a bond, wherein the alkylene is substituted or unsubstituted;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, or —C₀-C₁₀ alkylene-heterocycloalkyl, wherein the alkylene and heterocycloalkyl are each independently substituted or unsubstituted;
each of R⁴ is independently hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl; and
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl.

In some embodiments of Formula (Ia), if the structure carries more than one asymmetric C-atom, each asymmetric C-atom independently represents racemic, chirally pure R and/or chirally pure S isomer, or a combination thereof.

In some embodiments, a compound of Formula (I), Formula (I*), Formula (Ia), or Formula (Ib) comprises an unsymmetrical heteroatom on R¹ and/or R².

In some embodiments, each R¹ and R² in Formula (I), Formula (I*) or Formula (Ia) is independently C₅-C₃₀ alkyl, C₅-C₃₀ alkenyl, C₅-C₃₀ alkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, and cycloalkyl is independently substituted or unsubstituted. In some embodiments, each R¹ and R² in Formula (I), Formula (I*), Formula (Ia), or Formula (Ib) is independently C₇-C₂₈ alkyl, C₇-C₂₈ alkenyl, C₇-C₂₈ alkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, and cycloalkyl is independently substituted or unsubstituted. In some embodiments, each R¹ and R² in Formula (I), Formula (I*), Formula (Ia), or Formula (Ib) is independently C₇-C₂₅ alkyl, C₇-C₂₅ alkenyl, C₇-C₂₅ alkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, and cycloalkyl is independently substituted or unsubstituted. In some embodiments, each R¹ and R² in Formula (I), Formula (I*), Formula (Ia), or Formula (Ib) is independently C₇-C₂₂ alkyl, C₇-C₂₂ alkenyl, C₇-C₂₂ alkynyl, C₃-C₁₀ cycloalkyl, —C₀-C₁₀ alkylene-L-R⁶, or —C₂-C₁₀ alkenylene-L-R⁶, wherein each of the alkyl, alkylene, alkenyl, alkenylene, alkynyl, and cycloalkyl is independently substituted or unsubstituted. In some embodiments, each R¹ and R² in Formula (I), Formula (I*), Formula (Ia), or Formula (Ib) is independently C₁₀-C₂₀ alkyl, C₁₀-C₂₀ alkenyl, —C₄-C₈ alkylene-L-R⁶, or —C₄-C₈ alkylene-L-R⁶, wherein each of the alkyl, alkylene, alkenyl, and cycloalkyl is independently substituted or unsubstituted. In some embodiments, each R¹ and R² in Formula (I), Formula (I*), Formula (Ia), or Formula (Ib) is —C₀-C₁₀ alkylene-L-R⁶. In some embodiments, each R¹ and R² in Formula (I), Formula (I*), Formula (Ia), or Formula (Ib) is C₇-C₃₀ alkenyl. In some embodiments, each R¹ and R² in Formula (I), Formula (I*), Formula (Ia), or Formula (Ib) is C₇-C₂₈ alkenyl. In some embodiments, each R¹ and R² in Formula (I), Formula (I*), Formula (Ia), or Formula (Ib) is C₇-C₂₅ alkenyl. In some embodiments, each R¹ and R² in Formula (I), Formula (I*), Formula (Ia), or Formula (Ib) is C₇-C₂₂ alkenyl.

In some embodiments, each R¹ and R² in Formula (I), Formula (I*), Formula (Ia), or Formula (Ib) is independently substituted or unsubstituted linear or branched C₃-C₃₀ alkyl or substituted or unsubstituted linear or branched C₃-C₃₀ alkenyl. In some embodiments, each R¹ and R² in Formula (I), Formula (I*), Formula (Ia), or Formula (Ib) is independently substituted or unsubstituted linear or branched C₃-C₂₈ alkyl or substituted or unsubstituted linear or branched C₃-C₂₈ alkenyl. In some embodiments, each R¹ and R² in Formula (I), Formula (I*), Formula (Ia), or Formula (Ib) is independently substituted or unsubstituted C₃-C₂₅ alkyl or substituted or unsubstituted C₃-C₂₅ alkenyl. In some embodiments, each R¹ and R² in Formula (I), Formula (I*), Formula (Ia), or Formula (Ib) is independently substituted or unsubstituted C₃-C₂₅ alkyl. In some embodiments, each R¹ and R² in Formula (I), Formula (I*), Formula (Ia), or Formula (Ib) is independently C₃ alkyl, C₄ alkyl, C₅ alkyl, C₆ alkyl, C₇ alkyl, C₈ alkyl, C₉ alkyl, C₁₀ alkyl, C₁₁ alkyl, C₁₂ alkyl, C₁₃ alkyl, C₁₄ alkyl, C₁₅ alkyl, C₁₆ alkyl, C₁₇ alkyl, C₁₈ alkyl, C₁₉ alkyl, C₂₀ alkyl, C₂₁ alkyl, C₂₂ alkyl, C₂₃ alkyl, C₂₄ alkyl, or C₂₅ alkyl. In some embodiments, each R¹ and R² in Formula (I), Formula (I*), Formula (Ia), or Formula (Ib) is independently at least C₃ alkyl, at least C₄ alkyl, at least C₅ alkyl, at least C₆ alkyl, at least C₇ alkyl, at least C₈ alkyl, at least C₉ alkyl, at least C₁₀ alkyl, at least C₁₁ alkyl, at least C₁₂ alkyl, at least C₁₃ alkyl, at least C₁₄ alkyl, at least C₁₅ alkyl, at least C₁₆ alkyl, at least C₁₇ alkyl, at least C₁₈ alkyl, at least C₁₉ alkyl, at least C₂₀ alkyl, at least C₂₁ alkyl, at least C₂₂ alkyl, at least C₂₃ alkyl, at least C₂₄ alkyl, or at least C₂₅ alkyl. In some embodiments, each R¹ and R² in Formula (I), Formula (I*), Formula (Ia), or Formula (Ib) is independently at most C₃ alkyl, at most C₄ alkyl, at most C₅ alkyl, at most C₆ alkyl, at most C₇ alkyl, at most C₈ alkyl, at most C₉ alkyl, at most C₁₀ alkyl, at most C₁₁ alkyl, at most C₁₂ alkyl, at most C₁₃ alkyl, at most C₁₄ alkyl, at most Cis alkyl, at most C₁₆ alkyl, at most C₁₇ alkyl, at most C₁₈ alkyl, at most C₁₉ alkyl, at most C₂₀ alkyl, at most C₂₁ alkyl, at most C₂₂ alkyl, at most C₂₃ alkyl, at most C₂₄ alkyl, or at most C₂₅ alkyl. In some embodiments, each R¹ and R² in Formula (I), Formula (I*), Formula (Ia), or Formula (Ib) is independently C₈-C₂₄ alkyl. In some embodiments, each R¹ and R² in Formula (I), Formula (I*), Formula (Ia), or Formula (Ib) is independently C₁₂-C₂₄ alkyl. In some embodiments, each R¹ and R² in Formula (I), Formula (I*), Formula (Ia), or Formula (Ib) is independently C₆-C₁₅ alkyl. In some embodiments, each R¹ and R² in Formula (I), Formula (I*), Formula (Ia), or Formula (Ib) is independently substituted or unsubstituted C₃-C₂₅ alkenyl. In some embodiments, each R¹ and R² in Formula (I), Formula (I*), Formula (Ia), or Formula (Ib) is independently C₃ alkenyl, C₄ alkenyl, C₅ alkenyl, C₆ alkenyl, C₇ alkenyl, C₈ alkenyl, C₉ alkenyl, C₁₀ alkenyl, C_{II} alkenyl, C₁₂ alkenyl, C₁₃ alkenyl, C₁₄ alkenyl, C₁₅ alkenyl, C₁₆ alkenyl, C₁₇ alkenyl, C₁₈ alkenyl, C₁₉ alkenyl, C₂₀ alkenyl, C₂₁ alkenyl, C₂₂ alkenyl, C₂₃ alkenyl, C₂₄ alkenyl, or C₂₅ alkenyl. In some embodiments, each R¹ and R² in Formula (I), Formula (I*), Formula (Ia), or Formula (Ib) is independently at least C₃ alkenyl, at least C₄ alkenyl, at least C₅ alkenyl, at least C₆ alkenyl, at least C₇ alkenyl, at least C₈ alkenyl, at least C₉ alkenyl, at least C₁₀ alkenyl, at least C₁₁ alkenyl, at least C₁₂ alkenyl, at least C₁₃ alkenyl, at least C₁₄ alkenyl, at least C₁₅ alkenyl, at least C₁₆ alkenyl, at least C₁₇ alkenyl, at least C₁₈ alkenyl, at least C₁₉ alkenyl, at least C₂₀ alkenyl, at least C₂₁ alkenyl, at least C₂₂ alkenyl, at least C₂₃ alkenyl, at least C₂₄ alkenyl, or at least C₂₅ alkenyl. In some embodiments, each R¹ and R² in Formula (I), Formula (I*), Formula (Ia), or Formula (Ib) is independently at most C₃ alkenyl, at most C₄ alkenyl, at most C₅ alkenyl, at most C₆ alkenyl, at most C₇ alkenyl, at most C₈ alkenyl, at most C₉ alkenyl, at most C₁₀ alkenyl, at most C₁ alkenyl, at most C₁₂ alkenyl, at most C₁₃ alkenyl, at most C₁₄ alkenyl, at most C₁₅ alkenyl, at most C₁₆ alkenyl, at most C₁₇ alkenyl, at most C₁₈ alkenyl, at most C₁₉ alkenyl, at most C₂₀ alkenyl, at most C₂₁ alkenyl, at most C₂₂ alkenyl, at most C₂₃ alkenyl, at most C₂₄ alkenyl, or at most C₂₅ alkenyl.

In some embodiments of a compound of Formula (I), Formula (I*), Formula (Ia), or Formula (Ib), R¹ and R² are each substituted or unsubstituted Cy-C₃₋₃₀ alkyl. In some embodiments, Cy is a bicyclic. In some embodiments, Cy is a monocylic. In some embodiments, Cy is bicyclic heteroaryl with 0-2 Nitrogen and 0-1 oxygen. In some embodiments, Cy is bicyclic heteroalkyl with 0-2 Nitrogen and 0-1 oxygen.

In some embodiments, R¹ and/or R² in Formula (I), Formula (I*), Formula (Ia), or Formula (Ib) is independently

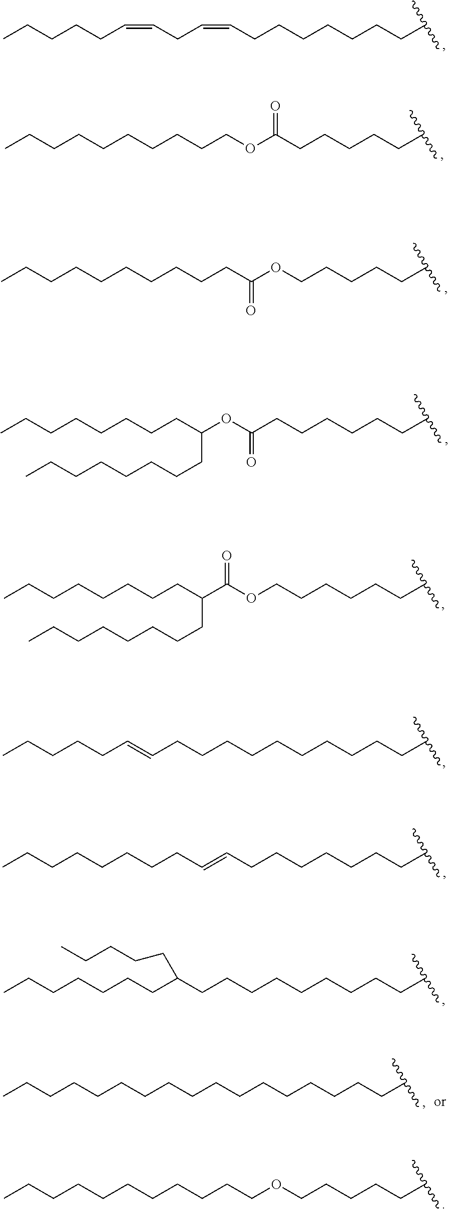

In some embodiments, R¹ and/or R² in Formula (I), Formula (I*), Formula (Ia), or Formula (Ib) is

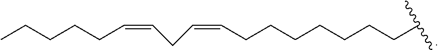 [SMILES: *CCCCCCC/C=C\C/C=C\CCCCC]

In some embodiments, R¹ and/or R² in Formula (I), Formula (I*), Formula (Ia), or Formula (Ib) is

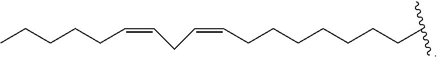 [SMILES: *CCCCCCC/C=C\C/C=C\CCCCC]

In some embodiments, R¹ and/or R² in Formula (I), Formula (I*) Formula (Ia) or Formula (Ib) is

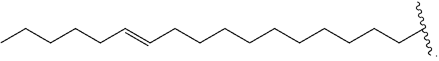 [SMILES: *CCCCCCCCCC/C=C/CCCCC]

In some embodiments, R¹ and/or R² in Formula (I), Formula (*), Formula (Ia), or Formula (Ib) is

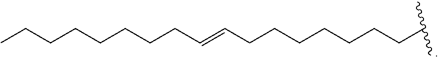 [SMILES: *CCCCCCC/C=C/CCCCCCCC]

In some embodiments, R¹ and/or R² in Formula (I), Formula (I*) Formula (Ia) or Formula (Ib) is

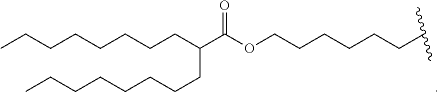 [SMILES: *CCCCCCOC(=O)C(CCCCCCCC)CCCCCCCC]

In some embodiments, R¹ and/or R² in Formula (I), Formula (I*), Formula (Ia), or Formula (Ib) is

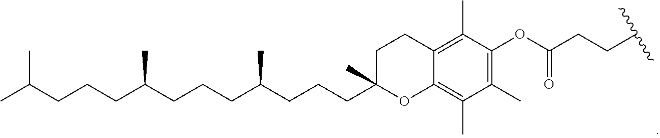 [SMILES: *CCC(=O)Oc1c(C)c(C)c2c(c1C)CC[C@@](C)(CCC[C@H](C)CCC[C@H](C)CCCC(C)C)O2]

In some embodiments, R¹ and/or R² in Formula (I), Formula (I*), Formula (Ia), or Formula (Ib) is

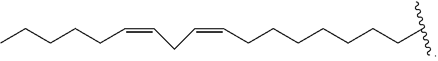 [SMILES: *CCCCCCC/C=C\C/C=C\CCCCC]

In some embodiments, R¹ and/or R² in Formula (I), Formula (I*), Formula (Ia), or Formula (Ib) is

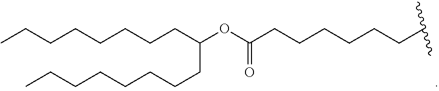 [SMILES: *CCCCCCC(=O)OC(CCCCCCCC)CCCCCCCC]

In some embodiments, L in Formula (I), Formula (I*), Formula (Ia), or Formula (Ib) is independently —O—, —S—, —C₁-C₁₀ alkylene-O—, —C₁-C₁₀ alkylene-C(═O)O—, —C₁-C₁₀ alkylene-OC(═O)—, —C(═O)O—, —OC(═O)—, —C(═O)NR⁴—, —NR⁴C(═O)—, or a bond, wherein the alkylene is substituted or unsubstituted. In some embodiments, at least one Lin Formula (I), Formula (I*), Formula (Ia), or Formula (Ib) is independently —O—, —S—, —C₁-C₃ alkylene-O—, —C₁-C₃ alkylene-C(═O)O—, —C₁-C₃ alkylene-OC(═O)—, —C(═O)O—, —OC(═O)—, —C(═O)NR⁴—, —NR⁴C(═O)—, or a bond, wherein the alkylene is substituted or unsubstituted. In some embodiments, at least one Lin Formula (I), Formula (I*), Formula (Ia), or Formula (Ib) is independently —O—, —S—, —C₁-C₃ alkylene-O—, —C₁-C₃ alkylene-C(═O)O—, —C₁-C₃ alkylene-OC(═O)—, or a bond, wherein the alkylene is linear or branched unsubstituted alkylene. In some embodiments, at least one L in Formula (I), Formula (I*), Formula (Ia), or Formula (Ib) is —C(═O)O—, —OC(═O)—, —C(═O)NR⁴—, —NR⁴C(═O)—. In some embodiments, at least one L in Formula (I), Formula (I*), Formula (Ia), or Formula (Ib) is —C(═O)O—. In some embodiments, at least one L in Formula (I), Formula (I*), Formula (Ia), or Formula (Ib) is —OC(═O)—. In some embodiments, at least one L in Formula (I), Formula (I*), Formula (Ia), or Formula (Ib) is —C(═O)NR⁴—. In some embodiments, at least one L in Formula (I), Formula (I*), Formula (Ia), or Formula (Ib) is —C(═O)NH—. In some embodiments, at least one L in Formula (I), Formula (I*), Formula (Ia), or Formula (Ib) is —C(═O)N(CH₃)—. In some embodiments, at least one L in Formula (I), Formula (I*), Formula (Ia), or Formula (Ib) is —NR⁴C(═O)—. In some embodiments, at least one L in Formula (I), Formula (I*), Formula (Ia), or Formula (Ib) is —NHC(═O)—. In some embodiments, at least one L in Formula (I), Formula (I*), Formula (Ia), or Formula (Ib) is —N(CH₃)C(═O)—. In some embodiments, at least one L in Formula (I), Formula (I*), Formula (Ia), or Formula (Ib) is —O—N═CR⁴— or —CR⁴=N—O—. In some embodiments, at least one L in Formula (I), Formula (I*), Formula (Ia), or Formula (Ib) Formula (I), Formula (I*), Formula (Ia), or Formula (Ib), R¹ and R² are the same. In some embodiments, at least one L in Formula (I), Formula (I*), Formula (Ia), or Formula (Ib) Formula (I), Formula (I*), Formula (Ia), or Formula (Ib), R¹ and R² are different.

In some embodiments of Formula (I), Formula (I*), Formula (Ia), or Formula (Ib), R¹ is —C₀-C₁₀ alkylene-L-R⁶. In some embodiments of Formula (I), Formula (I*), Formula (Ia), or Formula (Ib), R² is —C₀-C₁₀ alkylene-L-R⁶. In some embodiments of Formula (I), Formula (I*), Formula (Ia), or Formula (Ib), X is —C(═O)O—. In some embodiments of Formula (I), Formula (I*), Formula (Ia), or Formula (Ib), X is —OC(═O)—. In some embodiments of Formula (I), Formula (I*), Formula (Ia), or Formula (Ib), X is —OC(═O)NR⁴—. In some embodiments of Formula (I), Formula (I*), Formula (Ia), or Formula (Ib), Y is —C(═O)O—. In some embodiments of Formula (I), Formula (I*), Formula (Ia), or Formula (Ib), Y is —OC(═O)—. In some embodiments of Formula (I), Formula (I*), Formula (Ia), or Formula (Ib), Y is —OC(═O)NR⁴—. In some embodiments of Formula (I), Formula (I*), Formula (Ia), or Formula (Ib), L is —OC(═O)—. In some embodiments of Formula (I), Formula (I*), Formula (Ia), or Formula (Ib), L is —C(═O)—. In some embodiments of Formula (I), Formula (I*), Formula (Ia), or Formula (Ib), R¹ is —C₄-C₈ alkylene-L-R⁶. In some embodiments of Formula (I), Formula (I*), Formula (Ia), or Formula (Ib), R² is —C₄-C₈ alkylene-L-R⁶.

In some embodiments of Formula (I), Formula (I*), Formula (Ia), or Formula (Ib), R¹ is optionally substituted by oxo. In some embodiments of Formula (I), Formula (I*), Formula (Ia), or Formula (Ib), R¹ or R² does not contain any hydrylozable bonds. In some embodiments of Formula (I), Formula (I*), Formula (Ia), or Formula (Ib), R¹ or R² does not contain any C═C bonds. In some embodiments of Formula (I), Formula (I*), Formula (Ia), or Formula (Ib), R¹ or R² does not contain any carbon-carbon triple bonds.

In some embodiments, p in Formula (Ib) is 1 to 6. In some embodiments, p in Formula (Ib) is 1 to 2, 1 to 3, 1 to 4, 1 to 5, 1 to 6, 2 to 3, 2 to 4, 2 to 5, 2 to 6, 3 to 4, 3 to 5, 3 to 6, 4 to 5, 4 to 6, or 5 to 6. In some embodiments, p in Formula (Ib) is 1, 2, 3, 4, 5, or 6. In some embodiments, p in Formula (Ib) is at least 1, 2, 3, 4, or 5. In some embodiments, p in Formula (Ib) is at most 2, 3, 4, 5, or 6. In some embodiments, p in Formula (Ib) is 1-3. In some embodiments, p in Formula (Ib) is 1. In some embodiments, p in Formula (Ib) is 2. In some embodiments, p in Formula (Ib) is 3. In some embodiments, p in Formula (Ib) is 4. In some embodiments, p in Formula (Ib) is 5. In some embodiments, p in Formula (Ib) is 6.

In some embodiments, at least one R⁶ in Formula (I), Formula (I*), Formula (Ia), or Formula (Ib) is independently substituted or unsubstituted linear or branched C₃-C₃₀ alkyl or substituted or unsubstituted linear or branched C₃-C₃₀ alkenyl. In some embodiments, at least one R⁶ in Formula (I), Formula (I*), Formula (Ia), or Formula (Ib) is independently substituted or unsubstituted linear or branched C₃-C₂₈ alkyl or substituted or unsubstituted linear or branched C₃-C₂₈ alkenyl. In some embodiments, at least one R⁶ in Formula (I), Formula (I*), Formula (Ia), or Formula (Ib) is independently substituted or unsubstituted C₃-C₂₅ alkyl or substituted or unsubstituted C₃-C₂₅ alkenyl. In some embodiments, at least one R⁶ in Formula (I), Formula (I*), Formula (Ia), or Formula (Ib) is independently substituted or unsubstituted C₃-C₂₅ alkyl. In some embodiments, at least one R⁶ in Formula (I), Formula (I*), Formula (Ia), or Formula (Ib) is independently C₃ alkyl, C₄ alkyl, C₅ alkyl, C₆ alkyl, C₇ alkyl, C₈ alkyl, C₉ alkyl, C₁₀ alkyl, C₁₁ alkyl, C₁₂ alkyl, C₁₃ alkyl, C₁₄ alkyl, C₁₅ alkyl, C₁₆ alkyl, C₁₇ alkyl, C₁₈ alkyl, C₁₉ alkyl, C₂₀ alkyl, C₂₁ alkyl, C₂₂ alkyl, C₂₃ alkyl, C₂₄ alkyl, or C₂₅ alkyl. In some embodiments, at least one R⁶ in Formula (I), Formula (I*), Formula (Ia), or Formula (Ib) is independently at least C₃ alkyl, at least C₄ alkyl, at least C₅ alkyl, at least C₆ alkyl, at least C₇ alkyl, at least C₈ alkyl, at least C₉ alkyl, at least C₁₀ alkyl, at least C₁₁ alkyl, at least C₁₂ alkyl, at least C₁₃ alkyl, at least C₁₄ alkyl, at least C₁₅ alkyl, at least C₁₆ alkyl, at least C₁₇ alkyl, at least C₁₈ alkyl, at least C₁₉ alkyl, at least C₂₀ alkyl, at least C₂₁ alkyl, at least C₂₂ alkyl, at least C₂₃ alkyl, at least C₂₄ alkyl, or at least C₂₅ alkyl. In some embodiments, at least one R⁶ in Formula (I), Formula (I*), Formula (Ia), or Formula (Ib) is independently at most C₃ alkyl, at most C₄ alkyl, at most C₅ alkyl, at most C₆ alkyl, at most C₇ alkyl, at most C₈ alkyl, at most C₉ alkyl, at most C₁₀ alkyl, at most C₁₁ alkyl, at most C₁₂ alkyl, at most C₁₃ alkyl, at most C₁₄ alkyl, at most Cis alkyl, at most C₁₆ alkyl, at most C₁₇ alkyl, at most C₁₈ alkyl, at most C₁₉ alkyl, at most C₂₀ alkyl, at most C₂₁ alkyl, at most C₂₂ alkyl, at most C₂₃ alkyl, at most C₂₄ alkyl, or at most C₂₅ alkyl. In some embodiments, at least one R⁶ in Formula (I), Formula (I*), Formula (Ia), or Formula (Ib) is independently substituted or unsubstituted C₃-C₂₅ alkenyl. In some embodiments, at least one R⁶ in Formula (I), Formula (I*), Formula (Ia), or Formula (Ib) is independently C₃ alkenyl, C₄ alkenyl, C₅ alkenyl, C₆ alkenyl, C₇ alkenyl, C₈ alkenyl, C₉ alkenyl, C₁₀ alkenyl, C_{II} alkenyl, C₁₂ alkenyl, C₁₃ alkenyl, C₁₄ alkenyl, C₁₅ alkenyl, C₁₆ alkenyl, C₁₇ alkenyl, C₁₈ alkenyl, C₁₉ alkenyl, C₂₀ alkenyl, C₂₁ alkenyl, C₂₂ alkenyl, C₂₃ alkenyl, C₂₄ alkenyl, or C₂₅ alkenyl. In some embodiments, at least one R⁶ in Formula (I), Formula (I*), Formula (Ia), or Formula (Ib) is independently at least C₃ alkenyl, at least C₄ alkenyl, at least C₅ alkenyl, at least C₆ alkenyl, at least C₇ alkenyl, at least C₈ alkenyl, at least C₉ alkenyl, at least C₁₀ alkenyl, at least C₁₁ alkenyl, at least C₁₂ alkenyl, at least C₁₃ alkenyl, at least C₁₄ alkenyl, at least Cis alkenyl, at least C₁₆ alkenyl, at least C₁₇ alkenyl, at least C₁₈ alkenyl, at least C₁₉ alkenyl, at least C₂₀ alkenyl, at least C₂₁ alkenyl, at least C₂₂ alkenyl, at least C₂₃ alkenyl, at least C₂₄ alkenyl, or at least C₂₅ alkenyl. In some embodiments, at least one R⁶ in Formula (I), Formula (I*), Formula (Ia), or Formula (Ib) is independently at most C₃ alkenyl, at most C₄ alkenyl, at most C₅ alkenyl, at most C₆ alkenyl, at most C₇ alkenyl, at most C₈ alkenyl, at most C₉ alkenyl, at most C₁₀ alkenyl, at most C₁ alkenyl, at most C₁₂ alkenyl, at most C₁₃ alkenyl, at most C₁₄ alkenyl, at most Cis alkenyl, at most C₁₆ alkenyl, at most C₁₇ alkenyl, at most C₁₈ alkenyl, at most C₁₉ alkenyl, at most C₂₀ alkenyl, at most C₂₁ alkenyl, at most C₂₂ alkenyl, at most C₂₃ alkenyl, at most C₂₄ alkenyl, or at most C₂₅ alkenyl. In some embodiments, at least one R⁶ in Formula (I), Formula (I*), Formula (Ia), or Formula (Ib) is independently substituted or unsubstituted C₃-C₃₀ alkynyl. In some embodiments, at least one R⁶ in Formula (I), Formula (I*), Formula (Ia), or Formula (Ib) is independently Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted. In some embodiments, R⁶ in Cy-C₃₋₃₀ alkyl, wherein the Cy is substituted or unsubstituted cycloalkyl, substituted or unsubstituted heterocycloalkyl, substituted or unsubstituted aryl, substituted or unsubstituted heteroaryl. In some embodiments, Cy is a bicyclic. In some embodiments, Cy is a monocylic. In some embodiments, Cy is bicyclic heteroaryl with 0-2 Nitrogen and 0-1 oxygen. In some embodiments, Cy is bicyclic heteroalkyl with 0-2 Nitrogen and 0-1 oxygen.

In some embodiments, R⁶ in Formula (I), Formula (I*), Formula (Ia), or Formula (Ib) is independently

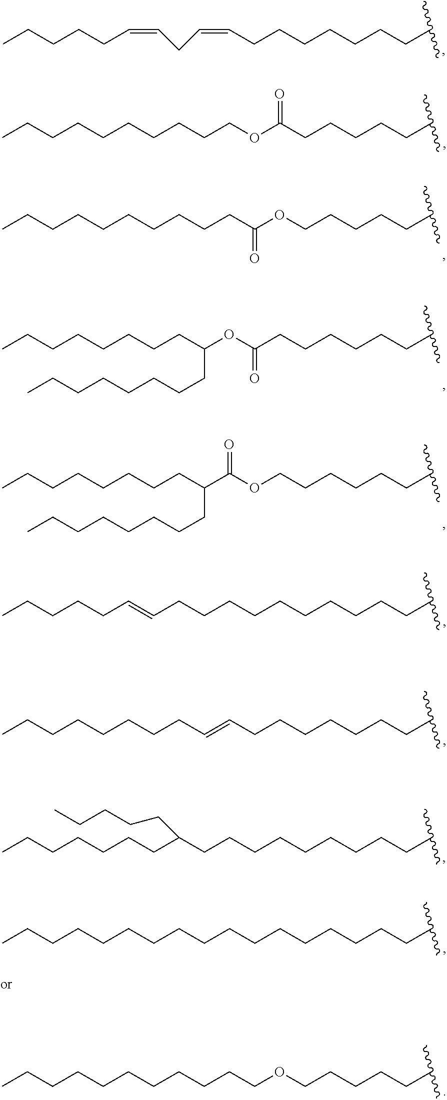

In some embodiments, at least one R⁶ in Formula (I), Formula (I*), Formula (Ia), or Formula (Ib) is

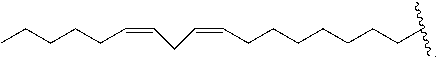 [SMILES: *CCCCCCC/C=C\C/C=C\CCCCC]

In some embodiments, at least one R⁶ in Formula (I), Formula (I*), Formula (Ia), or Formula (Ib) is

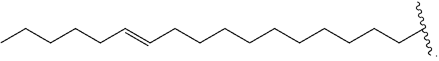 [SMILES: *CCCCCCCCCC/C=C/CCCCC]

In some embodiments, at least one R⁶ in Formula (T) Formula (T*) Formula (Ta) or Formula (Th) is

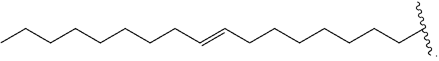 [SMILES: *CCCCCCC/C=C/CCCCCCCC]

In some embodiments, at least one R⁶ in Formula (I), Formula (I*), Formula (Ia), or Formula (Ib) is

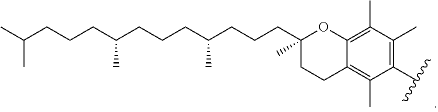 [SMILES: *c1c(C)c(C)c2c(c1C)CC[C@@](C)(CCC[C@H](C)CCC[C@H](C)CCCC(C)C)O2]

In some embodiments, at least one R⁶ in Formula (I), Formula (I*), Formula (Ia), or Formula (Ib) is

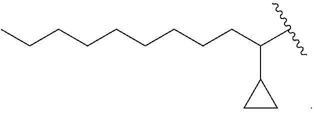 [SMILES: *C(CCCCCCCCC)C1CC1]

In some embodiments, at least one R⁶ in Formula (I), Formula (I*), Formula (Ia), or Formula (Ib) is

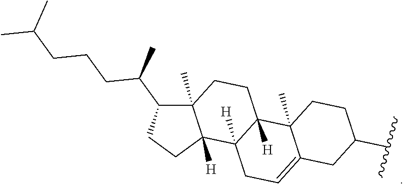

In some embodiments, R⁴ in Formula (I), Formula (I*), Formula (Ia), or Formula (Ib) is independently H or substituted or unsubstituted C₁-C₄ alkyl. In some embodiments, at least one R⁴ in Formula (I), Formula (I*), Formula (Ia), or Formula (Ib) is independently substituted or unsubstituted linear or branched C₁-C₄ alkyl. In some embodiments, at least one R⁴ in Formula (I), Formula (I*), Formula (Ia), or Formula (Ib) is H. In some embodiments, at least one R⁴ in Formula (I), Formula (I*), Formula (Ia), or Formula (Ib) is independently H, —CH₃, —CH₂CH₃, —CH₂CH₂CH₃, or —CH(CH₃)₂. In some embodiments, at least one R⁴ in Formula (I), Formula (I*), Formula (Ia), or Formula (Ib) is independently H or —CH₃. In some embodiments, at least one R⁴ in Formula (I), Formula (I*), Formula (Ia), or Formula (Ib) is —CH₃.

In some embodiments, W, X, and Y in Formula (I), Formula (I*), Formula (Ia), or Formula (Ib) is independently —C(═O)O— or —OC(═O)—. In some embodiments, at least one W, X, and Y in Formula (I), Formula (I*), Formula (Ia), or Formula (Ib) is —C(═O)O—. In some embodiments, at least one W, X, and Y in Formula (I), Formula (I*), Formula (Ia), or Formula (Ib) is —OC(═O)—. In some embodiments, at least one W, X, and Y in Formula (I), Formula (I*), Formula (Ia), or Formula (Ib) is independently —C(═O)NR⁴— or —NR⁴C(═O)—. In some embodiments, at least one W, X, and Y in Formula (I), Formula (I*), Formula (Ia), or Formula (Ib) is independently —C(═O)N(CH₃)—, —N(CH₃)C(═O)—, —C(═O)NH—, or —NHC(═O)—. In some embodiments, at least one W, X, and Y in Formula (I), Formula (I*), Formula (Ia), or Formula (Ib) is independently —C(═O)NH—, —C(═O)N(CH₃)—. —OC(═O)—, —NHC(═O)—, —N(CH₃)C(═O)—, —C(═O)O—, —OC(═O)O—, —NHC(═O)O—, —N(CH₃)C(═O)O—, —OC(═O)NH—, —OC(═O)N(CH₃)—, —NHC(═O)NH—, —N(CH₃)C(═O)NH—, —NHC(═O)N(CH₃)—, —N(CH₃)C(═O)N(CH₃)—, NHC(═NH)NH—, —N(CH₃)C(═NH)NH—, —NHC(═NH)N(CH₃)—, —N(CH₃)C(═NH)N(CH₃)—, NHC(═NMe)NH—, —N(CH₃)C(═NMe)NH—, —NHC(═NMe)N(CH₃)—, or —N(CH₃)C(═NMe)N(CH₃)—. In some embodiments, at least one W, X, and Y in Formula (I), Formula (I*), Formula (Ia), or Formula (Ib) is independently —OC(═O)O—, —NR⁴C(═O)O—, —OC(═O)NR⁴—, or —NR⁴C(═O)NR⁴—. In some embodiments, at least one W, X, and Y in Formula (I), Formula (I*), Formula (Ia), or Formula (Ib) is independently —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)— or —N(CH₃)C(═O)NH—. In some embodiments, at least one W, X, and Y in Formula (I), Formula (I*), Formula (Ia), or Formula (Ib) is independently —OC(═O)O—, —NHC(═O)O—, —OC(═O)NH—, or —NHC(═O)NH—.

In some embodiments, R³ in Formula (I), Formula (I*), Formula (Ia), or Formula (Ib) is —C₀-C₁₀ alkylene-NR⁷R⁸ or —C₀-C₁₀ alkylene-heterocycloalkyl, wherein the alkylene and heterocycloalkyl is independently substituted or unsubstituted. In some embodiments, R³ in Formula (I), Formula (I*), Formula (Ia), or Formula (Ib) is —C₀-C₁₀ alkylene-NR⁷R⁸. In some embodiments, R³ in Formula (I), Formula (I*), Formula (Ia), or Formula (Ib) is —C₁-C₆ alkylene-NR⁷R⁸. In some embodiments, R³ in Formula (I), Formula (I*), Formula (Ia), or Formula (Ib) is —C₁-C₄ alkylene-NR⁷R⁸. In some embodiments, R³ in Formula (I), Formula (I*), Formula (Ia), or Formula (Ib) is —C₁-alkylene-NR⁷R⁸. In some embodiments, R³ in Formula (I), Formula (I*), Formula (Ia), or Formula (Ib) is —C₂-alkylene-NR⁷R⁸. In some embodiments, R³ in Formula (I), Formula (I*), Formula (Ia), or Formula (Ib) is —C₃-alkylene-NR⁷R⁸. In some embodiments, R³ in Formula (I), Formula (I*), Formula (Ia), or Formula (Ib) is —C₄-alkylene-NR⁷R⁸. In some embodiments, R³ in Formula (I), Formula (I*), Formula (Ia), or Formula (Ib) is —C₅— alkylene-NR⁷R⁸. In some embodiments, R³ in Formula (I), Formula (I*), Formula (Ia), or Formula (Ib) is —C₀-C₁₀ alkylene-heterocycloalkyl. In some embodiments, R³ in Formula (I), Formula (I*), Formula (Ia), or Formula (Ib) is —C₁-C₆ alkylene-heterocycloalkyl, wherein the heterocycloalkyl comprises 1 to 3 nitrogen and 0-2 oxygen. In some embodiments, R³ in Formula (I), Formula (I*), Formula (Ia), or Formula (Ib) is quaternized.

In some embodiments, R⁷ and R⁸ in Formula (I), Formula (I*), Formula (Ia), or Formula (Ib) is independently hydrogen or substituted or unsubstituted C₁-C₆ alkyl. In some embodiments, R⁷ and R⁸ in Formula (I), Formula (I*), Formula (Ia), or Formula (Ib) is independently substituted or unsubstituted C₁-C₆ alkyl. In some embodiments, R⁷ and R⁸ is independently hydrogen or substituted or unsubstituted C₁-C₃ alkyl. In some embodiments, at least one R⁷ and R⁸ is independently substituted or unsubstituted C₁-C₃ alkyl. In some embodiments, at least one R⁷ and R⁸ is independently —CH₃, —CH₂CH₃, —CH₂CH₂CH₃, or —CH(CH₃)₂. In some embodiments, at least one R⁷ and R⁸ is CH₃. In some embodiments, at least one R⁷ and R⁸ is —CH₂CH₃.

In some embodiments, R⁷ and R⁸ in Formula (I), Formula (I*), Formula (Ia), or Formula (Ib) Formula (I), Formula (I*), Formula (Ia), or Formula (Ib), taken together with the nitrogen to which they are attached, form a substituted or unsubstituted C₂-C₆ heterocyclyl. In some embodiments, R⁷ and R⁸ taken together with the nitrogen to which they are attached form a substituted or unsubstituted C₂-C₆ heterocycloalkyl. In some embodiments, R⁷ and R⁸ taken together with the nitrogen to which they are attached form a substituted or unsubstituted 3-7 membered heterocycloalkyl.

In some embodiments, R³ in Formula (I), Formula (I*), Formula (Ia), or Formula (Ib) is

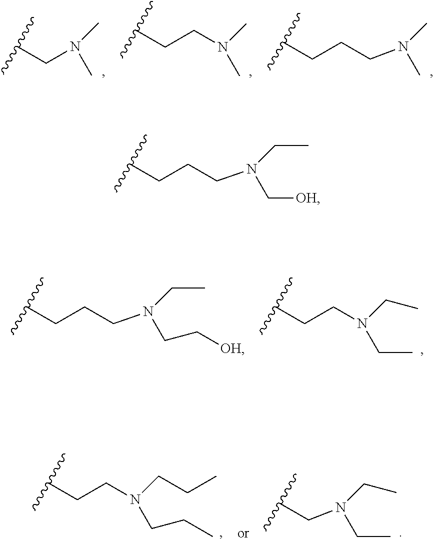

In some embodiments, R³ in Formula (I), Formula (I*), Formula (Ia), or Formula (Ib) is

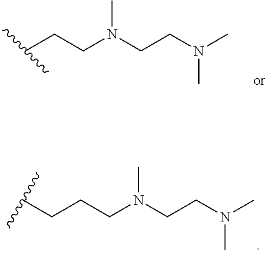

In some embodiments, R³ in Formula (I), Formula (I*), Formula (Ia), or Formula (Ib) is

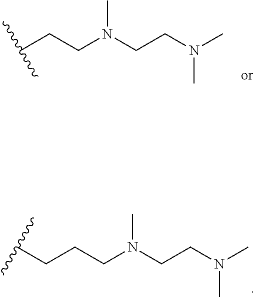

In some embodiments R³ in Formula (I), Formula (I*) Formula (Ia), or Formula (Ib) is

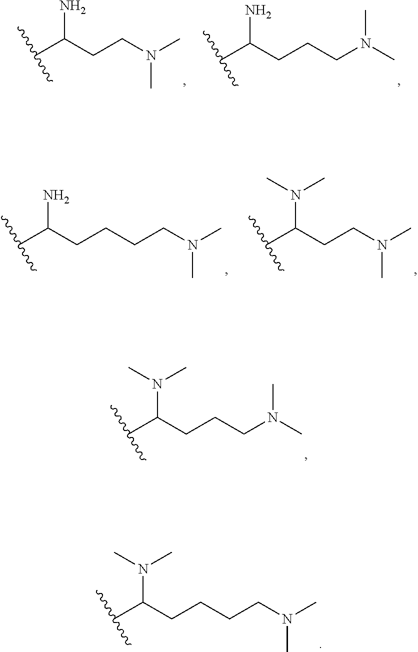

In some embodiments R³ in Formula (I) Formula (I*), Formula (Ia) or Formula (Ib is

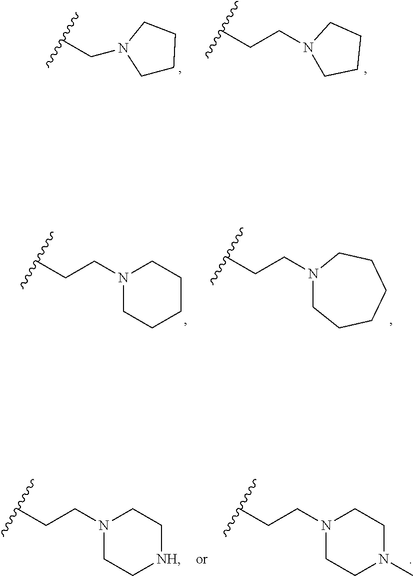

In some embodiments, R³ in Formula (I), Formula (I*), Formula (Ia), or Formula (Ib) is

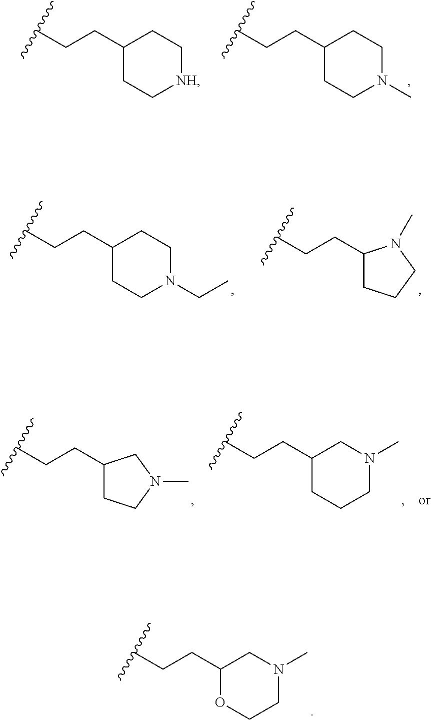

In some embodiments, R³ in Formula (I), Formula (I*), Formula (Ia), or Formula (Ib) is

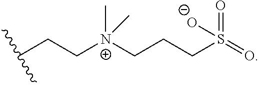 [SMILES: *CC[N+](C)(C)CCCS(=O)(=O)[O-]]

In some embodiments, Z in Formula (I), Formula (I*), Formula (Ia), or Formula (Ib) is —C(═O)O— or —OC(═O)—. In some embodiments, Z in Formula (I), Formula (I*), Formula (Ia), or Formula (Ib) is —C(═O)O—. In some embodiments, Z in Formula (I), Formula (I*), Formula (Ia), or Formula (Ib) is —OC(═O)—. In some embodiments, Z in Formula (I), Formula (I*), Formula (Ia), or Formula (Ib) is —C(═O)NR⁴— or —NR⁴C(═O)—. In some embodiments, Z in Formula (I), Formula (I*), Formula (Ia), or Formula (Ib) is —C(═O)NR⁴—. In some embodiments, Z in Formula (I), Formula (I*), Formula (Ia), or Formula (Ib) is —NR⁴C(═O)—. In some embodiments, R⁴ in Z is hydrogen or C₁-C₄ alkyl. In some embodiments, R⁴ in Z is hydrogen. In some embodiments, R⁴ in Z is C₁-C₄ alkyl. In some embodiments, Z in Formula (I), Formula (I*), Formula (Ia), or Formula (Ib) is —C(═O)N(CH₃)—, —N(CH₃)C(═O)—, —C(═O)NH—, or —NHC(═O)—. In some embodiments, Z in Formula (I), Formula (I*), Formula (Ia), or Formula (Ib) is —OC(═O)O—, —NR⁴C(═O)O—, —OC(═O)NR⁴—, or —NR⁴C(═O)NR⁴—. In some embodiments, Z in Formula (I), Formula (I*), Formula (Ia), or Formula (Ib) is —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)—, —NHC(═O)N(CH₃)— or —N(CH₃)C(═O)NH—. In some embodiments, Z in Formula (I), Formula (I*), Formula (Ia), or Formula (Ib) is —OC(═O)O—, —NHC(═O)O—, —OC(═O)NH—, or —NHC(═O)NH—. In some embodiments, Z in Formula (I), Formula (I*), Formula (Ia), or Formula (Ib) is a bond.

In some embodiments, R⁵ in Formula (I), Formula (I*), Formula (Ia), or Formula (Ib) is hydrogen or substituted or unsubstituted C₁-C₃ alkyl. In some embodiments, R⁵ in Formula (I), Formula (I*), Formula (Ia), or Formula (Ib) is H, —CH₃, —CH₂CH₃, —CH₂CH₂CH₃, or —CH(CH₃)₂. In some embodiments, R⁵ in Formula (I), Formula (I*), Formula (Ia), or Formula (Ib) is H. In some embodiments, R⁵ is substituted or unsubstituted —C₀-C₁₀ alkylene-L-R⁴. In some embodiments, R⁵ is substituted or unsubstituted C₁-C₁₆ alkyl.

In another aspect, disclosed herein is an amino lipid having structure of Formula (Ib), or a pharmaceutically acceptable salt thereof,

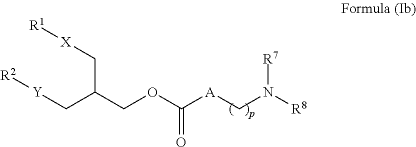

wherein
each of X and Y are the same as in Formulas (I), (I*) and (Ia) and are selected from the group consisting of: —OC(═O)—, —OC(═O)O—, —OC(═O)NR⁴—, —C(═S)O—, —OC(═S)—, OC(═S)O—, —NR⁴C(═S)O—, —OC(═S)NR⁴—, SC(═O)—, —OC(═O)S—, —SC(═S)O—, —O—, —C₁-C₁₀ alkylene-O—, and a bond, wherein the alkylene is substituted or unsubstituted
R¹ and R² are the same as in Formulas (I), (I*) and (Ia) and are selected from the group consisting of: hydrogen, C₁-C₃₀ alkyl, C₁-C₃₀ heteroalkyl, and Cy-C₃₋₃₀ alkyl, wherein the alkyl and heteroalkyl are each independently substituted or unsubstituted;
each of R⁴ is independently hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl; and
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl.

A is —O—, —CH₂—, —S—, or —NR¹²—.

R¹² is hydrogen, C₁-C₁₀ alkyl, or C₁-C₁₀ heteroalkyl, wherein the alkyl and heteroalkyl are each independently substituted or unsubstituted; and
Cy is substituted or unsubstituted cycloalkyl, substituted or unsubstituted heterocycloalkyl, substituted or unsubstituted aryl, or substituted or unsubstituted heteroaryl.
p is 1, 2, 3, 4, 5 or 6

In some embodiments, the amino lipid of Formula (I), Formula (I*), Formula (Ia), or Formula (Ib) is

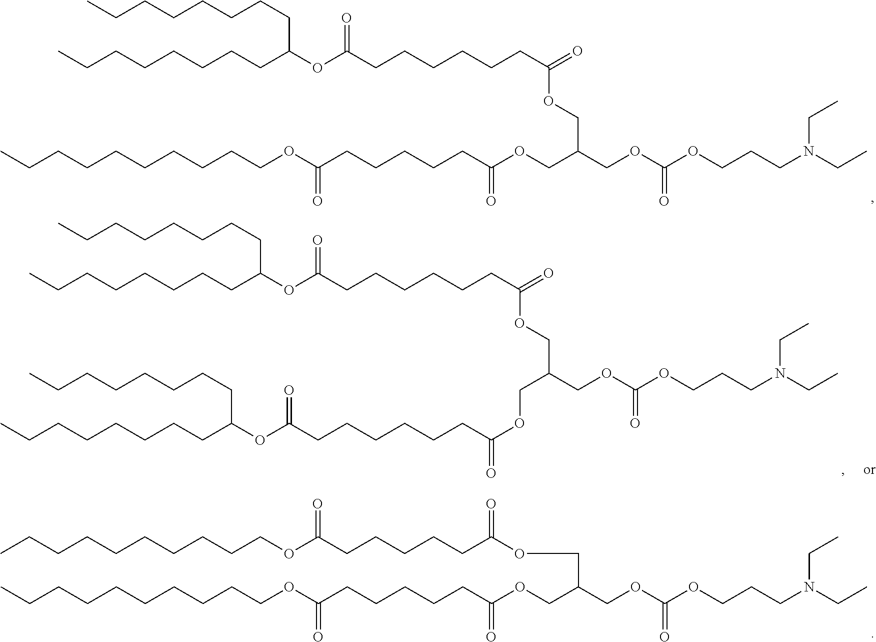

In another aspect, disclosed herein is an amino lipid having the structure of Formula (II), or a pharmaceutically acceptable salt or solvate thereof,

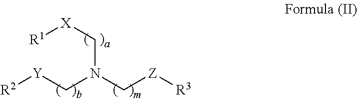

wherein
each of R¹ and R² is independently C₃-C₃₀ alkyl, C₃-C₃₀ alkenyl, C₃-C₃₀ alkynyl, C₃-C₁₀ cycloalkyl, —C₀-C₁₀ alkylene-L-R⁶, or —C₁-C₁₀ alkenylene-L-R⁶, wherein each of the alkyl, alkylene, alkenyl, alkenylene, alkynyl, and cycloalkyl is independently substituted or unsubstituted;
each of X, Y, and Z is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, or a bond;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is —C₀-C₁₀ alkylene-NR⁷R⁸, —C₀-C₁₀ alkylene-heterocycloalkyl, or —C₀-C₁₀ alkylene-heterocycloaryl, wherein the alkylene, heterocycloalkyl and heterocycloaryl is independently substituted or unsubstituted;
each of R⁴ is independently hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl;
each of a and b is independently 1, 2, 3, 4, 5, 6, 7, 8, 9, or 10; and
m is 0, 1, 2, 3, 4, or 5.

In one aspect, disclosed herein is an amino lipid having a structure of Formula (II*), or a pharmaceutically acceptable salt or solvate thereof,

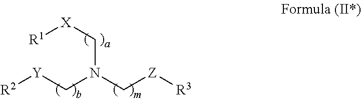

wherein
each of R¹ and R² is independently hydrogen, C₃-C₃₀ alkyl, C₃-C₃₀ alkenyl, C₃-C₃₀ alkynyl, C₃-C₃₀ heteroalkyl, C₃-C₃₀ heteroalkenyl, C₃-C₃₀ heteroalkynyl, C₃-C₁₀ cycloalkyl, —C₀-C₁₀ alkylene-L-R⁶, or —C₁-C₁₀ alkenylene-L-R⁶, wherein each of the alkyl, alkylene, alkenyl, alkenylene, alkynyl, heteroalkyl, heteroalkenyl, heteroalkynyl, and cycloalkyl is independently substituted or unsubstituted;
each of X, Y, and Z is independently —C(═O)NR⁴—, —C(═O)—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—, —S—, or a bond;
each of L is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O—N═CR⁴—, —CR⁴=N—O—, —O—, —S—, —C₁-C₁₀ alkylene-O—, —C₁-C₁₀ alkylene-C(═O)O—, —C₁-C₁₀ alkylene-OC(═O)—, or a bond, wherein the alkylene is substituted or unsubstituted;
R³ is hydrogen, —C₁-C₆ alkyl, —C₀-C₁₀ alkylene-NR⁷R⁸, —C₀-C₁₀ alkylene-heterocycloalkyl, or —C₀-C₁₀ alkylene-heterocycloaryl, wherein the alkyl, alkylene, heterocycloalkyl and heterocycloaryl is independently substituted or unsubstituted;
each of R⁴ is independently hydrogen or substituted or unsubstituted C₁-C₆ alkyl;
each of R⁶ is independently C₃-C₃₀ alkyl, C₃-C₃₀ alkenyl, C₃-C₃₀ alkynyl, Cy-C₃₋₃₀ alkyl, Cy-C₃₋₃₀ alkenyl, or Cy-C₃₋₃₀ alkynyl, wherein the alkyl, the alkenyl and alkynyl are each independently substituted or unsubstituted;
each of Cy is substituted or unsubstituted cycloalkyl, substituted or unsubstituted heterocycloalkyl, substituted or unsubstituted aryl, substituted or unsubstituted heteroaryl;
each of R⁷ and R⁸ is independently hydrogen or substituted or unsubstituted C₁-C₆ alkyl, or R⁷ and R⁸ taken together with the nitrogen to which they are attached form a substituted or unsubstituted C₂-C₆ heterocyclyl;
each of a and b is independently 1, 2, 3, 4, 5, 6, 7, 8, 9, or 10; and
m is 0, 1, 2, 3, 4, or 5.

In some embodiments of Formula (II) or (II*), if the structure carries more than one asymmetric C-atom, each asymmetric C-atom independently represents racemic, chirally pure R and/or chirally pure S isomer, or a combination thereof.

In some embodiments, each of a and b in Formula (II) or (II*) is independently 3, 4, 5, 6, 7, 8, 9, or 10. In some embodiments, a is 1. In some embodiments, a is 2. In some embodiments, a is 3. In some embodiments, a is 4. In some embodiments, a is 5. In some embodiments, b is 1. In some embodiments, b is 2. In some embodiments, b is 3. In some embodiments, b is 4. In some embodiments, b is 5.

In some embodiments, m in Formula (II) or (II*) is 1, 2, 3, 4, or 5. In some embodiments, m is 1. In some embodiments, m is 2. In some embodiments, m is 3. In some embodiments, m is 4. In some embodiments, m is 5.

In some embodiments, each R¹ and R² in Formula (II) or (II*) is independently C₅-C₂₈ alkyl, C₅-C₂₈ alkenyl, C₅-C₂₈ alkynyl, C₃-C₁₀ cycloalkyl, —C₁-C₁₀ alkylene-L-R⁶, or —C₁-C₁₀ alkenylene-L-R⁶, wherein each of the alkyl, alkylene, alkenyl, alkenylene, alkynyl, and cycloalkyl is independently substituted or unsubstituted. In some embodiments, each R¹ and R² in Formula (II) or (II*) is independently C₇-C₂₅ alkyl, C₇-C₂₅ alkenyl, C₇-C₂₅ alkynyl, C₃-C₁₀ cycloalkyl, —C₁-C₁₀ alkylene-L-R⁶, or —C₁-C₁₀ alkenylene-L-R⁶, wherein each of the alkyl, alkylene, alkenyl, alkenylene, alkynyl, and cycloalkyl is independently substituted or unsubstituted. In some embodiments, each R¹ and R² in Formula (II) or (II*) is independently C₇-C₂₂ alkyl, C₇-C₂₂ alkenyl, C₇-C₂₂ alkynyl, C₃-C₁₀ cycloalkyl, —C₁-C₁₀ alkylene-L-R⁶, or —C₁-C₁₀ alkenylene-L-R⁶, wherein each of the alkyl, alkylene, alkenyl, alkenylene, alkynyl, and cycloalkyl is independently substituted or unsubstituted. In some embodiments, each R¹ and R² in Formula (II) or (II*) is independently C₁₀-C₂₀ alkyl, C₁₀-C₂₀ alkenyl, —C₄-C₈ alkylene-L-R⁶, or —C₄-C₈ alkylene-L-R⁶, wherein each of the alkyl, alkylene, alkenyl, and cycloalkyl is independently substituted or unsubstituted. In some embodiments, each R¹ and R² in Formula (II) or (II*) is —C₁-C₁₀ alkylene-L-R⁶. In some embodiments, each R¹ and R² in Formula (II) or (II*) is C₇-C₃₀ alkenyl. In some embodiments, each R¹ and R² in Formula (II) or (II*) is C₇-C₂₈ alkenyl. In some embodiments, each R¹ and R² in Formula (II) or (II*) is C₇-C₂₅ alkenyl. In some embodiments, each R¹ and R² in Formula (II) or (II*) is C₇-C₂₂ alkenyl.

In some embodiments, each R¹ and R² in Formula (II) or (II*) is independently substituted or unsubstituted linear or branched C₃-C₃₀ alkyl or substituted or unsubstituted linear or branched C₃-C₃₀ alkenyl. In some embodiments, each R¹ and R² in Formula (II) or (II*) is independently substituted or unsubstituted linear or branched C₃-C₂₈ alkyl or substituted or unsubstituted linear or branched C₃-C₂₈ alkenyl. In some embodiments, each R¹ and R² in Formula (II) or (II*) is independently substituted or unsubstituted C₃-C₂₅ alkyl or substituted or unsubstituted C₃-C₂₅ alkenyl. In some embodiments, each R¹ and R² in Formula (II) or (II*) is independently substituted or unsubstituted C₃-C₂₂ alkyl or substituted or unsubstituted C₃-C₂₂ alkenyl. In some embodiments, each R¹ and R² in Formula (II) or (II*) is independently substituted or unsubstituted C₃-C₂₅ alkyl. In some embodiments, each R¹ and R² in Formula (II) or (II*) is independently C₃ alkyl, C₄ alkyl, C₅ alkyl, C₆ alkyl, C₇ alkyl, C₈ alkyl, C₉ alkyl, C₁₀ alkyl, C₁₁ alkyl, C₁₂ alkyl, C₁₃ alkyl, C₁₄ alkyl, C₁₅ alkyl, C₁₆ alkyl, C₁₇ alkyl, C₁₈ alkyl, C₁₉ alkyl, C₂₀ alkyl, C₂₁ alkyl, C₂₂ alkyl, C₂₃ alkyl, C₂₄ alkyl, or C₂₅ alkyl. In some embodiments, each R¹ and R² in Formula (II) or (II*) is independently at least C₃ alkyl, at least C₄ alkyl, at least C₅ alkyl, at least C₆ alkyl, at least C₇ alkyl, at least C₈ alkyl, at least C₉ alkyl, at least C₁₀ alkyl, at least C₁₁ alkyl, at least C₁₂ alkyl, at least C₁₃ alkyl, at least C₁₄ alkyl, at least C₁₅ alkyl, at least C₁₆ alkyl, at least C₁₇ alkyl, at least C₁₈ alkyl, at least C₁₉ alkyl, at least C₂₀ alkyl, at least C₂₁ alkyl, at least C₂₂ alkyl, at least C₂₃ alkyl, at least C₂₄ alkyl, or at least C₂₅ alkyl. In some embodiments, each R¹ and R² in Formula (II) or (II*) is independently at most C₃ alkyl, at most C₄ alkyl, at most C₅ alkyl, at most C₆ alkyl, at most C₇ alkyl, at most C₈ alkyl, at most C₉ alkyl, at most C₁₀ alkyl, at most C₁₁ alkyl, at most C₁₂ alkyl, at most C₁₃ alkyl, at most C₁₄ alkyl, at most C₁₅ alkyl, at most C₁₆ alkyl, at most C₁₇ alkyl, at most C₁₈ alkyl, at most C₁₉ alkyl, at most C₂₀ alkyl, at most C₂₁ alkyl, at most C₂₂ alkyl, at most C₂₃ alkyl, at most C₂₄ alkyl, or at most C₂₅ alkyl. In some embodiments, each R¹ and R² in Formula (II) or (II*) is independently substituted or unsubstituted C₃-C₂₅ alkenyl. In some embodiments, each R¹ and R² in Formula (II) or (II*) is independently C₃ alkenyl, C₄ alkenyl, C₅ alkenyl, C₆ alkenyl, C₇ alkenyl, C₈ alkenyl, C₉ alkenyl, C₁₀ alkenyl, C₁₁ alkenyl, C₁₂ alkenyl, C₁₃ alkenyl, C₁₄ alkenyl, C₁₅ alkenyl, C₁₆ alkenyl, C₁₇ alkenyl, C₁₈ alkenyl, C₁₉ alkenyl, C₂₀ alkenyl, C₂₁ alkenyl, C₂₂ alkenyl, C₂₃ alkenyl, C₂₄ alkenyl, or C₂₅ alkenyl. In some embodiments, each R¹ and R² in Formula (II) or (II*) is independently at least C₃ alkenyl, at least C₄ alkenyl, at least C₅ alkenyl, at least C₆ alkenyl, at least C₇ alkenyl, at least C₈ alkenyl, at least C₉ alkenyl, at least C₁₀ alkenyl, at least C_{II} alkenyl, at least C₁₂ alkenyl, at least C₁₃ alkenyl, at least C₁₄ alkenyl, at least Cis alkenyl, at least C₁₆ alkenyl, at least C₁₇ alkenyl, at least C₁₈ alkenyl, at least C₁₉ alkenyl, at least C₂₀ alkenyl, at least C₂₁ alkenyl, at least C₂₂ alkenyl, at least C₂₃ alkenyl, at least C₂₄ alkenyl, or at least C₂₅ alkenyl. In some embodiments, each R¹ and R² in Formula (II) or (II*) is independently at most C₃ alkenyl, at most C₄ alkenyl, at most C₅ alkenyl, at most C₆ alkenyl, at most C₇ alkenyl, at most C₈ alkenyl, at most C₉ alkenyl, at most C₁₀ alkenyl, at most C₁₁ alkenyl, at most C₁₂ alkenyl, at most C₁₃ alkenyl, at most C₁₄ alkenyl, at most Cis alkenyl, at most C₁₆ alkenyl, at most C₁₇ alkenyl, at most C₁₈ alkenyl, at most C₁₉ alkenyl, at most C₂₀ alkenyl, at most C₂₁ alkenyl, at most C₂₂ alkenyl, at most C₂₃ alkenyl, at most C₂₄ alkenyl, or at most C₂₅ alkenyl.

In some embodiments, R¹ and/or R² in Formula (II) or (II*) is independently

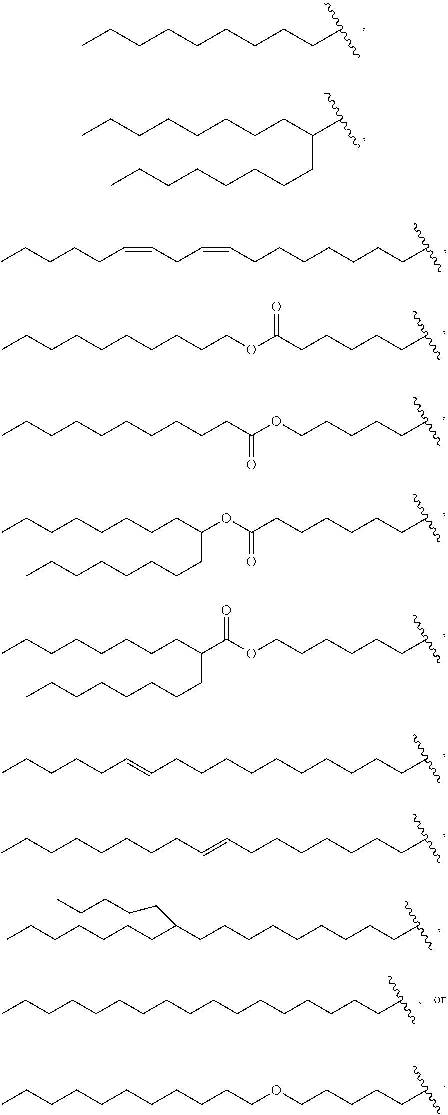

In some embodiments, R¹ and/or R² in Formula (II) or (II*) is

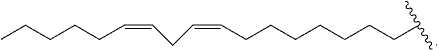 [SMILES: *CCCCCCC/C=C\C/C=C\CCCCC]

In some embodiments, R¹ and/or R² in Formula (II) or (II*) is

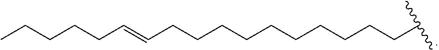 [SMILES: *CCCCCCCCCC/C=C/CCCCC]

In some embodiments, R¹ and/or R² in Formula (II) or (II*) is

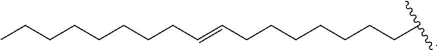 [SMILES: *CCCCCCC/C=C/CCCCCCCC]

In some embodiments, R¹ and/or R² in Formula (II) or (II*) is

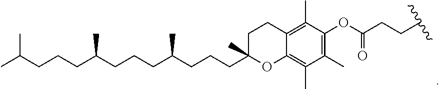 [SMILES: *CCC(=O)Oc1c(C)c(C)c2c(c1C)CC[C@@](C)(CCC[C@H](C)CCC[C@H](C)CCCC(C)C)O2]

In some embodiments, R¹ in Formula (II) or (II*) is

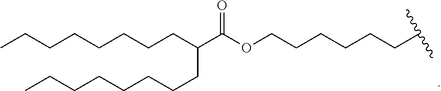 [SMILES: *CCCCCCOC(=O)C(CCCCCCCC)CCCCCCCC]

In some embodiments, R² in Formula (II) or (II*) is

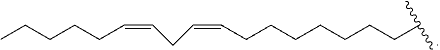 [SMILES: *CCCCCCC/C=C\C/C=C\CCCCC]

In some embodiments, R¹ in Formula (II) or (II*) is

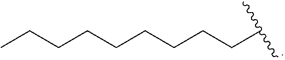 [SMILES: *CCCCCCCCC]

In some embodiments, R² in Formula (II) or (II*) is

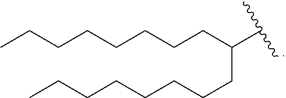 [SMILES: *C(CCCCCCCC)CCCCCCCC]

In some embodiments, each of L in Formula (II) or (II*) is independently —O—, —S—, —C₁-C₁₀ alkylene-O—, —C₁-C₁₀ alkylene-C(═O)O—, —C₁-C₁₀ alkylene-OC(═O)—, —C(═O)O—, —OC(═O)—, —C(═O)NR⁴—, —NR⁴C(═O)—, or a bond, wherein the alkylene is substituted or unsubstituted. In some embodiments, at least one Lin Formula (II) or (II*) is independently —O—, —S—, —C₁-C₃ alkylene-O—, —C₁-C₃ alkylene-C(═O)O—, —C₁-C₃ alkylene-OC(═O)—, —C(═O)O—, —OC(═O)—, —C(═O)NR⁴—, —NR⁴C(═O)—, or a bond, wherein the alkylene is substituted or unsubstituted. In some embodiments, at least one L in Formula (II) or (II*) is independently —O—, —S—, —C₁-C₃ alkylene-O—, —C₁-C₃ alkylene-C(═O)O—, —C₁-C₃ alkylene-OC(═O)—, or a bond, wherein the alkylene is linear or branched unsubstituted alkylene. In some embodiments, at least one L in Formula (II) or (II*) is —C(═O)O—, —OC(═O)—, —C(═O)NR⁴—, —NR⁴C(═O)—. In some embodiments, at least one Lin Formula (II) or (II*) is —C(═O)O—. In some embodiments, at least one Lin Formula (II) or (II*) is —OC(═O)—. In some embodiments, at least one L in Formula (II) or (II*) is —C(═O)NR⁴—. In some embodiments, at least one L in Formula (II) or (II*) is —C(═O)NH—. In some embodiments, at least one L in Formula (II) or (II*) is —C(═O)N(CH₃)—. In some embodiments, at least one Lin Formula (II) or (II*) is —NR⁴C(═O)—. In some embodiments, at least one L in Formula (II) or (II*) is —NHC(═O)—. In some embodiments, at least one L in Formula (II) or (II*) is —N(CH₃)C(═O)—.

In some embodiments, at least one R⁶ in Formula (II) or (II*) is independently substituted or unsubstituted linear or branched C₃-C₃₀ alkyl or substituted or unsubstituted linear or branched C₃-C₃₀ alkenyl. In some embodiments, each of R⁶ in Formula (II) or (II*) is independently substituted or unsubstituted linear or branched C₃-C₂₈ alkyl or substituted or unsubstituted linear or branched C₃-C₂₈ alkenyl. In some embodiments, each of R⁶ in Formula (II) or (II*) is independently substituted or unsubstituted C₃-C₂₅ alkyl or substituted or unsubstituted C₃-C₂₅ alkenyl. In some embodiments, at least one R⁶ in Formula (II) or (II*) is independently substituted or unsubstituted C₃-C₂₅ alkyl. In some embodiments, at least one R⁶ in Formula (II) or (II*) is independently C₃ alkyl, C₄ alkyl, C₅ alkyl, C₆ alkyl, C₇ alkyl, C₈ alkyl, C₉ alkyl, C₁₀ alkyl, C₁₁ alkyl, C₁₂ alkyl, C₁₃ alkyl, C₁₄ alkyl, C₁₅ alkyl, C₁₆ alkyl, C₁₇ alkyl, C₁₈ alkyl, C₁₉ alkyl, C₂₀ alkyl, C₂₁ alkyl, C₂₂ alkyl, C₂₃ alkyl, C₂₄ alkyl, or C₂₅ alkyl. In some embodiments, at least one R⁶ in Formula (II) or (II*) is independently at least C₃ alkyl, at least C₄ alkyl, at least C₅ alkyl, at least C₆ alkyl, at least C₇ alkyl, at least C₈ alkyl, at least C₉ alkyl, at least C₁₀ alkyl, at least C₁₁ alkyl, at least C₁₂ alkyl, at least C₁₃ alkyl, at least C₁₄ alkyl, at least C₁₅ alkyl, at least C₁₆ alkyl, at least C₁₇ alkyl, at least C₁₈ alkyl, at least C₁₉ alkyl, at least C₂₀ alkyl, at least C₂₁ alkyl, at least C₂₂ alkyl, at least C₂₃ alkyl, at least C₂₄ alkyl, or at least C₂₅ alkyl. In some embodiments, at least one R⁶ in Formula (II) or (II*) is independently at most C₃ alkyl, at most C₄ alkyl, at most C₅ alkyl, at most C₆ alkyl, at most C₇ alkyl, at most C₈ alkyl, at most C₉ alkyl, at most C₁₀ alkyl, at most C₁₁ alkyl, at most C₁₂ alkyl, at most C₁₃ alkyl, at most C₁₄ alkyl, at most C₁₅ alkyl, at most C₁₆ alkyl, at most C₁₇ alkyl, at most C₁₈ alkyl, at most C₁₉ alkyl, at most C₂₀ alkyl, at most C₂₁ alkyl, at most C₂₂ alkyl, at most C₂₃ alkyl, at most C₂₄ alkyl, or at most C₂₅ alkyl. In some embodiments, at least one R⁶ in Formula (II) or (II*) is independently substituted or unsubstituted C₃-C₂₅ alkenyl. In some embodiments, at least one R⁶ in Formula (II) or (II*) is independently C₃ alkenyl, C₄ alkenyl, C₅ alkenyl, C₆ alkenyl, C₇ alkenyl, C₈ alkenyl, C₉ alkenyl, C₁₀ alkenyl, C₁₁ alkenyl, C₁₂ alkenyl, C₁₃ alkenyl, C₁₄ alkenyl, C₁₅ alkenyl, C₁₆ alkenyl, C₁₇ alkenyl, C₁₈ alkenyl, C₁₉ alkenyl, C₂₀ alkenyl, C₂₁ alkenyl, C₂₂ alkenyl, C₂₃ alkenyl, C₂₄ alkenyl, or C₂₅ alkenyl. In some embodiments, at least one R⁶ in Formula (II) or (II*) is independently at least C₃ alkenyl, at least C₄ alkenyl, at least C₅ alkenyl, at least C₆ alkenyl, at least C₇ alkenyl, at least C₈ alkenyl, at least C₉ alkenyl, at least C₁₀ alkenyl, at least C₁₁ alkenyl, at least C₁₂ alkenyl, at least C₁₃ alkenyl, at least C₁₄ alkenyl, at least Cis alkenyl, at least C₁₆ alkenyl, at least C₁₇ alkenyl, at least C₁₈ alkenyl, at least C₁₉ alkenyl, at least C₂₀ alkenyl, at least C₂₁ alkenyl, at least C₂₂ alkenyl, at least C₂₃ alkenyl, at least C₂₄ alkenyl, or at least C₂₅ alkenyl. In some embodiments, at least one R⁶ in Formula (II) or (II*) is independently at most C₃ alkenyl, at most C₄ alkenyl, at most C₅ alkenyl, at most C₆ alkenyl, at most C₇ alkenyl, at most C₈ alkenyl, at most C₉ alkenyl, at most C₁₀ alkenyl, at most C₁₁ alkenyl, at most C₁₂ alkenyl, at most C₁₃ alkenyl, at most C₁₄ alkenyl, at most C₁₅ alkenyl, at most C₁₆ alkenyl, at most C₁₇ alkenyl, at most C₁₈ alkenyl, at most C₁₉ alkenyl, at most C₂₀ alkenyl, at most C₂₁ alkenyl, at most C₂₂ alkenyl, at most C₂₃ alkenyl, at most C₂₄ alkenyl, or at most C₂₅ alkenyl.

In some embodiments, each R⁶ in Formula (II) or (II*) is independently

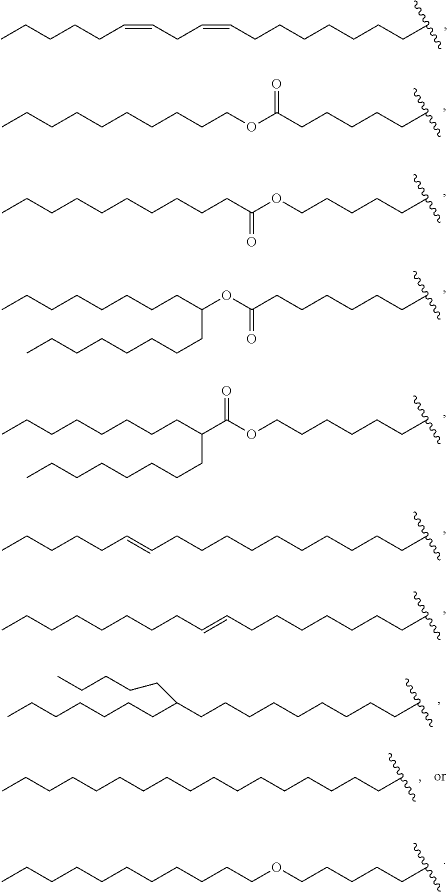

In some embodiments, each R⁶ in Formula (II) or (II*) is

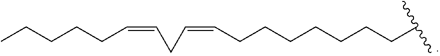 [SMILES: *CCCCCCC/C=C\C/C=C\CCCCC]

In some embodiments, each R⁶ in Formula (II) or (II*) is

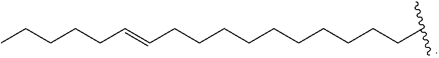 [SMILES: *CCCCCCCCCC/C=C/CCCCC]

In some embodiments, each R⁶ in Formula (II) or (II*) is

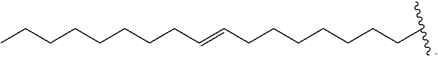 [SMILES: *CCCCCCC/C=C/CCCCCCCC]

In some embodiments, R⁴ in Formula (II) or (II*) is independently H or substituted or unsubstituted C₁-C₄ alkyl. In some embodiments, at least one R⁴ in Formula (II) or (II*) is independently substituted or unsubstituted linear C₁-C₄ alkyl. In some embodiments, at least one R⁴ in Formula (II) or (II*) is H. In some embodiments, at least one R⁴ in Formula (II) or (II*) is independently H, —CH₃, —CH₂CH₃, —CH₂CH₂CH₃, or —CH(CH₃)₂. In some embodiments, at least one R⁴ in Formula (II) or (II*) is independently H or —CH₃. In some embodiments, at least one R⁴ in Formula (II) or (II*) is —CH₃.

In some embodiments, each X and Y in Formula (II) or (II*) is independently —C(═O)O— or —OC(═O)—. In some embodiments, each X and Y in Formula (II) or (II*) is —C(═O)O—. In some embodiments, each X and Y in Formula (II) or (II*) is —OC(═O)—. In some embodiments, Y in Formula (II) or (II*) is —C(═O)O—. In some embodiments, Y in Formula (II) or (II*) is —OC(═O)—. In some embodiments, each X and Y in Formula (II) or (II*) is independently —C(═O)NR⁴— or —NR⁴C(═O)—. In some embodiments, X in Formula (II) or (II*) is —C(═O)NR⁴— or —NR⁴C(═O)—. In some embodiments, X in Formula (II) or (II*) is —C(═O)NR⁴—. In some embodiments, X in Formula (II) or (II*) is —NR⁴C(═O)—. In some embodiments, each X and Y in Formula (II) or (II*) is independently —C(═O)N(CH₃)—, —N(CH₃)C(═O)—, —C(═O)NH—, or —NHC(═O)—. In some embodiments, each X and Y in Formula (II) or (II*) is independently —C(═O)NH—, —C(═O)N(CH₃)—. —OC(═O)—, —NHC(═O)—, —N(CH₃)C(═O)—, —C(═O)O—, —OC(═O)O—, —NHC(═O)O—, —N(CH₃)C(═O)O—, —OC(═O)NH—, —OC(═O)N(CH₃)—, —NHC(═O)NH—, —N(CH₃)C(═O)NH—, —NHC(═O)N(CH₃)—, —N(CH₃)C(═O)N(CH₃)—, NHC(═NH)NH—, —N(CH₃)C(═NH)NH—, —NHC(═NH)N(CH₃)—, —N(CH₃)C(═NH)N(CH₃)—, NHC(═NMe)NH—, —N(CH₃)C(═NMe)NH—, —NHC(═NMe)N(CH₃)—, or —N(CH₃)C(═NMe)N(CH₃)—. In some embodiments, each X and Y in Formula (II) or (II*) is independently —OC(═O)O—, —NR⁴C(═O)O—, —OC(═O)NR⁴—, or —NR⁴C(═O)NR⁴—. In some embodiments, each X and Y in Formula (II) or (II*) is independently —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)— or —N(CH₃)C(═O)NH—. In some embodiments, each X and Y in Formula (II) or (II*) is independently —OC(═O)O—, —NHC(═O)O—, —OC(═O)NH—, or —NHC(═O)NH—.

In some embodiments, R³ in Formula (II) or (II*) is —C₀-C₁₀ alkylene-NR⁷R⁸ or —C₀-C₁₀ alkylene-heterocycloalkyl, wherein the alkylene and heterocycloalkyl is independently substituted or unsubstituted. In some embodiments, R³ in Formula (II) or (II*) is —C₀-C₁₀ alkylene-NR⁷R⁸. In some embodiments, R³ in Formula (II) or (II*) is —C₁-C₆ alkylene-NR⁷R⁸. In some embodiments, R³ in Formula (II) or (II*) is —C₁-C₄ alkylene-NR⁷R⁸. In some embodiments, R³ in Formula (II) or (II*) is —C₁-alkylene-NR⁷R⁸. In some embodiments, R³ in Formula (II) or (II*) is —C₂-alkylene-NR⁷R⁸. In some embodiments, R³ in Formula (II) or (II*) is —C₃-alkylene-NR⁷R⁸. In some embodiments, R³ in Formula (II) or (II*) is —C₄-alkylene-NR⁷R⁸. In some embodiments, R³ in Formula (II) or (II*) is —C₅-alkylene-NR⁷R⁸. In some embodiments, R³ in Formula (II) or (II*) is —C₀-C₁₀ alkylene-heterocycloalkyl. In some embodiments, R³ in Formula (II) or (II*) is —C₁-C₆ alkylene-heterocycloalkyl, wherein the heterocycloalkyl comprises 1 to 3 nitrogen and 0-2 oxygen. In some embodiments, R³ in Formula (II) or (II*) is quaternized.

In some embodiments, R⁷ and R⁸ in Formula (II) or (II*) is independently hydrogen or substituted or unsubstituted C₁-C₆ alkyl. In some embodiments, at least one R⁷ and R⁸ in Formula (II) or (II*) is independently substituted or unsubstituted C₁-C₆ alkyl. In some embodiments, at least one R⁷ and R⁸ is independently hydrogen or substituted or unsubstituted C₁-C₃ alkyl. In some embodiments, at least one R⁷ and R⁸ is independently substituted or unsubstituted C₁-C₃ alkyl. In some embodiments, at least one R⁷ and R⁸ is independently —CH₃, —CH₂CH₃, —CH₂CH₂CH₃, or —CH(CH₃)₂. In some embodiments, at least one R⁷ and R⁸ is CH₃. In some embodiments, at least one R⁷ and R⁸ is —CH₂CH₃.

In some embodiments, R⁷ and R⁸ in Formula (II) or (II*), taken together with the nitrogen to which they are attached, form a substituted or unsubstituted C₂-C₆ heterocyclyl. In some embodiments, R⁷ and R⁸ taken together with the nitrogen to which they are attached form a substituted or unsubstituted C₂-C₆ heterocycloalkyl. In some embodiments, R⁷ and R⁸ taken together with the nitrogen to which they are attached form a substituted or unsubstituted 3-7 membered heterocycloalkyl.

In some embodiments, R³ in Formula (II) or (II*) is

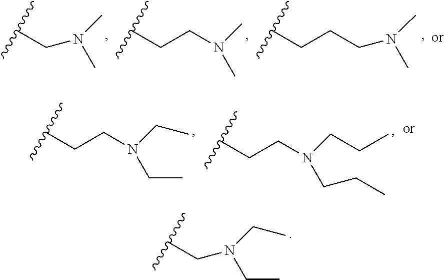

In some embodiments, R³ in Formula (II) or (II*) is

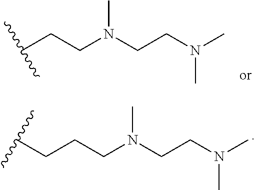

In some embodiments, R³ in Formula (II) or (II*) is

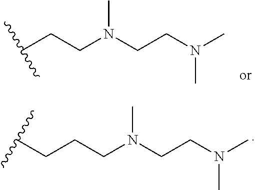

In some embodiments, R³ in Formula (II) or (II*) is

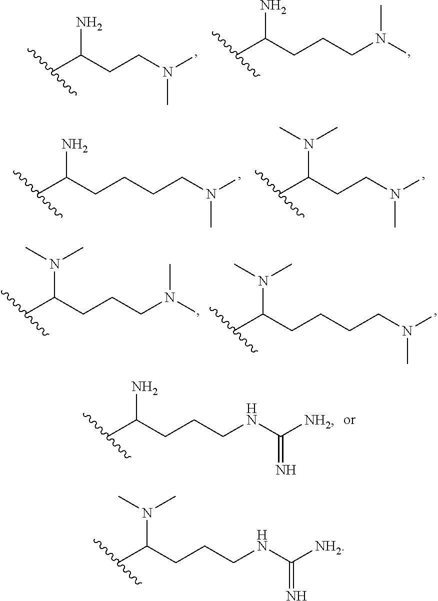

In some embodiments, R³ in Formula (II) or (II*) is

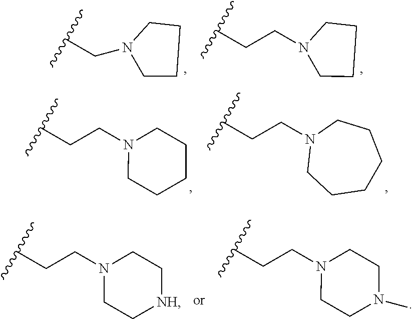

In some embodiments, R³ in Formula (II) or (II*) is

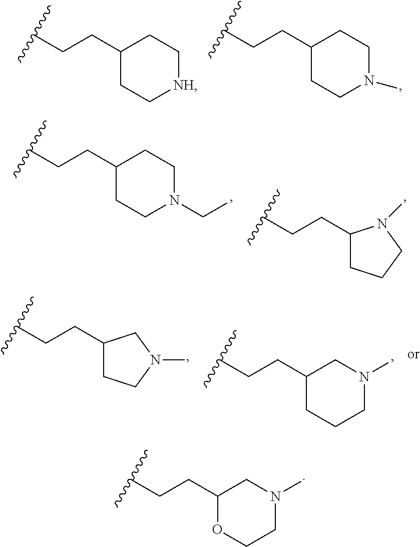

In some embodiments, R³ in Formula (II) or (II*) is

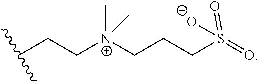 [SMILES: *CC[N+](C)(C)CCCS(=O)(=O)[O-]]

In some embodiments, Z in Formula (II) or (II*) is —C(═O)O— or —OC(═O)—. In some embodiments, Z in Formula (II) or (II*) is —C(═O)O—. In some embodiments, Z in Formula (II) or (II*) is —OC(═O)—. In some embodiments, Z in Formula (II) or (II*) is —C(═O)NR⁴— or —NR⁴C(═O)—. In some embodiments, Z in Formula (II) or (II*) is —C(═O)NR⁴—. In some embodiments, Z in Formula (II) or (II*) is —NR⁴C(═O)—. In some embodiments, R⁴ in Z is hydrogen or C₁-C₄ alkyl. In some embodiments, R⁴ in Z is hydrogen. In some embodiments, R⁴ in Z is C₁-C₄ alkyl. In some embodiments, Z in Formula (II) or (II*) is —C(═O)N(CH₃)—, —N(CH₃)C(═O)—, —C(═O)NH—, or —NHC(═O)—. In some embodiments, Z in Formula (II) or (II*) is —OC(═O)O—, —NR⁴C(═O)O—, —OC(═O)NR⁴—, or —NR⁴C(═O)NR⁴—. In some embodiments, Z in Formula (II) or (II*) is —OC(═O)O—, —NHC(═O)O—, —OC(═O)NH—, —NHC(═O)NH—, —N(CH₃)C(═O)O—, —OC(═O)N(CH₃)—, —N(CH₃)C(═O)N(CH₃)—, —NHC(═O)N(CH₃)— or —N(CH₃)C(═O)NH—. In some embodiments, Z in Formula (II) or (II*) is —OC(═O)O—, —NHC(═O)O—, —OC(═O)NH—, or —NHC(═O)NH—.

In one aspect, disclosed herein is an amino lipid of Table 1A, or a salt or solvate thereof.

TABLE 1A
Amino lipids for constituting lipid nanoparticles. The chiral carbon atom(s) in any of the
structures indicate(s) racemic, and/or chirally pure R and S stereoisomers. 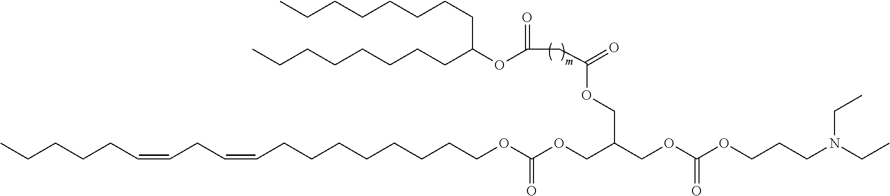
m = 1-10 VL011-VL020 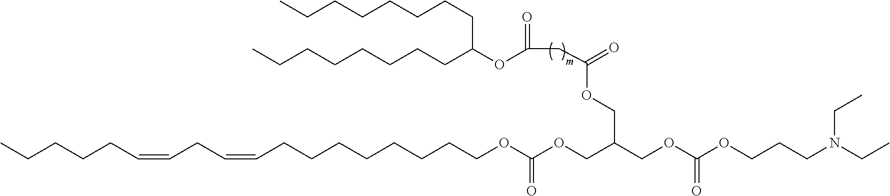
m = 1-10 VL021-VL030 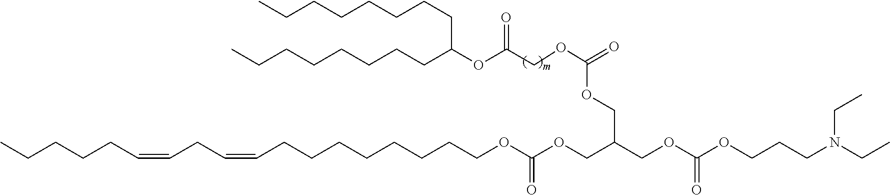
m = 1-10 VL031-VL040 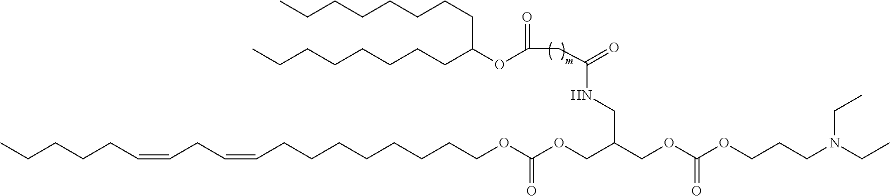
m = 1-10 VL041-VL050 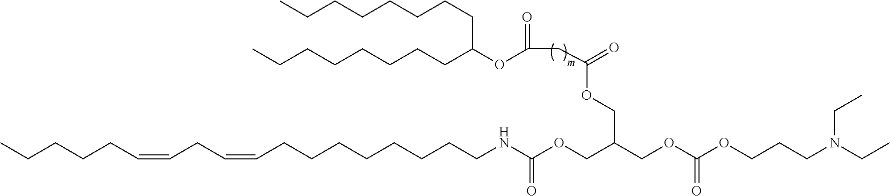
m = 1-10 VL051-VL060 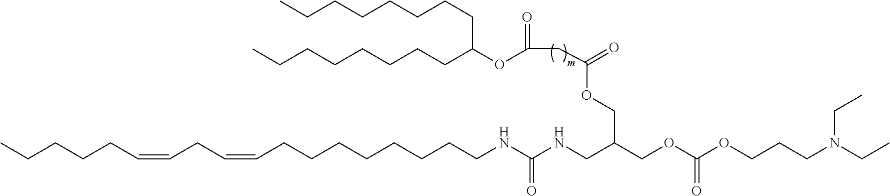
m = 1-10 VL061-VL070 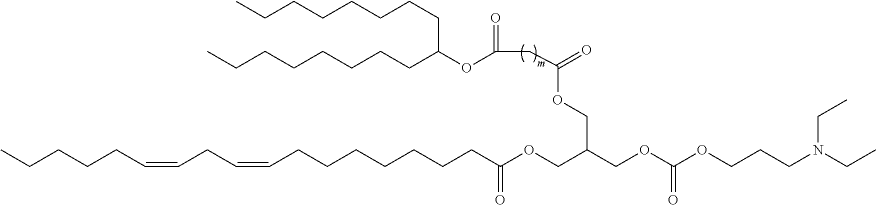
m = 1-10 VL071-VL080 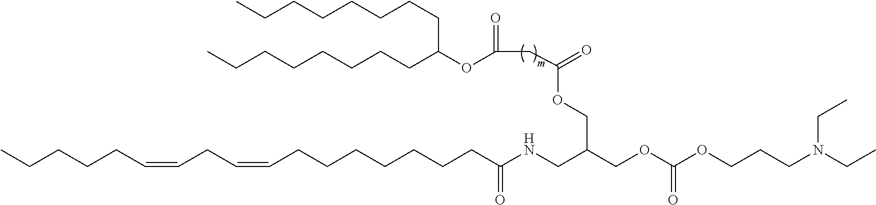
m = 1-10 VL081-VL090 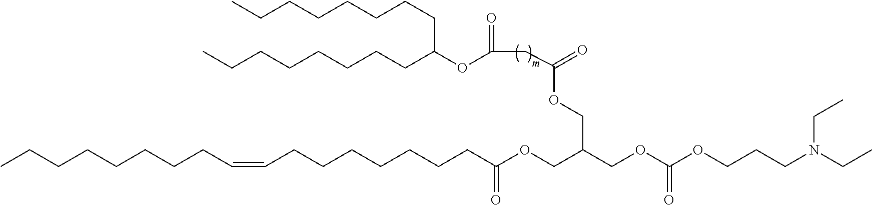
m = 1-10 VL091-VL100 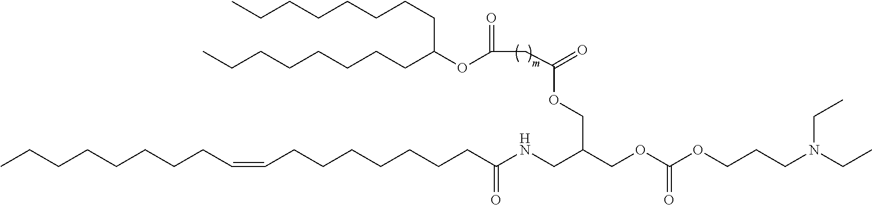
m = 1-10 VL101-VL110 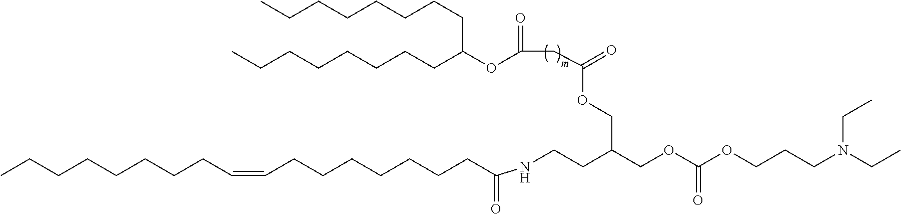
m = 1-10 VL111-VL120 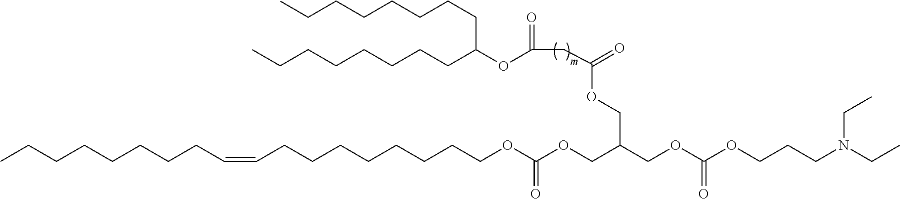
m = 1-10 VL121-VL130 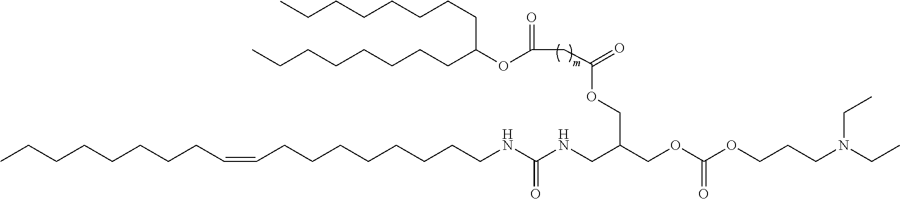
m = 1-10 VL131-VL140 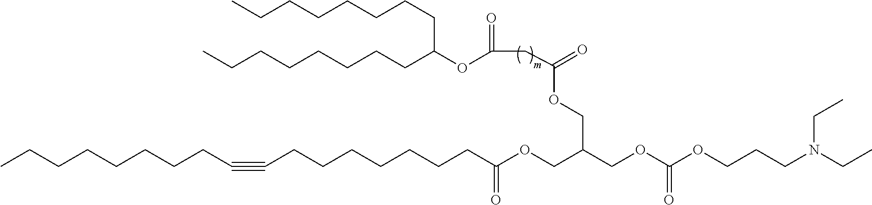
m = 1-10 VL141-VL150 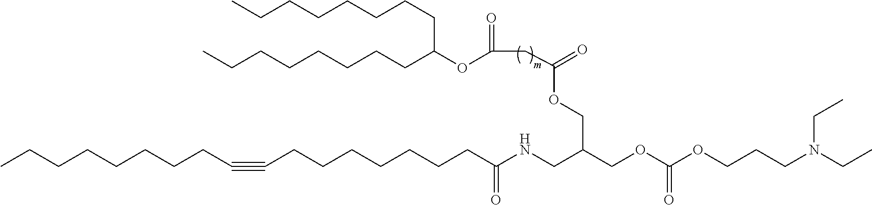
m = 1-10 VL151-VL160 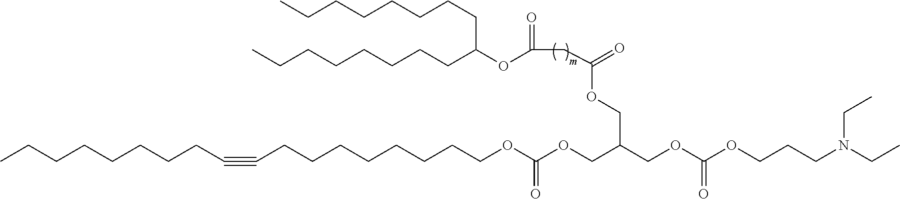
m = 1-10 VL161-VL170 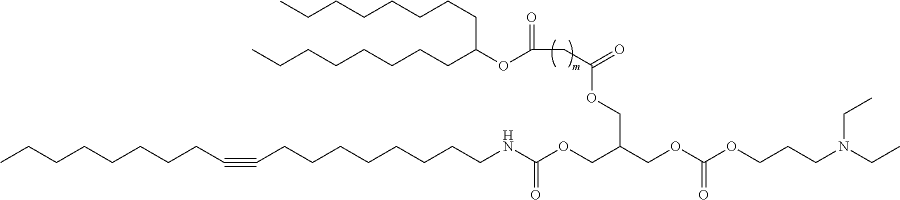
m = 1-10 VL171-VL180 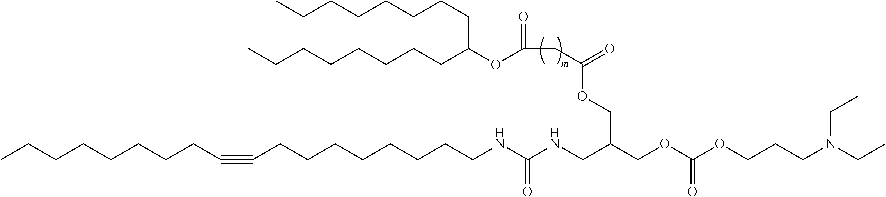
m = 1-10 VL181-VL190 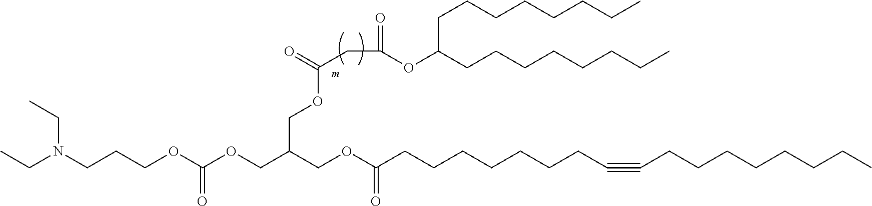
m = 1-10 VL191-VL200 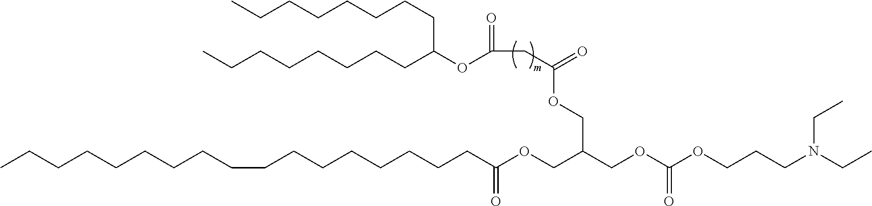
m = 1-10 VL201-VL210 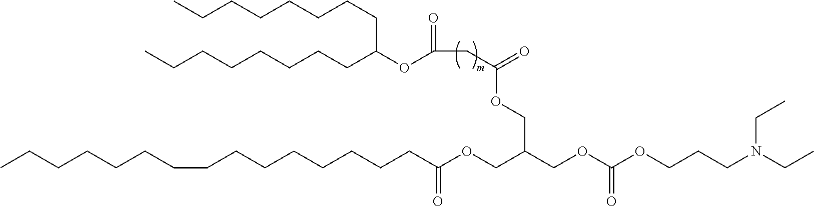
m = 1-10 VL211-VL220 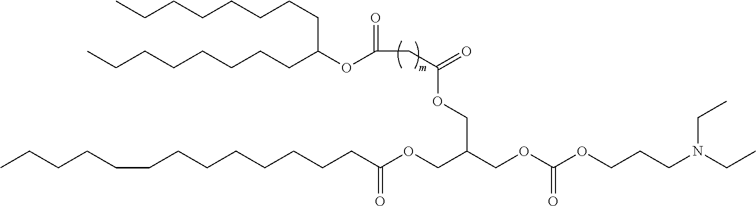
m = 1-10 VL221-VL230 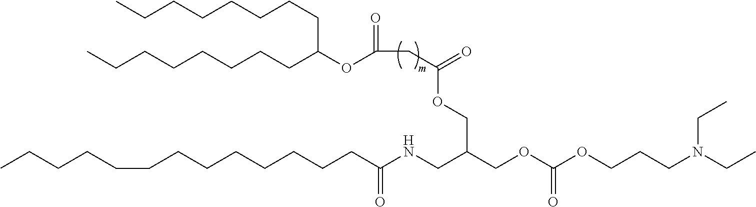
m = 1-10 VL251-260 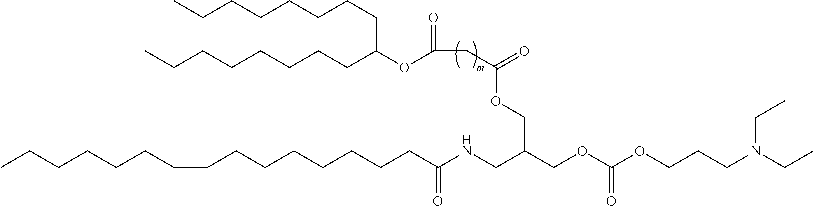
m = 1-10 VL261-270 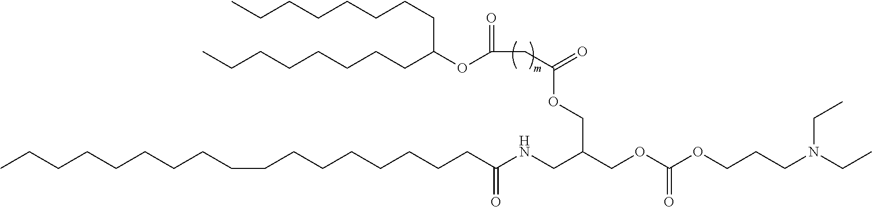
m = 1-10 VL271-280 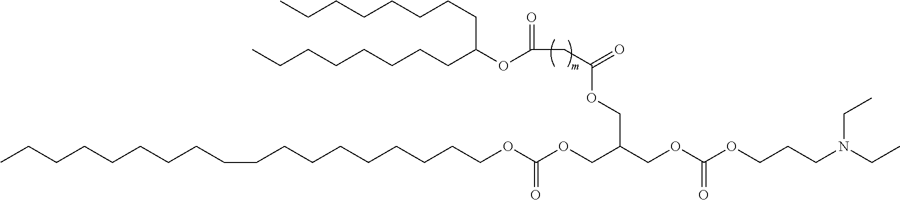
m = 1-10 VL281-290 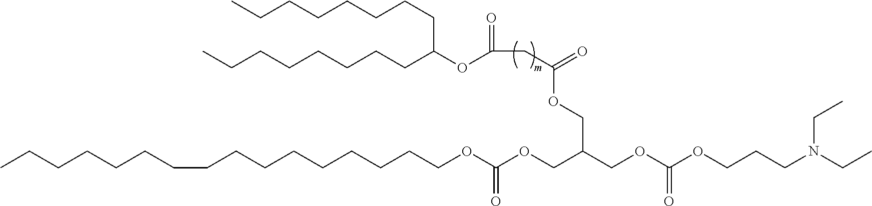
m = 1-10 VL291-VL300 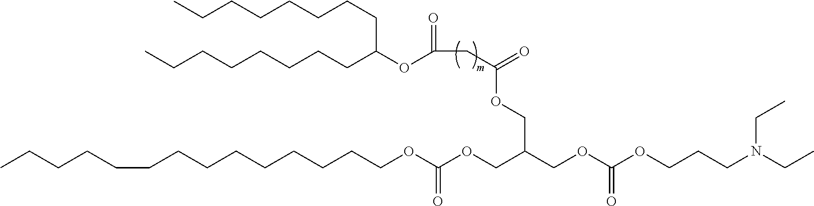
m = 1-10 VL301-VL310 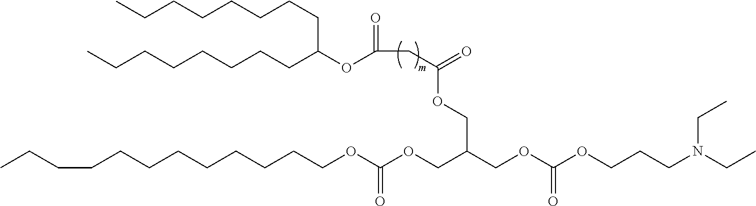
m = 1-10 VL311-VL320 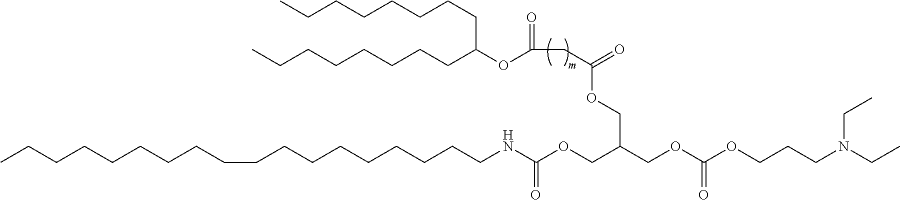
m = 1-10 VL321-VL330 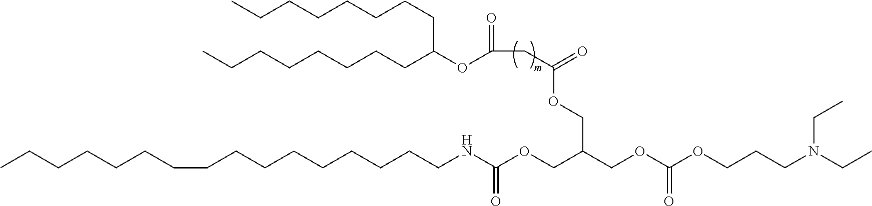
m = 1-10 VL331-VL340 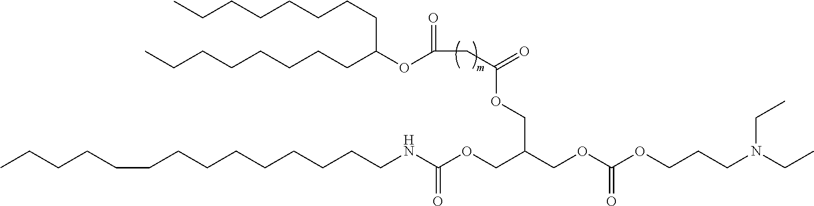
m = 1-10 VL341-VL350 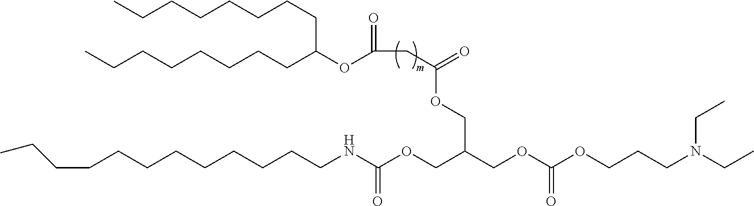
m = 1-10 VL351-VL360 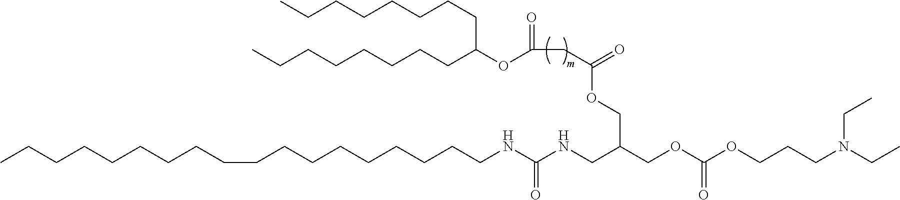
m = 1-10 VL361-VL370 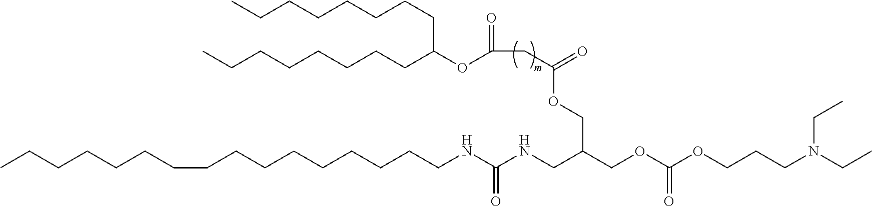
m = 1-10 VL371-VL380 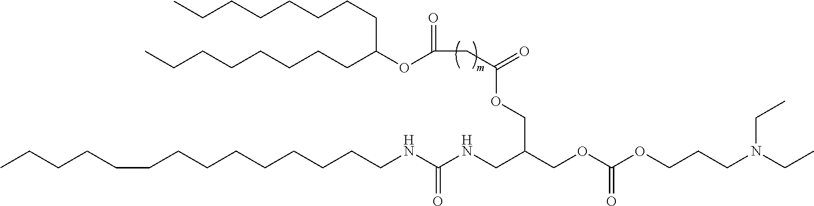
m = 1-10 VL381-VL390 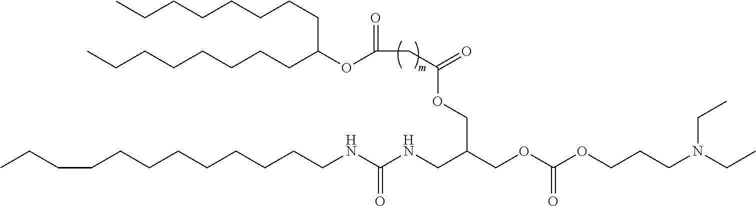
m = 1-10 VL391-VL400 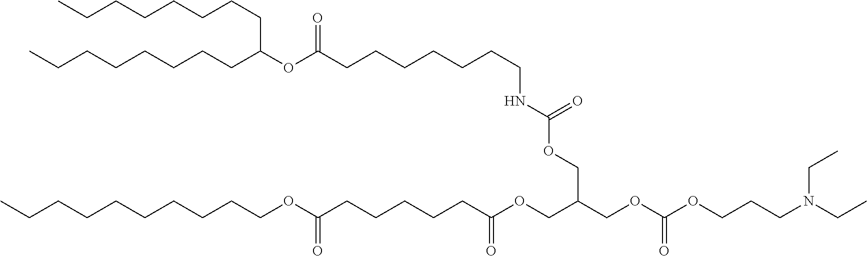 [SMILES: CCCCCCCCCCOC(=O)CCCCCC(=O)OCC(COC(=O)NCCCCCCCC(=O)OC(CCCCCCCC)CCCCCCCC)COC(=O)OCCCN(CC)CC]
VL401 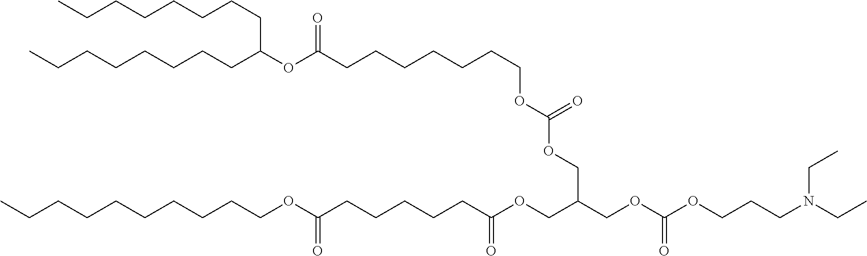 [SMILES: CCCCCCCCCCOC(=O)CCCCCC(=O)OCC(COC(=O)OCCCCCCCC(=O)OC(CCCCCCCC)CCCCCCCC)COC(=O)OCCCN(CC)CC]
VL402 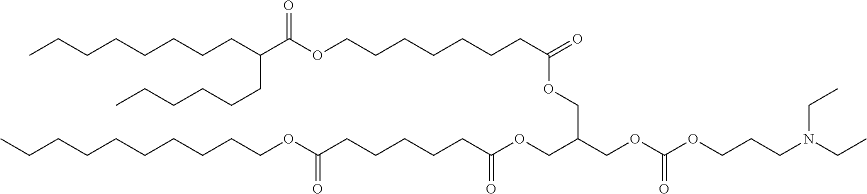 [SMILES: CCCCCCCCCCOC(=O)CCCCCC(=O)OCC(COC(=O)CCCCCCCOC(=O)C(CCCCCC)CCCCCCCC)COC(=O)OCCCN(CC)CC]
VL403 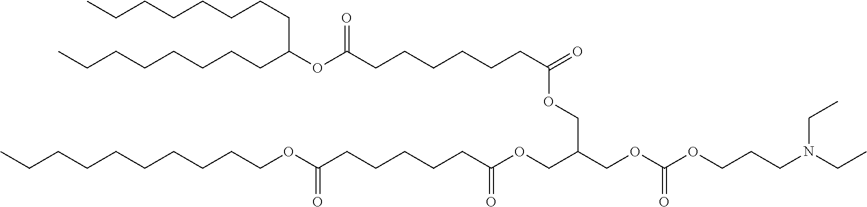 [SMILES: CCCCCCCCCCOC(=O)CCCCCC(=O)OCC(COC(=O)CCCCCCC(=O)OC(CCCCCCCC)CCCCCCCC)COC(=O)OCCCN(CC)CC]
VL404 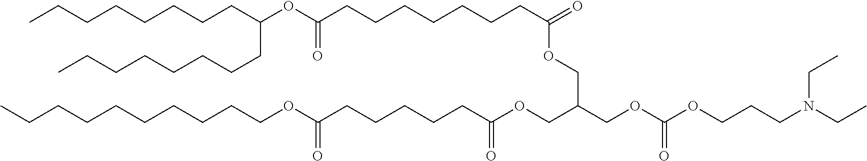 [SMILES: CCCCCCCCCCOC(=O)CCCCCC(=O)OCC(COC(=O)CCCCCCCC(=O)OC(CCCCCCCC)CCCCCCCC)COC(=O)OCCCN(CC)CC]
VL405 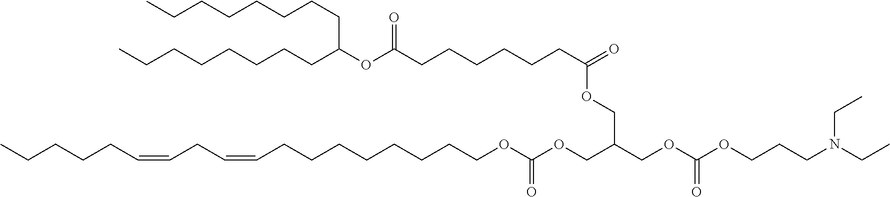 [SMILES: CCCCC/C=C\C/C=C\CCCCCCCCOC(=O)OCC(COC(=O)CCCCCCC(=O)OC(CCCCCCCC)CCCCCCCC)COC(=O)OCCCN(CC)CC]
VL406

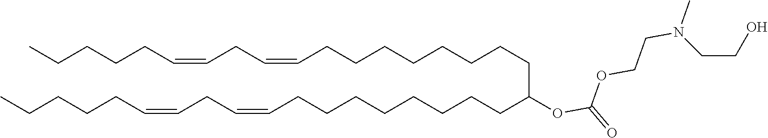 [SMILES: CCCCC/C=C\C/C=C\CCCCCCCCC(CCCCCCCC/C=C\C/C=C\CCCCC)OC(=O)OCCN(C)CCO]

VL407 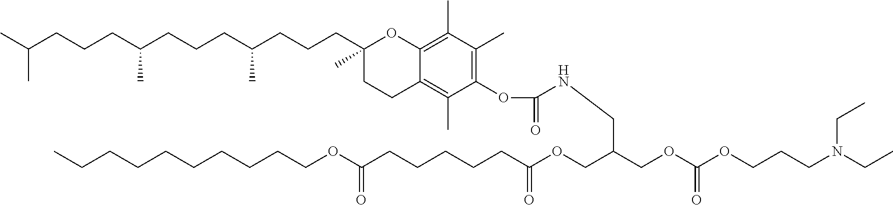 [SMILES: CCCCCCCCCCOC(=O)CCCCCC(=O)OCC(CNC(=O)Oc1c(C)c(C)c2c(c1C)CC[C@@](C)(CCC[C@H](C)CCC[C@H](C)CCCC(C)C)O2)COC(=O)OCCCN(CC)CC]
VL408 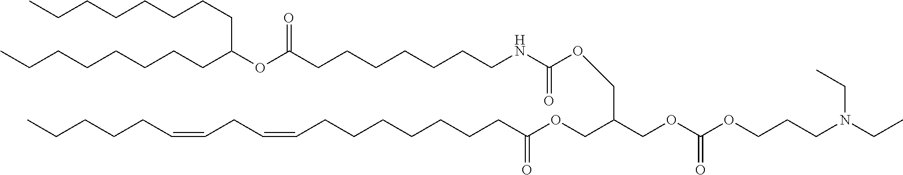 [SMILES: CCCCC/C=C\C/C=C\CCCCCCCC(=O)OCC(COC(=O)NCCCCCCCC(=O)OC(CCCCCCCC)CCCCCCCC)COC(=O)OCCCN(CC)CC]
VL409 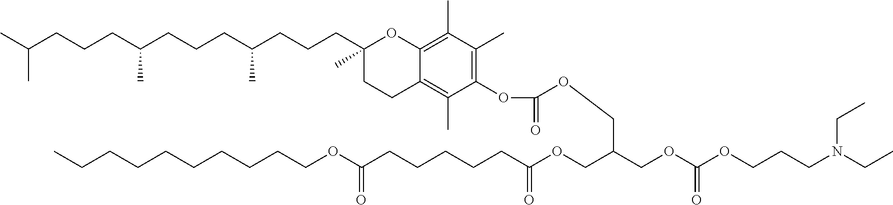 [SMILES: CCCCCCCCCCOC(=O)CCCCCC(=O)OCC(COC(=O)OCCCN(CC)CC)COC(=O)Oc1c(C)c(C)c2c(c1C)CC[C@@](C)(CCC[C@H](C)CCC[C@H](C)CCCC(C)C)O2]
VL410 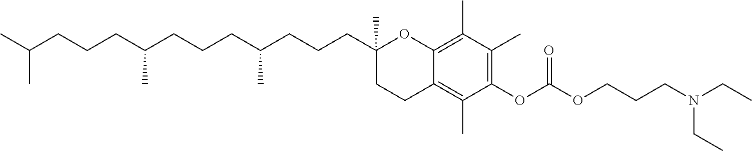 [SMILES: CCN(CC)CCCOC(=O)Oc1c(C)c(C)c2c(c1C)CC[C@@](C)(CCC[C@H](C)CCC[C@H](C)CCCC(C)C)O2]
VL411 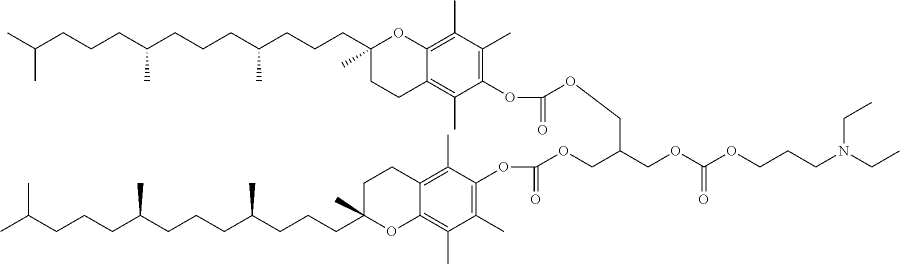 [SMILES: CCN(CC)CCCOC(=O)OCC(COC(=O)Oc1c(C)c(C)c2c(c1C)CC[C@@](C)(CCC[C@H](C)CCC[C@H](C)CCCC(C)C)O2)COC(=O)Oc1c(C)c(C)c2c(c1C)CC[C@@](C)(CCC[C@H](C)CCC[C@H](C)CCCC(C)C)O2]
VL412 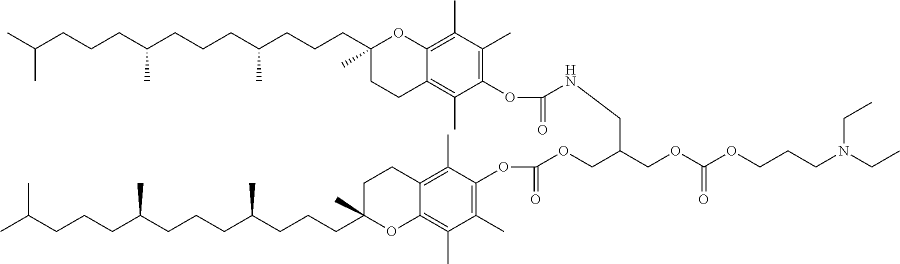 [SMILES: CCN(CC)CCCOC(=O)OCC(CNC(=O)Oc1c(C)c(C)c2c(c1C)CC[C@@](C)(CCC[C@H](C)CCC[C@H](C)CCCC(C)C)O2)COC(=O)Oc1c(C)c(C)c2c(c1C)CC[C@@](C)(CCC[C@H](C)CCC[C@H](C)CCCC(C)C)O2]
VL413 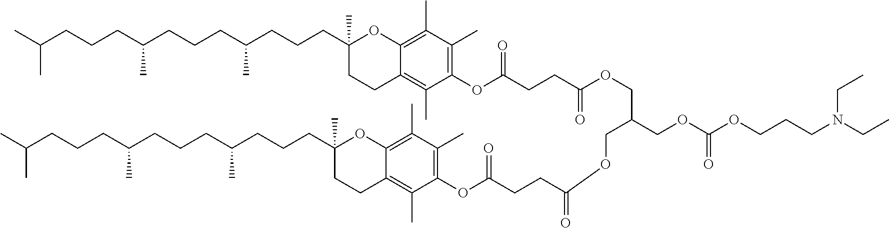 [SMILES: CCN(CC)CCCOC(=O)OCC(COC(=O)CCC(=O)Oc1c(C)c(C)c2c(c1C)CC[C@@](C)(CCC[C@H](C)CCC[C@H](C)CCCC(C)C)O2)COC(=O)CCC(=O)Oc1c(C)c(C)c2c(c1C)CC[C@@](C)(CCC[C@H](C)CCC[C@H](C)CCCC(C)C)O2]
VL414 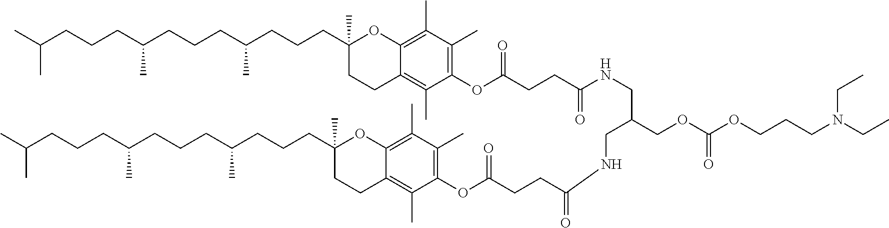 [SMILES: CCN(CC)CCCOC(=O)OCC(CNC(=O)CCC(=O)Oc1c(C)c(C)c2c(c1C)CC[C@@](C)(CCC[C@H](C)CCC[C@H](C)CCCC(C)C)O2)CNC(=O)CCC(=O)Oc1c(C)c(C)c2c(c1C)CC[C@@](C)(CCC[C@H](C)CCC[C@H](C)CCCC(C)C)O2]
VL415 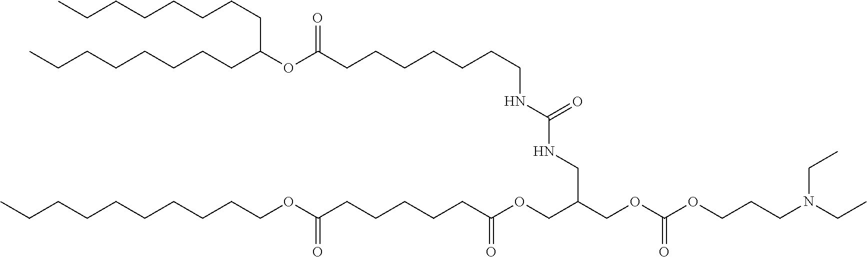 [SMILES: CCCCCCCCCCOC(=O)CCCCCC(=O)OCC(CNC(=O)NCCCCCCCC(=O)OC(CCCCCCCC)CCCCCCCC)COC(=O)OCCCN(CC)CC]
VL416 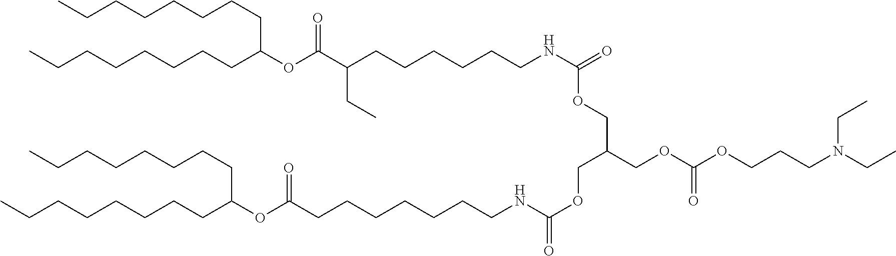 [SMILES: CCCCCCCCC(CCCCCCCC)OC(=O)CCCCCCCNC(=O)OCC(COC(=O)NCCCCCCC(CC)C(=O)OC(CCCCCCCC)CCCCCCCC)COC(=O)OCCCN(CC)CC]
VL417 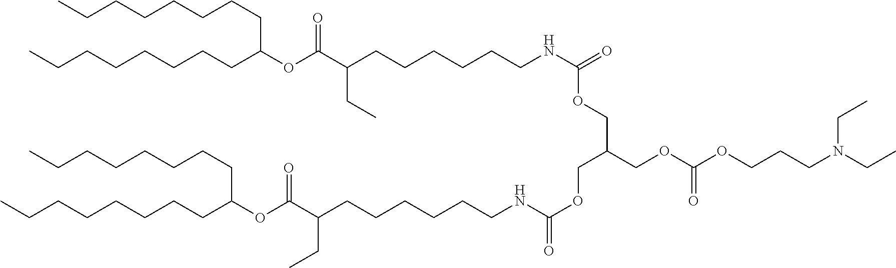 [SMILES: CCCCCCCCC(CCCCCCCC)OC(=O)C(CC)CCCCCCNC(=O)OCC(COC(=O)NCCCCCCC(CC)C(=O)OC(CCCCCCCC)CCCCCCCC)COC(=O)OCCCN(CC)CC]
VL418 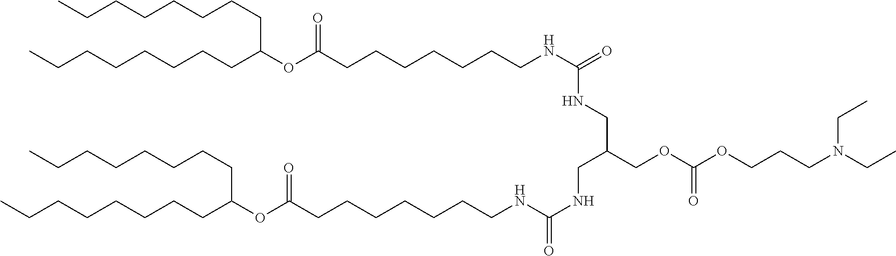 [SMILES: CCCCCCCCC(CCCCCCCC)OC(=O)CCCCCCCNC(=O)NCC(CNC(=O)NCCCCCCCC(=O)OC(CCCCCCCC)CCCCCCCC)COC(=O)OCCCN(CC)CC]
VL419 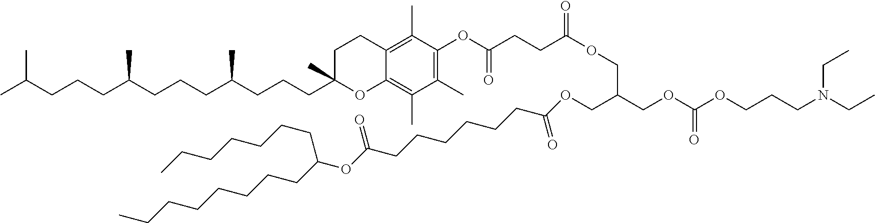 [SMILES: CCCCCCCCC(CCCCCCC)OC(=O)CCCCCCC(=O)OCC(COC(=O)CCC(=O)Oc1c(C)c(C)c2c(c1C)CC[C@@](C)(CCC[C@H](C)CCC[C@H](C)CCCC(C)C)O2)COC(=O)OCCCN(CC)CC]
VL420 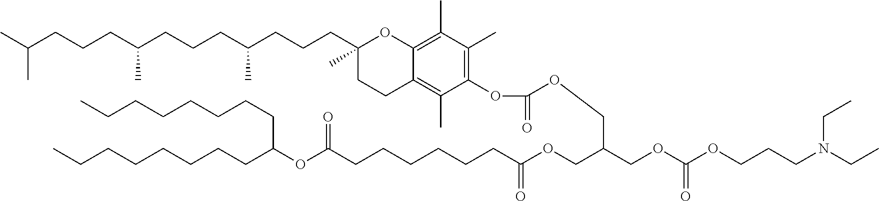 [SMILES: CCCCCCCCC(CCCCCCCC)OC(=O)CCCCCCC(=O)OCC(COC(=O)OCCCN(CC)CC)COC(=O)Oc1c(C)c(C)c2c(c1C)CC[C@@](C)(CCC[C@H](C)CCC[C@H](C)CCCC(C)C)O2]
VL421 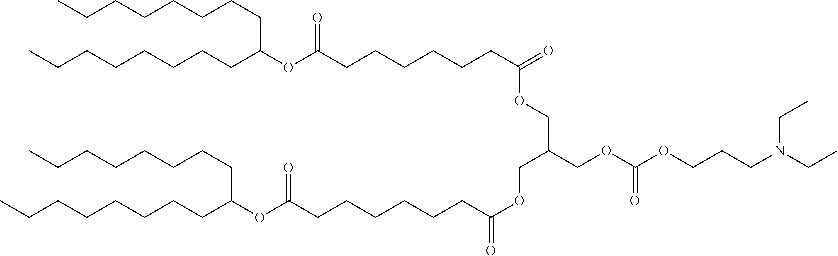 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC]
VL422 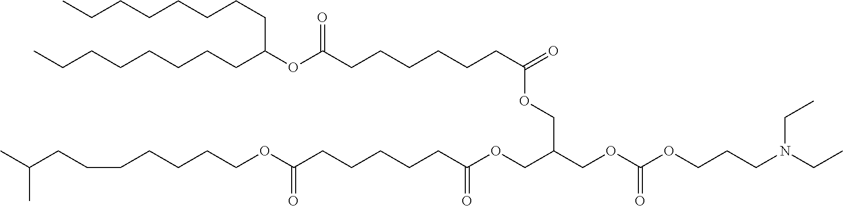 [SMILES: CCCCCCCCC(CCCCCCCC)OC(=O)CCCCCCC(=O)OCC(COC(=O)CCCCCC(=O)OCCCCCCCC(C)C)COC(=O)OCCCN(CC)CC]
VL423 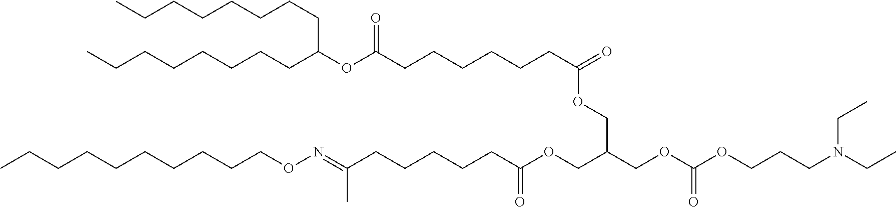 [SMILES: CCCCCCCCCCO/N=C(\C)CCCCCC(=O)OCC(COC(=O)CCCCCCC(=O)OC(CCCCCCCC)CCCCCCCC)COC(=O)OCCCN(CC)CC]
VL424 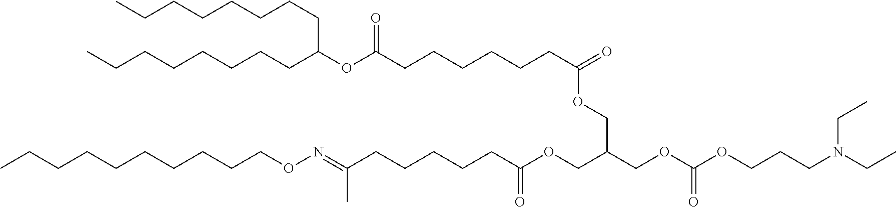 [SMILES: CCCCCCCCCCO/N=C(\C)CCCCCC(=O)OCC(COC(=O)CCCCCCC(=O)OC(CCCCCCCC)CCCCCCCC)COC(=O)OCCCN(CC)CC]
VL424 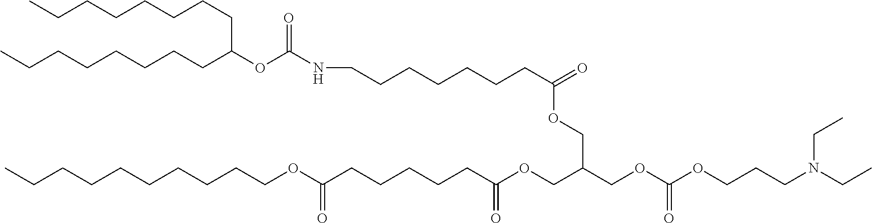 [SMILES: CCCCCCCCCCOC(=O)CCCCCC(=O)OCC(COC(=O)CCCCCCCNC(=O)OC(CCCCCCCC)CCCCCCCC)COC(=O)OCCCN(CC)CC]
VL425 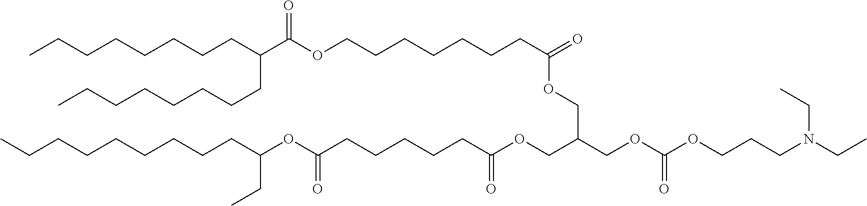 [SMILES: CCCCCCCCCC(CC)OC(=O)CCCCCC(=O)OCC(COC(=O)CCCCCCCOC(=O)C(CCCCCCCC)CCCCCCCC)COC(=O)OCCCN(CC)CC]
VL426 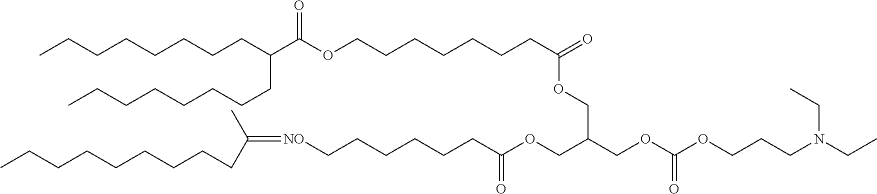
VL427 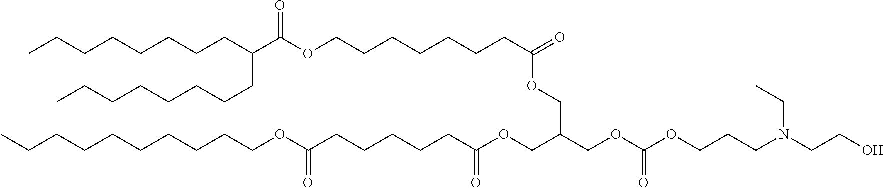 [SMILES: CCCCCCCCCCOC(=O)CCCCCC(=O)OCC(COC(=O)CCCCCCCOC(=O)C(CCCCCCCC)CCCCCCCC)COC(=O)OCCCN(CC)CCO]
VL428 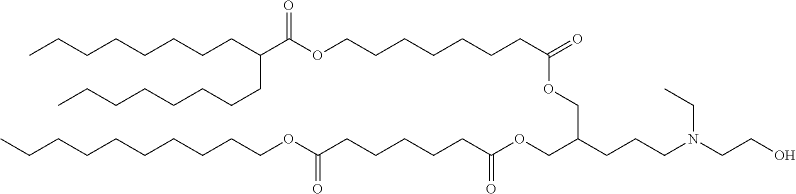 [SMILES: CCCCCCCCCCOC(=O)CCCCCC(=O)OCC(CCCN(CC)CCO)COC(=O)CCCCCCCOC(=O)C(CCCCCCCC)CCCCCCCC]
VL429 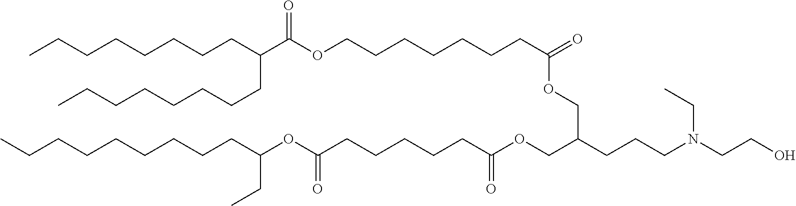 [SMILES: CCCCCCCCCC(CC)OC(=O)CCCCCC(=O)OCC(CCCN(CC)CCO)COC(=O)CCCCCCCOC(=O)C(CCCCCCCC)CCCCCCCC]
VL430 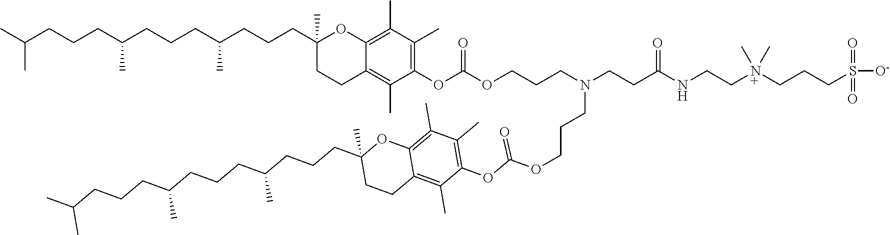 [SMILES: Cc1c(C)c2c(c(C)c1OC(=O)OCCCN(CCCOC(=O)Oc1c(C)c(C)c3c(c1C)CC[C@@](C)(CCC[C@H](C)CCC[C@H](C)CCCC(C)C)O3)CCC(=O)NCC[N+](C)(C)CCCS(=O)(=O)[O-])CC[C@@](C)(CCC[C@H](C)CCC[C@H](C)CCCC(C)C)O2]
VL431 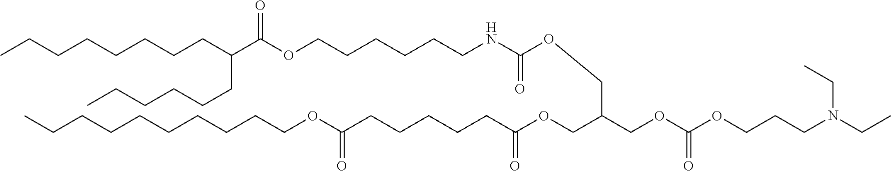 [SMILES: CCCCCCCCCCOC(=O)CCCCCC(=O)OCC(COC(=O)NCCCCCCOC(=O)C(CCCCCC)CCCCCCCC)COC(=O)OCCCN(CC)CC]
VL432 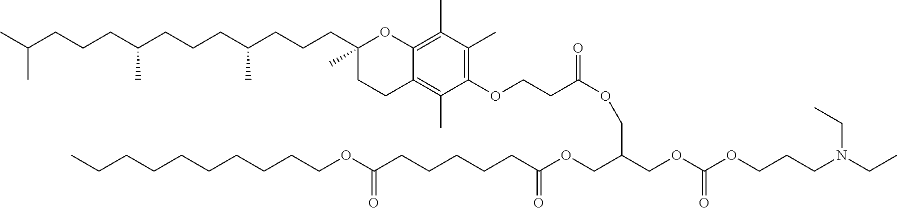 [SMILES: CCCCCCCCCCOC(=O)CCCCCC(=O)OCC(COC(=O)CCOc1c(C)c(C)c2c(c1C)CC[C@@](C)(CCC[C@H](C)CCC[C@H](C)CCCC(C)C)O2)COC(=O)OCCCN(CC)CC]
VL433 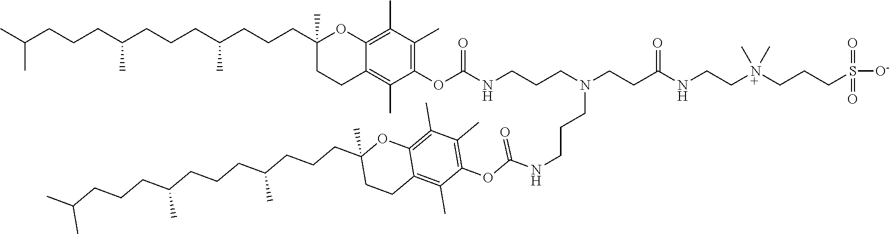 [SMILES: Cc1c(C)c2c(c(C)c1OC(=O)NCCCN(CCCNC(=O)Oc1c(C)c(C)c3c(c1C)CC[C@@](C)(CCC[C@H](C)CCC[C@H](C)CCCC(C)C)O3)CCC(=O)NCC[N+](C)(C)CCCS(=O)(=O)[O-])CC[C@@](C)(CCC[C@H](C)CCC[C@H](C)CCCC(C)C)O2]
VL434 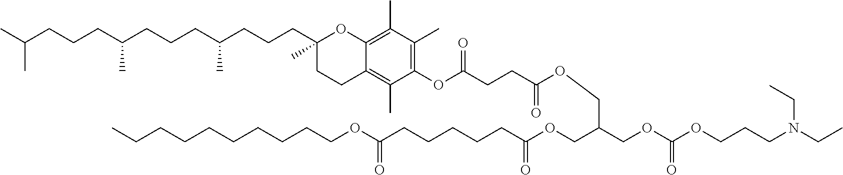 [SMILES: CCCCCCCCCCOC(=O)CCCCCC(=O)OCC(COC(=O)CCC(=O)Oc1c(C)c(C)c2c(c1C)CC[C@@](C)(CCC[C@H](C)CCC[C@H](C)CCCC(C)C)O2)COC(=O)OCCCN(CC)CC]
VL435 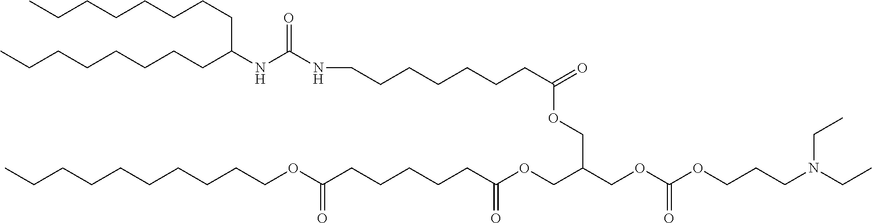 [SMILES: CCCCCCCCCCOC(=O)CCCCCC(=O)OCC(COC(=O)CCCCCCCNC(=O)NC(CCCCCCCC)CCCCCCCC)COC(=O)OCCCN(CC)CC]
VL436 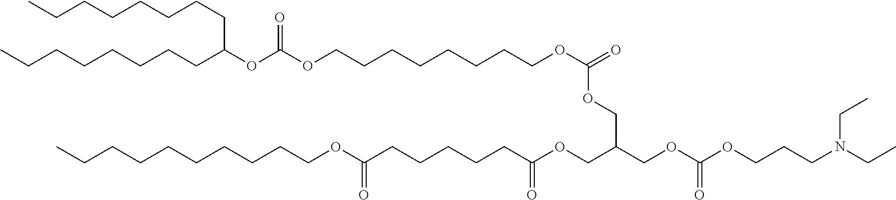 [SMILES: CCCCCCCCCCOC(=O)CCCCCC(=O)OCC(COC(=O)OCCCCCCCCOC(=O)OC(CCCCCCCC)CCCCCCCC)COC(=O)OCCCN(CC)CC]
VL437 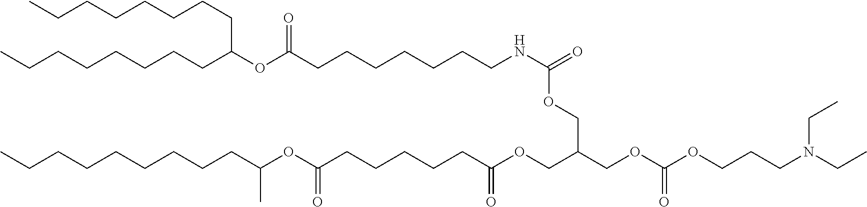 [SMILES: CCCCCCCCCC(C)OC(=O)CCCCCC(=O)OCC(COC(=O)NCCCCCCCC(=O)OC(CCCCCCCC)CCCCCCCC)COC(=O)OCCCN(CC)CC]
VL438 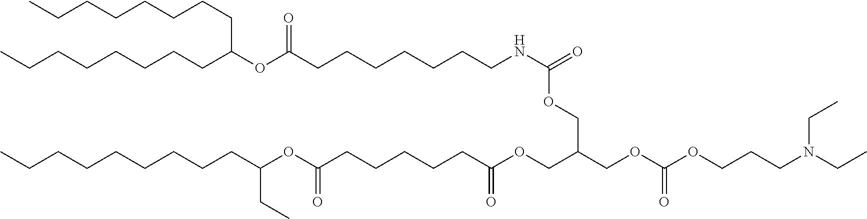 [SMILES: CCCCCCCCCC(CC)OC(=O)CCCCCC(=O)OCC(COC(=O)NCCCCCCCC(=O)OC(CCCCCCCC)CCCCCCCC)COC(=O)OCCCN(CC)CC]
VL439 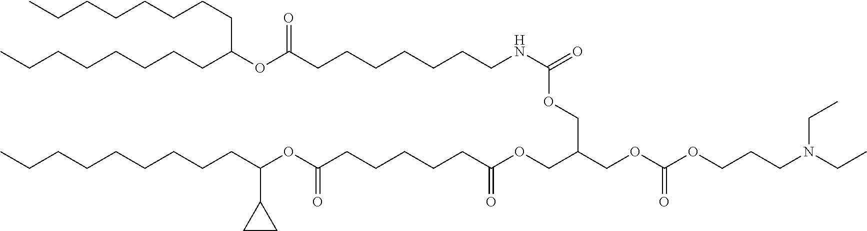 [SMILES: CCCCCCCCCC(OC(=O)CCCCCC(=O)OCC(COC(=O)NCCCCCCCC(=O)OC(CCCCCCCC)CCCCCCCC)COC(=O)OCCCN(CC)CC)C1CC1]
VL440 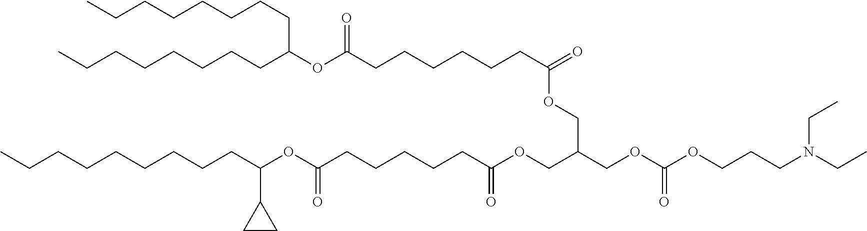 [SMILES: CCCCCCCCCC(OC(=O)CCCCCC(=O)OCC(COC(=O)CCCCCCC(=O)OC(CCCCCCCC)CCCCCCCC)COC(=O)OCCCN(CC)CC)C1CC1]
VL441 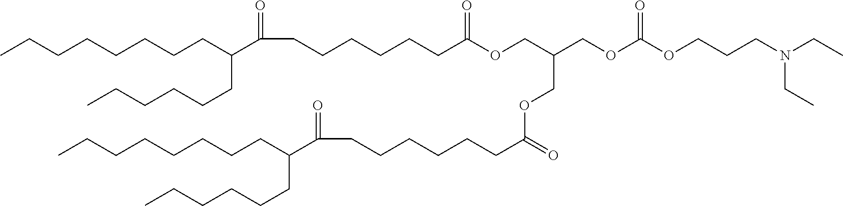 [SMILES: CCCCCCCCC(CCCCCC)C(=O)CCCCCCC(=O)OCC(COC(=O)CCCCCCC(=O)C(CCCCCC)CCCCCCCC)COC(=O)OCCCN(CC)CC]
VL442 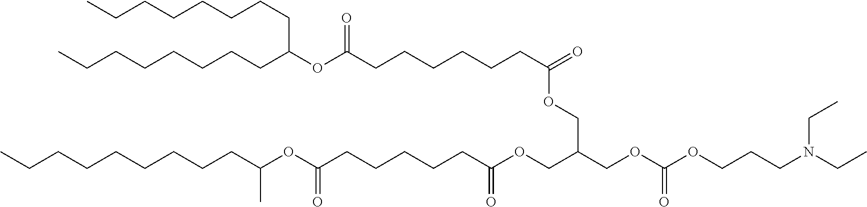 [SMILES: CCCCCCCCCC(C)OC(=O)CCCCCC(=O)OCC(COC(=O)CCCCCCC(=O)OC(CCCCCCCC)CCCCCCCC)COC(=O)OCCCN(CC)CC]
VL443 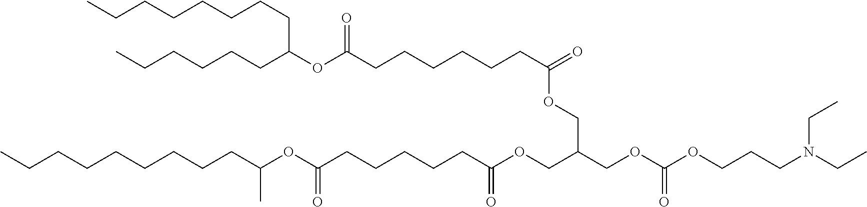 [SMILES: CCCCCCCCCC(C)OC(=O)CCCCCC(=O)OCC(COC(=O)CCCCCCC(=O)OC(CCCCCC)CCCCCCCC)COC(=O)OCCCN(CC)CC]
VL444 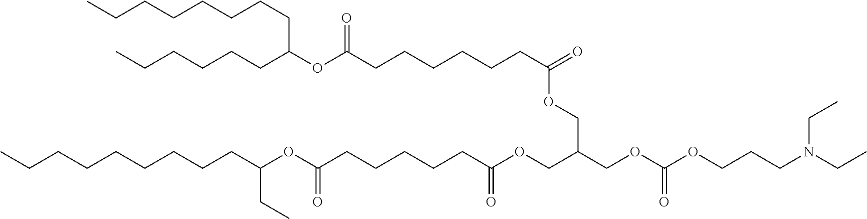 [SMILES: CCCCCCCCCC(CC)OC(=O)CCCCCC(=O)OCC(COC(=O)CCCCCCC(=O)OC(CCCCCC)CCCCCCCC)COC(=O)OCCCN(CC)CC]
VL445 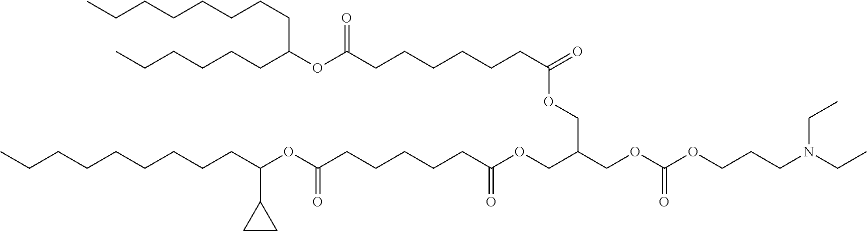 [SMILES: CCCCCCCCCC(OC(=O)CCCCCC(=O)OCC(COC(=O)CCCCCCC(=O)OC(CCCCCC)CCCCCCCC)COC(=O)OCCCN(CC)CC)C1CC1]
VL446 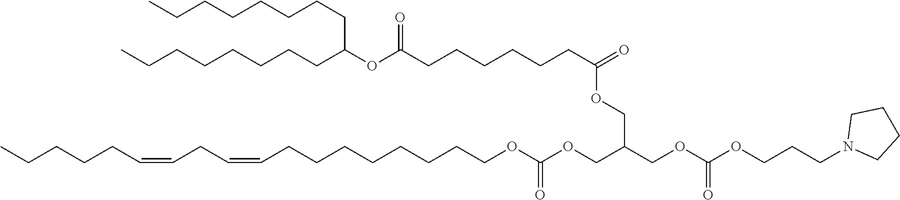 [SMILES: CCCCC/C=C\C/C=C\CCCCCCCCOC(=O)OCC(COC(=O)CCCCCCC(=O)OC(CCCCCCCC)CCCCCCCC)COC(=O)OCCCN1CCCC1]
VL447 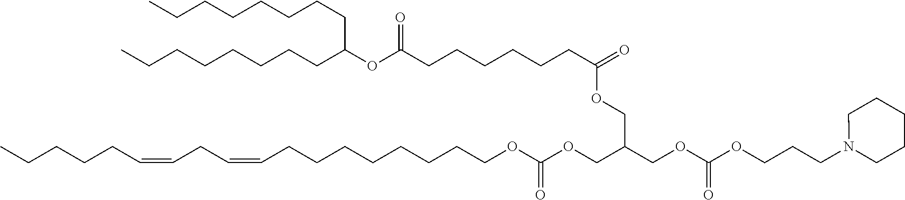 [SMILES: CCCCC/C=C\C/C=C\CCCCCCCCOC(=O)OCC(COC(=O)CCCCCCC(=O)OC(CCCCCCCC)CCCCCCCC)COC(=O)OCCCN1CCCCC1]
VL448 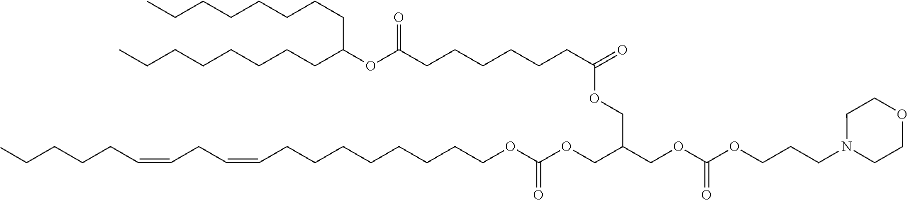 [SMILES: CCCCC/C=C\C/C=C\CCCCCCCCOC(=O)OCC(COC(=O)CCCCCCC(=O)OC(CCCCCCCC)CCCCCCCC)COC(=O)OCCCN1CCOCC1]
VL449 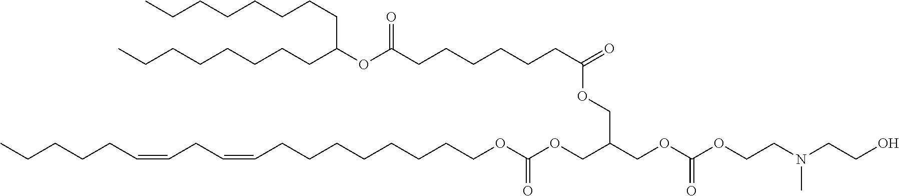 [SMILES: CCCCC/C=C\C/C=C\CCCCCCCCOC(=O)OCC(COC(=O)CCCCCCC(=O)OC(CCCCCCCC)CCCCCCCC)COC(=O)OCCN(C)CCO]
VL450 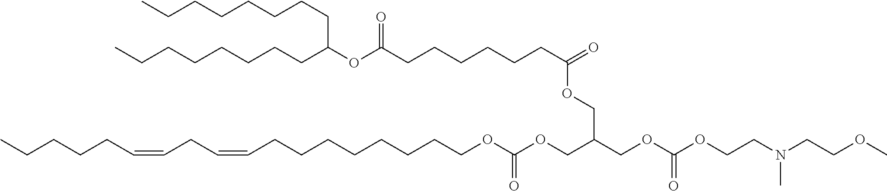 [SMILES: CCCCC/C=C\C/C=C\CCCCCCCCOC(=O)OCC(COC(=O)CCCCCCC(=O)OC(CCCCCCCC)CCCCCCCC)COC(=O)OCCN(C)CCOC]
VL451 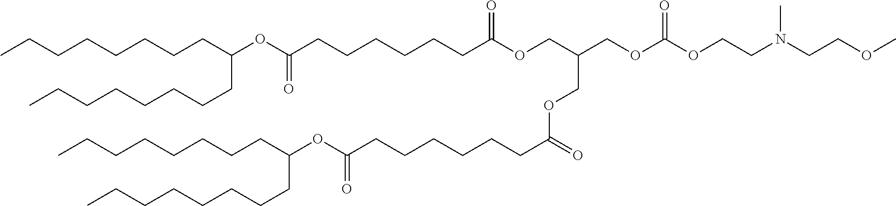 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCN(C)CCOC]
VL452 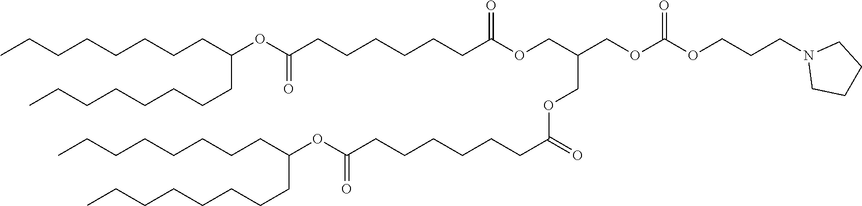 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN1CCCC1]
VL453 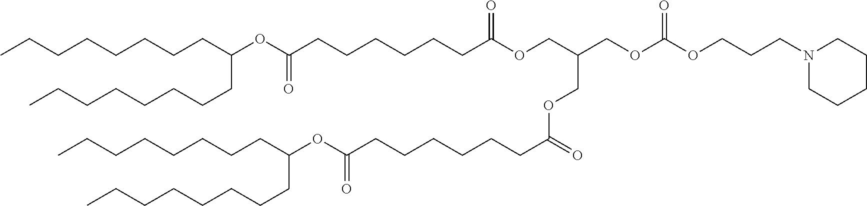 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN1CCCCC1]
VL454 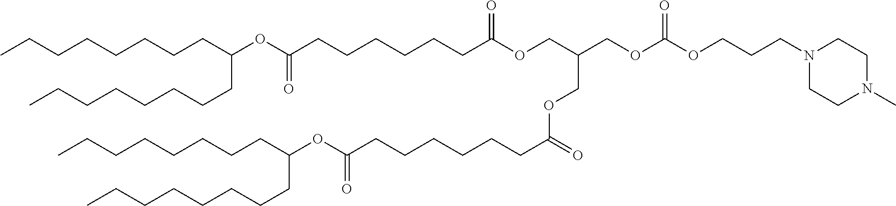 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN1CCN(C)CC1]
VL455 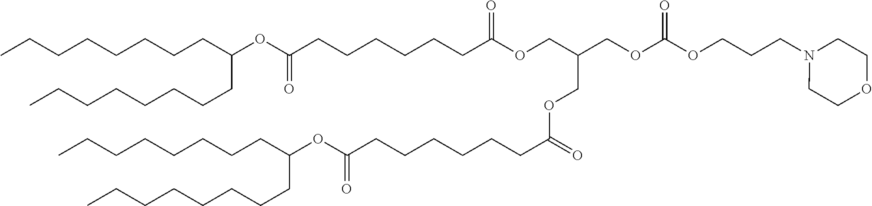 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN1CCOCC1]
VL456 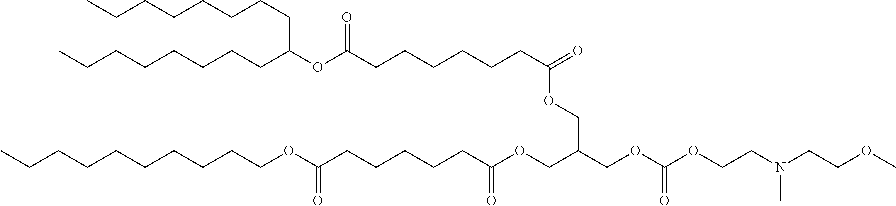 [SMILES: CCCCCCCCCCOC(=O)CCCCCC(=O)OCC(COC(=O)CCCCCCC(=O)OC(CCCCCCCC)CCCCCCCC)COC(=O)OCCN(C)CCOC]
VL457 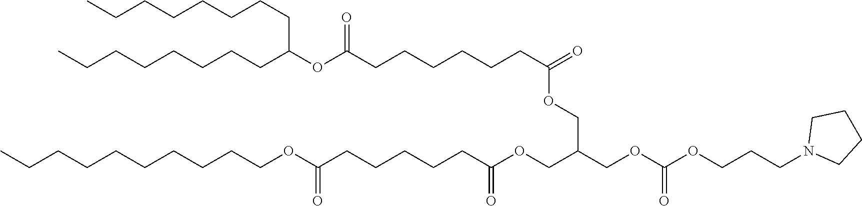 [SMILES: CCCCCCCCCCOC(=O)CCCCCC(=O)OCC(COC(=O)CCCCCCC(=O)OC(CCCCCCCC)CCCCCCCC)COC(=O)OCCCN1CCCC1]
VL458 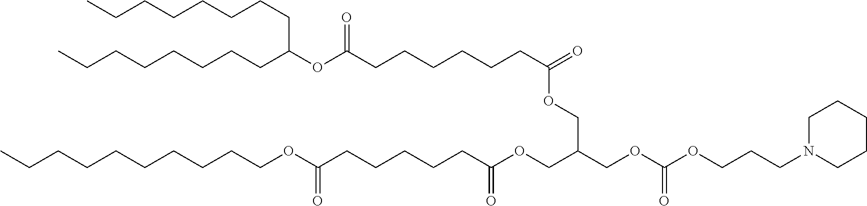 [SMILES: CCCCCCCCCCOC(=O)CCCCCC(=O)OCC(COC(=O)CCCCCCC(=O)OC(CCCCCCCC)CCCCCCCC)COC(=O)OCCCN1CCCCC1]
VL459 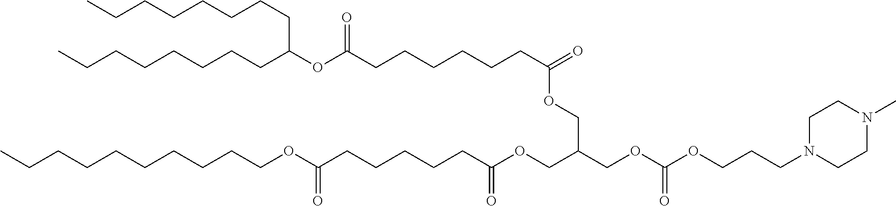 [SMILES: CCCCCCCCCCOC(=O)CCCCCC(=O)OCC(COC(=O)CCCCCCC(=O)OC(CCCCCCCC)CCCCCCCC)COC(=O)OCCCN1CCN(C)CC1]
VL460 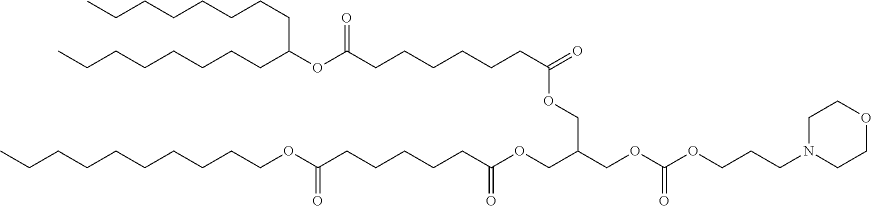 [SMILES: CCCCCCCCCCOC(=O)CCCCCC(=O)OCC(COC(=O)CCCCCCC(=O)OC(CCCCCCCC)CCCCCCCC)COC(=O)OCCCN1CCOCC1]
VL461 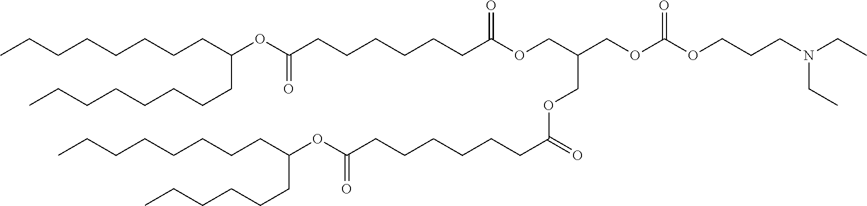 [SMILES: CCCCCCCCC(CCCCCC)OC(=O)CCCCCCC(=O)OCC(COC(=O)CCCCCCC(=O)OC(CCCCCCCC)CCCCCCCC)COC(=O)OCCCN(CC)CC]
VL462 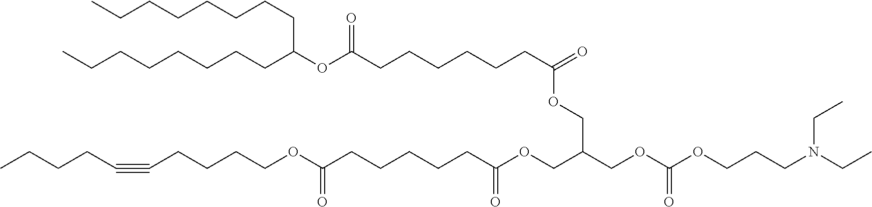 [SMILES: CCCCC#CCCCCOC(=O)CCCCCC(=O)OCC(COC(=O)CCCCCCC(=O)OC(CCCCCCCC)CCCCCCCC)COC(=O)OCCCN(CC)CC]
VL463 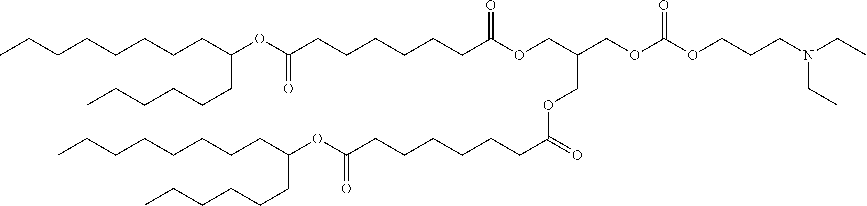 [SMILES: CCCCCCCCC(CCCCCC)OC(=O)CCCCCCC(=O)OCC(COC(=O)CCCCCCC(=O)OC(CCCCCC)CCCCCCCC)COC(=O)OCCCN(CC)CC]
VL464 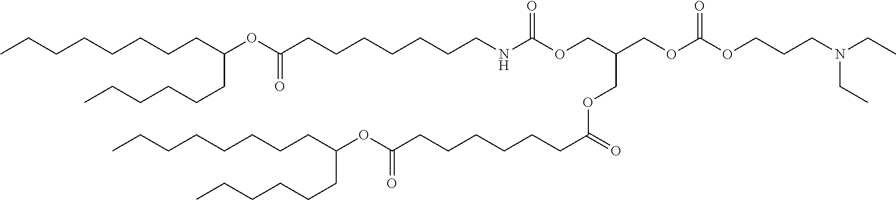 [SMILES: CCCCCCCCC(CCCCCC)OC(=O)CCCCCCCNC(=O)OCC(COC(=O)CCCCCCC(=O)OC(CCCCCC)CCCCCCCC)COC(=O)OCCCN(CC)CC]
VL465 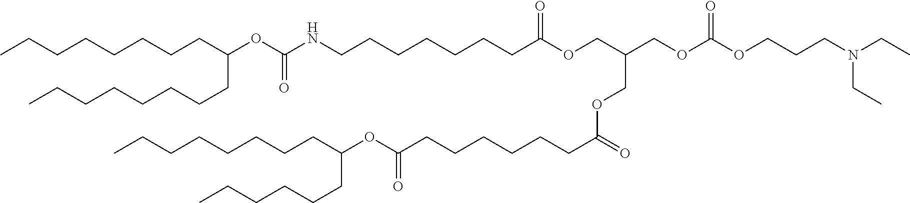 [SMILES: CCCCCCCCC(CCCCCC)OC(=O)CCCCCCC(=O)OCC(COC(=O)CCCCCCCNC(=O)OC(CCCCCCCC)CCCCCCCC)COC(=O)OCCCN(CC)CC]
VL466

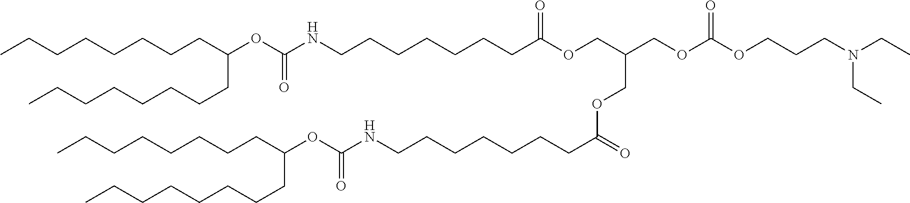 [SMILES: CCCCCCCCC(CCCCCCCC)OC(=O)NCCCCCCCC(=O)OCC(COC(=O)CCCCCCCNC(=O)OC(CCCCCCCC)CCCCCCCC)COC(=O)OCCCN(CC)CC]

VL467 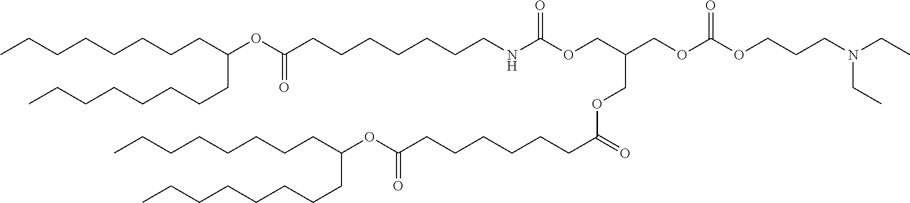 [SMILES: CCCCCCCCC(CCCCCCCC)OC(=O)CCCCCCCNC(=O)OCC(COC(=O)CCCCCCC(=O)OC(CCCCCCCC)CCCCCCCC)COC(=O)OCCCN(CC)CC]
VL468 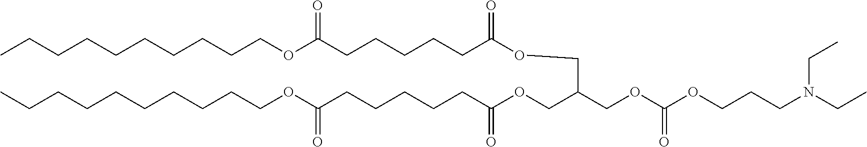 [SMILES: CCCCCCCCCCOC(=O)CCCCCC(=O)OCC(COC(=O)CCCCCC(=O)OCCCCCCCCCC)COC(=O)OCCCN(CC)CC]
VL469 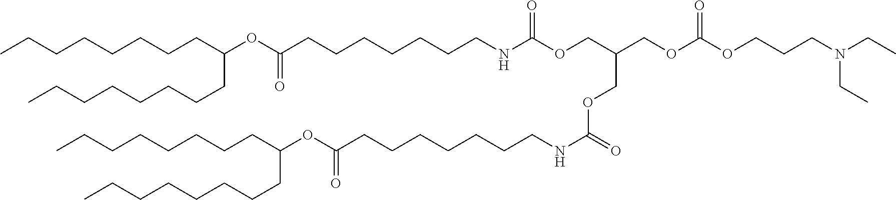 [SMILES: CCCCCCCCC(CCCCCCCC)OC(=O)CCCCCCCNC(=O)OCC(COC(=O)NCCCCCCCC(=O)OC(CCCCCCCC)CCCCCCCC)COC(=O)OCCCN(CC)CC]
VL470 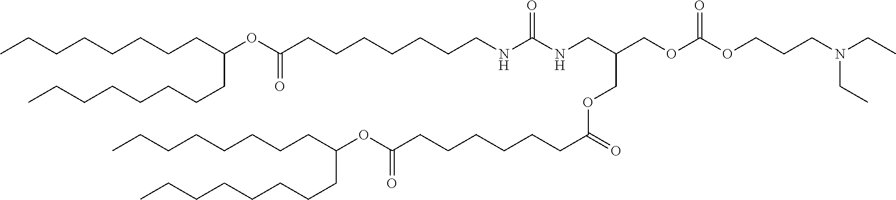 [SMILES: CCCCCCCCC(CCCCCCCC)OC(=O)CCCCCCCNC(=O)NCC(COC(=O)CCCCCCC(=O)OC(CCCCCCCC)CCCCCCCC)COC(=O)OCCCN(CC)CC]
VL471 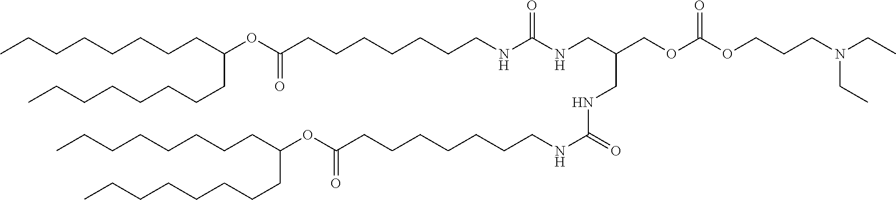 [SMILES: CCCCCCCCC(CCCCCCCC)OC(=O)CCCCCCCNC(=O)NCC(CNC(=O)NCCCCCCCC(=O)OC(CCCCCCCC)CCCCCCCC)COC(=O)OCCCN(CC)CC]
VL472 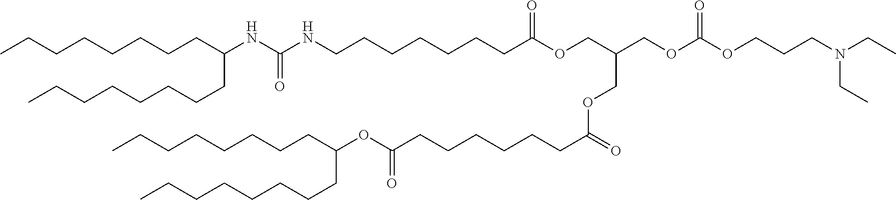 [SMILES: CCCCCCCCC(CCCCCCCC)NC(=O)NCCCCCCCC(=O)OCC(COC(=O)CCCCCCC(=O)OC(CCCCCCCC)CCCCCCCC)COC(=O)OCCCN(CC)CC]
VL473 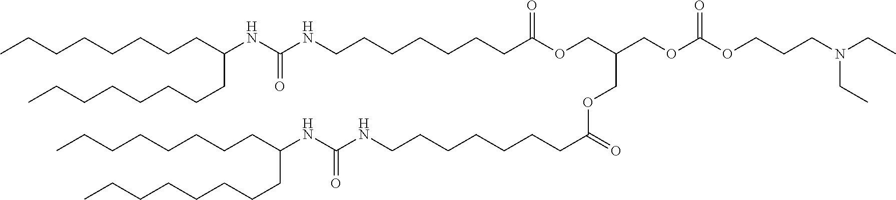 [SMILES: CCCCCCCCC(CCCCCCCC)NC(=O)NCCCCCCCC(=O)OCC(COC(=O)CCCCCCCNC(=O)NC(CCCCCCCC)CCCCCCCC)COC(=O)OCCCN(CC)CC]
VL474 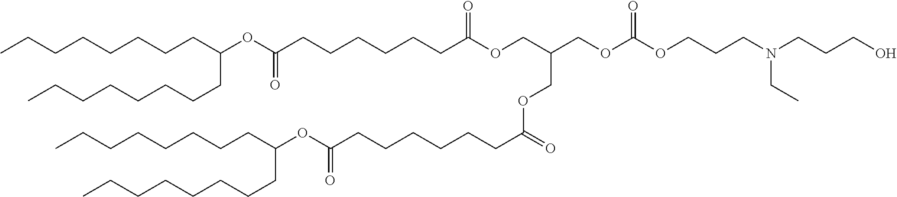 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CCCO]
VL475 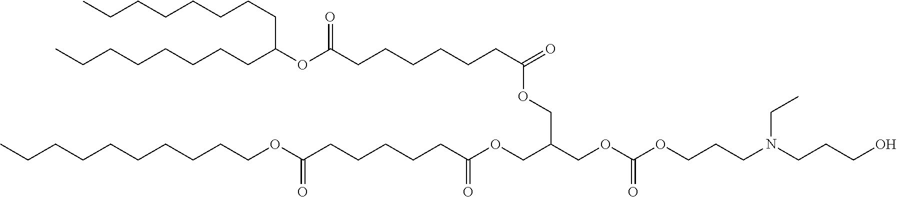 [SMILES: CCCCCCCCCCOC(=O)CCCCCC(=O)OCC(COC(=O)CCCCCCC(=O)OC(CCCCCCCC)CCCCCCCC)COC(=O)OCCCN(CC)CCCO]
VL476 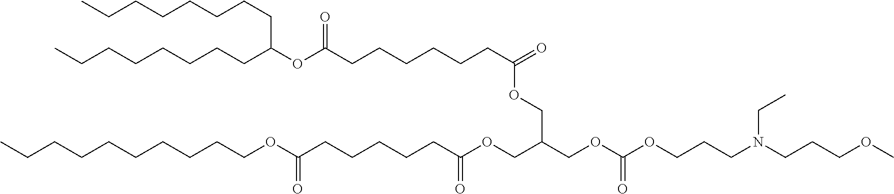 [SMILES: CCCCCCCCCCOC(=O)CCCCCC(=O)OCC(COC(=O)CCCCCCC(=O)OC(CCCCCCCC)CCCCCCCC)COC(=O)OCCCN(CC)CCCOC]
VL477 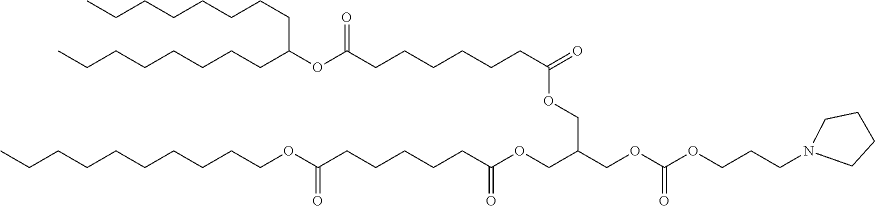 [SMILES: CCCCCCCCCCOC(=O)CCCCCC(=O)OCC(COC(=O)CCCCCCC(=O)OC(CCCCCCCC)CCCCCCCC)COC(=O)OCCCN1CCCC1]
VL478 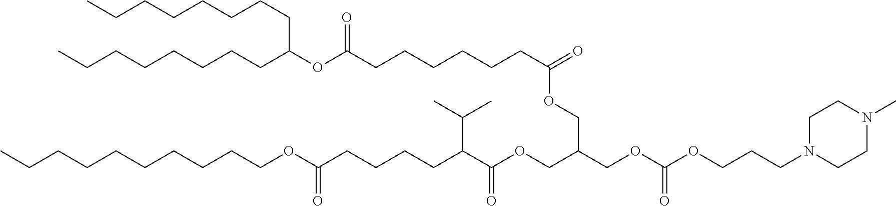 [SMILES: CCCCCCCCCCOC(=O)CCCCC(C(=O)OCC(COC(=O)CCCCCCC(=O)OC(CCCCCCCC)CCCCCCCC)COC(=O)OCCCN1CCN(C)CC1)C(C)C]
VL479 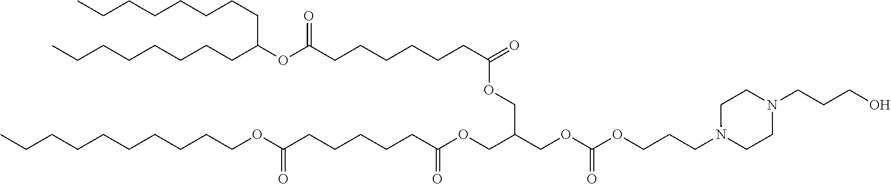 [SMILES: CCCCCCCCCCOC(=O)CCCCCC(=O)OCC(COC(=O)CCCCCCC(=O)OC(CCCCCCCC)CCCCCCCC)COC(=O)OCCCN1CCN(CCCO)CC1]
VL480 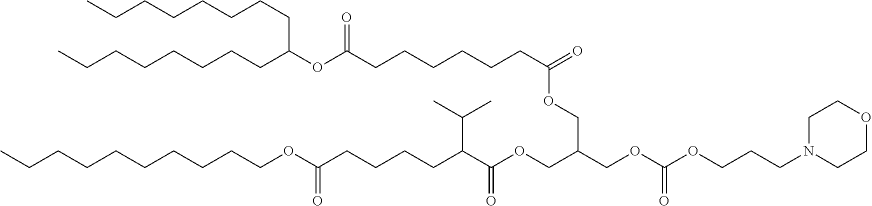 [SMILES: CCCCCCCCCCOC(=O)CCCCC(C(=O)OCC(COC(=O)CCCCCCC(=O)OC(CCCCCCCC)CCCCCCCC)COC(=O)OCCCN1CCOCC1)C(C)C]
VL481 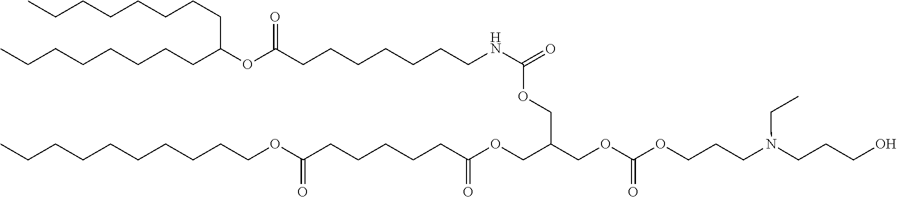 [SMILES: CCCCCCCCCCOC(=O)CCCCCC(=O)OCC(COC(=O)NCCCCCCCC(=O)OC(CCCCCCCC)CCCCCCCC)COC(=O)OCCCN(CC)CCCO]
VL482 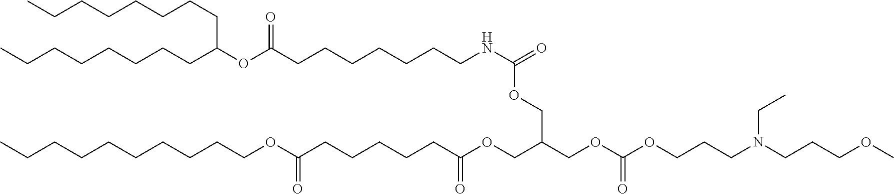 [SMILES: CCCCCCCCCCOC(=O)CCCCCC(=O)OCC(COC(=O)NCCCCCCCC(=O)OC(CCCCCCCC)CCCCCCCC)COC(=O)OCCCN(CC)CCCOC]
VL483 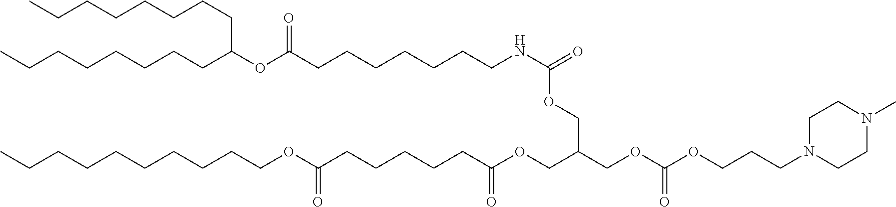 [SMILES: CCCCCCCCCCOC(=O)CCCCCC(=O)OCC(COC(=O)NCCCCCCCC(=O)OC(CCCCCCCC)CCCCCCCC)COC(=O)OCCCN1CCN(C)CC1]
VL484 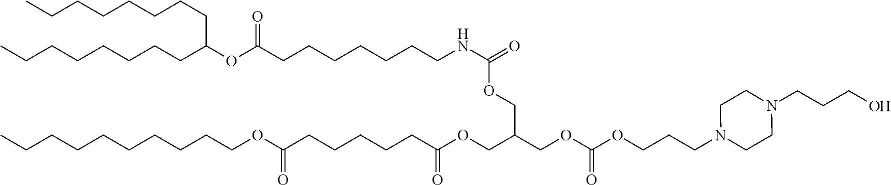 [SMILES: CCCCCCCCCCOC(=O)CCCCCC(=O)OCC(COC(=O)NCCCCCCCC(=O)OC(CCCCCCCC)CCCCCCCC)COC(=O)OCCCN1CCN(CCCO)CC1]
VL485 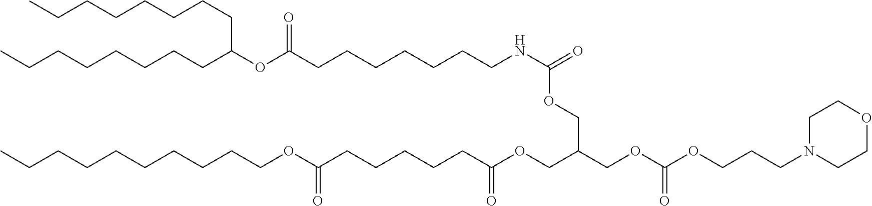 [SMILES: CCCCCCCCCCOC(=O)CCCCCC(=O)OCC(COC(=O)NCCCCCCCC(=O)OC(CCCCCCCC)CCCCCCCC)COC(=O)OCCCN1CCOCC1]
VL486 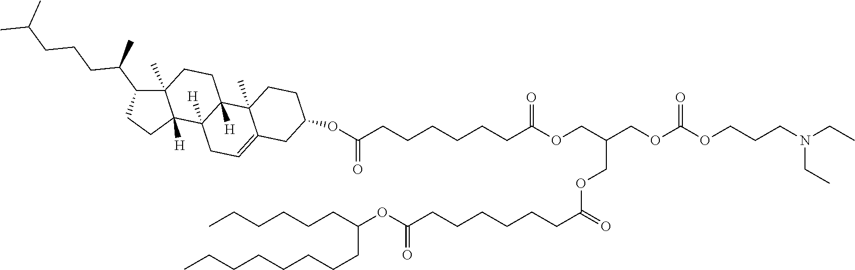
VL487 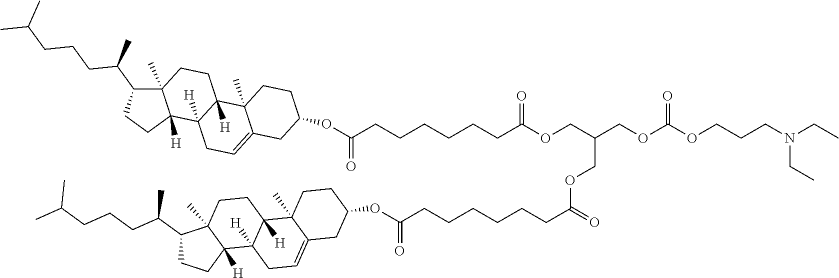
VL488 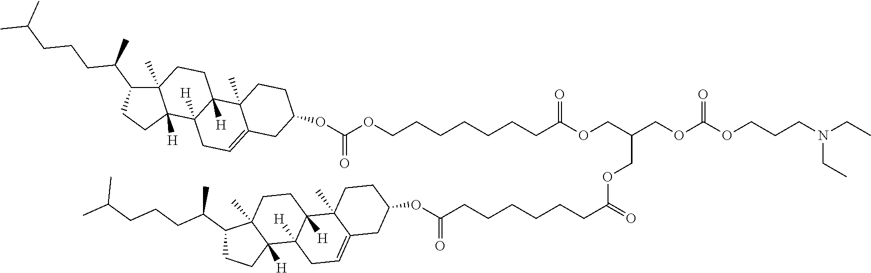
VL489 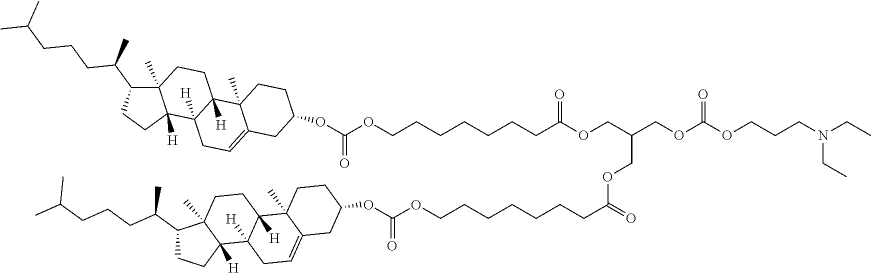
VL490 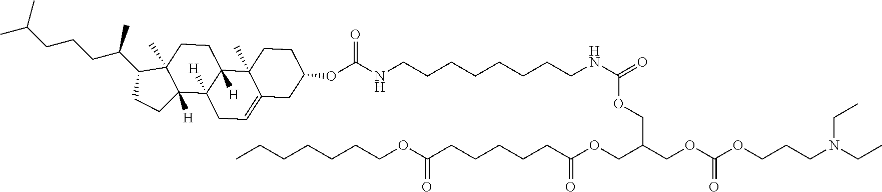
VL491 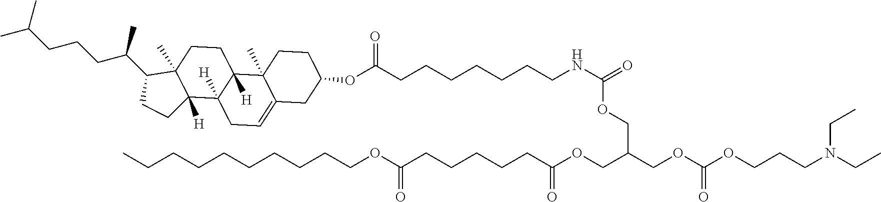
VL492 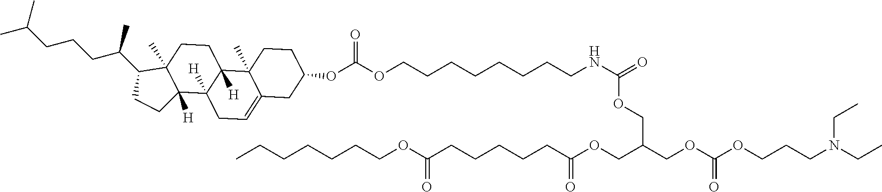
VL493 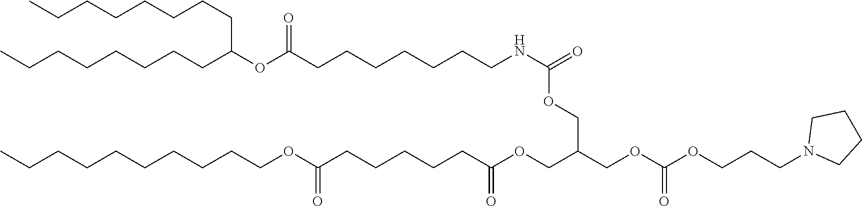 [SMILES: CCCCCCCCCCOC(=O)CCCCCC(=O)OCC(COC(=O)NCCCCCCCC(=O)OC(CCCCCCCC)CCCCCCCC)COC(=O)OCCCN1CCCC1]
VL494 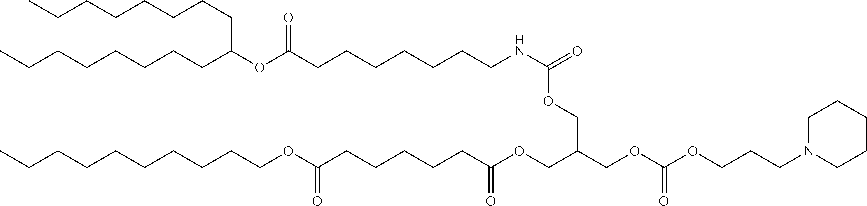 [SMILES: CCCCCCCCCCOC(=O)CCCCCC(=O)OCC(COC(=O)NCCCCCCCC(=O)OC(CCCCCCCC)CCCCCCCC)COC(=O)OCCCN1CCCCC1]
VL495 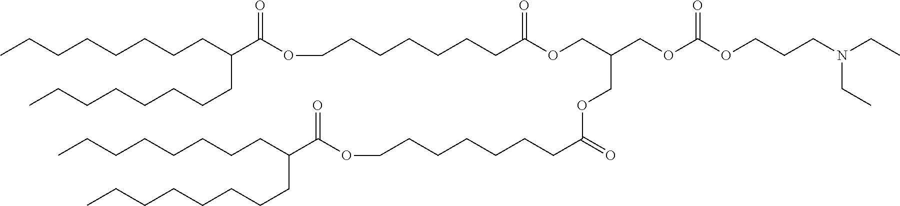 [SMILES: CCCCCCCCC(CCCCCCCC)C(=O)OCCCCCCCC(=O)OCC(COC(=O)CCCCCCCOC(=O)C(CCCCCCCC)CCCCCCCC)COC(=O)OCCCN(CC)CC]
VL496 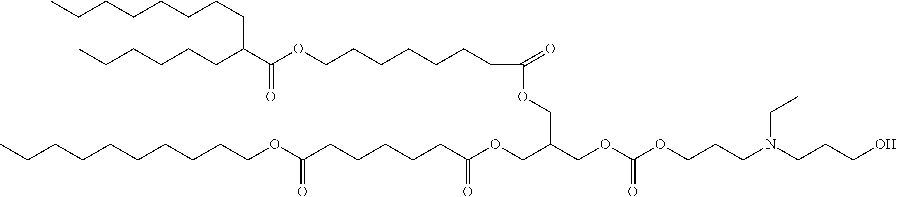 [SMILES: CCCCCCCCCCOC(=O)CCCCCC(=O)OCC(COC(=O)CCCCCCCOC(=O)C(CCCCCC)CCCCCCCC)COC(=O)OCCCN(CC)CCCO]
VL497 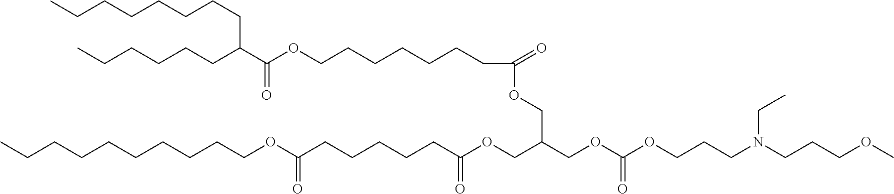 [SMILES: CCCCCCCCCCOC(=O)CCCCCC(=O)OCC(COC(=O)CCCCCCCOC(=O)C(CCCCCC)CCCCCCCC)COC(=O)OCCCN(CC)CCCOC]
VL498 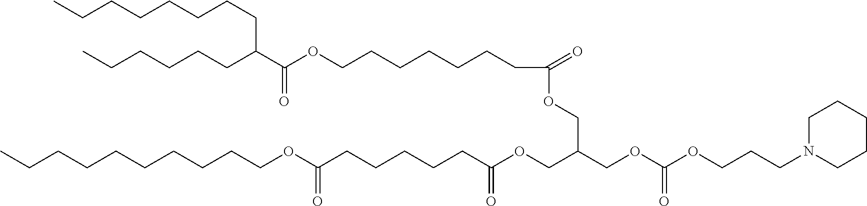 [SMILES: CCCCCCCCCCOC(=O)CCCCCC(=O)OCC(COC(=O)CCCCCCCOC(=O)C(CCCCCC)CCCCCCCC)COC(=O)OCCCN1CCCCC1]
VL499 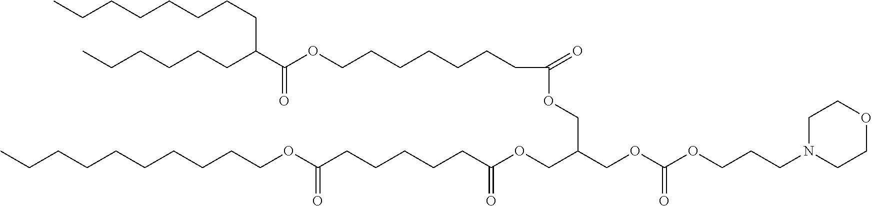 [SMILES: CCCCCCCCCCOC(=O)CCCCCC(=O)OCC(COC(=O)CCCCCCCOC(=O)C(CCCCCC)CCCCCCCC)COC(=O)OCCCN1CCOCC1]
VL500 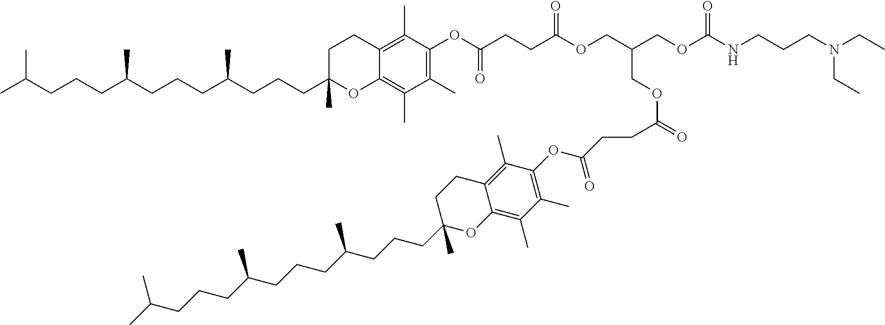 [SMILES: CCN(CC)CCCNC(=O)OCC(COC(=O)CCC(=O)Oc1c(C)c(C)c2c(c1C)CC[C@@](C)(CCC[C@H](C)CCC[C@H](C)CCCC(C)C)O2)COC(=O)CCC(=O)Oc1c(C)c(C)c2c(c1C)CC[C@@](C)(CCC[C@H](C)CCC[C@H](C)CCCC(C)C)O2]
VL501 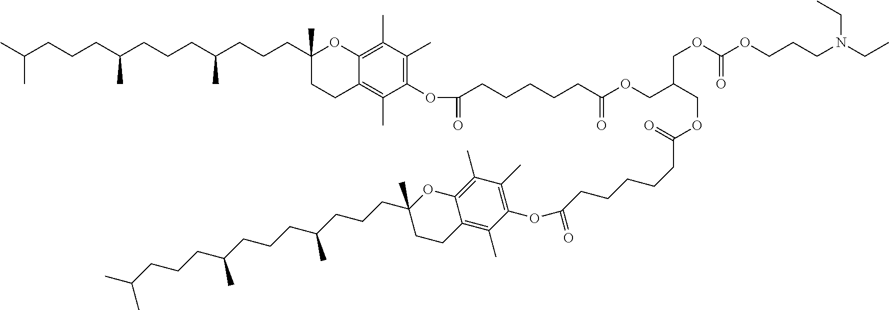 [SMILES: CCN(CC)CCCOC(=O)OCC(COC(=O)CCCCCC(=O)Oc1c(C)c(C)c2c(c1C)CC[C@](C)(CCC[C@@H](C)CCC[C@@H](C)CCCC(C)C)O2)COC(=O)CCCCCC(=O)Oc1c(C)c(C)c2c(c1C)CC[C@](C)(CCC[C@@H](C)CCC[C@@H](C)CCCC(C)C)O2]
VL502 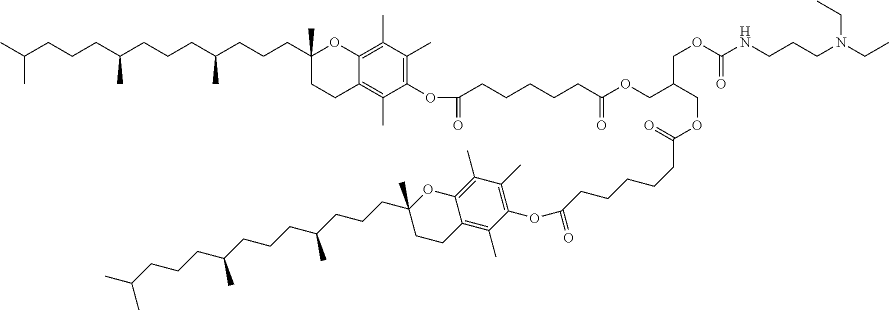 [SMILES: CCN(CC)CCCNC(=O)OCC(COC(=O)CCCCCC(=O)Oc1c(C)c(C)c2c(c1C)CC[C@](C)(CCC[C@@H](C)CCC[C@@H](C)CCCC(C)C)O2)COC(=O)CCCCCC(=O)Oc1c(C)c(C)c2c(c1C)CC[C@](C)(CCC[C@@H](C)CCC[C@@H](C)CCCC(C)C)O2]
VL503 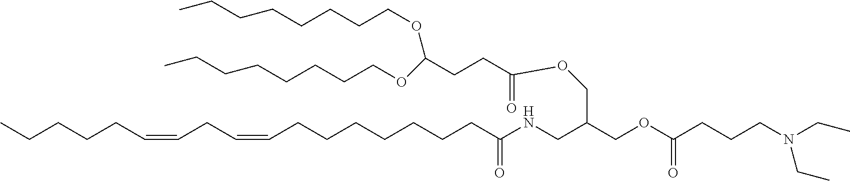 [SMILES: CCCCC/C=C\C/C=C\CCCCCCCC(=O)NCC(COC(=O)CCCN(CC)CC)COC(=O)CCC(OCCCCCCCC)OCCCCCCCC]
VL506 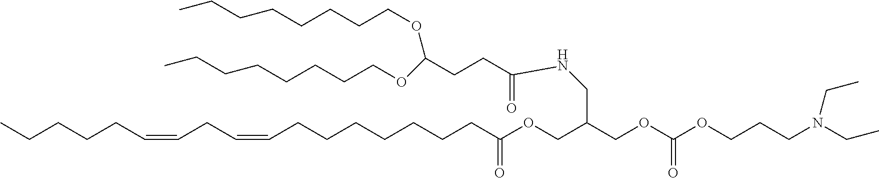 [SMILES: CCCCC/C=C\C/C=C\CCCCCCCC(=O)OCC(CNC(=O)CCC(OCCCCCCCC)OCCCCCCCC)COC(=O)OCCCN(CC)CC]
VL507 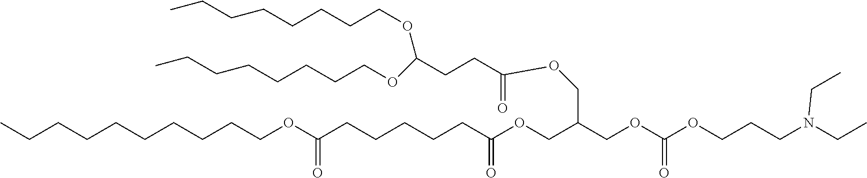 [SMILES: CCCCCCCCCCOC(=O)CCCCCC(=O)OCC(COC(=O)CCC(OCCCCCCCC)OCCCCCCCC)COC(=O)OCCCN(CC)CC]
VL508 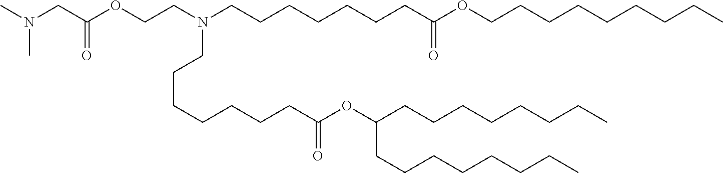 [SMILES: CCCCCCCCCOC(=O)CCCCCCCN(CCCCCCCC(=O)OC(CCCCCCCC)CCCCCCCC)CCOC(=O)CN(C)C]
VL509 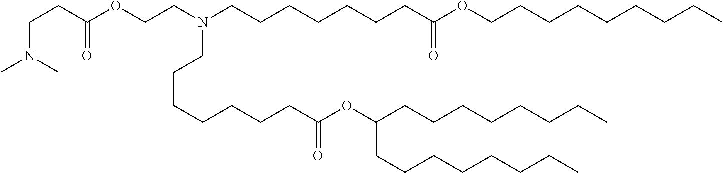 [SMILES: CCCCCCCCCOC(=O)CCCCCCCN(CCCCCCCC(=O)OC(CCCCCCCC)CCCCCCCC)CCOC(=O)CCN(C)C]
VL510 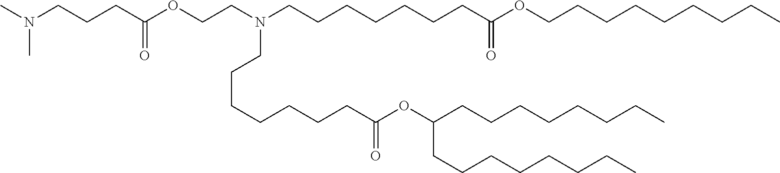 [SMILES: CCCCCCCCCOC(=O)CCCCCCCN(CCCCCCCC(=O)OC(CCCCCCCC)CCCCCCCC)CCOC(=O)CCCN(C)C]
VL511 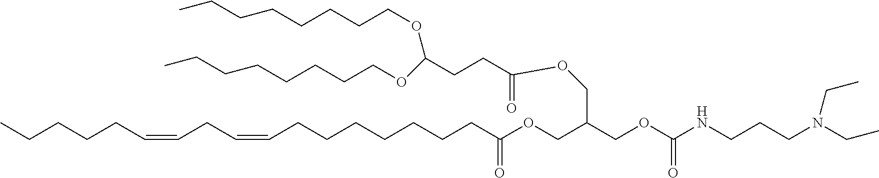 [SMILES: CCCCC/C=C\C/C=C\CCCCCCCC(=O)OCC(COC(=O)CCC(OCCCCCCCC)OCCCCCCCC)COC(=O)NCCCN(CC)CC]
VL512 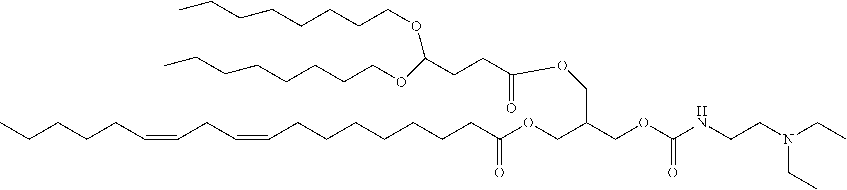 [SMILES: CCCCC/C=C\C/C=C\CCCCCCCC(=O)OCC(COC(=O)CCC(OCCCCCCCC)OCCCCCCCC)COC(=O)NCCN(CC)CC]
VL513 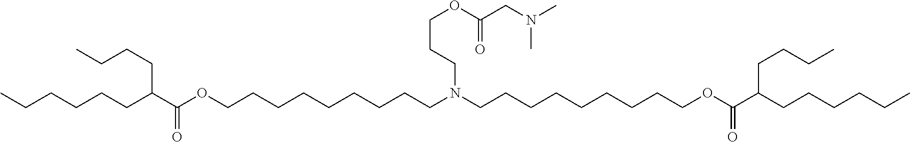 [SMILES: CCCCCCC(CCCC)C(=O)OCCCCCCCCCN(CCCCCCCCCOC(=O)C(CCCC)CCCCCC)CCCOC(=O)CN(C)C]
VL514 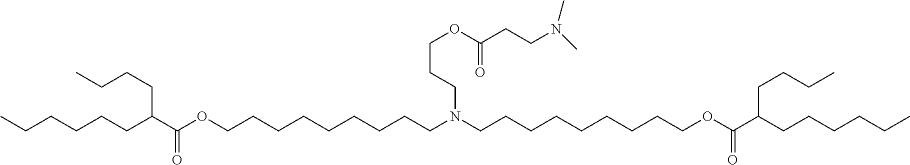 [SMILES: CCCCCCC(CCCC)C(=O)OCCCCCCCCCN(CCCCCCCCCOC(=O)C(CCCC)CCCCCC)CCCOC(=O)CCN(C)C]
VL515 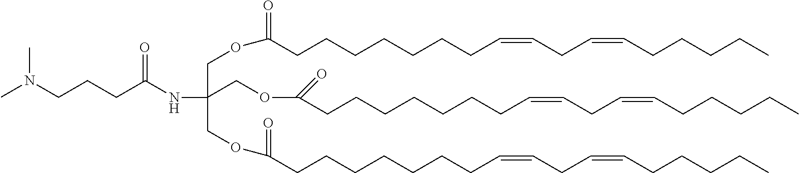 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NC(=O)CCCN(C)C]
VL516 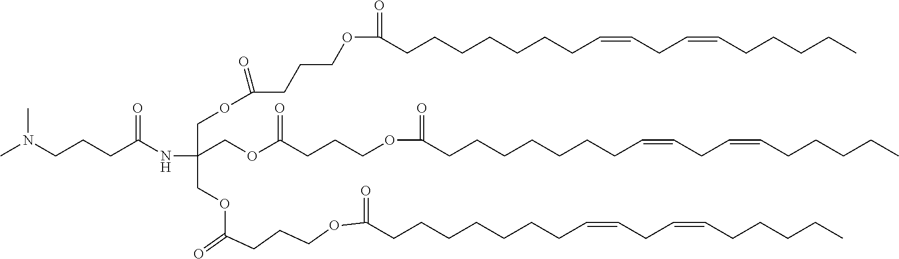 [SMILES: CCCCC/C=C\C/C=C\CCCCCCCC(=O)OCCCC(=O)OCC(COC(=O)CCCOC(=O)CCCCCCC/C=C\C/C=C\CCCCC)(COC(=O)CCCOC(=O)CCCCCCC/C=C\C/C=C\CCCCC)NC(=O)CCCN(C)C]
VL517 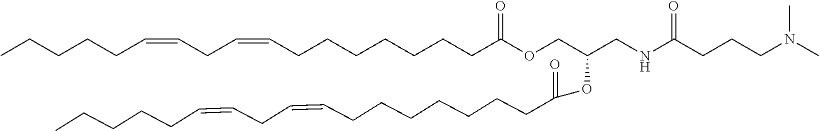 [SMILES: CCCCC/C=C\C/C=C\CCCCCCCC(=O)OC[C@H](CNC(=O)CCCN(C)C)OC(=O)CCCCCCC/C=C\C/C=C\CCCCC]
VL518 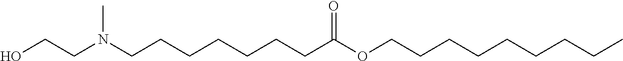 [SMILES: CCCCCCCCCOC(=O)CCCCCCCN(C)CCO]
VL519 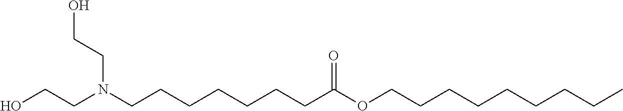 [SMILES: CCCCCCCCCOC(=O)CCCCCCCN(CCO)CCO]
VL520 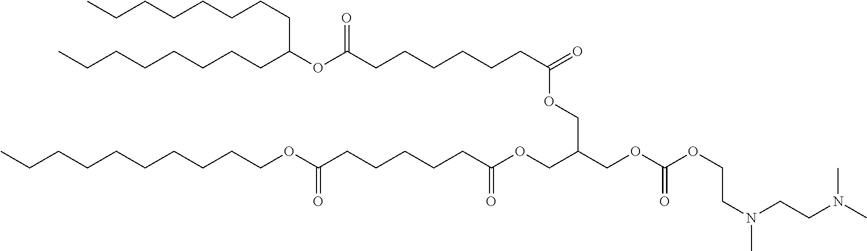 [SMILES: CCCCCCCCCCOC(=O)CCCCCC(=O)OCC(COC(=O)CCCCCCC(=O)OC(CCCCCCCC)CCCCCCCC)COC(=O)OCCN(C)CCN(C)C]
VL471A 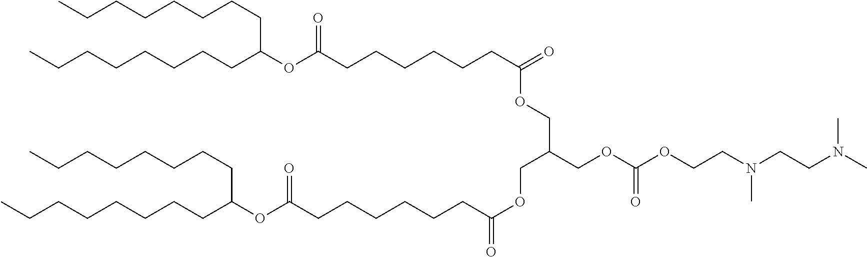 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCN(C)CCN(C)C]
VL472A 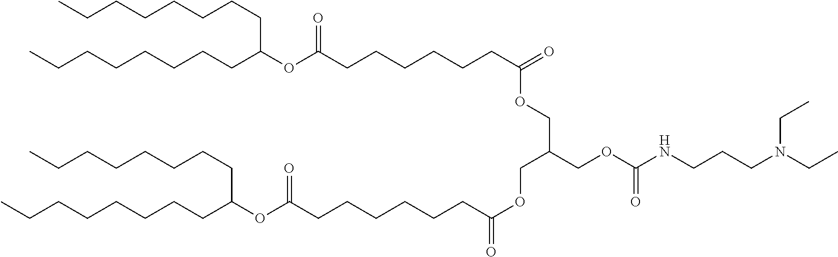 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)NCCCN(CC)CC]
VL473A 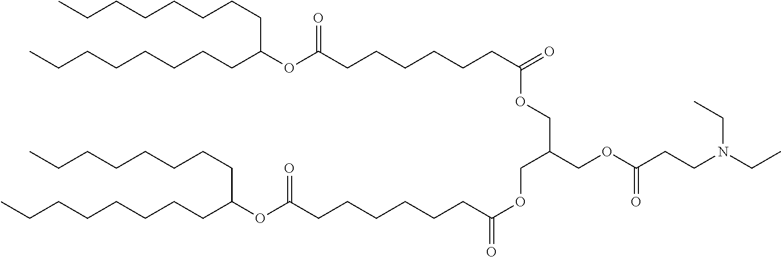 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)CCN(CC)CC]
VL474A 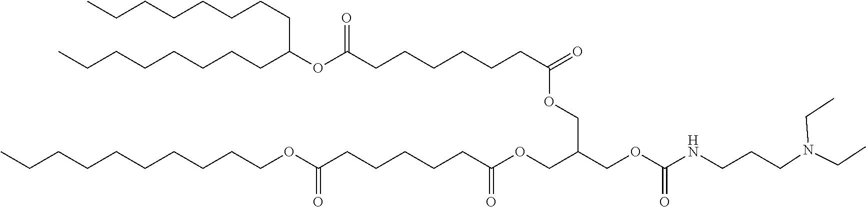 [SMILES: CCCCCCCCCCOC(=O)CCCCCC(=O)OCC(COC(=O)CCCCCCC(=O)OC(CCCCCCCC)CCCCCCCC)COC(=O)NCCCN(CC)CC]
VL475A 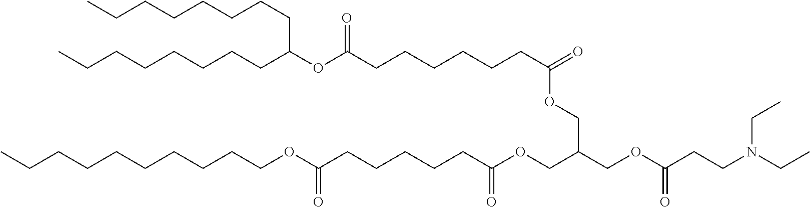 [SMILES: CCCCCCCCCCOC(=O)CCCCCC(=O)OCC(COC(=O)CCCCCCC(=O)OC(CCCCCCCC)CCCCCCCC)COC(=O)CCN(CC)CC]
VL476A 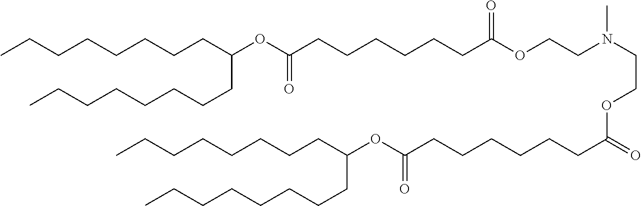 [SMILES: CCCCCCCCC(CCCCCCCC)OC(=O)CCCCCCC(=O)OCCN(C)CCOC(=O)CCCCCCC(=O)OC(CCCCCCCC)CCCCCCCC]
VL529 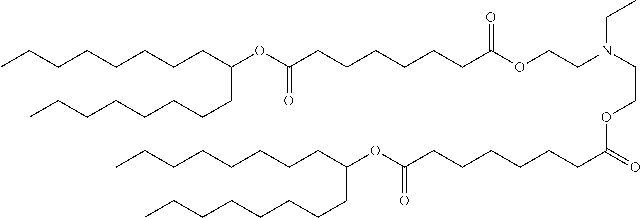 [SMILES: CCCCCCCCC(CCCCCCCC)OC(=O)CCCCCCC(=O)OCCN(CC)CCOC(=O)CCCCCCC(=O)OC(CCCCCCCC)CCCCCCCC]
VL530 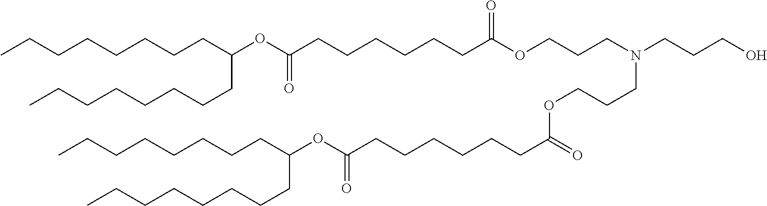 [SMILES: CCCCCCCCC(CCCCCCCC)OC(=O)CCCCCCC(=O)OCCCN(CCCO)CCCOC(=O)CCCCCCC(=O)OC(CCCCCCCC)CCCCCCCC]
VL522 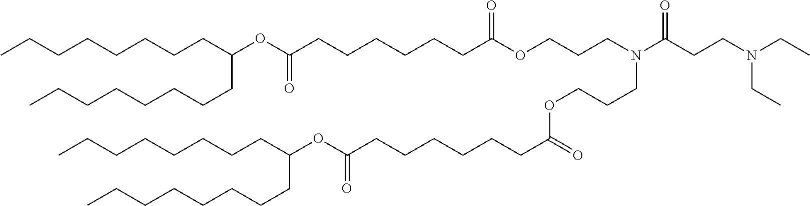 [SMILES: CCCCCCCCC(CCCCCCCC)OC(=O)CCCCCCC(=O)OCCCN(CCCOC(=O)CCCCCCC(=O)OC(CCCCCCCC)CCCCCCCC)C(=O)CCN(CC)CC]
VL531

In some embodiments of a compound of Table 1A, m is 1-10. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In some embodiments, an asymmetric carbon atom in Table 1A represents racemic, chirally pure R or chirally S. In some embodiments, if a compound of Table 1A contains two or more chiral centers, all the combinations of the stereochemistries for each of the chiral centers are encompassed by the disclosure.

In some embodiments, the disclosed amino lipids can be converted to N-oxides. In some embodiments, N-oxides are formed by a treatment with an oxidizing agent (e.g., 3-chloroperoxybenzoic acid and/or hydrogen peroxides). Accordingly, disclosed herein are N-oxide compounds of the described amino lipids, when allowed by valency and structure, which can be designated as N→0 or N⁺-0⁻. In some embodiments, the nitrogen in the compounds of the disclosure can be converted to N-hydroxy or N-alkoxy. For example, N-hydroxy compounds can be prepared by oxidation of the parent amine by an oxidizing agent such as ra-CPBA. All shown and claimed nitrogen-containing compounds are also considered. Accordingly, also disclosed herein are N-hydroxy and N-alkoxy (e.g., N—OR, wherein R is substituted or unsubstituted C1-C6 alkyl, C2-C6 alkenyl, C2-C6 alkynyl, 3-14-membered carbocycle or 3-14-membered heterocycle) derivatives of the described amino lipids.

In some embodiments, an amino lipid described herein can take the form of a salt, such as a pharmaceutically acceptable salt. All pharmaceutically acceptable salts of the amino lipid are encompassed by this disclosure. As used herein, the term “amino lipid” also includes its pharmaceutically acceptable salts, and its diastereomeric, enantiomeric, and epimeric forms.

In some embodiments, an amino lipid described herein, possesses one or more stereocenters and each stereocenter exists independently in either the R or S configuration. The lipids presented herein include all diastereomeric, enantiomeric, and epimeric forms as well as the appropriate mixtures thereof. The lipids provided herein include all cis, trans, syn, anti, entgegen (E), and zusammen (Z) isomers as well as the appropriate mixtures thereof. In certain embodiments, lipids described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, the lipids such as the amino lipids are substituted based on the structures disclosed herein. In some embodiments, the amino lipid is a lipid of Table 1A with one or more substituents. In some embodiments, the lipids such as the amino lipids are unsubstituted. In another embodiment, the lipids described herein are labeled isotopically (e.g. with a radioisotope) or by another other means, including, but not limited to, the use of chromophores or fluorescent moieties, bioluminescent labels, or chemiluminescent labels.

Lipids described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present lipids include isotopes of hydrogen, carbon, nitrogen, oxygen, sulfur, fluorine and chlorine, such as, for example, ²H, ₃h, ¹³C, ¹⁴C, ¹⁵N, ¹⁸O, ¹⁷O, ³⁵S, ¹⁸F, ³₆ cl. In one aspect, isotopically-labeled lipids described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the asymmetric carbon atom of the amino lipid is present in enantiomerically enriched form. In certain embodiments, the asymmetric carbon atom of the amino lipid has at least 5000 enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (S)- or (R)-configuration.

Also disclosed herein is a lipid of Table 11B, or a salt or solvate thereof.

TABLE 1B
Amino lipids and excipients for constituting lipid nanoparticles

| | |
|---|---|
| 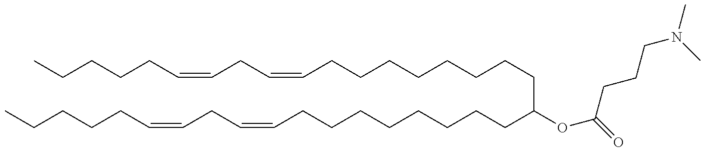 [SMILES: CCCCC/C=C\C/C=C\CCCCCCCCC(CCCCCCCC/C=C\C/C=C\CCCCC)OC(=O)CCCN(C)C] | 501 |
| 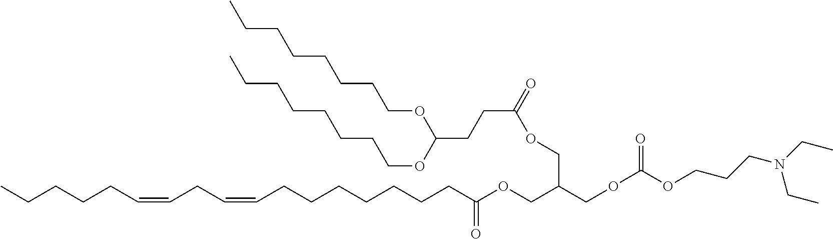 [SMILES: CCCCC/C=C\C/C=C\CCCCCCCC(=O)OCC(COC(=O)CCC(OCCCCCCCC)OCCCCCCCC)COC(=O)OCCCN(CC)CC] | 502 |
| 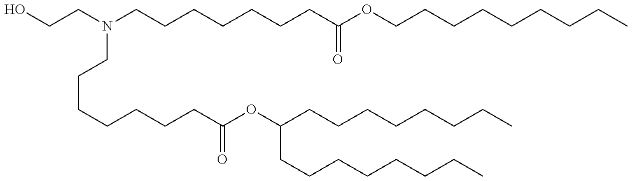 [SMILES: CCCCCCCCCOC(=O)CCCCCCCN(CCO)CCCCCCCC(=O)OC(CCCCCCCC)CCCCCCCC] | 503 |
| 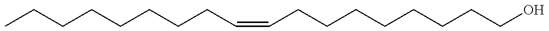 [SMILES: CCCCCCCC/C=C\CCCCCCCCO] | 504 |
| 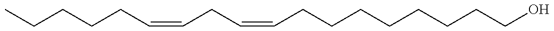 [SMILES: CCCCC/C=C\C/C=C\CCCCCCCCO] | 505 |
| 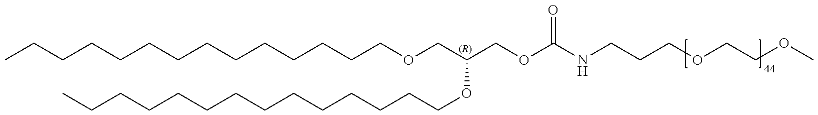 | 506 |
| 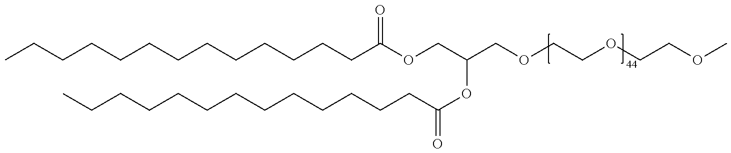 | 507 |
| 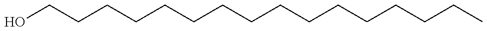 [SMILES: CCCCCCCCCCCCCCCCO] | 508 |
| 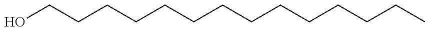 | 509 |
| 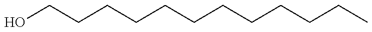 | 510 |
| 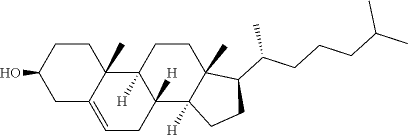 | 511 |
| 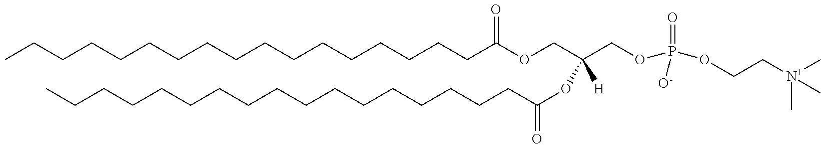 [SMILES: CCCCCCCCCCCCCCCCCC(=O)OC[C@H](COP(=O)([O-])OCC[N+](C)(C)C)OC(=O)CCCCCCCCCCCCCCCCC] | 512 |
| 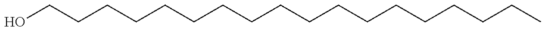 [SMILES: CCCCCCCCCCCCCCCCCCO] | 513 |
| 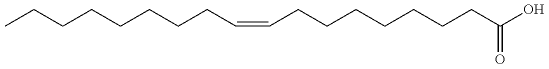 [SMILES: CCCCCCCC/C=C\CCCCCCCC(=O)O] | 514 |
| 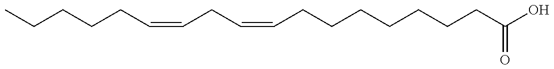 [SMILES: CCCCC/C=C\C/C=C\CCCCCCCC(=O)O] | 515 |
| 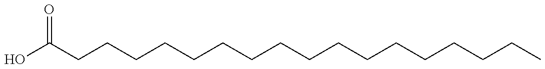 [SMILES: CCCCCCCCCCCCCCCCCC(=O)O] | 516 |
| 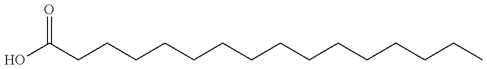 [SMILES: CCCCCCCCCCCCCCCC(=O)O] | 517 |
| 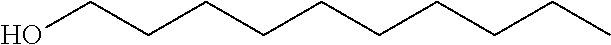 | 518 |
| 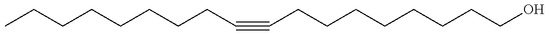 [SMILES: CCCCCCCCC#CCCCCCCCCO] | 519 |
| 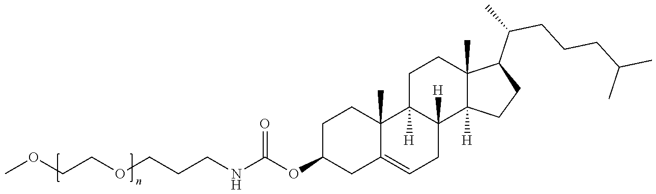 | 520 |
| 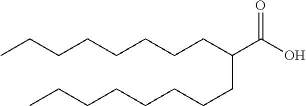 [SMILES: CCCCCCCCC(CCCCCCCC)C(=O)O] | 522 |
| 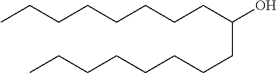 [SMILES: CCCCCCCCC(O)CCCCCCCC] | 521 |
| 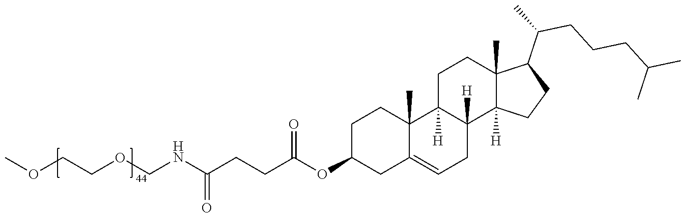 | 523 |
| 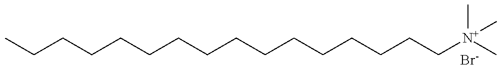 [SMILES: CCCCCCCCCCCCCCCC[N+](C)(C)C ; [Br-]] | 524 |

In some embodiments, an amino lipid (or other lipids) provided herein can be designated by more than one compound TD number in different parts of the disclosure.

### PEG-Lipid

In some embodiments, the described LNP composition comprises a PEG-lipid. In some embodiments, the described LNP composition comprises two or more PEG-lipids. Exemplary PEG-lipids include, but are not limited to, the lipids in Table 2. Exemplary PEG-lipids also include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG-lipids can comprise PEG-c-DOMG, PEG-DMG, PEG-DLPE, PEG-DMPE, PEG-DPPC, a PEG-DSPE lipid, or a combination thereof. In some embodiments, PEG moiety is an optionally substituted linear or branched polymer of ethylene glycol or ethylene oxide. In some embodiments, the PEG moiety is substituted, e.g., by one or more alkyl, alkoxy, acyl, hydroxy, or aryl groups. In some embodiments, the PEG moiety includes PEG copolymer such as PEG-polyurethane or PEG-polypropylene (see, e.g., j. Milton Harris, Poly(ethylene glycol) chemistry: biotechnical and biomedical applications (1992)). In some embodiments, the PEG moiety does not include PEG copolymers, e.g., it may be a PEG monopolymer. Exemplary PEG-lipids include, but are not limited to, PEG-dilauroylglycerol, PEG-dimyristoylglycerol (PEG-DMG), PEG-dipalmitoylglycerol, PEG-distearoylgiycerol (PEG-DSPE), PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol, and PEG-DMB (3,4-Ditetradecoxylbenzyl-[omega]-methyl-poly(ethylene glycol) ether), 1,2-dimyristoyl-sn-glycero-3-phosphoethanolamine-N-[methoxy(polyethylene glycol)-2000]).

In some embodiments, a PEG-lipid is a PEG-lipid conjugate, for example, PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG-lipids, polyoxazoline (POZ)-lipid conjugates (e.g., POZ-DAA conjugates; see, e.g., WO 2010/006282), polyamide oligomers (e.g., ATTA-lipid conjugates), and mixtures thereof.

In one aspect, disclosed herein is a PEG-lipid having the structure of Formula (III), or a pharmaceutically acceptable salt or solvate thereof,

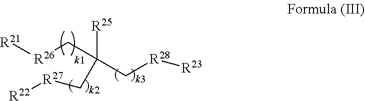

wherein
each of R²¹ and R²² is independently substituted or unsubstituted C₃-C₃₀ alkyl, substituted or unsubstituted C₃-C₃₀ alkenyl, or substituted or unsubstituted C₃-C₃₀ alkynyl;
each of R²⁶, R²⁷ and R²⁸ is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH—, —O—, —S—, or a bond;
each of R⁴ is independently hydrogen or substituted or unsubstituted C₁-C₆ alkyl;
R²³ is —C₀-C₁₀ alkylene-(CH₂—CH₂—O)ₖ₄—R²⁴ or —C₁-C₁₀ heteroalkylene-(CH₂—CH₂—O)ₖ₄—R²⁴, wherein the alkylene and heteroalkylene are each independently substituted or unsubstituted;
R²⁴ is independently hydrogen or substituted or unsubstituted C₁-C₃ alkyl;
R²⁵ is hydrogen or substituted or unsubstituted C₁-C₆ alkyl;
each of k1, k2, and k3 is independently 0, 1, 2, 3, 4, or 5; and
k4 is an integer selected from 1 to 100.

In one aspect, disclosed herein is a PEG-lipid having the structure of Formula (III*), or a pharmaceutically acceptable salt or solvate thereof,

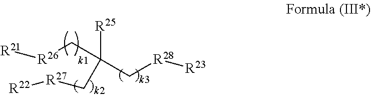

wherein
R²¹ is hydrogen, substituted or unsubstituted C₁-C₃₀ alkyl, substituted or unsubstituted C₃-C₃₀ alkenyl, or substituted or unsubstituted C₃-C₃₀ alkynyl;
R²² is hydrogen, substituted or unsubstituted C₁-C₃₀ alkyl, substituted or unsubstituted C₃-C₃₀ alkenyl, or substituted or unsubstituted C₃-C₃₀ alkynyl;
each of R²⁶, R²⁷ and R²⁸ is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —N(Ac)—, —OC(O)OCH₂C(O)O—, —OC(O)OCH₂C(O)NH— —OC(O)NHCH₂C(O)O—, —OC(O)NHCH₂C(O)NH—, —OC(O)O(CH₂)₂C(O)O—, —OC(O)O(CH₂)₂C(O)NH—, —OC(O)NH(CH₂)₂C(O)O—, —OC(O)NH(CH₂)₂C(O)NH—, —OC(O)O(CH₂)₃C(O)O—, —OC(O)O(CH₂)₃C(O)NH—, —OC(O)NH(CH₂)₃C(O)O—, —OC(O)NH(CH₂)₃C(O)NH—, —C(O)OCH₂C(O)NH— —C(O)NHCH₂C(O)O—, —C(O)NHCH₂C(O)NH—, —C(O)O(CH₂)₂C(O)O—, —C(O)O(CH₂)₂C(O)NH—, —C(O)NH(CH₂)₂C(O)O—, —C(O)NH(CH₂)₂C(O)NH—, —C(O)O(CH₂)₃C(O)O—, —C(O)O(CH₂)₃C(O)NH—, —C(O)NH(CH₂)₃C(O)O—, —C(O)NH(CH₂)₃C(O)NH—, —OC(═O)NR⁴(CH₂)₁₋₃C(═O)NH—, —OC(═O)NR⁴(CH₂)₁₋₃C(═O)O—, —C(═O)NR⁴(CH₂)₁₋₃C(═O)NH—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)—, —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH—, —O—, —S—, or a bond;
each of R⁴ is independently hydrogen or substituted or unsubstituted C₁-C₆ alkyl;
R²³ is —C₀-C₁₀ alkylene-(CH₂—CH₂—O)ₖ₄—R²⁴, —C₁-C₁₀ heteroalkylene-(CH₂—CH₂—O)ₖ₄—R²⁴, —C₀-C₁₀ alkylene-(O—CH₂—CH₂)ₖ₄—R²⁴ or —C₁-C₁₀ heteroalkylene-(O—CH₂—CH₂)ₖ₄—R²⁴, wherein the alkylene and heteroalkylene are each independently substituted or unsubstituted;
R²⁴ is independently hydrogen, substituted or unsubstituted C₁-C₃ alkyl, —O—R⁴, —C(═O)OH, or —C(═O)R⁴;
R²⁵ is hydrogen or substituted or unsubstituted C₁-C₂₂ alkyl;
each of k1, k2, and k3 is independently 0, 1, 2, 3, 4, or 5; and
k4 is an integer selected from 1 to 100.

In some embodiments, the PEG-lipid of Formula (III) or (III*), or a pharmaceutically acceptable salt or solvate thereof, wherein
R²¹ is substituted or unsubstituted C₃-C₃₀ alkyl, substituted or unsubstituted C₃-C₃₀ alkenyl, or substituted or unsubstituted C₃-C₃₀ alkynyl;
R²² is hydrogen, substituted or unsubstituted C₃-C₃₀ alkyl, substituted or unsubstituted C₃-C₃₀ alkenyl, or substituted or unsubstituted C₃-C₃₀ alkynyl;
each of R²⁶, R²⁷ and R²⁸ is independently —C(═O)NR⁴—, —NR⁴C(═O)—, —C(═O)O—, —OC(═O)—, —OC(═O)O—, —NR⁴C(═O)O—, —OC(═O)NR⁴—, —NR⁴C(═O)NR⁴—, —NR⁴C(═NR⁴)NR⁴—, —C(═S)NR⁴—, —NR⁴C(═S)—, —C(═S)O—, —OC(═S)—, OC(═S)O—, —NR⁴C(═S)O—, —OC(═S)NR⁴—, —NR⁴C(═S)NR⁴—, —C(═O)S—, —SC(═O)—, —OC(═O)S—, —NR⁴C(═O)S—, —OC(═O)NR⁴C(═O)NH—, —OC(═O)NR⁴C(═O)O—, —C(═O)NR⁴C(═O)NH—, —SC(═O)NR⁴—, —C(═S)S—, —SC(═S)—, —SC(═S)O—, —NR⁴C(═S)S—, —SC(═S)NR⁴—, —C(═S)S—, —SC(═S)—, —SC(═O)S—, —SC(═S)S—, —NR⁴C(═S)S—, —SC(═S)NR⁴—, —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)—, —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH—, —O—, —S—, or a bond;
each of R⁴ is independently hydrogen or substituted or unsubstituted C₁-C₆ alkyl;
R²³ is —C₀-C₁₀ alkylene-(CH₂—CH₂—O)ₖ₄—R²⁴ or —C₁-C₁₀ heteroalkylene-(CH₂—CH₂—O)ₖ₄—R²⁴, wherein the alkylene and heteroalkylene are each independently substituted or unsubstituted;
R²⁴ is independently hydrogen, substituted or unsubstituted C₁-C₃ alkyl, —O—R⁴, —C(═O)OR⁴, or —C(═O)R⁴;
R²⁵ is hydrogen or substituted or unsubstituted C₁-C₆ alkyl;
each of k1, k2, and k3 is independently 0, 1, 2, 3, 4, or 5; and
k4 is an integer selected from 1 to 100.

In some embodiments of a compound of Formula (III) or (III*), or a pharmaceutically acceptable salt or solvate thereof, each of R²¹ and R²² is independently unsubstituted or substituted, linear or branched C₁₂-C₃₀ alkyl. In some embodiments, each of R²¹ and R²² is independently unsubstituted, linear C₁₂-C₂₅ alkyl. In some embodiments of a compound of Formula (III), or a pharmaceutically acceptable salt or solvate thereof, each of R²¹ and R²² is independently unsubstituted or substituted, linear or branched C₁₂-C₃₀ alkenyl. In some embodiments, each of R²¹ and R²² is independently unsubstituted, linear C₁₂-C₂₅ alkenyl.

In some embodiments of a compound of Formula (III) or (III*), or a pharmaceutically acceptable salt or solvate thereof, each of R²⁶ and R²⁷ is —O—, —C(═O)O—, or —OC(═O)—. In some embodiments, each of R²⁶ and R²⁷ is —O—. In some embodiments, each of R²⁶ and R²⁷ is a bond.

In some embodiments of a compound of Formula (III) or (III*), or a pharmaceutically acceptable salt or solvate thereof, R²⁸ is —O—, —C(═O)O—, —OC(═O)—, or —OC(═O)NR⁴. In some embodiments of a compound of Formula (III*), R²⁸ is —OC(═O)NR⁴. In some embodiments of a compound of Formula (III) or (III*), or a pharmaceutically acceptable salt or solvate thereof, R²⁸ is —C(═O)O— or —OC(═O)—.

In some embodiments of a compound of Formula (III*), or a pharmaceutically acceptable salt or solvate thereof, R²⁶ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH, —N(Ac)—, —OC(O)O(CH₂)C(O)O—, —OC(O)O(CH₂)C(O)(NH)— —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, —C(O)(NH)(CH₂)₃C(O)(NH)— or a bond.

In some embodiments of a compound of Formula (III) or (III*), or a pharmaceutically acceptable salt or solvate thereof, R²⁶ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —C(O)NH(CH₂)₂O—, —C(O)N(Me)(CH₂)₂NH—, —O₂C(NH)(CH₂)N(CH₃)—, or —O(CH₂)₂C(NH)NH—.

In some embodiments of a compound of Formula (III*), or a pharmaceutically acceptable salt or solvate thereof, R²⁷ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH, —N(Ac)—, —OC(O)O(CH₂)C(O)O—, —OC(O)O(CH₂)C(O)(NH)— —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, —C(O)(NH)(CH₂)₃C(O)(NH)—, or a bond.

In some embodiments of a compound of Formula (III) or (III*), or a pharmaceutically acceptable salt or solvate thereof, R²⁷ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, or —O(CH₂)₂C(NH)NH—.

In some embodiments of a compound of Formula (III*), or a pharmaceutically acceptable salt or solvate thereof, R²⁸ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH, —N(Ac)—, —OC(O)O(CH₂)C(O)O—, —OC(O)O(CH₂)C(O)(NH)— —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, or —C(O)(NH)(CH₂)₃C(O)(NH)—.

In some embodiments of a compound of Formula (III) or (III*), or a pharmaceutically acceptable salt or solvate thereof, R²⁸ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, or —O(CH₂)₂C(NH)NH—.

In some embodiments of a compound of Formula (III) or (III*), or a pharmaceutically acceptable salt or solvate thereof, k1 is 0, 1, or 2. In some embodiment, k1 is 0. In some embodiment, k1 is 1. In some embodiment, k1 is 2.

In some embodiments of a compound of Formula (III) or (III*), or a pharmaceutically acceptable salt or solvate thereof, k2 is 0, 1, or 2. In some embodiment, k2 is 0. In some embodiment, k2 is 1. In some embodiment, k2 is 2.

In some embodiments of a compound of Formula (III) or (III*), or a pharmaceutically acceptable salt or solvate thereof, k3 is 0, 1, or 2. In some embodiment, k3 is 0. In some embodiment, k3 is 1. In some embodiment, k3 is 2.

In some embodiments of a compound of Formula (III) or (III*), or a pharmaceutically acceptable salt or solvate thereof, R²³ is —C₀-C₃ alkylene-(CH₂—CH₂—O)ₖ₄—R²⁴, wherein R²⁴ is H, ethyl or methyl. In some embodiments of a compound of Formula (III) or (III*), or a pharmaceutically acceptable salt or solvate thereof, R²³ is —C₁-C₅ heteroalkylene-(CH₂—CH₂—O)ₖ₄—R²⁴, wherein R²⁴ is H or methyl. In some embodiments of a compound of Formula (III) or (III*), or a pharmaceutically acceptable salt or solvate thereof, R²³ is —C₂ alkylene-(CH₂—CH₂—O)ₖ₄—R²⁴; wherein R²⁴ is —O—CH₃. In some embodiments of a compound of Formula (III) or (III*), or a pharmaceutically acceptable salt or solvate thereof, R²³ is —C₃ alkylene-(CH₂—CH₂₋₀)ₖ₄—R²⁴; wherein R²⁴ is —O—CH₃.

In some embodiments of a compound of Formula (III) or (III*), or a pharmaceutically acceptable salt or solvate thereof, k4 is 1 to 75. In some embodiments, k4 is 1 to 50, 1 to 30, 1 to 25, 1 to 20, 1 to 15, 1 to 10, 1 to 5, 20 to 50, or 5 to 50. In some embodiments, k4 is 30 to 50. In some embodiments, k4 is 35 to 45. In some embodiments, k4 is 40 to 50. In some embodiments, k4 is 36 to 48.

In some embodiments of a compound of Formula (III) or (III*), or a pharmaceutically acceptable salt or solvate thereof, R²⁵ is hydrogen.

In some embodiments of a compound of Formula (III*), R²² is the the unsubstituted C₂₈ alkenyl:

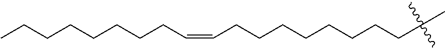 [SMILES: *CCCCCCCC/C=C\CCCCCCCC]

In some embodiments of a compound of Formula (III*), or a pharmaceutically acceptable salt or solvate thereof, R²¹ is C₁₂₋₂₂ substituted or unsubstituted alkyl; R²² is C₁₂₋₂₂ substituted or unsubstituted alkyl; R²⁵ is H; R²⁶ is —O—; R²⁷ is —O—; R²⁸ is —N(Ac)—, —OC(O)O(CH₂)C(O)O—, —OC(O)O(CH₂)C(O)(NH)—, —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, or —C(O)(NH)(CH₂)₃C(O)(NH)—; k1 is 1, k2 is zero and k3 is 1

In some embodiments of a compound of Formula (III*), or a pharmaceutically acceptable salt or solvate thereof, R²⁵ is C₁₀₋₂₂ substituted or unsubstituted alkyl, R²⁸ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —OC(NH)(CH₂)₂O—, —OC(NMe)(CH₂)₂NH—, —O₂C(NH)(CH₂)N(CH₃)—, —O(CH₂)₂C(NH)NH, —N(Ac)—, —OC(O)O(CH₂)C(O)O—, —OC(O)O(CH₂)C(O)(NH)— —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, or —C(O)(NH)(CH₂)₃C(O)(NH)— and k3 is 1, wherein R²¹ is hydrogen, R²² is hydrogen, R²⁶ is a bond, R²⁷ is a bond, k1 is 0, and k2 is 0.

In some embodiments of a compound of Formula (III*), or a pharmaceutically acceptable salt or solvate thereof, R²⁵ is C₁₀₋₂₂ alkyl; R²⁸ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —O C(NH)(CH₂)₂O—, —OC(NMe)(CH₂)₂NH—, —O₂C(NH)(CH₂)N(CH₃)—, —O(CH₂)₂C(NH)NH, —N(Ac)—, —OC(O)O(CH₂)C(O)O—, —OC(O)O(CH₂)C(O)(NH)— —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, or —C(O)(NH)(CH₂)₃C(O)(NH)— and k3 is 1; wherein R²¹ is a hydrogen, R²² is hydrogen, R²⁶ is a bond, R²⁷ is a bond, k1 is 0, and k2 is 0.

In some embodiments of a compound of Formula (III*), or a pharmaceutically acceptable salt or solvate thereof, R²¹ is C₁₄₋₁₈ substituted or unsubstituted alkyl; R²² is C₁₄₋₁₈ substituted or unsubstituted alkyl; R²⁵ is H; R²⁶ is —O—; R²⁷ is —O—; R²⁸ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —O C(NH)(CH₂)₂O—, —OC(NMe)(CH₂)₂NH—, —O₂C(NH)(CH₂)N(CH₃)—, —O(CH₂)₂C(NH)NH, —N(Ac)—, —OC(O)O(CH₂)C(O)O—, —OC(O)O(CH₂)C(O)(NH)— —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, or —C(O)(NH)(CH₂)₃C(O)(NH)—; k1 is 1, k2 is zero and k3 is 1

In some embodiments of a compound of Formula (III*), or a pharmaceutically acceptable salt or solvate thereof, R²¹ is C₁₄ substituted or unsubstituted alkyl; R²² is C₁₄ substituted or unsubstituted alkyl; R²⁵ is H; R²⁶ is —O—; R²⁷ is —O—; R²⁸ is —O(CH₂)₂C(O)O—, —O(CH₂)₂C(O)NH—, —OC(O)(CH₂)₂O—, —OC(O)(CH₂)₂NH—, —O(CH₂)₂C(O)N(CH₃)—, —O(CH₂)₂C(NH)O—, —O(CH₂)₂C(NAc)NH—, —O(CH₂)₂C(NH)N(CH₃)—, —O(CH₂)₂C(NH)NH—, —O(CH₂)₂C(NH)N(CH₃)—, —NH(CH₂)₂C(O)O—, —NH(CH₂)₂C(O)NH—, NH(CH₂)₂C(O)N(CH₃)—, —NH(CH₂)₂C(NH)O—, —NH(CH₂)₂C(NAc)NH—, —NH(CH₂)₂C(NH)N(CH₃)—, —NH(CH₂)₂C(NH)NH—, —NH(CH₂)₂C(NH)N(CH₃)— —OC(O)(CH₂)₂N(CH₃)—, —O C(NH)(CH₂)₂O—, —OC(NMe)(CH₂)₂NH—, —O₂C(NH)(CH₂)N(CH₃)—, —O(CH₂)₂C(NH)NH, —N(Ac)—, —OC(O)O(CH₂)C(O)O—, —OC(O)O(CH₂)C(O)(NH)— —OC(O)(NH)(CH₂)C(O)O—, —OC(O)(NH)(CH₂)C(O)(NH)—, —OC(O)O(CH₂)₂C(O)O—, —OC(O)O(CH₂)₂C(O)(NH)—, —OC(O)(NH)(CH₂)₂C(O)O—, —OC(O)(NH)(CH₂)₂C(O)(NH)—, —OC(O)O(CH₂)₃C(O)O—, —OC(O)O(CH₂)₃C(O)(NH)—, —OC(O)(NH)(CH₂)₃C(O)O—, —OC(O)(NH)(CH₂)₃C(O)(NH)—, —C(O)O(CH₂)C(O)(NH)— —C(O)(NH)(CH₂)C(O)O—, —C(O)(NH)(CH₂)C(O)(NH)—, —C(O)O(CH₂)₂C(O)O—, —C(O)O(CH₂)₂C(O)(NH)—, —C(O)(NH)(CH₂)₂C(O)O—, —C(O)(NH)(CH₂)₂C(O)(NH)—, —C(O)O(CH₂)₃C(O)O—, —C(O)O(CH₂)₃C(O)(NH)—, —C(O)(NH)(CH₂)₃C(O)O—, or —C(O)(NH)(CH₂)₃C(O)(NH)—; k1 is 1, k2 is zero and k3 is 1.

In some embodiments, disclosed herein is a PEG-lipid comprising a structure of —O(CH₂)₂C(O)O— or —O(CH₂)₂C(O)NH—.

In one aspect, disclosed herein is a PEG-lipid of Table 2.

TABLE 2
Exemplary PEG-Lipids for constituting LNP 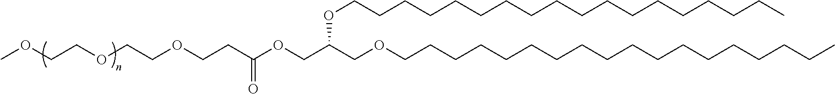
VP101 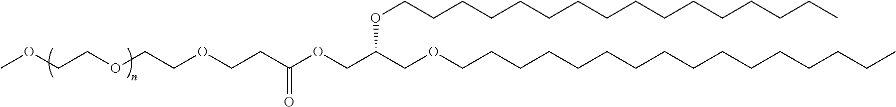
VP102 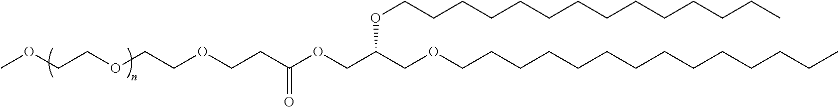
VP103 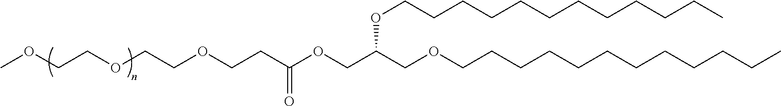
VP104 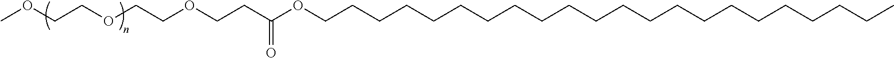
VP105 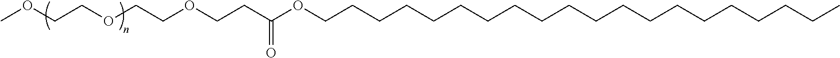
VP107 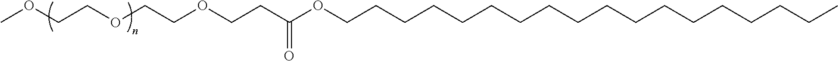
VP108 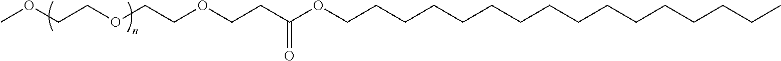
VP109 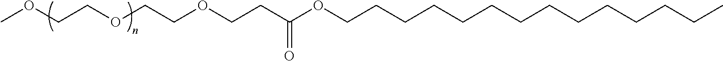
VP110 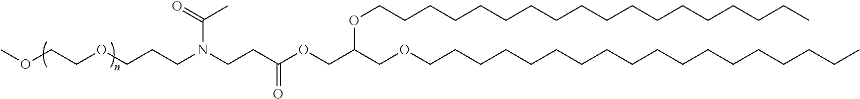
VP111 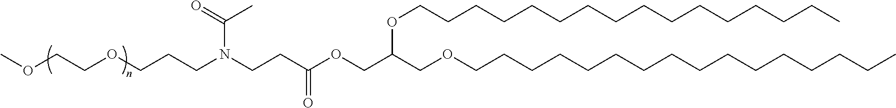
VP112 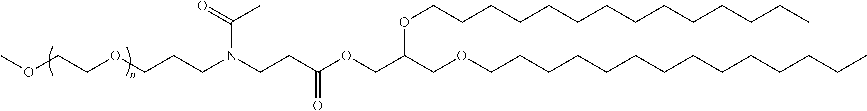
VP113 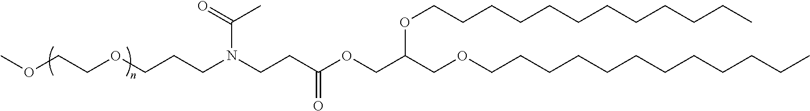
VP114 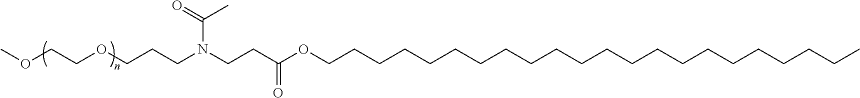
VP115 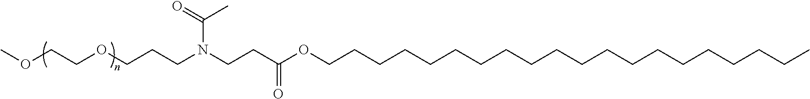
VP116 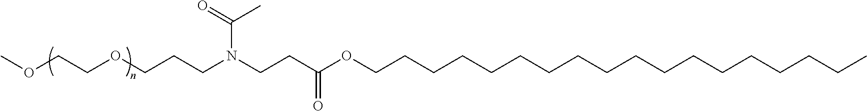
VP117 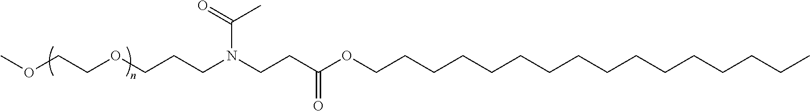
VP118 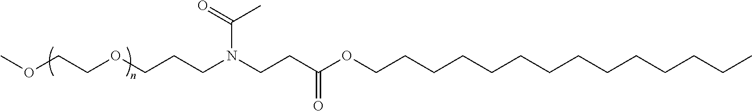
VP119 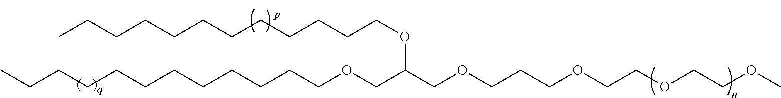
VP120 (p, q = 7) 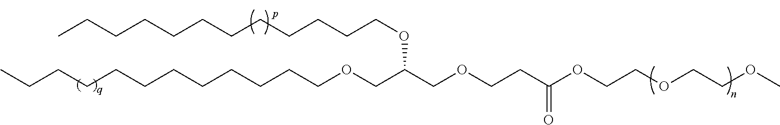
VP121 (p, q = 7) 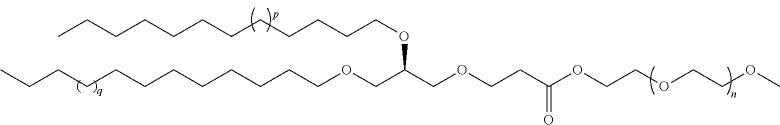
VP122 (p, q = 7) 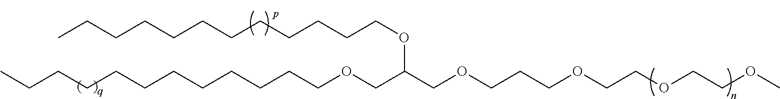
VP123 (p, q = 5) 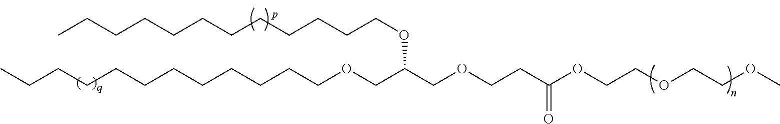
VP124 (p, q = 5) 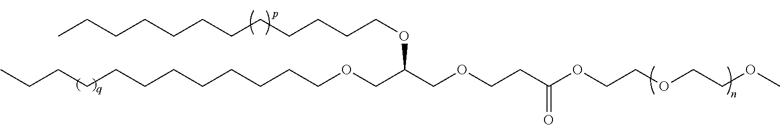
VP125 (p, q = 5) 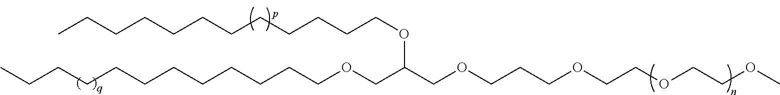
VP126 (p, q = 3) 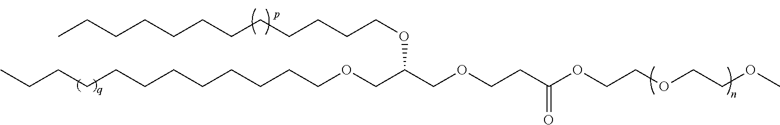
VP127 (p, q = 3) 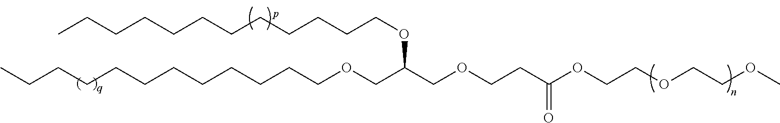
VP128 (p, q = 3) 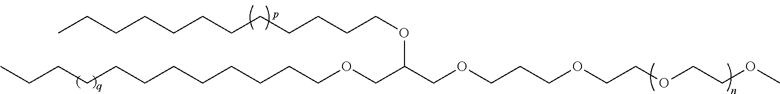
VP129 (p, q = 1) 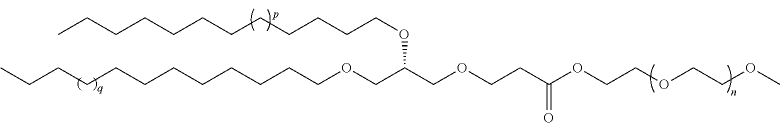
VP130 (p, q = 1) 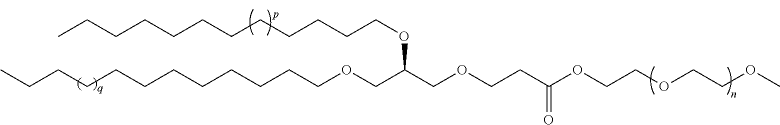
VP131 (p, q = 1) 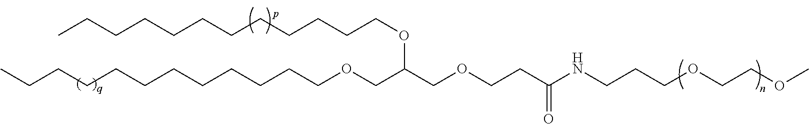
VP132 (p, q = 7) 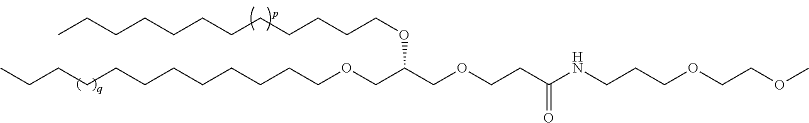
VP133 (p, q = 7) 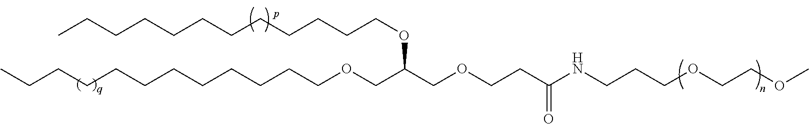
VP134 (p, q = 7) 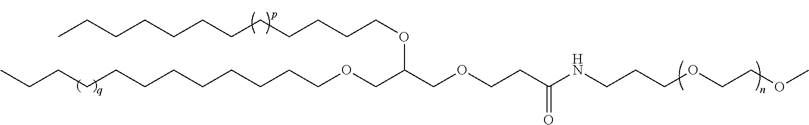
VP135 (p, q = 5) 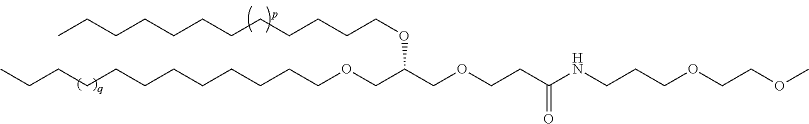
VP136 (p, q = 5) 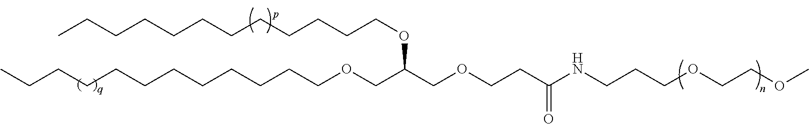
VP137 (p, q = 5) 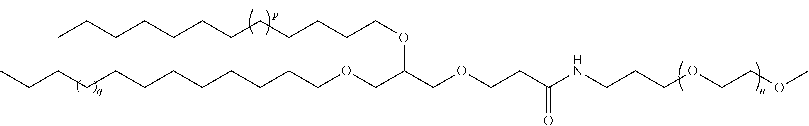
VP138 (p, q = 3) 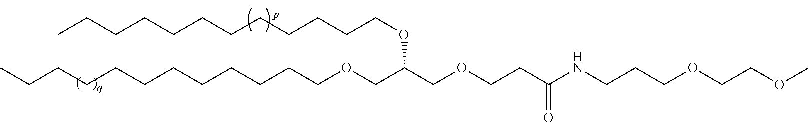
VP139 (p, q = 3) 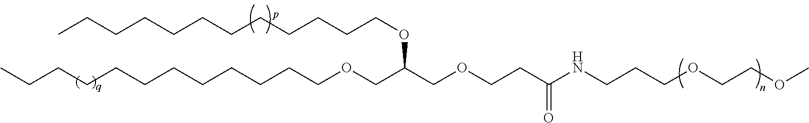
VP140 (p, q = 3) 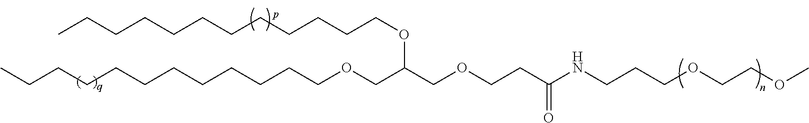
VP141 (p, q = 1) 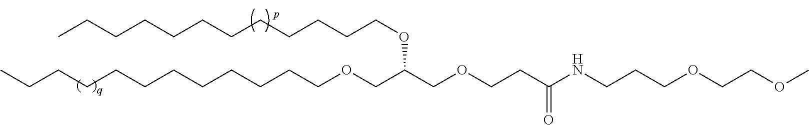
VP142 (p, q = 1)

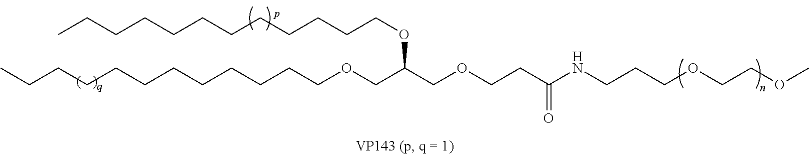

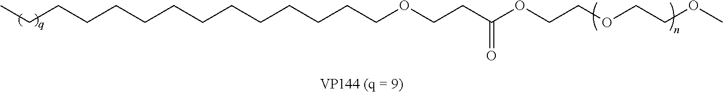

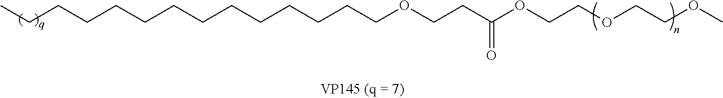

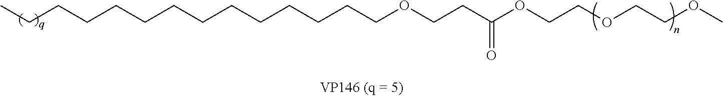

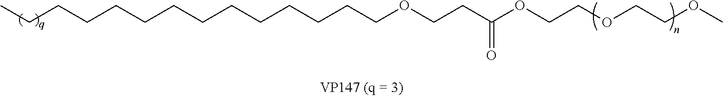

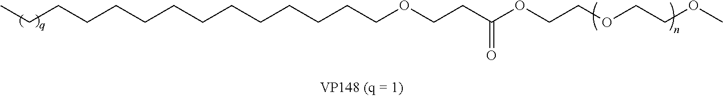

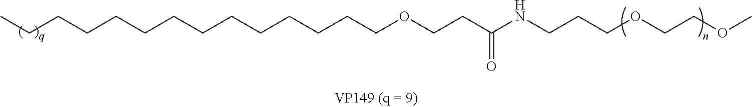

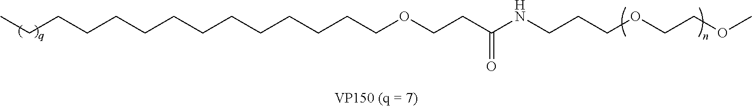

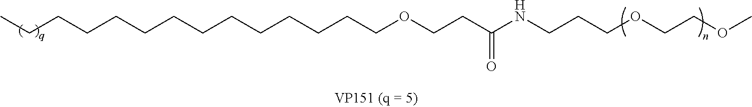

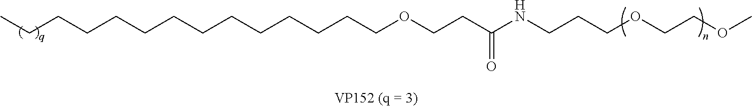

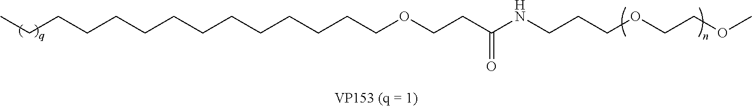

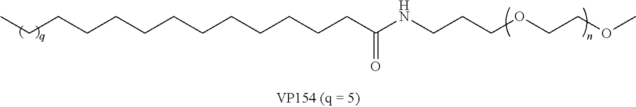

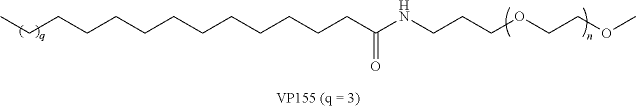

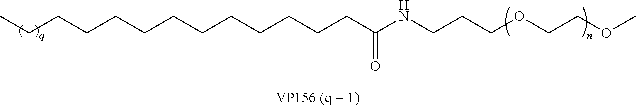

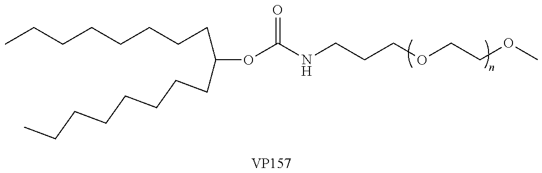

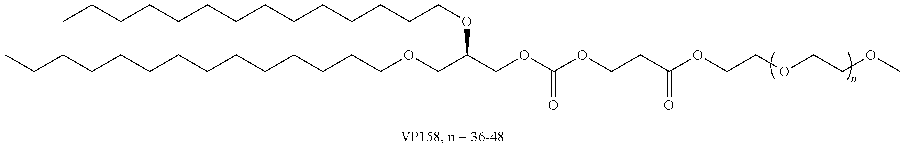

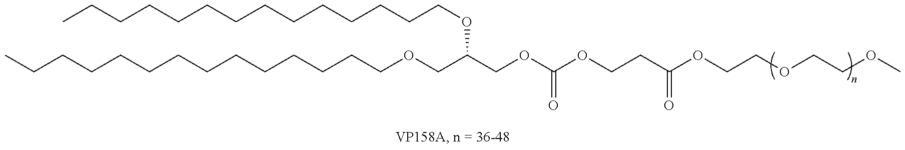

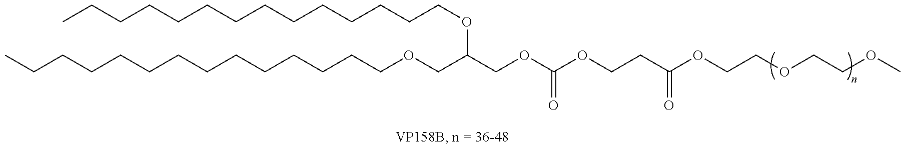

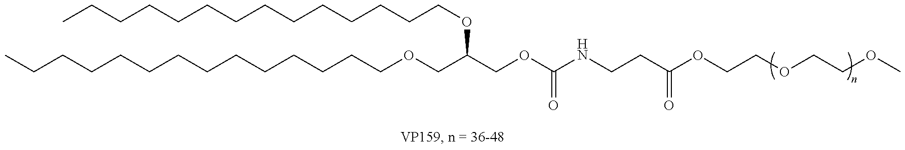

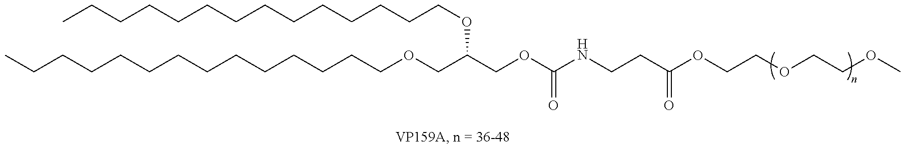

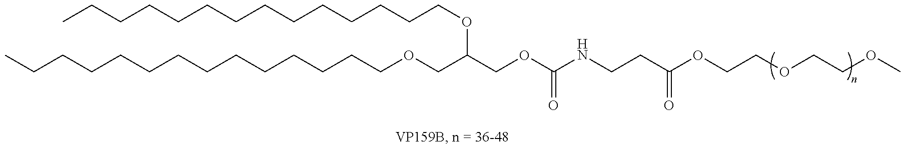

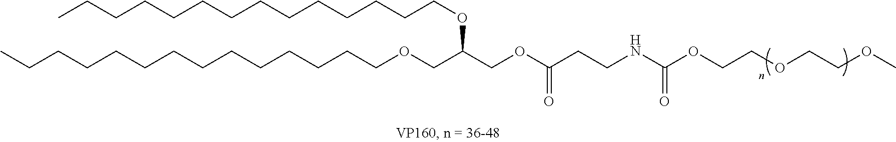

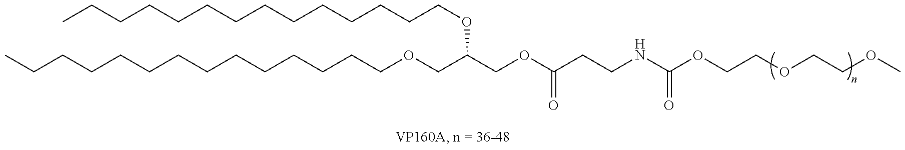

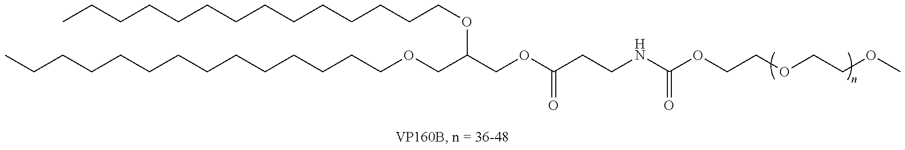

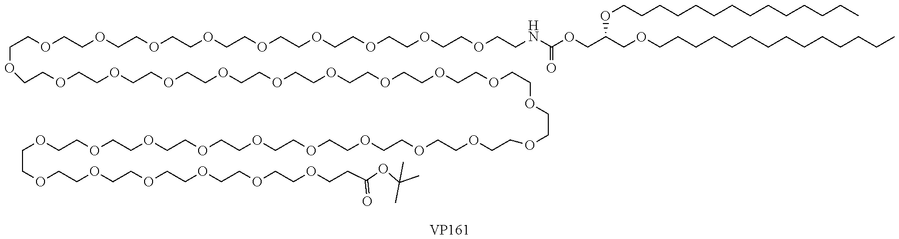 [SMILES: CCCCCCCCCCCCCCOC[C@H](COC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)OC(C)(C)C)OCCCCCCCCCCCCCC ; "VP161"]

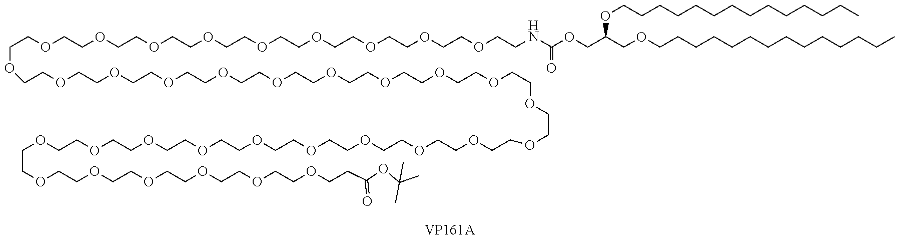 [SMILES: CCCCCCCCCCCCCCOC[C@@H](COC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)OC(C)(C)C)OCCCCCCCCCCCCCC ; "VP161A"]

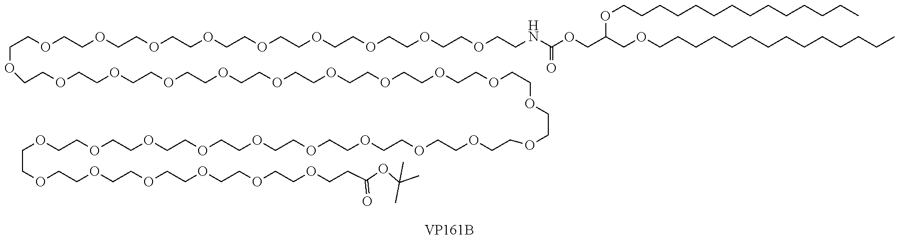 [SMILES: CCCCCCCCCCCCCCOCC(COC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)OC(C)(C)C)OCCCCCCCCCCCCCC ; "VP161B"]

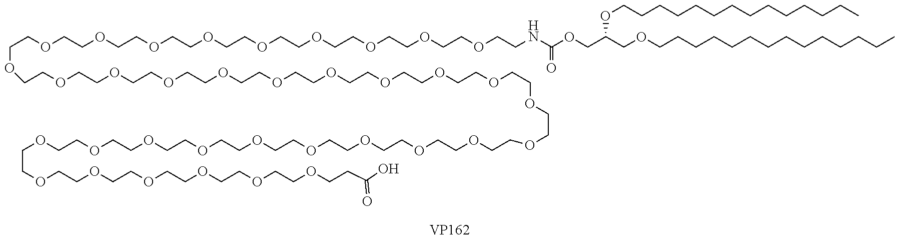 [SMILES: CCCCCCCCCCCCCCOC[C@H](COC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCC(=O)O)OCCCCCCCCCCCCCC ; "VP162"]

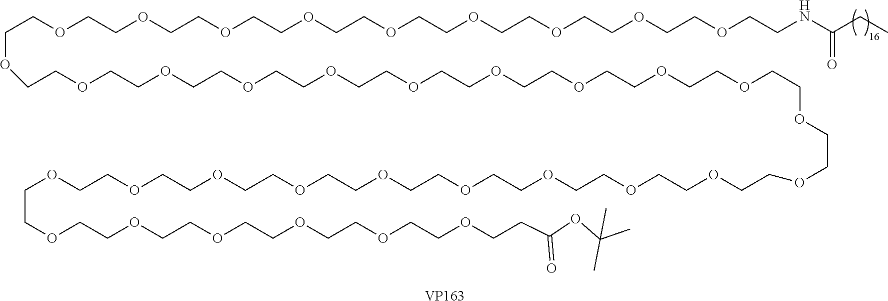

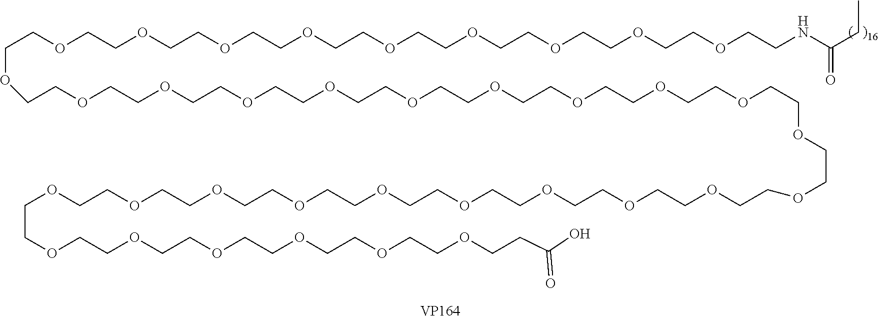

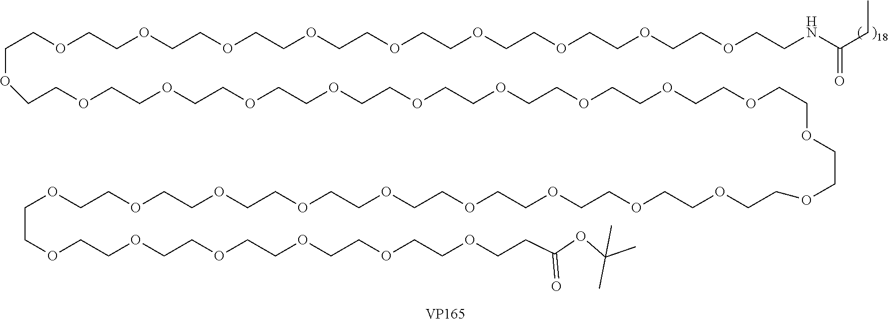

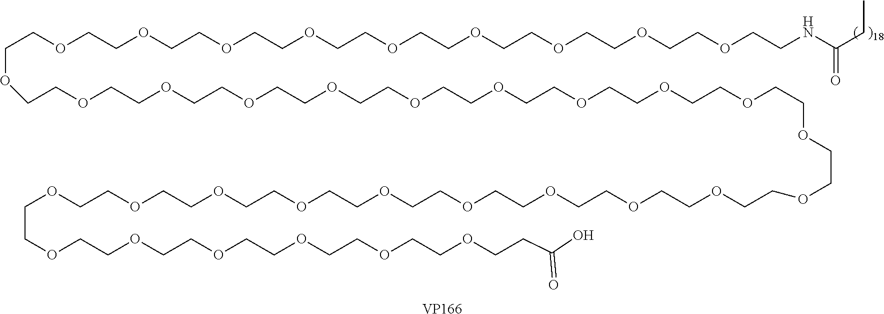

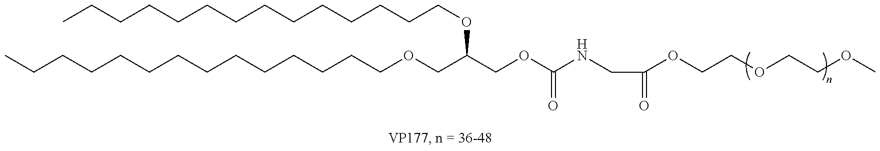

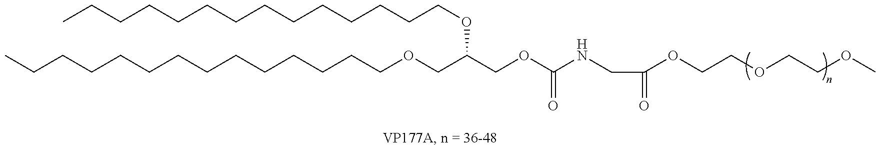

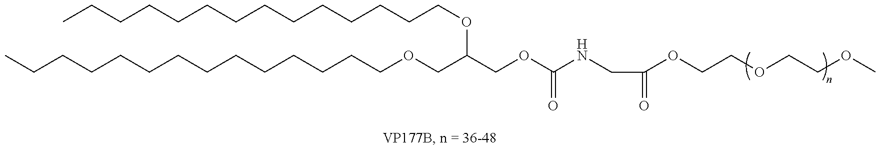

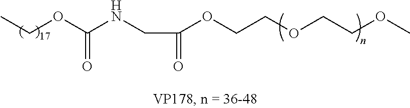

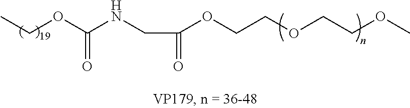

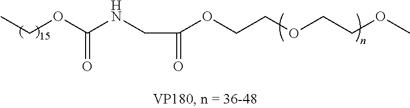

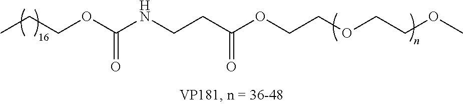

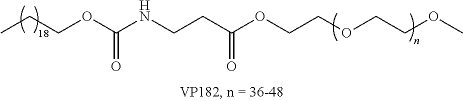

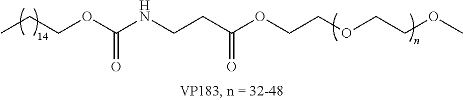

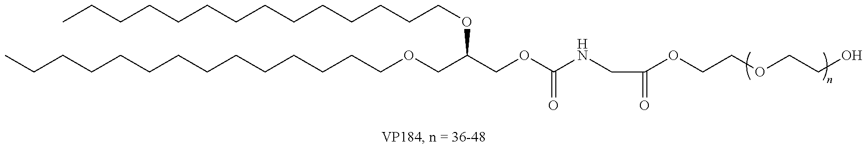

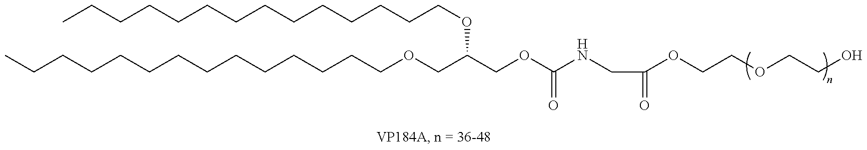

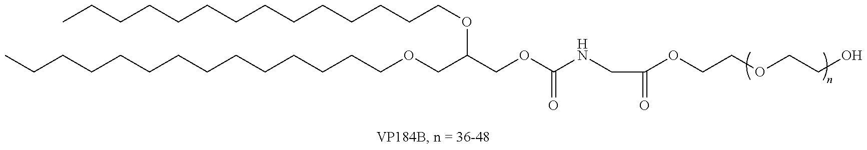

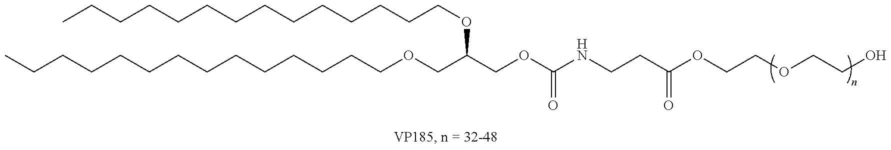

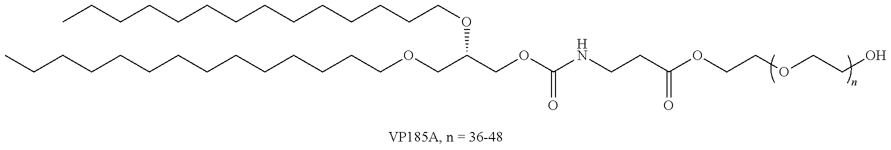

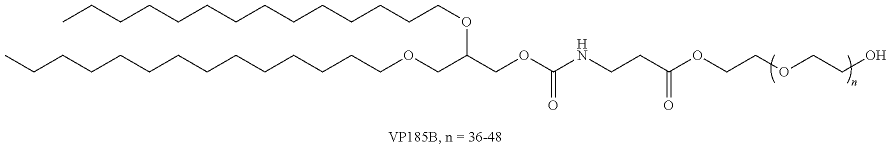

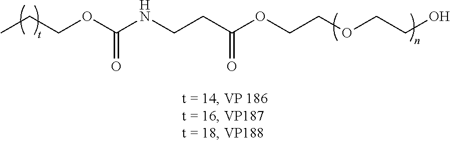

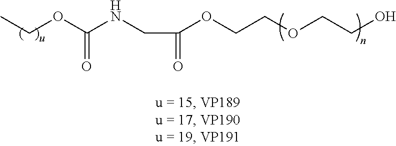

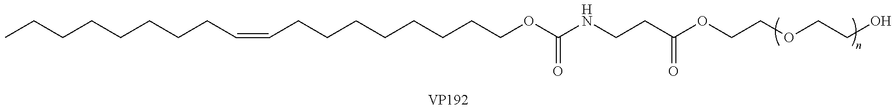

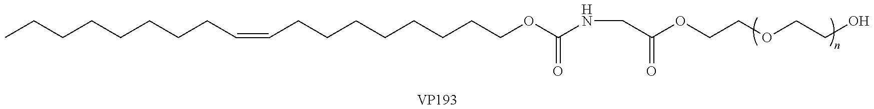

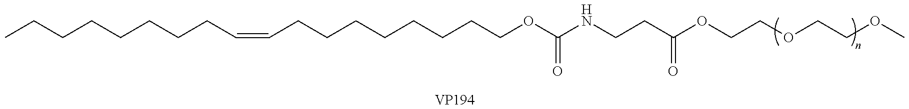

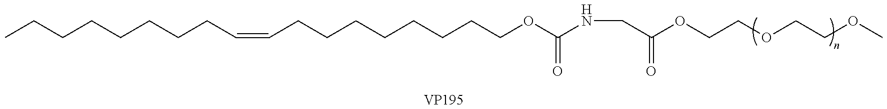

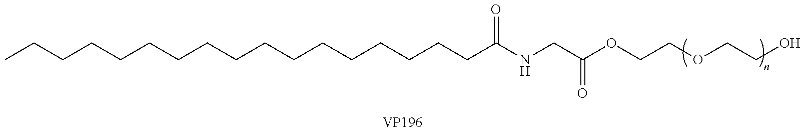

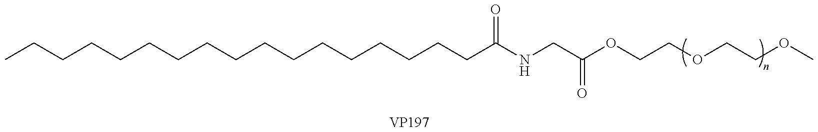

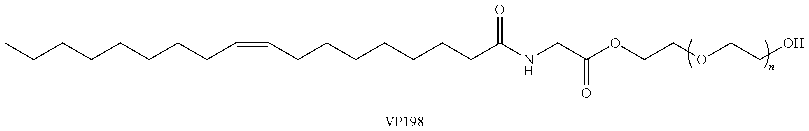

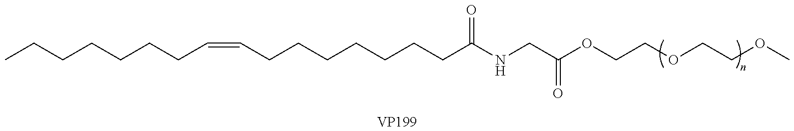

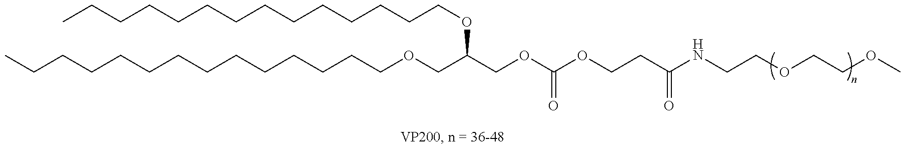

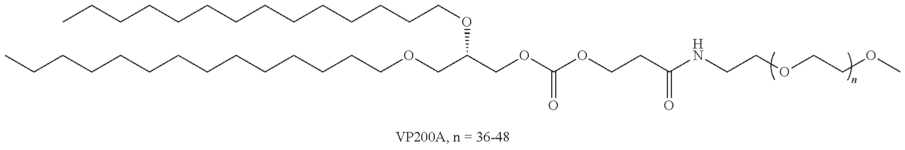

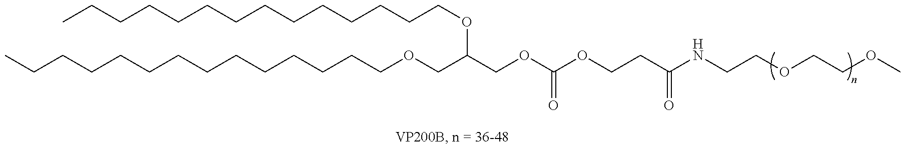

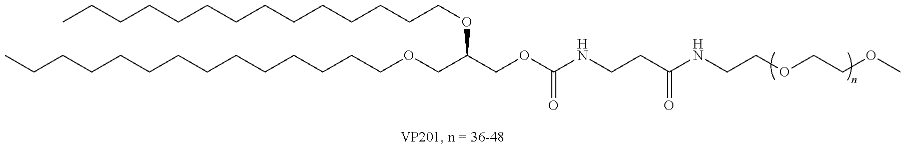

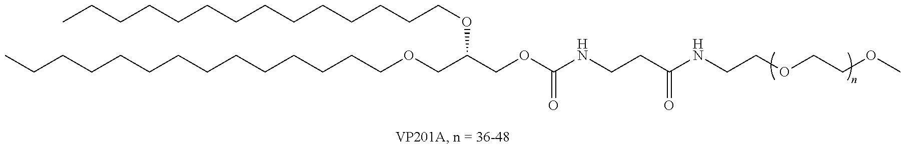

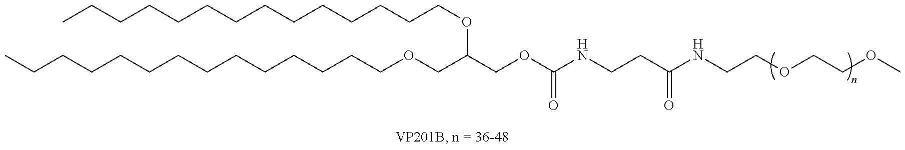

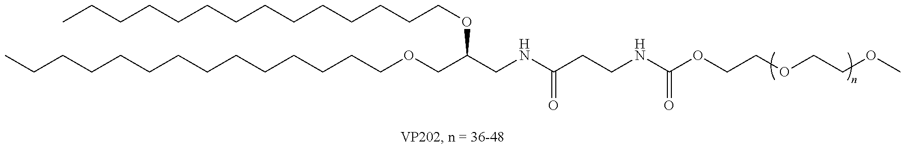

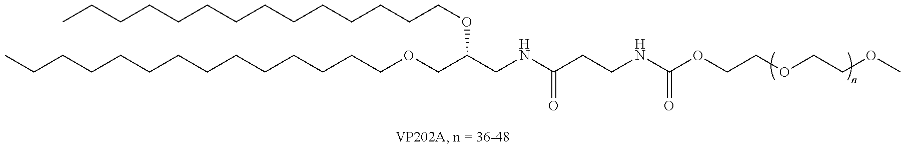

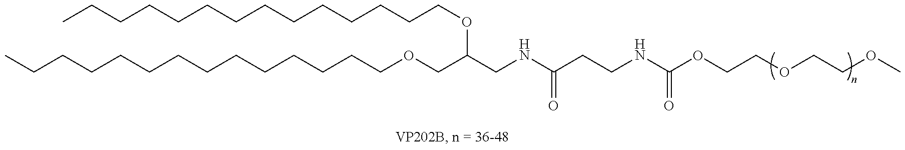

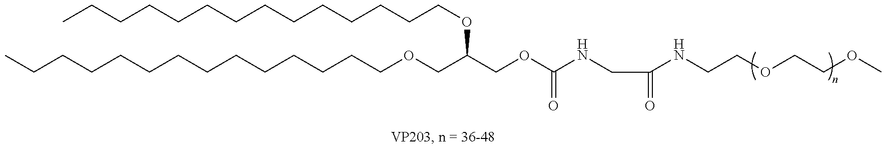

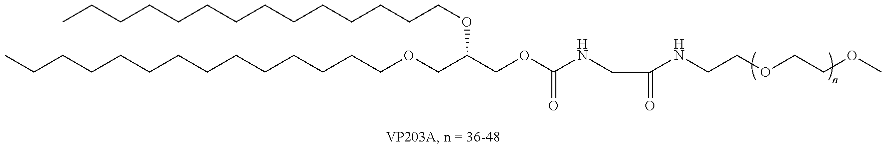

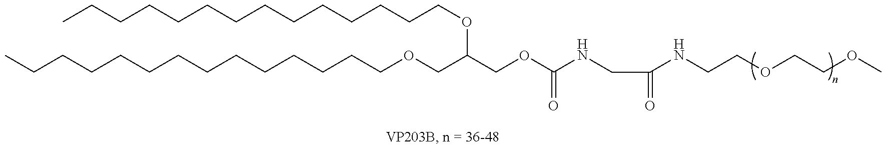

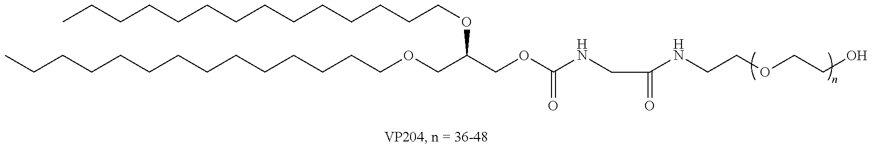

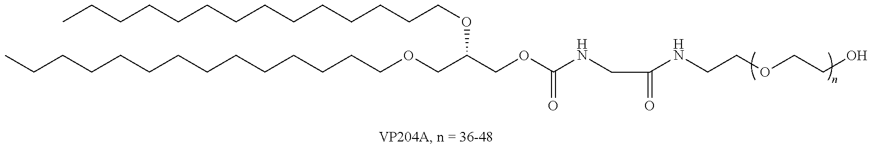

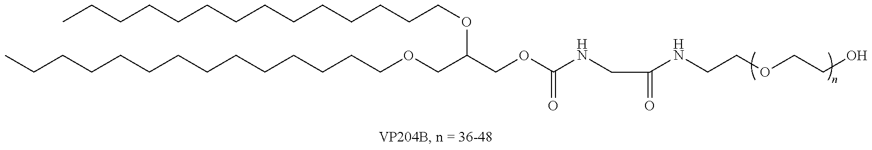

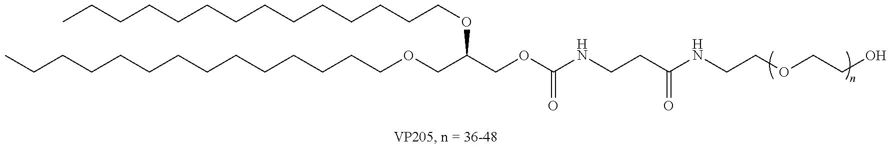

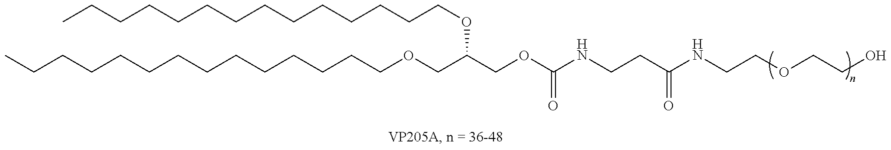

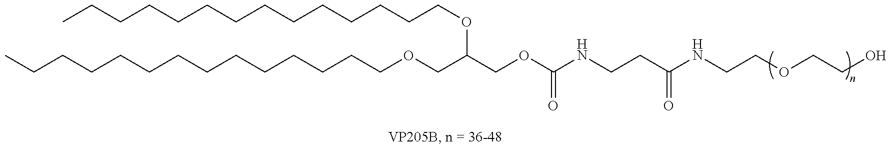

In some embodiments, the n in Table 2 is an integer from 2 to 200, from 10 to 100, from 25 to 50, or from 42 to 48. In some embodiments, the n in Table 2 is 25, 26, 27, 28, 29, 30, 31, 32, 33, 34, 35, 36, 37, 38, 39, 40, 41, 42, 43, 44, 45, 46, 47, 48, 49, 50, or more.

A PEG-lipid can comprise one or more ethylene glycol units, for example, at least 1, at least 2, at least 5, at least 10, at least 20, at least 30, at least 40, at least 50, at least 60, at least 70, at least 80, at least 90, at least 100, at least 120, or at least 150 ethylene glycol units. In some embodiments, a number average molecular weight of the PEG-lipids is from about 200 Da to about 5000 Da. In some embodiments, a number average molecular weight of the PEG-lipids is from about 500 Da to about 3000 Da. In some embodiments, a number average molecular weight of the PEG-lipids is from about 750 Da to about 2500 Da. In some embodiments, a number average molecular weight of the PEG-lipids is from about 750 Da to about 2500 Da. In some embodiments, a number average molecular weight of the PEG-lipids is about 500 Da, about 750 Da, about 1000 Da, about 1250 Da, about 1500 Da, about 1750 Da, or about 2000 Da. In some embodiments, a polydispersity index (PDI) of the one or more PEG-lipids is smaller than 2. In some embodiments, a PDI of the one or more PEG-lipids is at most 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0. In some embodiments, a PDI of the one or more PEG-lipids is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0.

In some embodiments, the PEG-lipid comprises from about 0.1 mol % to about 10 mol % of the total lipid present in the particle. In some embodiments, the PEG-lipid comprises from about 0.1 mol % to about 6 mol % of the total lipid present in the particle. In some embodiments, the PEG-lipid comprises from about 0.5 mol % to about 5 mol % of the total lipid present in the particle. In some embodiments, the PEG-lipid comprises from about 1 mol % to about 3 mol % of the total lipid present in the particle. In some embodiments, the PEG-lipid comprises about 2.0 mol % to about 2.5 mol % of the total lipid present in the particle. In some embodiments, the PEG-lipid comprises about 1 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, or about 3.0 mol % of the total lipid present in the particle.

In some embodiments, the LNP composition comprises a plurality of PEG-lipids, for example, at least 2, 3, 4, 5, 6, 7, 8, 9, 10, or more distinct PEG-lipids. In some embodiments, at least one of the plurality of PEG-lipids is selected from Table 2.

### Phospholipid

In some embodiments, the described LNP composition comprises a phospholipid. In some embodiments, the phospholipid comprises a lipid selected from the group consisting of: phosphatidylcholine (PC), phosphatidylethanolamine amine, glycerophospholipid, sphingophospholipids, Guriserohosuhono, sphingolipids phosphono lipids, natural lecithins, and hydrogenated phospholipid. In some embodiments, the phospholipid comprises a phosphatidylcholine. Exemplary phosphatidylcholines include, but are not limited to, soybean phosphatidylcholine, egg yolk phosphatidylcholine (EPC), distearoylphosphatidylcholine, 1,2-distearoyl-sn-glycero-3-phosphocholine (DSPC), dipalmitoyl phosphatidylcholine, dipalmitoyl-sn-glycero-3-phosphocholine (DPPC), palmitoyl oleoyl phosphatidylcholine (POPC), dimyristoyl phosphatidylcholine (DMPC), and dioleoyl phosphatidylcholine (DOPC). In certain specific embodiments, the phospholipid is DSPC.

In some embodiments, the phospholipid comprises a phosphatidylethanolamine amine. In some embodiments, the phosphatidylethanolamine amine is distearoyl phosphatidylethanolamine (DSPE), dipalmitoyl phosphatidyl ethanolamine (DPPE), dioleoyl phosphatidylethanolamine (DOPE), dimyristoyl phosphoethanolamine (DMPE), 16-O-Monome Le PE, 16-O-dimethyl PE, 18-1-trans PE, palmitoyl oleoyl-phosphatidylethanolamine (POPE), or 1-stearoyl-2-oleoyl-phosphatidyl ethanolamine (SOPE). In some embodiments, the phospholipid comprises a glycerophospholipid. In some embodiments, the glycerophospholipid is plasmalogen, phosphatidate, or phosphatidylcholine. In some embodiments, the glycerophospholipid is phosphatidylserine, phosphatidic acid, phosphatidylglycerol, phosphatidylinositol, palmitoyl oleoyl phosphatidylglycerol (POPG), or lysophosphatidylcholine. In some embodiments, the phospholipid comprises a sphingophospholipid. In some embodiments, the sphingophospholipid is sphingomyelin, ceramide phosphoethanolamine, ceramide phosphoglycerol, or ceramide phosphoglycerophosphoric acid. In some embodiments, the phospholipid comprises a natural lecithin. In some embodiments, the natural lecithin is egg yolk lecithin or soybean lecithin. In some embodiments, the phospholipid comprises a hydrogenated phospholipid. In some embodiments, the hydrogenated phospholipid is hydrogenated soybean phosphatidylcholine. In some embodiments, the phospholipid is selected from the group consisting of: phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine.

In some embodiments, the phospholipid comprises a lipid selected from: 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

A phospholipid can comprise a phospholipid moiety and one or more fatty acid moieties. A phospholipid moiety can comprise phosphatidyl choline, phosphatidyl ethanolamine, phosphatidyl glycerol, phosphatidyl serine, phosphatidic acid, 2-lysophosphatidyl choline, or a sphingomyelin. A fatty acid moiety can comprise lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, or docosahexaenoic acid. In some specific embodiments, a phospholipid can be functionalized with or cross-linked to one or more alkynes, which may undergo a copper-catalyzed cycloaddition upon exposure to an azide.

In some embodiments, the LNP composition comprises a plurality of phospholipids, for example, at least 2, 3, 4, 5, or more distinct phospholipids. In some embodiments, the phospholipid comprises from 1 mol % to 20 mol % of the total lipid present in the particle. In some embodiments, the phospholipid comprises from about 5 mol % to about 15 mol % of the total lipid present in the particle. In some embodiments, the phospholipid comprises from about 8 mol % to about 12 mol % of the total lipid present in the particle. In some embodiments, the phospholipid comprises from about 9 mol %, 10 mol %, or 11 mol % of the total lipid present in the particle.

### Cholesterol

In some embodiments, the LNP composition comprises a cholesterol or a derivative thereof. In some embodiments, the LNP composition comprises a structural lipid. The structural lipid can be selected from steroid, sterol, alkyl resoreinol, cholesterol or derivative thereof, fecosterol, sitosterol, ergosterol, campesterol, stigmasterol, brassicasterol, tomatidine, tomatine, ursolic acid, alpha-tocopherol, and a combination thereof. In some embodiments, the structural lipid is a corticosteroid such as prednisolone, dexamethasone, prednisone, and hydrocortisone. In some embodiments, the cholesterol or derivative thereof is cholesterol, 5-heptadecylresorcinol, or cholesterol hemisuccinate. In some embodiments, the cholesterol or derivative thereof is cholesterol.

In some embodiments, the cholesterol or derivative thereof is a cholesterol derivative. In some embodiments, the cholesterol derivative is a polar cholesterol analogue. In some embodiments, the polar cholesterol analogue is 5α-cholestanol, 5β-coprostanol, cholesteryl-(2′-hydroxy)-ethyl ether, cholesteryl-(4′-hydroxy)-butyl ether, or 6-ketocholestanol. In some embodiments, the polar cholesterol analogue is cholesteryl-(4′-hydroxy)-butyl ether. In some embodiments, the cholesterol derivative is a non-polar cholesterol analogue. In some embodiments, the non-polar cholesterol analogue is 5α-cholestane, cholestenone, 5α-cholestanone, 5β-cholestanone, or cholesteryl decanoate.

In some embodiments, the cholesterol or the derivative thereof comprises from 20 mol % to 50 mol % of the total lipid present in the particle. In some embodiments, the cholesterol or the derivative thereof comprises about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, or about 50 mol % of the total lipid present in the particle.

### Antioxidants

In some embodiments, the LNP described herein comprises one or more antioxidants. In some embodiments, the one or more antioxidants function to reduce a degradation of the cationic lipids, the payload, or both. In some embodiments, the one or more antioxidants comprise a hydrophilic antioxidant. In some embodiments, the one or more antioxidants is a chelating agent such as ethylenediaminetetraacetic acid (EDTA) and citrate. In some embodiments, the one or more antioxidants is EDTA. In some embodiments, the one or more antioxidants comprise a lipophilic antioxidant. In some embodiments, the lipophilic antioxidant comprises a vitamin E isomer or a polyphenol. In some embodiments, the one or more antioxidants are present in the LNP composition at a concentration of at least 1 mM, at least 10 mM, at least 20 mM, at least 50 mM, or at least 100 mM. In some embodiments, the one or more antioxidants are present in the particle at a concentration of about 20 mM.

### Payload

The LNPs described herein can be designed to deliver a payload, such as a therapeutic agent, or a target of interest. Exemplary therapeutic agents include, but are not limited to, antibodies (e.g., monoclonal, chimeric, humanized, nanobodies, and fragments thereof etc.), cholesterol, hormones, peptides, proteins, chemotherapeutics and other types of antineoplastic agents, low molecular weight drugs, vitamins, co-factors, nucleosides, nucleotides, oligonucleotides, enzymatic nucleic acids, antisense nucleic acids, triplex forming oligonucleotides, antisense DNA or RNA compositions, chimeric DNA:RNA compositions, allozymes, aptamers, ribozyme, decoys and analogs thereof, plasmids and other types of expression vectors, and small nucleic acid molecules, RNAi agents, short interfering nucleic acid (siRNA), messenger ribonucleic acid (messenger RNA, mRNA), short interfering RNA (siRNA), double-stranded RNA (dsRNA), micro-RNA (miRNA), and short hairpin RNA (shRNA) molecules, peptide nucleic acid (PNA), a locked nucleic acid ribonucleotide (LNA), morpholino nucleotide, threose nucleic acid (TNA), glycol nucleic acid (GNA), siRNA (small internally segmented interfering RNA), aiRNA (asymmetric interfering RNA), and siRNA with 1, 2 or more mismatches between the sense and anti-sense strand to relevant cells and/or tissues, such as in a cell culture, subject or organism. Therapeutic agents can be purified or partially purified, and can be naturally occurring or synthetic, or chemically modified. In some embodiments, the therapeutic agent is an RNAi agent, short interfering nucleic acid (siNA), short interfering RNA (siRNA), double-stranded RNA (dsRNA), micro-RNA (miRNA), or a short hairpin RNA (shRNA) molecule. In some embodiments, the therapeutic agent is an mRNA.

In some embodiments, the payload comprises one or more nucleic acid(s) (i.e., one or more nucleic acid molecular entities). In some embodiments, the nucleic acid is a single-stranded nucleic acid. In some embodiments, single-stranded nucleic acid is a DNA. In some embodiments, single-stranded nucleic acid is an RNA. In some embodiments, the nucleic acid is a double-stranded nucleic acid. In some embodiments, the double-stranded nucleic acid is a DNA. In some embodiments, the double-stranded nucleic acid is an RNA. In some embodiments, the double-stranded nucleic acid is a DNA-RNA hybrid. In some embodiments, the nucleic acid is a messenger RNA (mRNA), a microRNA, an asymmetrical interfering RNA (aiRNA), a small hairpin RNA (shRNA), or a Dicer-Substrate dsRNA.

In some embodiments, the payload comprises an mRNA. In some embodiments, the payload comprises an mRNA molecule encoding a Cas nuclease, i.e., a Cas nuclease mRNA. In some embodiments, the payload comprises one or more guide RNAs or nucleic acids encoding guide RNAs. In some embodiments, the payload comprises a template nucleic acid for repair or recombination. In some embodiments, the payload comprises an mRNA encoding a gene editor nuclease. In some embodiments, the payload comprises an mRNA encoding a base editor nuclease. In some embodiments, the payload comprises an mRNA encoding a restriction enzyme. In some embodiments, the payload comprises zinc-finger nuclease or TALEN nuclease.

In some embodiments, the mRNA payload, such as a Cas nuclease mRNA, can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. Additional modifications to improve stability, expression, and immunogenicity can also be made. The mRNA encoding a Cas nuclease can be codon optimized for expression in a particular cell type, such as a eukaryotic cell, a mammalian cell, or more specifically, a human cell. In some embodiments, the mRNA encodes a human codon optimized Cas9 nuclease or human codon optimized Cpf nuclease as the Cas nuclease. In some embodiments, the mRNA encodes a gene editor (i.e., genome editor) nuclease and is called a gene editor mRNA. In some embodiments, the gene editor is a Cas protein, such as the ones described herein. In some embodiments, the gene editor is an engineered nuclease. In some embodiments, the gene editor introduces a double stranded break in a gene of interest. In some embodiments, the gene editor introduces a double stranded break at a targeted point within a gene of interest. In some embodiments, the gene editor introduces a single stranded break in a gene of interest. In some embodiments, the gene editor is a base editor. In some embodiments, the gene editor inserts a nucleic acid sequence into a gene of interest. In some embodiments, the gene editor deletes a targeted sequence from a gene of interest. In some embodiments, the gene editor mRNA encodes Cas9 nuclease. In some embodiments, the gene editor mRNA encodes base editor nuclease. In some embodiments, the gene editor mRNA encodes a restriction enzyme. In some embodiments, the gene editor mRNA encodes zinc-finger nuclease. In some embodiments, the gene editor mRNA encodes transcription activator-like effector-based nucleases (TALEN). In some embodiments, the gene editor mRNA encodes a meganuclease. In some embodiments, the gene editor mRNA encodes an Argonaute protein. In some embodiments, the mRNA is purified. In some embodiments, the mRNA is purified using a precipitation method (e.g., LiCl precipitation, alcohol precipitation, or an equivalent method, e.g., as described herein) or a chromatography-based method (e.g., an HPLC-based method or an equivalent method).

In some embodiments, the Cas nuclease mRNA comprises a 3′ or 5′ untranslated region (UTR). In some embodiments, the 3′ or 5′ UTR can be derived from a human gene sequence. Exemplary 3′ and 5′ UTRs include α- and β-globin, albumin, HSD17B4, and eukaryotic elongation factor 1a. In addition, viral-derived 5′ and 3′ UTRs can also be used and include orthopoxvirus and cytomegalovirus UTR sequences. In certain embodiments, the mRNA includes a 5′ cap, such as m7G(5′)ppp(5′)N. In certain embodiments, this cap can be a cap-0 where nucleotide N does not contain 2′OMe, or cap-1 where nucleotide N contains 2′OMe, or cap-2 where nucleotides N and N+1 contain 2′OMe. In some embodiments, the 5′ cap can regulate nuclear export; prevent degradation by exonucleases; promote translation; and promote 5′ proximal intron excision. In addition, caps can also contain a non-nucleic acid entity that acts as the binding element for eukaryotic translation initiation factor 4E, eIF4E. In certain embodiments, the mRNA includes a poly(A) tail. This tail can be about 40 to about 300 nucleotides in length. In some embodiments, the tail is about 40 to about 100 nucleotides in length. In some embodiments, the tail is about 100 to about 300 nucleotides in length. In some embodiments, the tail is about 100 to about 300 nucleotides in length. In some embodiments, the tail is about 50 to about 200 nucleotides in length. In some embodiments, the tail is about 50 to about 250 nucleotides in length. In certain embodiments, the tail is about 100, 150, or 200 nucleotides in length. The poly(A) tail can contain modifications to prevent exonuclease degradation including phosphorotioate linkages and modifications to the nucleobase. In some embodiments, the poly(A) tail contains a 3′ “cap” which could include modified or non-natural nucleobases or other synthetic moieties. In some embodiments, the mRNA comprises at least one element that is capable of modifying the intracellular half-life of the RNA. The half-life of the RNA can be increased or decreased. In some embodiments, the element is capable of increasing or decreasing the stability of the RNA. In some embodiments the element may promote RNA decay. In some embodiments, the element can activate translation. In some embodiments, the element may be within the 3′ UTR of the RNA. For example, the element may be an mRNA decay signal or may include a polyadenylation signal (PA).

In some embodiments, the Cas nuclease mRNA encodes a Cas protein from a CRISPR/Cas system. In some embodiments, the Cas protein comprises at least one domain that interacts with a guide RNA (“gRNA”). In some embodiments, the Cas protein is directed to a target sequence by a guide RNA. The guide RNA can interact with the Cas protein as well as the target sequence such that, it can direct binding to the target sequence. In some embodiments, the guide RNA provides the specificity for the targeted cleavage, and the Cas protein may be universal and paired with different guide RNAs to cleave different target sequences. In certain embodiments, the Cas protein may cleave single or double-stranded DNA. In certain embodiments, the Cas protein may cleave RNA. In certain embodiments, the Cas protein may nick RNA. In some embodiments, the Cas protein comprises at least one DNA binding domain and at least one nuclease domain. In some embodiments, the nuclease domain may be heterologous to the DNA binding domain. In certain embodiments, the Cas protein may be modified to reduce or eliminate nuclease activity. The Cas protein may be used to bind to and modulate the expression or activity of a DNA sequence.

In some embodiments, the CRISPR/Cas system comprises Class 1 or Class 2 system components, including ribonucleic acid protein complexes. The Class 2 Cas nuclease families of proteins are enzymes with DNA endonuclease activity, and they can be directed to cleave a desired nucleic acid target by designing an appropriate guide RNA, as described further herein. A Class 2 CRISPR/Cas system component may be from a Type-IIA, Type-JIB, Type-IIC, Type V, or Type VI system. Class 2 Cas nucleases include, for example, Cas9, Cpf1, C₂c1, C₂c2, and C₂c3 proteins. In some embodiments, the Cas protein is from a Type-II CRISPR/Cas system, i.e., a Cas9 protein from a CRISPR/Cas9 system, or a Type-V CRISPR/Cas system, e.g., a Cpf1 protein. In some embodiments, the Cas protein is from a Class 2 CRISPR/Cas system, i.e., a single-protein Cas nuclease such as a Cas9 protein or a Cpf1 protein.

Exemplary species that the Cas9 protein or other components can be from include, but are not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polar omonas naphthalenivorans, Polar omonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionium, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodular ia spumigena, Nostoc sp., Arthrospira maxima, Arthrospira platensis, Arthrospira sp., Lyngbya sp., Microcoleus chthonoplastes, Oscillator ia sp., Petrotoga mobilis, Thermosipho africanus, Streptococcus pasteurianus, Neisseria cinerea, Campylobacter lari, Parvibaculum lavamentivorans, Coryne bacterium diphtheria, or Acaryochloris marina. In some embodiments, the Cas9 protein is from Streptococcus pyogenes. In some embodiments, the Cas9 protein may be from Streptococcus thermophilus. In some embodiments, the Cas9 protein is from Staphylococcus aureus.

In some embodiments, the payload comprises at least one guide RNA. The guide RNA may guide the Class 2 Cas nuclease to a target sequence on a target nucleic acid molecule, where the guide RNA hybridizes with and the Cas nuclease cleaves or modulates the target sequence. In some embodiments, a guide RNA binds with and provides specificity of cleavage by a Class 2 nuclease. In some embodiments, the guide RNA and the Cas protein may form a ribonucleoprotein (RNP), e.g., a CRISPR/Cas complex. In some embodiments, the CRISPR complex may be a Type-II CRISPR/Cas9 complex. In some embodiments, the CRISPR/Cas complex may be a Type-V CRISPR/Cas complex, such as a Cpf1/guide RNA complex. In some embodiments, the Cas nuclease may be a single-protein Cas nuclease, e.g. a Cas9 protein or a Cpf 1 protein. In some embodiments, the guide RNA targets cleavage by a Cas9 protein. In some embodiments, the payload comprises two or more guide RNA molecules. In some embodiments, the two or more guide RNA molecules target the same disease-causing gene. In some embodiments, the two or more guide RNA molecules target different genes. In some specific embodiments, the two guide RNA molecules target two separate disease-causing genes of interest.

A guide RNA for a CRISPR/Cas9 nuclease system comprises a CRISPR RNA (crRNA) and a tracr RNA (tracr). In some embodiments, the crRNA may comprise a targeting sequence that is complementary to and hybridizes with the target sequence on the target nucleic acid molecule. The crRNA may also comprise a flagpole that is complementary to and hybridizes with a portion of the tracrRNA. In some embodiments, the crRNA may parallel the structure of a naturally occurring crRNA transcribed from a CRISPR locus of a bacteria, where the targeting sequence acts as the spacer of the CRISPR/Cas9 system, and the flagpole corresponds to a portion of a repeat sequence flanking the spacers on the CRISPR locus. The guide RNA may target any sequence of interest via the targeting sequence of the crRNA. In some embodiments, the degree of complementarity between the targeting sequence of the guide RNA and the target sequence on the target nucleic acid molecule is at least about 60%, 65%, 70%, 75%, 80%, 85%, 90%, 95%, 97%, 98%, 99%, or 100%. In some embodiments, the targeting sequence of the guide RNA and the target sequence on the target nucleic acid molecule may be 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches.

In some embodiments, the length of the targeting sequence depends on the CRISPR/Cas system and components used. For example, different Cas proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence comprised 18-24 nucleotides in length. In some embodiments, the targeting sequence comprises 19-21 nucleotides in length. In some embodiments, the targeting sequence comprises 20 nucleotides in length.

In some embodiments, the guide RNA is a “dual guide RNA” or “dgRNA”. In some embodiments, the dgRNA comprises a first RNA molecule comprising a crRNA, and a second RNA molecule comprising a tracr RNA. The first and second RNA molecules may form a RNA duplex via the base pairing between the flagpole on the crRNA and the tracr RNA. In some embodiments, the guide RNA is a “single guide RNA” or “sgRNA”. In some embodiments, the sgRNA may comprise a crRNA covalently linked to a tracr RNA. In some embodiments, the crRNA and the tracr RNA may be covalently linked via a linker. In some embodiments, the single-molecule guide RNA may comprise a stem-loop structure via the base pairing between the flagpole on the crRNA and the tracr RNA. In some embodiments, the sgRNA is a “Cas9 sgRNA” capable of mediating RNA-guided DNA cleavage by a Cas9 protein. In certain embodiments, the guide RNA comprises a crRNA and tracr RNA sufficient for forming an active complex with a Cas9 protein and mediating RNA-guided DNA cleavage. In some embodiments, the payload comprises more than one guide RNAs; each guide RNA contains a different targeting sequence, such that the CRISPR/Cas system cleaves more than one target sequence. In some embodiments, one or more guide RNAs may have the same or differing properties such as activity or stability within a CRISPR/Cas complex. Where more than one guide RNA is used, each guide RNA can be encoded on the same or on different expression cassettes. The promoters used to drive expression of the more than one guide RNA may be the same or different.

In some embodiments, the nucleic acid payload, such as RNAs, is modified. Modified nucleosides or nucleotides can be present in a guide RNA or mRNA. A guide RNA or Cas nuclease encoding mRNA comprising one or more modified nucleosides or nucleotides is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide. Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

In some embodiments, the payload can include a template nucleic acid. The template can be used to alter or insert a nucleic acid sequence at or near a target site for a Cas nuclease. In some embodiments, the template is used in homologous recombination. In some embodiments, the homologous recombination may result in the integration of the template sequence or a portion of the template sequence into the target nucleic acid molecule. In some embodiments, a single template is provided. In other embodiments, two or more templates are provided such that homologous recombination may occur at two or more target sites.

In some embodiments, the payload, such as one or more RNAs, are fully encapsulated within the lipid portion of the particle, thereby protecting the RNAs from nuclease degradation. Fully encapsulated can indicate that the RNA in the nucleic acid-lipid particle is not significantly degraded after exposure to serum or a nuclease assay that would significantly degrade free DNA or RNA. In some embodiments, the nucleic acid-lipid particle composition comprises a RNA molecule that is fully encapsulated within the lipid portion of the particles, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, or 99% (or any fraction thereof or range therein) of the particles have the RNA encapsulated therein.

In some embodiments, the payload comprises an mRNA and one or more guide RNA. In some embodiments, the mRNA encodes a gene editor nuclease and is called a gene editor mRNA. In some embodiments, the gene editor mRNA encodes Cas9 nuclease. In some embodiments, the mRNA encodes base editor nuclease. In some embodiments, the gene editor mRNA encodes zinc-finger nuclease. In some embodiments, the gene editor mRNA encodes TALEN nuclease.

### Surfactants and Other Components

Nanoparticles described herein can comprise one or more surfactants. In some embodiments, the one or more surfactants comprise one or more anionic surfactants. Exemplary anionic surfactants include but are not limited to 2-acrylamido-2-methylpropane sulfonic acid, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, and sulfolipid. In some embodiments, the one or more surfactants comprise one or more cationic surfactants. Exemplary cationic surfactants include but are not limited to behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, and thonzonium bromide. In some embodiments, the one or more surfactants comprise one or more zwitterionic surfactants. Exemplary zwitterionic surfactants include but are not limited to cocamidopropyl betaine, cocamidopropyl hydroxysultaine, dipalmitoylphosphatidylcholine, egg lecithin, hydroxysultaine, lecithin, myristamine oxide, peptitergents, and sodium lauroamphoacetate.

In some embodiments, the one or more surfactants comprise one or more non-ionic surfactants. Exemplary non-ionic surfactants include but are not limited to alkyl polyglycoside, cetomacrogol 1000, cetostearyl alcohol, cetyl alcohol, cocamide dea, cocamide mea, decyl glucoside, decyl polyglucose, glycerol monostearate, igepal ca-630, isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, n-octyl beta-d-thioglucopyranoside, octyl glucoside, oleyl alcohol, peg-10 sunflower glycerides, pentaethylene glycol monododecyl ether, polidocanol, poloxamer (e.g., poloxamer 188 an dpoloxamer 407), polyethoxylated tallow amine, polyglycerol polyricinoleate, polysorbate (e.g., polysorbate 20, polysorbate 40, polysorbate 60, or polysorbate 80), sorbitan, sorbitan monolaurate, sorbitan monostearate, sorbitan tristearate, stearyl alcohol, surfactin, triton x-100.

In some embodiments, a nanoparticle described herein comprises one or more non-ionic surfactants. In some embodiments, the one or more non-ionic surfactants comprise a fatty alcohol, a fatty acid, or both. In some embodiments, the fatty alcohol is a C₁₂-C₂₄ fatty alcohol. In some embodiments, the fatty alcohol is lauryl alcohol, tridecyl alcohol, myristyl alcohol, pentadecyl alcohol, cetyl alcohol, palmitoleyl alcohol, heptadecyl alcohol, stearyl alcohol, oleyl alcohol, nonadecyl alcohol, arachidyl alcohol, or a combination thereof. In some embodiments, the C₁₂-C₂₄ fatty alcohol is oleyl alcohol, stearyl alcohol, or a mixture thereof. In some embodiments, the nanoparticle comprises oleyl alcohol. In some embodiments, the nanoparticle comprises stearyl alcohol.

In some embodiments, the one or more surfactants (such as fatty alcohol) are present in a herein described nanoparticle composition in an amount of about 0.5 mol % to about 20 mol % of a total lipid content present in the nanoparticle composition, or any ranges therebetween. In some embodiments, the one or more surfactants (such as fatty alcohol) are present in the nanoparticle composition in an amount of about 1 mol % to about 10 mol % of a total lipid content present in the nanoparticle composition. In some embodiments, the one or more surfactants (such as fatty alcohol) are present in the nanoparticle composition in an amount of about 3 mol % to about 8 mol % of a total lipid content present in the nanoparticle composition. In some embodiments, the one or more surfactants (such as fatty alcohol) are present in the nanoparticle composition at about 3 mol %, about 4 mol %, about 5 mol %, about 6 mol %, about 7 mol %, about 8 mol %, about 9 mol %, or about 10 mol % of a total lipid content present in the nanoparticle composition. In some embodiments, the one or more surfactants (such as fatty alcohol) are present in the nanoparticle composition in a range from about 0.5 mol %, 1 mol %, 1.5 mol %, 2 mol %, 2.5 mol %, 3 mol %, or 3.5 mol % to about 4 mol %, 4.5 mol %, 5 mol %, 5.5 mol %, 6 mol %, 7 mol %, 8 mol %, 9 mol %, or 10 mol % of a total lipid content present in the nanoparticle composition. In some embodiments, the one or more surfactants (such as fatty alcohol) are present in the nanoparticle composition in a range from about 1 mol %, 2 mol %, 3 mol %, 4 mol %, 5 mol %, 6 mol %, or 7 mol % to about 8 mol %, 9 mol %, 10 mol %, 11 mol %, 12 mol %, 13 mol %, 14 mol %, 15 mol %, or 20 mol % of a total lipid content present in the nanoparticle composition.

Nanoparticle compositions described herein can comprise one or more permeability enhancer molecules, carbohydrates, polymers, or other components. A permeability enhancer molecule can be, e.g., a molecule described by U.S. patent application publication No. 2005/0222064. The carbohydrates can be simple sugars (e.g., glucose) and polysaccharides (e.g., glycogen and derivatives and analogs thereof).

In some embodiments, a nanoparticle composition described herein comprises a polymer. The polymer can be biodegradable and/or biocompatible. Exemplary polymers include but are not limited to polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. In some embodiments, a nanoparticle composition described herein comprises poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), polyglycerol, or a combination thereof.

### Nucleic Acid Stabilizer

In some embodiments, nanoparticle compositions described herein comprise excipients that stabilize lipid nanoparticles encapsulating nucleic acid payload (e.g., nucleic acid therapeutics). For example, the nucleic acid therapeutics can be mRNA to modulate disease causing proteins; mRNA and guide RNA to edit therapeutic genes of interest; RNA-editing nucleic acid payloads; siRNA; antisense oligonucleotides such as those to elicit RNase H-mediated gene silencing; microRNA and anti-microRNA; RNA activators; aptamers and the like.

Lipid nanoparticles (LNPs) represent an efficacious non-viral delivery system for nucleic acid therapeutics. Efficacy of an LNP treatment can be determined by numerous variables, including the stability and functionality of the encapsulated RNA cargo. If the RNA cargo experiences nicking, degradation, or a structural change during long-term storage or freeze/thaw, a partial or complete loss in therapeutic potency may occur. As such, the herein disclosed nanoparticle compositions are advantageous in the delivering of nucleic acid payloads, at least partially due to the enhanced stability of the nanoparticle compositions and/or encapsulated compositions including e.g. nucleic acid therapeutics. In some embodiments, the enhanced stability can prolong the shelf-storage life and improve the freeze-thaw resistance of a pharmaceutical composition comprising the nanoparticle. In some embodiments, the enhanced stability is achieved, at least partially, by the selection of the type, the pH, the osmolarity, and the counter ions of the buffer used to solubilize the nucleic acid payload. In some embodiments, the enhanced stability is achieved, at least partially, by the addition of a nucleic acid stabilizer. The nucleic acid stabilizer can be selected from excipients that are designated by the FDA as generally recognized as safe (GRAS). Accordingly, in one aspect, the present disclosure describes the innovative use of commonly used GRAS excipients to improve RNA stability and delivery by LNPs

Nanoparticle compositions described herein can comprise a nucleic acid stabilizer. In some embodiments, the nucleic acid stabilizer is polyethylene glycol. In some embodiments, the nucleic acid stabilizer is a cationic surfactant such as cetrimonium bromide and cetrimonium chloride. In some embodiments, the nucleic acid stabilizer is cetrimonium bromide (i.e., cetyltrimethylammonium bromide or CTAB). In some embodiments, the nucleic acid stabilizer is polysaccharide or oligosaccharide such as low molecular weight chitosan. Exemplary low molecular weight chitosan can have a molecular weight of 50,000 to 190,000 Da, or lower than 50,000 Da. In some embodiments, the nucleic acid stabilizer is a cryoprotectant. Exemplary cryoprotectants include, but are not limited to, a polyol (e.g., a diol or a triol such as propylene glycol (i.e., 1,2-propanediol), 1,3-propanediol, glycerol, 2-methyl-2,4-pentanediol, 1,6-hexanediol, 1,2-butanediol, 2,3-butanediol, ethylene glycol, or diethylene glycol), a nondetergent sulfobetaine (e.g., NDSB-201 (3-(1-pyridino)-1-propane sulfonate), an osmolyte (e.g., L-proline or trimethylamine N-oxide dihydrate), a water soluble polymer (e.g., polyethylene glycol, polyethylene glycol monomethyl ether (mPEG) such as mPEG 550, mPEG 600, mPEG 2000, mPEG 3350, mPEG 4000, and mPEG 5000, polyvinylpyrrolidone, pentaerythritol propoxylate, and a block polymer of polyethylene glycol and polypropylene glycol), an organic solvent (e.g., dimethyl sulfoxide (DMSO) or ethanol), a sugar (e.g., D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), a salt (e.g., lithium acetate, lithium chloride, lithium formate, lithium nitrate, lithium sulfate, magnesium acetate, sodium chloride, sodium formate, sodium malonate, sodium nitrate, sodium sulfate, or any hydrate thereof), or any combination thereof. In some embodiments, the nanoparticle composition comprises two or more nucleic acid stabilizers.

In some embodiments, the nucleic acid stabilizer is polyethylene glycol (PEG). In some embodiments, the polyethylene glycol is PEG 200, PEG 400, PEG 600, PEG 1000, PEG 3350, PEG 4000, PEG 8000, PEG 10000, PEG 20000, or a combination thereof. In some embodiments, the polyethylene glycol is PEG 200, PEG 400, PEG 600, or a combination thereof. In some embodiments, the polyethylene glycol has a number average molecular weight (Mn) of from about 120 to about 5000 Da, or any numbers or ranges therebetween. In some embodiments, the polyethylene glycol has a Mn of about 120 to about 200 Da, about 200 to about 800 Da, about 200 to about 600 Da, about 120 to about 1000 Da, or about 40 to about 1200 Da. In some embodiments, the polyethylene glycol has a number average molecular weight of about 120, about 160, or about 200 to about 300, about 400, about 500, about 600, about 700, or about 800 Da. In some embodiments, the nucleic acid stabilizer comprise PEG 400. In some embodiments, a herein described nanoparticle composition comprises a polyethylene glycol (such as PEG400) and a nucleic acid payload (such as mRNA, guide RNA, and/or siRNA).

In one aspect, disclosed herein is a method of making a nanoparticle composition that comprises a nucleic acid stabilizer. The nucleic acid stabilizer (e.g., polyethylene glycol) can be added into the nanoparticle composition before, concurrently, or after with addition of nucleic acid payload. In some embodiments, the nucleic acid stabilizer is added to the nanoparticle composition concurrently with the nucleic acid. In some embodiments, the nucleic acid stabilizer is pre-mixed with the nucleic acid payload. In some embodiments, the nucleic acid stabilizer is combined with the nucleic acid payload in a buffer solution. In some embodiments, the nucleic acid stabilizer (such as PEG400) is combined with a nucleic acid payload (such as mRNA, guide RNA, and/or siRNA) in a buffer solution. In some embodiments, the nucleic acid stabilizer (such as PEG400) is mixed with a nucleic acid payload in a buffer solution. In some embodiments, the nucleic acid stabilizer (such as PEG400) is added into a buffer that comprises the nucleic acid payload. In some embodiments, adding the nucleic acid stabilizer such as PEG-400 along with the RNA cargo to the drug substance buffer before lipid mixing can allow it to incorporate into the core of the LNP, where it is expected to confer several advantages: 1) increase long-term stability both at 2-8° C. and at freezing temperature such as −20, −40 and −80° C. by providing cushion to the RNA core, 2) improve RNA encapsulation (also known to those skilled in the art as “entrapment”) efficiency, and 3) preserve RNA potency and prevent nicking/degradation. In some embodiments, the nucleic acid stabilizer (such as PEG400) is added into the nanoparticle composition after the addition of the lipids and the nucleic acid payload. In some embodiments, the nucleic acid stabilizer (such as PEG400) is added into the nanoparticle composition after formulation and before freezing. In some embodiments, the method of making a nanoparticle composition comprises collecting or buffer exchanging LNPs into a drug product or freezing buffer solution containing the target amount of a nucleic acid stabilizer such as PEG400. In some embodiments, adding a nucleic acid stabilizer such as PEG400 in the freezing buffer solution as well as in the drug substance buffer can increase long-term stability both at 2-8° C. and at freezing temperature such as −20, −40 and −80° C. by providing cushion to the RNA core and preserve RNA potency and prevent nicking/degradation.

In some embodiments, the nucleic acid stabilizer (such as PEG400) is present in the described nanoparticle composition in an amount of about 0.001% to about 50% by total weight. In some embodiments, the nucleic acid stabilizer (such as PEG400) is present in the nanoparticle composition in an amount of about 0.01% to about 20% by total weight. In some embodiments, the nucleic acid stabilizer (such as PEG400) is present in the nanoparticle composition in an amount of about 0.01% to about 5% by total weight. In some embodiments, the nucleic acid stabilizer (such as PEG400) is present in the nanoparticle composition in an amount up to about 15% by total weight. In some embodiments, the nucleic acid stabilizer is present in the nanoparticle composition at about 0.01%, about 0.05%, about 0.1%, about 0.15%, or about 0.2% to about 0.3%, about 0.4%, about 0.5%, about 0.6%, about 0.7%, about 0.8%, about 0.9%, about 1.0% or about 2.0% by total weight. In some embodiments, the nucleic acid stabilizer is present in the nanoparticle composition within a range of about 0.01%, 0.25%, about 0.5%, about 0.75%, about 1%, or about 5% to about 7.5%, about 10%, about 15%, or about 20% by total weight. In some embodiments, the nucleic acid stabilizer is present in the nanoparticle composition in an amount of about 0.01% to about 0.5%, 0.01% to about 2%, 0.01% to about 3%, about 0.2% to about 0.8%, about 0.4% to about 0.6%, about 0.6% to about 0.8%, about 0.5% to about 1.0%, about 0.5% to about 2.0%, about 1% to about 2%, about 1% to about 5%, about 5% to about 10%, about 10% to about 15%, about 15% to about 20%, about 2.5% to about 7.5%, about 7.5% to about 12.5%, about 12.5% to about 17.5%, or about 15% to about 25% by total weight. In some embodiments, the nucleic acid stabilizer is present in the nanoparticle composition in an amount of at least 0.1%, at least 0.2%, at least 0.25%, at least 0.5%, at least 0.75%, at least 1%, at least 2%, at least 5%, at least 10%, or at least 15% by total weight. In some embodiments, the nucleic acid stabilizer is present in the nanoparticle composition in an amount of at most 0.25%, at most 0.5%, at most 0.75%, at most 1%, at most 2%, at most 5%, at most 7.5%, at most 10%, at most 15%, at most 20%, at most 25%, or at most 30% by total weight. In some embodiments, the nucleic acid stabilizer is present in the nanoparticle composition in an amount of about 0.25%, about 0.5%, about 0.75%, about 5%, about 10%, or about 15% by total weight.

In some embodiments, adding the nucleic acid stabilizer (such as PEG-400) to the drug substance buffer prior to or co-mixed with nucleic acid payload, the w/w % in the non-RNA containing drug substance buffer is higher than the target w/w % in the final RNA containing drug substance buffer, such that eventual dilution with RNA dilutes the nucleic acid stabilizer % to the target w/w %. In some embodiments, the nucleic acid stabilizer (e.g., PEG-400) is trapped within the core of the particle at an amount proportional to its weight percentage in the drug substance buffer.

### Other Lipids

In some embodiments, the disclosed LNP compositions comprise a helper lipid. In some embodiments, the disclosed LNP compositions comprise a neutral lipid. In some embodiments, the disclosed LNP compositions comprise a stealth lipid. In some embodiments, the disclosed LNP compositions comprises additional lipids.

As used herein, “neutral lipids” suitable for use in a lipid composition of the disclosure include, for example, a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), pohsphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In some embodiments, the neutral phospholipid may be selected from the group consisting of SPC and dimyristoyl phosphatidyl ethanolamine (DMPE). In some embodiments, the neutral phospholipid is DSPC. Neutral lipids can function to stabilize and improve processing of the LNPs.

“Helper lipids” can refer to lipids that enhance transfection (e.g. transfection of the nanoparticle including the biologically active agent). The mechanism by which the helper lipid enhances transfection includes enhancing particle stability. In some embodiments, the helper lipid enhances membrane fusogenicity. In some embodiments, the helper lipid is a neutral lipid.

“Stealth lipids” can refer to lipids that alter the length of time the nanoparticles can exist in vivo {e.g., in the blood). Stealth lipids can assist in the formulation process by, for example, reducing particle aggregation and controlling particle size. Stealth lipids used herein may modulate pharmacokinetic properties of the LNP. Stealth lipids suitable for use in a lipid composition of the disclosure can include, but are not limited to, stealth lipids having a hydrophilic head group linked to a lipid moiety. Stealth lipids suitable for use in a lipid composition of the present disclosure and information about the biochemistry of such lipids can be found in Romberg et al, Pharmaceutical Research, Vol. 25, No. 1, 2008, pg. 55-71 and Hoekstra et al, Biochimica et Biophysica Acta 1660 (2004) 41-52. Additional suitable PEG-lipids are disclosed, e.g., in WO 2006/007712.

In some embodiments, the stealth lipid is a PEG-lipid. In one embodiment, the hydrophilic head group of stealth lipid comprises a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids and poly N-(2-hydroxypropyl)methacrylamide]. Stealth lipids can comprise a lipid moiety. In some embodiments, the lipid moiety of the stealth lipid may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

The structures and properties of helper lipids, neutral lipids, stealth lipids, and/or other lipids are further described in WO2017173054A1, WO2019067999A1, US20180290965A1, US20180147298A1, US20160375134A1, U.S. Pat. Nos. 8,236,770, 8,021,686, 8,236,770B2, U.S. Pat. No. 7,371,404B2, U.S. Pat. No. 7,780,983B2, U.S. Pat. No. 7,858,117B2, US20180200186A1, US20070087045A1, WO2018119514A1, and WO2019067992A1, all of which are hereby incorporated by reference in their entirety.

### LNP Formulations

The LNPs described herein can be designed for one or more specific applications or targets. The elements of a nanoparticle composition can be selected based on a particular application or target, and/or based on the efficacy, toxicity, expense, ease of use, and availability. Similarly, the particular formulation of a nanoparticle composition may be selected for the particular application or target. Exemplary LNP formulations include, but are not limited to, the formulations described in Tables 4-6, 8-15 and 17. Exemplary LNP formulations also include compositions comprising an amino lipid of Formula (I), (I*), (Ia) (Ib), (II), (II*) and a PEG lipid of Formula (III) and (III*).

The described LNP formulations can be designed for one or more specific applications or targets. For example, a nanoparticle composition may be designed to deliver a therapeutic agent such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic agent included in a nanoparticle composition may also be selected based on the desired delivery target or targets. For example, a therapeutic agent may be selected for a particular indication, condition, disease, or disorder and/or for delivery to a particular cell, tissue, organ, or system or group thereof (e.g., localized or specific delivery). In certain embodiments, a nanoparticle composition may include an mRNA encoding a polypeptide of interest capable of being translated within a cell to produce the polypeptide of interest. Such a composition may be designed to be specifically delivered to a particular organ.

The amount of a therapeutic agent in an LNP composition may depend on the size, composition, desired target and/or application, or other properties of the nanoparticle composition. For example, the amount of an RNA comprised in a nanoparticle composition may depend on the size, sequence, and other characteristics of the RNA. The relative amounts of a therapeutic agent and other elements (e.g., lipids) in a nanoparticle composition may also vary. In some embodiments, the wt/wt ratio of the lipid component to a therapeutic agent in a nanoparticle composition may be from about 5:1 to about 60:1, such as about 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic agent may be from about 10:1 to about 40:1. In certain embodiments, the wt/wt ratio is about 20:1. The amount of a therapeutic agent in a nanoparticle composition can be measured using absorption spectroscopy (e.g., ultraviolet-visible spectroscopy).

In some embodiments, an LNP composition comprises one or more nucleic acids such as RNAs. In some embodiments, the one or more RNAs, lipids, and amounts thereof may be selected to provide a specific N/P ratio. The N/P ratio can be selected from about 1 to about 30. The N/P ratio can be selected from about 2 to about 10. In some embodiments, the N/P ratio is from about 0.1 to about 50. In some embodiments, the N/P ratio is from about 2 to about 8. In some embodiments, the N/P ratio is from about 2 to about 15, from about 2 to about 10, from about 2 to about 8, from about 2 to about 6, from about 3 to about 15, from about 3 to about 10, from about 3 to about 8, from about 3 to about 6, from about 4 to about 15, from about 4 to about 10, from about 4 to about 8, or from about 4 to about 6. In some embodiments, the N/P ratio is from about 2 to about 8. In some embodiments, the N/P ratio is about 2, about 2.5, about 3, about 3.5, about 4, about 4.5, about 5, about 5.5, about 6, or about 6.5. In some embodiments, the N/P ratio is from about 4 to about 6. In some embodiments, the N/P ratio is about 4, about 4.5, about 5, about 5.5, or about 6.

In some embodiments, the LNPs are formed with an average encapsulation efficiency ranging from about 50% to about 70%, from about 70% to about 90%, or from about 90% to about 100%. In some embodiments, the LNPs are formed with an average encapsulation efficiency ranging from about 75% to about 95%. In some embodiments, a nucleic acid (e.g., RNA) entrapment efficiency of a nanoparticle described herein is at least 80%, at least 85%, at least 90%, at least 91%, at least 92%, at least 93%, at least 94%, at least 95%, at least 96%, at least 97%, at least 98%, or at least 99%. In some embodiments, a nucleic acid entrapment efficiency of a nanoparticle described herein is from about 80% to about 100%, from about 85% to about 100%, from about 90% to about 100%, from about 95% to about 100%, from about 90% to about 99%, or from about 95% to about 99%. In some embodiments, a nucleic acid entrapment efficiency of a nanoparticle described herein is from about 90% to about 99%,

In some embodiments, nanoparticles described herein have a median diameter of about 10 nm to about 500 nm. In some embodiments, the median diameter of the nanoparticles described herein is from about 50 nm to about 150 nm, from about 60 nm to about 140 nm, from about 70 nm to about 130 nm, from about 80 nm to about 120 nm, or from about 90 nm to about 110 nm. In some embodiments, the median diameter of the nanoparticles described herein is about 60 nm, about 65 nm, about 70 nm, about 75 nm, about 80 nm, about 85 nm, about 90 nm, about 95 nm, about 100 nm, about 105 nm, about 110 nm, about 115 nm, or about 120 nm. Particle size and particle size distribution of the nanoparticles can be measured by light scattering using, for example, a Zetasizer Ultra ZSU 5700 (Malvern, USA). In some embodiments, the particle size distribution is unimodal.

### Preparation of LNP Formulation

In one aspect, described in the present disclosure are processes for making LNP compositions.

A process for making lipid nanoparticles can comprise several general steps: (i) providing a first solution, such as citrate or phosphate buffer, comprising one or more nucleic acid molecular entities in a first reservoir; (ii) providing a second solution comprising one or more lipids and an organic solvent, such as an alcohol (e.g., ethanol) in a second reservoir; and (iii) mixing the first solution with the second solution. The first reservoir is optionally in fluid communication with the second reservoir.

In some embodiments, disclosed herein is a method of preparing a formulation comprising lipid nanoparticles, wherein the nanoparticles comprise (i) one or more nucleic acid molecular entities, (ii) an amino lipid, and (iii) one or more lipids selected from a structural lipid, a neutral lipid, and a PEG-lipid. In some embodiments, the method comprises (a) combining a first faction of the amino lipid with the one or more nucleic acid molecular entities in a first solution, wherein the first fraction comprises 0.1 mol % to 99 mol % of the total amino lipid; (a) combining the remaining of the amino lipid with the one or more lipids selected from a structural lipid, a neutral lipid, and a PEG-lipid in a second solution; (c) mixing the first solution and the second solution, thereby producing the lipid nanoparticles. In some embodiments, the first fraction of the amino lipid is configured to neutralize between 0.1-99% of the phosphate (on an N:P basis) in the one or more nucleic acid molecular entities, or any numbers or ranges therebetween. In some embodiments, the first fraction of the amino lipid is configured to neutralize between 0.5-90% of the phosphate (on an N:P basis) in the one or more nucleic acid molecular entities. In some embodiments, the first fraction of the amino lipid is configured to neutralize about 5 to 90%, 10 to 75%, 25 to 50%, 25 to 75%, 50 to 75%, or 50 to 90% of the phosphate (on an N:P basis) in the one or more nucleic acid molecular entities. In some embodiments, the first fraction of the amino lipid is configured to neutralize about 10%, 15%, 25%, 50%, or 75% of the phosphate (on an N:P basis) in the one or more nucleic acid molecular entities. In some embodiments, the first solution is an aqueous buffer solution, e.g., a citrate buffer. In some embodiments, the first solution further comprises a nucleic acid stabilizer, e.g., PEG 400. In some embodiments, the first solution and the second solution are mixed in an inline mixer.

The process can optionally comprise one or more dilution steps, one or more incubation steps, one or more buffer exchange steps, one or more concentration steps, and/or one or more filtrations steps. In some embodiments, the dilution step involves dilution by adding a dilution buffer. In some embodiments, the dilution step involves dilution with aqueous buffer (e.g. citrate buffer or pure water) e.g., using a pumping apparatus (e.g. a peristaltic pump). In some embodiments, the dilution buffer is an organic solution such as alcohol. The dilution step can comprise a dilution that is 1 to 20 times of the initial volume, or any numbers or ranges therebetween. In some embodiments, the dilution step comprises a dilution that is 1 to 10 times of the initial volume. In some embodiments, the dilution step is followed by the buffer exchange step or the incubation step.

The incubation step comprises allowing a solution from the mixing step to stand in a vessel for about 0 to about 100 hours at about room temperature and optionally protected from light. In some embodiments, the incubation step runs from 0 to 24 hours, 1 minute to 2 hours, or 1 minute to 60 minutes. In some embodiments, the incubation step runs from 1 minutes to 120 minutes. In some embodiments, the incubation step is followed by the buffer exchange step. In some embodiments, the incubation step follows the buffer exchange step.

In some embodiments, the buffer exchange step comprises a solvent exchange that results in a higher concentration of phosphate buffered saline (PBS) buffer. In some embodiments, the buffer exchange step comprises removing all or a portion of organic solvent. In some embodiments, the buffer exchange step comprises dialysis through a suitable membrane (e.g. 10,000 mwc snakeskin membrane). In some embodiments, the buffer exchange step comprises filtration such as tangential flow filtration (TFF)). In some embodiments, the buffer exchange step comprises chromatography such as using a desalting column, e.g., PD10 column. In some embodiments, the buffer exchange step comprises ultrafiltration. Ultrafiltration comprises concentration of the diluted solution followed by diafiltration, e.g., using a suitable pumping system (e.g. pumping apparatus such as a peristaltic pump or equivalent thereof) in conjunction with a suitable ultrafiltration membrane (e.g. GE Hollow fiber cartridges or equivalent).

In some embodiments, the mixing step provides a clear single phase. In some embodiments, after the mixing step, the organic solvent is removed to provide a suspension of particles, wherein the one or more nucleic acid molecular entities are encapsulated by the lipid(s). The selection of an organic solvent can involve consideration of solvent polarity and the ease with which the solvent can be removed at the later stages of particle formation. The organic solvent, which can serve as a solubilizing agent, can be in an amount sufficient to provide a clear single phase mixture of the one or more nucleic acid molecular entities and lipid(s). The organic solvent may be selected from one or more (e.g., two) of chloroform, dichloromethane, diethylether, cyclohexane, cyclopentane, benzene, toluene, methanol, and other aliphatic alcohols (e.g. C₁ to C₈) such as ethanol, propanol, isopropanol, butanol, tert-butanol, iso-butanol, pentanol and hexanol. The methods used to remove the organic solvent can involve diafiltration or dialysis or evaporation at reduced pressures or blowing a stream of inert gas (e.g. nitrogen or argon) across the mixture.

In some embodiments, the method further comprises adding nonlipid polycations which are useful to effect the transformation of cells using the present compositions. Examples of suitable nonlipid polycations include, but are limited to, hexadimethrine bromide (sold under the brand name POLYBRENE®, from Aldrich Chemical Co., Milwaukee, Wis., USA) or other salts of hexadimethrine. Other suitable polycations include, e.g., salts of poly-L-ornithine, poly-L-arginine, poly-L-lysine, poly-D-lysine, polyallylamine and polyethyleneimine. In certain embodiments, the formation of the lipid nanoparticles can be carried out either in a mono-phase system (e.g. a Bligh and Dyer monophase or similar mixture of aqueous and organic solvents) or in a two-phase system with suitable mixing.

The lipid nanoparticles can be formed in a mono- or a bi-phase system. In some embodiments, in a mono-phase system, the amino lipid(s) and one or more nucleic acid molecular entities are each dissolved in a volume of the mono-phase mixture. Combining the two solutions provides a single mixture in which the complexes form. In some embodiments, in a bi-phase system, the amino lipids bind to the one or more nucleic acid molecular entities (which is present in the aqueous phase), and thus increasing the solubility in organic phase.

In some embodiments, the lipid nanoparticles are prepared in an apparatus comprising a first reservoir for holding an aqueous solution and a second reservoir for holding an organic lipid solution. In some embodiments, the apparatus comprises additional reservoirs for holding an aqueous solution (such as for a portion of the one or more nucleic acid molecular entities) and/or an organic solution. The apparatus can include a pump mechanism configured to pump the aqueous and the organic lipid solutions into a mixing region or mixing chamber at substantially equal flow rates. In some embodiments, the mixing region or mixing chamber comprises a T coupling or equivalent thereof, which allows the aqueous and organic fluid streams to combine as input into the T connector and the resulting combined aqueous and organic solutions to exit out of the T connector into a collection reservoir or equivalent thereof.

In some embodiments, the first solution comprises an aqueous buffer. In some embodiments, the first solution comprises a mixture of an aqueous buffer mixed with an organic solvent. In some embodiments, the organic solvent present in the aqueous buffer is ethanol. In some embodiments, the second solution comprises a mixture of an aqueous buffer mixed with an organic solvent. In some embodiments, the second solution comprises ethanol. In some embodiments, the second solution comprises ethanol and water. In some embodiments, the ethanol percentage in the aqueous buffer ranges from 0.1% to 50%, or any numbers or ranges therebetween. In some embodiments, the dilution buffer comprises an aqueous buffer. In some embodiments, the dilution buffer comprises an organic solvent. In some embodiments, the dilution buffer comprises ethanol and water. In some embodiments, the dilution buffer comprises 10% to 20% of ethanol in PBS buffer.

In some embodiments, the mixing comprises laminar mixing, vortex mixing, turbulent mixing, or a combination thereof. In some embodiments, the mixing comprises cross-mixing. In some embodiments, the mixing comprises inline mixing. In some embodiments, the mixing comprises introducing at least a portion of the first solution through a first inlet channel and at least a portion of the second solution through a second inlet channel, and wherein an angle between the first inlet channel and the second inlet channel is from about 0 to 180 degrees. In some embodiments, the angle between the first inlet channel and the second inlet channel is from about 15 to 180 degrees, from about 30 to 180 degrees, from about 45 to 180 degrees, from about 60 to 180 degrees, from about 90 to 180 degrees, or any numbers or ranges therebetween. In some embodiments, the mixing comprises introducing a portion of the first solution through a third inlet channel. The mixing step can take place by any number of methods, e.g., by mechanical means such as a vortex mixer. In some embodiments, the mixing step comprises inline mixing.

In some embodiments, a method of making a formulation comprising the herein-described nanoparticles comprises a filtration step. In some embodiments, a method of making a formulation comprising the herein-described nanoparticles comprises buffer exchange. In some embodiments, the buffer exchange comprises dialysis, chromatography, or tangential flow filtration (TFF).

### II. Pharmaceutical Compositions

In one aspect, disclosed herein are pharmaceutical compositions comprising one or more described particle compositions. For example, a pharmaceutical composition can include one or more LNP compositions including one or more different payloads. Pharmaceutical compositions can further include one or more pharmaceutically acceptable excipients, carrier, or accessory ingredients such as those described herein. General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, Md., 2006. Excipients or carriers can include any ingredient other than the compound(s) of the disclosure, the other lipid component(s) and the payload. An excipient may impart either a functional (e.g. drug release rate controlling) and/or a nonfunctional (e.g. processing aid or diluent) characteristic to the formulations. The choice of excipient and carrier can depend on factors such as the particular mode of administration, the effect of the excipient on solubility and stability, and the nature of the dosage form. Parenteral formulations are typically aqueous or oily solutions or suspensions. Excipients or carrier such as sugars (including but not restricted to glucose, mannitol, sorbitol, etc.), salts, carbohydrates and buffering agents (preferably to a pH of from 3 to 9) can be used. In some embodiments, the LNP compositions can be formulated with a sterile non-aqueous solution or as a dried form to be used in conjunction with a suitable vehicle such as sterile, pyrogen-free water (WFI).

In some embodiments, the excipient or carrier can make up greater than 50% of the total mass or volume of a pharmaceutical composition comprising a nanoparticle composition. For example, the excipient or carrier can make up 50%, 60%, 70%, 80%, 90%, or more of a pharmaceutical composition. In some embodiments, a pharmaceutically acceptable excipient or carrier is at least 95%, at least 96%, at least 97%, at least 98%, at least 99%, or 100% pure. In some embodiments, a pharmaceutical composition can comprise between 0.1% and 100% (wt/wt) of one or more nanoparticle compositions. In certain embodiments, the nanoparticle compositions and/or pharmaceutical compositions are refrigerated or frozen for storage and/or shipment (e.g., being stored at a temperature of 4° C. or lower, such as a temperature between about −150° C. and about 0° C. or between about −80° C. and about −20° C. In some embodiments, the nanoparticle compositions and/or pharmaceutical compositions are refrigerated or frozen at about −5° C., −10° C., −15° C., −20° C., −25° C., −30° C., −40° C., −50° C., −60° C., −70° C., −80° C., −90° C., −130° C., or −150° C.

The described LNP compositions and/or pharmaceutical compositions can be administered to any patient or subject, including those patients or subjects that may benefit from a therapeutic effect provided by the delivery of the payload to one or more particular cells, tissues, organs, or systems or groups thereof. In some embodiments, the subject is a mammal such as human. In some embodiments, the subject is non-human primates or mammals, including commercially relevant mammals such as cattle, pigs, hoses, sheep, cats, dogs, mice, and/or rats.

A pharmaceutical composition including one or more nanoparticle compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage. Pharmaceutical compositions may be prepared in a variety of forms suitable for a variety of routes and methods of administration. For example, pharmaceutical compositions may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

In some embodiments, the pharmaceutical composition comprises 1, 2, 3, 4, 5, 6, 7, 8, 9, 10 or more payloads. In some embodiments, the pharmaceutical composition comprises two distinct payloads, such guide RNA and mRNA. The guide RNA and mRNA can be located in the same LNP composition, or they can be located at separate LNP compositions. For example, a pharmaceutical composition can comprise two distinct LNP compositions, one comprising a guide RNA payload and the other comprising an mRNA payload. For another example, a pharmaceutical composition can comprise two distinct LNP compositions, one comprising a guide RNA (or mRNA) payload and the other comprising both an mRNA payload and a guide RNA payload. For yet another example, a pharmaceutical composition can comprise one LNP composition, which comprising an mRNA payload and a guide RNA payload. In some embodiments, the pharmaceutical composition comprises two or more distinct LNP compositions. In some embodiments, the two or more distinct LNP compositions are present in the pharmaceutical composition such that the mRNA molecule(s) and the guide RNA molecule(s) are at a mole or weight ratio described herein.

The gRNA and mRNA payloads can be present in the pharmaceutical composition at various molar or weight ratios. For example, the gRNA to mRNA ratio in the pharmaceutical composition can be from 0.01 to 100 by weight, and/or any value therebetween. For example, the gRNA to mRNA ratio in the pharmaceutical composition can be from 0.01 to 100 by mole, and/or any value therebetween. In some embodiments, the ratio of gRNA to mRNA in the pharmaceutical composition is from about 1 to about 50 by weight or by mole, and/or any value therebetween. In some embodiments, the ratio of gRNA to mRNA in the pharmaceutical composition is from about 0.1 to about 10 by weight or by mole, and/or any value therebetween. In some embodiments, the ratio of gRNA to mRNA in the pharmaceutical composition is from about 0.2 to about 5, from about 0.25 to about 4, from about 0.3 to about 3, or from about 0.5 to about 2 by weight. In some embodiments, the ratio of gRNA to mRNA in the pharmaceutical composition is from about 0.2 to about 5, from about 0.25 to about 4, from about 0.3 to about 3, or from about 0.5 to about 2 by mole. In some embodiments, the gRNA to mRNA ratio in the pharmaceutical composition is about 1:1, about 1:2, about 1:3, about 1:4, about 1:5, about 1:6, about 1:7, about 1:8, about 1:9, or about 1:10 by weight. In some embodiments, the gRNA to mRNA ratio in the pharmaceutical composition is about 1:1, about 1:2, about 1:3, about 1:4, about 1:5, about 1:6, about 1:7, about 1:8, about 1:9, or about 1:10 by mole. In some embodiments, the mRNA to gRNA ratio in the pharmaceutical composition is about 1:1, about 1:2, about 1:3, about 1:4, about 1:5, about 1:6, about 1:7, about 1:8, about 1:9, or about 1:10 by weight. In some embodiments, the mRNA to gRNA ratio in the pharmaceutical composition is about 1:1, about 1:2, about 1:3, about 1:4, about 1:5, about 1:6, about 1:7, about 1:8, about 1:9, or about 1:10 by mole. In some embodiments, the gRNA to mRNA ratio in the pharmaceutical composition is about 1:1 by weight. In some embodiments, the gRNA to mRNA ratio in the pharmaceutical composition is about 1:1 by mole.

In some embodiments, the gRNA in the pharmaceutical composition targets a disease-causing gene that is produced in the hepatocytes. In some embodiments, the target cells of the disclosed compositions are selected from hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells. In some embodiments, the pharmaceutical composition comprises more than one guide RNA. For example, the pharmaceutical composition can comprise 2, 3, 4, 5, or more distinct guide RNAs. In some embodiments, the pharmaceutical composition comprises two guide RNA molecules. In some embodiments, the pharmaceutical composition comprises one mRNA and two or more guide RNA molecules. In some embodiments, the two or more guide RNA molecules target the same disease-causing gene. In some embodiments, the two or more guide RNA molecules target different genes. In some specific embodiments, the two or more guide RNA molecules target two separate disease-causing genes of interest produced in the hepatocytes. In some embodiments, the gRNA is a sgRNA. In some embodiments, the gRNA is a dgRNA.

### IV. Method of Use

The LNP compositions and pharmaceutical compositions disclosed herein can be used in methods for gene editing, both in vivo and in vitro. In some embodiments, the methods comprise contacting a cell with an LNP composition or a pharmaceutical composition described herein. In some embodiments, the cell is a mammalian cell. In some embodiments, the cell is a rodent cell. In some embodiments, the cell is a human cell. In some embodiments, the cell is a liver cell. In certain embodiments, the cell is a human liver cell. In some embodiments, the liver cell is a hepatocyte. In some embodiments, the hepatocyte is a human hepatocyte. In some embodiments, the liver cell is a stem cell. In some embodiments, the human liver cell is a liver sinusoidal endothelial cell (LSEC). In some embodiments, the human liver cell is a Kupffer cell. In some embodiments, the human liver cell is a hepatic stellate cell. In some embodiments, the human liver cell is a tumor cell. In some embodiments, the human liver cell is a liver stem cell. In some embodiments, the cell comprises ApoE-binding receptors. In some embodiments, engineered cells are provided; for example an engineered cell can be derived from any one of the cell types as described herein. Such engineered cells can be produced according to the methods described herein. In some embodiments, the engineered cell resides within a tissue or organ, e.g., a liver within a subject.

In some embodiments, the cell comprises a modification, for example an insertion or deletion (“indel”) or substitution of nucleotides in a target sequence. In some embodiments, the modification comprises an insertion of 1, 2, 3, 4 or 5 or more nucleotides in a target sequence. In some embodiments, the modification comprises an insertion of either 1 or 2 nucleotides in a target sequence. In other embodiments, the modification comprises a deletion of 1, 2, 3, 4, 5, 6, 7, 8, 9, 10, 15, 20 or 25 or more nucleotides in a target sequence. In some embodiments, the modification comprises a deletion of either 1 or 2 nucleotides in a target sequence. In some embodiments, the modification comprises an indel which results in a frameshift mutation in a target sequence. In some embodiments, the modification comprises a substitution of 1, 2, 3, 4, 5, 6, 7, 8, 9, 10, 15, 20 or 25 or more nucleotides in a target sequence. In some embodiments, the modification comprises a substitution of either 1 or 2 nucleotides in a target sequence. In some embodiments, the modification comprises one or more of an insertion, deletion, or substitution of nucleotides resulting from the incorporation of a template nucleic acid, for example any of the template nucleic acids described herein.

In some embodiments, the method comprises contacting a population of cells, such as a population of engineered cells. In some embodiments, the population of cells comprises engineered cells cultured in vitro. In some embodiments, the population resides within a tissue or organ, e.g., a liver within a subject. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or more of the cells within the population is engineered. In certain embodiments, a method disclosed herein results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% editing efficiency (or “percent editing”), defined by detection of indels. In other embodiments, a method disclosed herein, results in at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% DNA modification efficiency, defined by detecting a change in sequence, whether by insertion, deletion, substitution or otherwise. In certain embodiments, a method disclosed herein results in an editing efficiency level or a DNA modification efficiency level of between about 5% to about 100%, about 10% to about 50%, about 20 to about 100%, about 20 to about 80%, about 40 to about 100%, or about 40 to about 80%.

In some embodiments, cells within the population comprise a modification. In some embodiments, the modification comprises an insertion of 1, 2, 3, 4 or 5 or more nucleotides in a target sequence. In some embodiments, the modification comprises an insertion of either 1 or 2 nucleotides in a target sequence. In other embodiments, the modification comprises a deletion of 1, 2, 3, 4, 5, 6, 7, 8, 9, 10, 15, 20 or 25 or more nucleotides in a target sequence. In some embodiments, the modification comprises a deletion of either 1 or 2 nucleotides in a target sequence. In some embodiments, the modification comprises an indel which results in a frameshift mutation in a target sequence. In some embodiments, at least 80%, at least 85%, at least 90%, at least 91%, at least 92%, at least 93%, at least 94%, at least 95%, at least 96%, at least 97%, at least 98%, or at least 99% or more of the engineered cells in the population comprise a frameshift mutation.

In some embodiments, the LNP compositions can be used to edit a gene resulting in a gene knockout. In some embodiments, the LNP compositions can be used to edit a gene resulting in a gene correction. In some embodiments, the LNP compositions can be used to edit a cell resulting in gene insertion. In some embodiments, disclosed are methods for silencing expression of a target gene in a cell. In some embodiments, the method comprises contacting a cell comprising an expressed target gene with an LNP composition or a pharmaceutical composition described herein under conditions whereby the gRNA enters the cell and silences the expression of the target gene within the cell. In certain embodiments, the cell is in a mammal, such as a human. In some embodiments, the method for silencing expression of a target gene comprises administering to the mammal a therapeutically effective amount of an LNP composition or pharmaceutical composition comprising one or more gRNAs described herein. In some embodiments, the administration of the LNP composition or pharmaceutical composition comprising one or more gRNAs described herein reduces target RNA levels by at least about 5%, 100%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100% (or any range therein) relative to RNA levels detected in the absence of the gRNA (e.g., buffer control or irrelevant gRNA control). In some embodiments, the administration of the LNP composition or pharmaceutical composition comprising one or more gRNAs reduces target RNA levels for at least about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 days or more (or any range therein) relative to a negative control such as, e.g., a buffer control or an irrelevant non-targeting gRNA control.

In some embodiments, administration of the LNP composition or pharmaceutical composition may result in gene editing which results in persistent response. For example, administration can result in a duration of response of a day, a month, a year, or longer. As used herein, “duration of response” means that, after cells have been edited using an LNP composition or pharmaceutical composition disclosed herein, the resulting modification is still present for a certain period of time after the administration. The modification can be detected by measuring target protein levels. The modification can be detected by detecting the target DNA. In some embodiments, the duration of response can be at least 1 week, at least 2 weeks, at least 1 month, at least 2 months, at least 4 months, at least 6 months, or at least 1 year. In certain embodiments, the duration of response may be about 26 weeks. In some embodiments, the duration of response can be at least 5 years or at least 10 years. A persistent response is detectable after at least 6 months, either by measuring target protein levels or by detection of the target DNA.

In one aspect, disclosed herein are methods for treating a disease or condition, including raising an immune response to an immunogen, in a subject. In one embodiment, the disease or condition is treatable by administering the payload. In some embodiments, the disease or condition is characterized by missing or aberrant protein or polypeptide activity. For example, an LNP composition comprising an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. A payload included in an LNP composition may also be capable of altering the rate of transcription of a given species, thereby affecting gene expression.

Diseases and/or conditions characterized by dysfunctional or aberrant protein or polypeptide activity can include, but are not limited to, rare diseases, infectious diseases (as both vaccines and therapeutics), cancer and proliferative diseases, genetic diseases (e.g., cystic fibrosis), autoimmune diseases, diabetes, neurodegenerative diseases, cardio- and reno-vascular diseases, and metabolic diseases. Multiple diseases and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene. In some embodiments, the present disclosure provides a method for treating such diseases and/or conditions in a subject by administering an LNP composition or pharmaceutical composition comprising an RNA payload, wherein the RNA can be an mRNA encoding a polypeptide that antagonizes or otherwise overcomes an aberrant protein activity present in the cell of the subject.

Although the present invention and its advantages have been described in detail, it should be understood that various changes, substitutions and alterations can be made herein without departing from the spirit and scope of the invention as defined in the appended claims.

Further embodiments of the present disclosure, for example, the LNP compositions (including the various components of the LNP composition), the pharmaceutical compositions, the methods of preparing the compositions, the methods of gene editing, the methods of treatment, and other methods of using the compositions, can be found in various WO and US patent/patent application publications, including U.S. Pat. No. 9,868,692B2, US20180353434A1, U.S. Pat. No. 8,492,359B2, U.S. Pat. No. 9,878,042B2, US20180148719, U.S. Pat. No. 9,687,448B2, U.S. Pat. No. 9,415,109B2, U.S. Pat. No. 7,858,117B2, U.S. Pat. No. 9,404,127B2, U.S. Pat. No. 9,504,651, US20070087045A1, US20180092848A1, US20170273907A1, US20180147298A1, WO2019067992A1, WO2019067999A1, WO2018185241, WO 2018170306A1, WO 2019046809A1, WO2017173054A1, WO2015095340, WO2016197133A1, and WO2018191750, all of which are hereby incorporated by reference in their entirety. Further description about the LNP compositions, the pharmaceutical compositions, and the methods are described in WO2016153012 A1, WO2018062413 A1, WO2019027055 A1, WO2017/173054A1, WO2015/095340A1, WO2013063468A1, WO2010054401A1, and WO 2018/170306, all of which are hereby incorporated by reference in their entirety.

The present invention is further illustrated in the following Examples which are given for illustration purposes only and are not intended to limit the invention in any way.

### EXAMPLES

### Example 1. Synthesis of VL401 and VL469

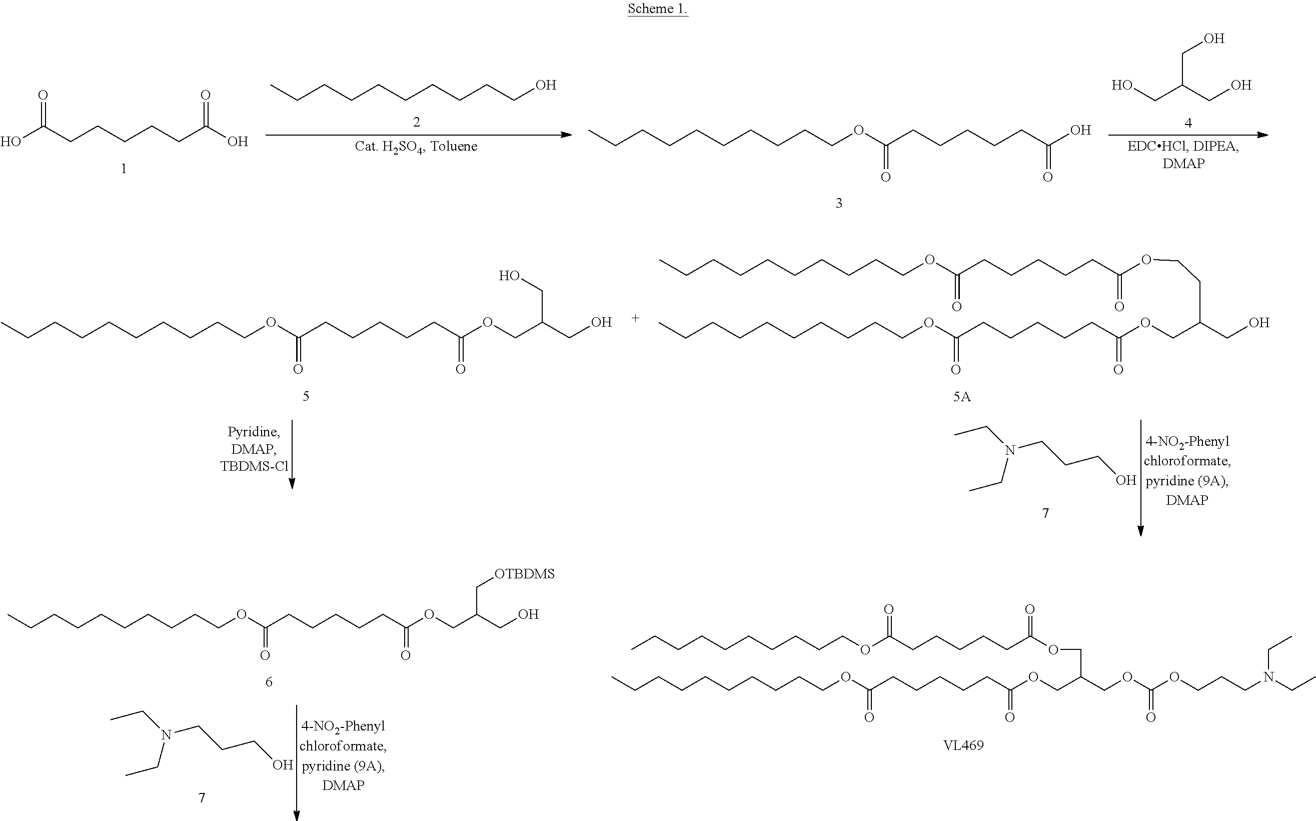

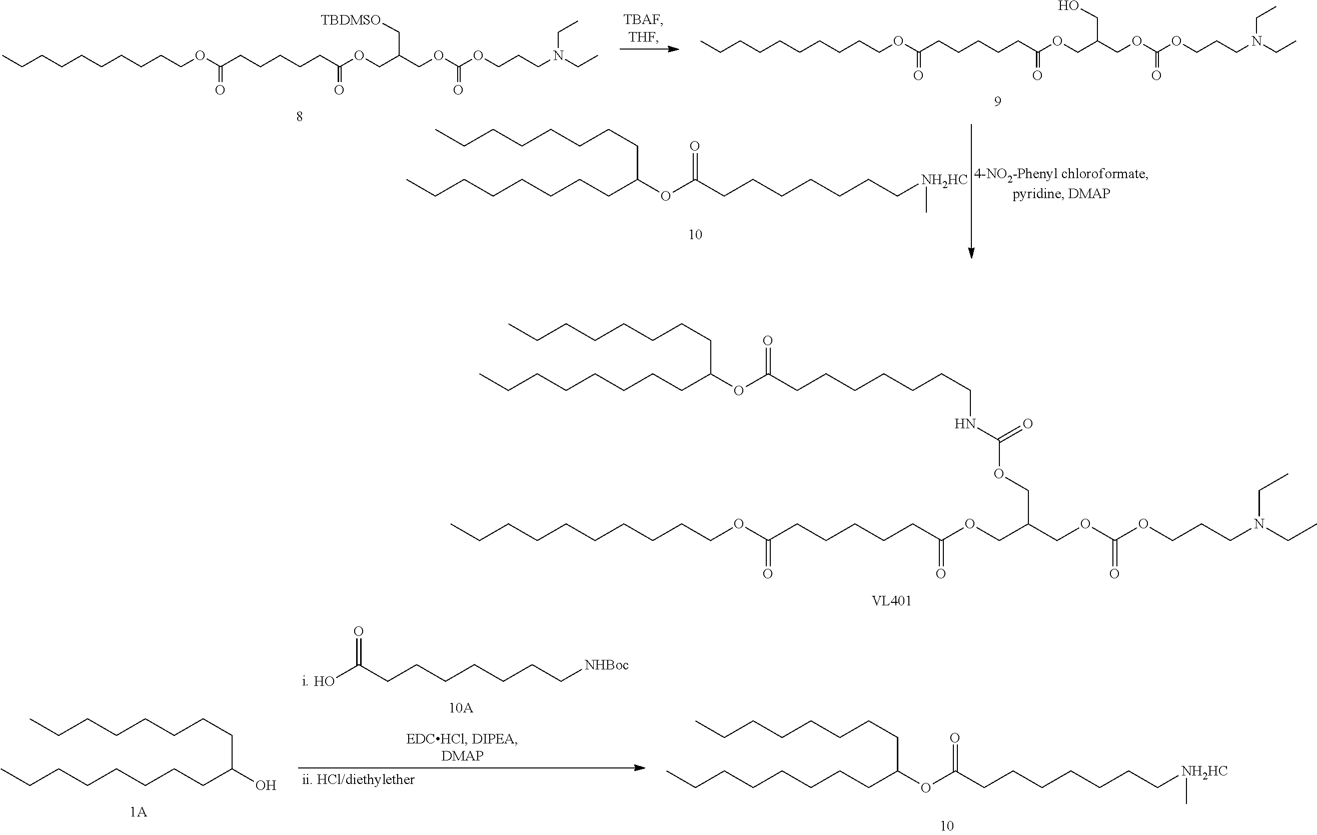

To a solution of compound 1 (16.0 g, 100 mmol) in toluene (90 mL) was added compound 2 (4 mL, 100 mmol) followed by H₂SO₄ (0.1 mL) as catalytic amount and refluxed for 6 h. After 6 h, TLC showed completion of compound 1 and the reaction mixture was quenched with a solution of Na₂CO₃ (100 mL) and the product was extracted into EtOAc (3×200 mL). The organic layer was separated and evaporated to get the crude. The crude mass was then purified by silica gel (60-120 mesh) column chromatography by eluting with 20% EtOAc in pet-ether to afford compound 3 (8.0 g, 27%) as white solid. ¹H NMR (400 MHz, DMSO-d₆): δ ppm 11.97 (s, 1H), 3.98 (t, J=6.4 Hz, 2H), 2.26 (t, J=7.6 Hz, 2H), 2.18 (t, J=7.6 Hz, 2H), 1.70-1.50 (m, 6H), 1.40-1.20 (m, 16H), 0.88 (t, J=6.4 Hz, 3H).

To a solution of compound 3 (6.0 g, 20 mmol) in CH₂Cl₂ (60 mL) was added 2-(hydroxymethyl)propane-1,3-diol (2.11 g, 20 mmol) followed by EDC·HCl (5.75 g, 30 mmol), DMAP (488 mg, 4 mmol) and DIPEA (4.83 mL, 30 mmol). Reaction mixture was stirred at room temperature (RT) for 24 h. The reaction mixture was diluted with water and was the product was extracted with DCM (2×200 mL). The organic layer was separated and evaporated to get the crude. The crude mass was then purified by silica gel (60-120 mesh) column chromatography by eluting with 50% EtOAc in pet-ether to afford compound 5 (3.5 g, 45%). ¹H NMR (400 MHz, DMSO-d₆): δ ppm 4.45 (t, J=5.2 Hz, 2H), 4.10-4.00 (m, 4H), 3.50-3.35 (m, 4H), 2.26 (m, t, J=7.2 Hz, 4H), 1.90-1.75 (m, 1H), 1.60-1.45 (m, 6H), 1.40-1.20 (m, 16H), 0.85 (t, J=6.4 Hz, 3H).

A solution of compound 5 (2.0 g, 5.15 mmol), DMAP (188 mg, 1.54 mmol), and TBDMS-Cl (850 mg, 5.67 mmol) in pyridine (10 mL) was stirred at RT for 3 h. The reaction mixture was diluted with water (20 mL) and the product was extracted with DCM (2×100 mL). The organic layer was separated and evaporated to get the crude. The crude mass was then purified by silica gel (60-120 mesh) chromatography by eluting with 30% EtOAc in pet-ether to obtain compound 6 as a colourless oil (1.2 g, 46%). ¹H NMR (400 MHz, DMSO-d₆): δ ppm 4.51 (t, J=5.2 Hz, 1H), 4.10-3.90 (m, 4H), 3.65-3.50 (m, 2H), 3.45-3.30 (m, 2H), 2.35-2.20 (m, 4H), 1.90-1.80 (m, 1H), 1.60-1.45 (m, 6H), 1.30-1.10 (m, 16H), 0.90-0.75 (m, 12H), 0.10 (s, 6H).

To a solution of compound 7 (375 mg, 3.10 mmol) in CH₂Cl₂ (13 mL) were added 4-nitrophenyl chloroformate (965 mg, 5.16 mmol) followed by pyridine (0.38 mL, 5.16 mmol) and DMAP (87 mg, 0.77 mmol) and the reaction was continued for 2 h. 6 was added (1.3 g, 2.58 mmol) and the reaction mixture was allowed to stir at 45° C. for 16 h. Reaction mixture was diluted with water (50 mL) and the product was extracted with CH₂Cl₂ (2×50 mL); the organic layer was separated and evaporated to get the crude mass. The crude mass was purified by silica gel column eluting with 50% EtOAc in pet-ether to get 8 (700 mg, 43%) as yellow oil. ¹H NMR (400 MHz, DMSO-d₆): δ ppm 4.20-3.98 (m, 8H), 3.61 (d, J=5.6 Hz, 2H), 2.50-2.35 (m, 6H), 2.35-2.10 (m, 6H), 1.80-1.70 (m, 2H), 1.60-1.45 (m, 6H), 1.60-1.45 (m, 5H), 1.40-1.20 (m, 16H), 1.00-0.89 (m, 14H), 0.10 (s, 6H).

To a stirred solution of 8 (700 mg, 1.06 mmol) in THE (4 mL) was added TBAF (0.46 mL, 1.593 mmol) at ice temperature and the reaction mixture was stirred at RT for 4 h. The reaction mixture was diluted with water (10 mL) and the product was extracted with CH₂Cl₂ (2×50 mL); the organic layer was separated and evaporated to get the crude. Crude material was purified by silica gel column eluting with 50% EtOAc in pet-ether yielded 9 (300 mg, 52%) as a colourless gummy liquid.

Compound VL401. To a stirred solution of 9 (200 mg, 0.366 mmol) in CH₂Cl₂ (2 mL) was added 4-nitrophenyl chloroformate (965 mg, 5.16 mmol) followed by pyridine (0.38 mL, 5.16 mmol) and DMAP (87 mg, 0.77 mmol); the reaction mixture was continued stirring for 2 h and added compound 10 (159 mg, 0.402 mmol) and the reaction mixture was allowed to stir at 90° C. for 16 h. Reaction mixture was diluted with water (50 mL) and the product was extracted with CH₂Cl₂ (2×50 mL); the organic layer was separated and evaporated to get the crude product. The crude material was purified by silica gel column eluting with 6% MeOH in CH₂Cl₂ to obtain VL401 (80 mg, 22%) as a gummy liquid. ¹H NMR (400 MHz, DMSO-d₆): δ ppm 7.20-7.10 (m, 1H), 4.85-4.75 (m, 1H), 4.30-4.00 (m, 10H), 3.60-3.30 (m, 5H), 3.00-2.90 (m, 2H), 2.40-2.20 (m, 10H), 1.80-0.80 (m, 75H). Mass [M+H]⁺ Calcd for 969.7; found 969.6.

Compound VL469. Compound 5A is reacted with 4-nitropehnyl chloroformate in dichloromethane in the presence of pyridine and DMAP. The 4-nitropehnyl carbonate thus formed is then reacted with compound 7 to obtain compound VL469.

### Example 2. Synthesis of VL403

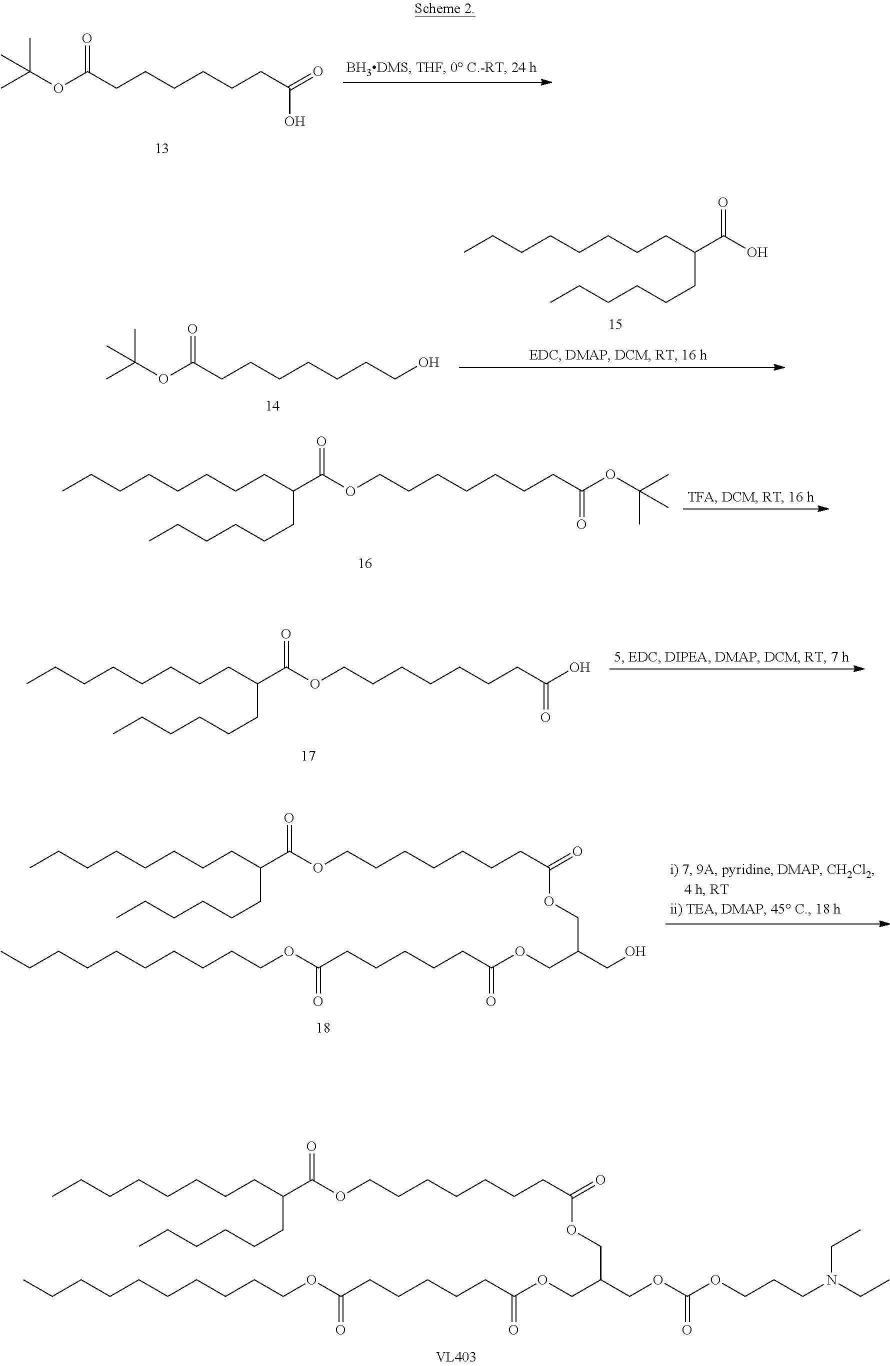

To a solution of commercially available compound 13 (11.5 g, 50 mmol) in THF (50 mL) was added BH₃·DMS (2 M in THF, 30 mL) at 0° C. Reaction mixture was stirred at RT for 24 h. The reaction mixture was quenched with MeOH and the organic layer was concentrated under reduce pressure to obtain compound 14 (10 g, 92.5%). ¹H NMR (400 MHz, CDCl₃): δ ppm 3.63 (t, J=6.4 Hz, 2H), 2.20 (t, J=6.4 Hz, 2H), 1.70-1.50 (m, 4H), 1.44 (s, 9H), 1.40-1.30 (m, 7H).

To a stirred solution of compound 14 (4.8 g, 22.2 mmol) in CH₂Cl₂ (70 mL) was added compound 15 (6.3 g, 24.4 mmol) followed by EDC·HCl (5.5 g, 28.8 mmol), DMAP (1.4 g, 11.1 mmol) and the reaction mixture was stirred for 16 h. The reaction mixture was diluted with water (50 mL) and the product was extracted with CH₂Cl₂ (2×100 mL); the organic layer was separated and evaporated to get the crude product. The crude product was purified by silica gel column by eluting with 10% EtOAc in pet-ether to obtain compound 16 (5 g, 50%) as a gummy liquid. ¹H NMR (400 MHz, CDCl₃): δ ppm 4.08 (t, J=6.8 Hz, 2H), 2.40-2.20 (m, 3H), 1.70-1.60 (m, 4H), 1.46 (s, 9H), 1.40-1.20 (m, 30H), 0.95-0.85 (m, 6H).

Compound 17. To a stirred solution of compound 16 (1.9 g, 4.18 mmol) in CH₂Cl₂ (20 mL) was added TFA (2 mL, 12.5 mmol) at 0° C. and the reaction mixture was stirred for 16 h. The reaction mixture was concentrated under reduce pressure to give compound 17 (1.8 g, 99%) as a gummy liquid. ¹H NMR (400 MHz, CDCl₃): δ ppm 4.06 (t, J=6.8 Hz, 2H), 2.40-2.25 (m, 3H), 1.80-1.50 (m, 4H), 1.50-1.20 (m, 30H), 1.00-0.85 (m, 6H).

To a stirred solution of compound 17 (1.5 g, 3.76 mmol) in CH₂Cl₂ (20 mL) was added compound 5 (2.1 g, 5.65 mmol) followed by EDC·HCl (1.1 g, 5.65 mmol), DIPEA (1 mL, 5.65 mmol) and DMAP (138 mg, 1.13 mmol), and the reaction mixture was stirred for 7 h. The reaction mixture was diluted with water (50 mL) and the product was extracted with CH₂Cl₂ (2×25 mL); the organic layer was separated and evaporated to get the crude product. The crude mass was purified by silica gel column by eluting with 20% EtOAc in pet-ether to obtain compound 18 (0.6 g, 21%) as a yellow oil. ¹H NMR (400 MHz, CDCl₃): δ ppm 4.25-4.10 (m, 4H), 4.10-4.00 (m, 4H), 3.70-3.60 (m, 2H), 2.60-2.50 (m, 7H), 2.40-2.30 (m, 7H), 1.80-1.50 (m, 10H), 1.50-1.20 (m, 41H), 0.95-0.85 (m, 9H).

To a solution of compound 7 (204 mg, 1.56 mmol) in CH₂Cl₂ (6 mL) were added 4-nitrophenyl chloroformate (470 mg, 2.34 mmol) followed by pyridine (0.1 mL, 1.17 mmol) and DMAP (14 mg, 0.11 mmol), and the reaction was stirred for 4 h; added compound 18 (300 mg, 0.39 mmol) and the reaction mixture was allowed to stirred at 45° C. for 18 h. Reaction mixture was diluted with water (20 mL) and the product was extracted with CH₂Cl₂ (2×50 mL); the organic layer was separated and evaporated to get the crude product. The crude mass was purified by silica gel column eluting with 50% EtOAc in pet-ether to get VL403 (130 mg, 35%) as a yellow oil. ¹H NMR (400 MHz, CDCl₃): δ ppm 4.25-4.10 (m, 8H), 4.10-4.00 (m, 4H), 2.60-2.50 (m, 7H), 2.40-2.25 (m, 7H), 1.85-1.75 (m, 2H), 1.70-1.60 (m, 10H), 1.50-1.20 (m, 46H), 1.10-0.95 (m, 6H), 0.95-0.85 (m, 9H). m/z: [M+H]⁺ Calcd for 926.7; found 926.6.

### Example 3. Synthesis of VL404

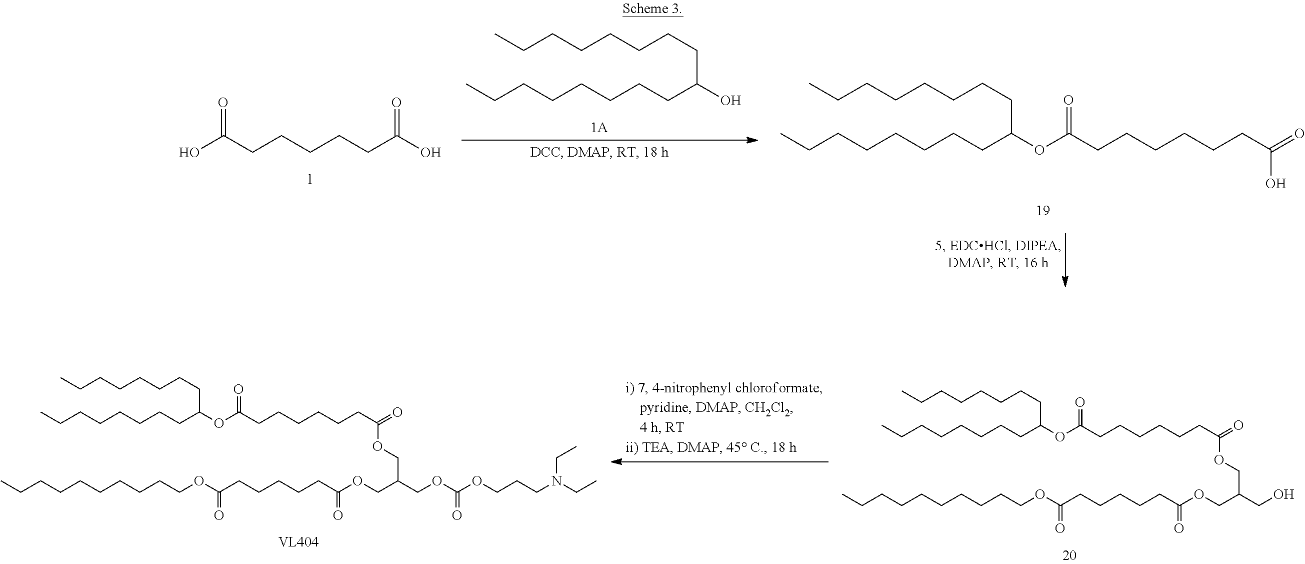 [SMILES: O=C(O)CCCCCC(=O)O ; CCCCCCCCC(O)CCCCCCCC ; CCCCCCCCC(CCCCCCCC)OC(=O)CCCCCCC(=O)O ; CCCCCCCCCCOC(=O)CCCCCC(=O)OCC(COC(=O)CCCCCCC(=O)OC(CCCCCCCC)CCCCCCCC)COC(=O)OCCCN(CC)CC ; CCCCCCCCCCOC(=O)CCCCCC(=O)OCC(CO)COC(=O)CCCCCCC(=O)OC(CCCCCCCC)CCCCCCCC]

To a stirred solution of compound 1 (9.6 g, 54.68 mmol) in CH₂Cl=2 (100 mL) was added compound 1A (10 g, 39.06 mmol) followed by DCC (8.85 g, 42.96 mmol) and DMAP (2.38 g, 19.52 mmol). The reaction mixture was stirred at RT for 18 h. After complete disappearance of compound 1 by TLC, the reaction mixture was diluted with water (100 mL) and the product was extracted into CH₂Cl₂ (2×100 mL). The organic layer was separated, evaporated in vacuo, and the crude product obtained was purified by silica gel column, eluting with 50% EtOAc in pet-ether to obtain compound 19 (6 g, 26%) as a gummy solid. ¹H NMR (400 MHz, DMSO-d₆): δ ppm 4.90-4.80 (m, 1H), 2.35 (t, J=5.2 Hz, 2H), 2.26 (t, J=5.2 Hz, 2H), 1.80-1.20 (m, 36H), 0.95-0.85 (m, 6H).

To a stirred solution of compound 19 (4.99 g, 9.99 mmol) in CH₂Cl₂ (20 mL) was added compound 5 (2.0 g, 6.666 mmol) followed by EDC·HCl (1.916 g, 9.99 mmol), DIPEA (3.48 mL, 19.99 mmol), DMAP (244 mg, 1.99 mmol) and the reaction mixture was stirred for 16 h. After complete disappearance of compound 6 by TLC, reaction mixture was diluted with water (50 mL) and the product was extracted with CH₂Cl₂ (2×50 mL); the organic layer was separated and evaporated to get the crude product. The crude mass was purified by silica gel column by eluting with 50% EtOAc in pet-ether to yield compound 20 (2.1 g, 52%) as a gummy liquid. ¹H NMR (400 MHz, DMSO-d₆): δ ppm 4.90-4.70 (m, 2H), 4.20-3.95 (m, 6H), 3.42 (t, J=5.6 Hz, 2H), 2.40-2.20 (m, 8H), 2.10-2.00 (m, 1H), 1.60-1.40 (m, 14H), 1.40-1.10 (m, 44H), 0.85 (t, J=6.4 Hz, 9H).

Compound VL404. To a solution of compound 7 (672 mg, 5.12 mmol) in CH₂Cl₂ (20 mL) were added 4-nitrophenyl chloroformate (1.53 g, 7.62 mmol) followed by pyridine (0.5 mL, 6.38 mmol) and DMAP (50 mg, 0.384 mmol) and the reaction was stirred for 5 h. After 5 h, added compound 20 (1.0 g, 1.28 mmol) and the reaction mixture was allowed to stir at 45° C. for 18 h. Reaction mixture was diluted with water (20 mL) and the product was extracted with CH₂Cl₂ (2×50 mL); the organic layer was separated and evaporated to get the crude product. The crude mass was purified by silica gel column eluting with 50% EtOAc in pet-ether to obtain VL404 (510 mg, 42%) as a gummy liquid. ¹H NMR (400 MHz, CDCl₃): δ ppm 4.90-4.80 (m, 1H), 4.25-4.00 (m, 10H), 2.60-2.50 (m, 7H), 2.40-2.25 (m, 8H), 1.85-1.75 (m, 2H), 1.70-1.60 (m, 8H) (1.60-1.45 (m, 4H), 1.40-1.15 (m, 46H), 1.00 (t, J=6.8 Hz, 6H), 0.95-0.85 (m, 9H). ELSD-MS m z calc. 939.74; found: 940.6.

### Example 4. Synthesis of VL406 and VL422

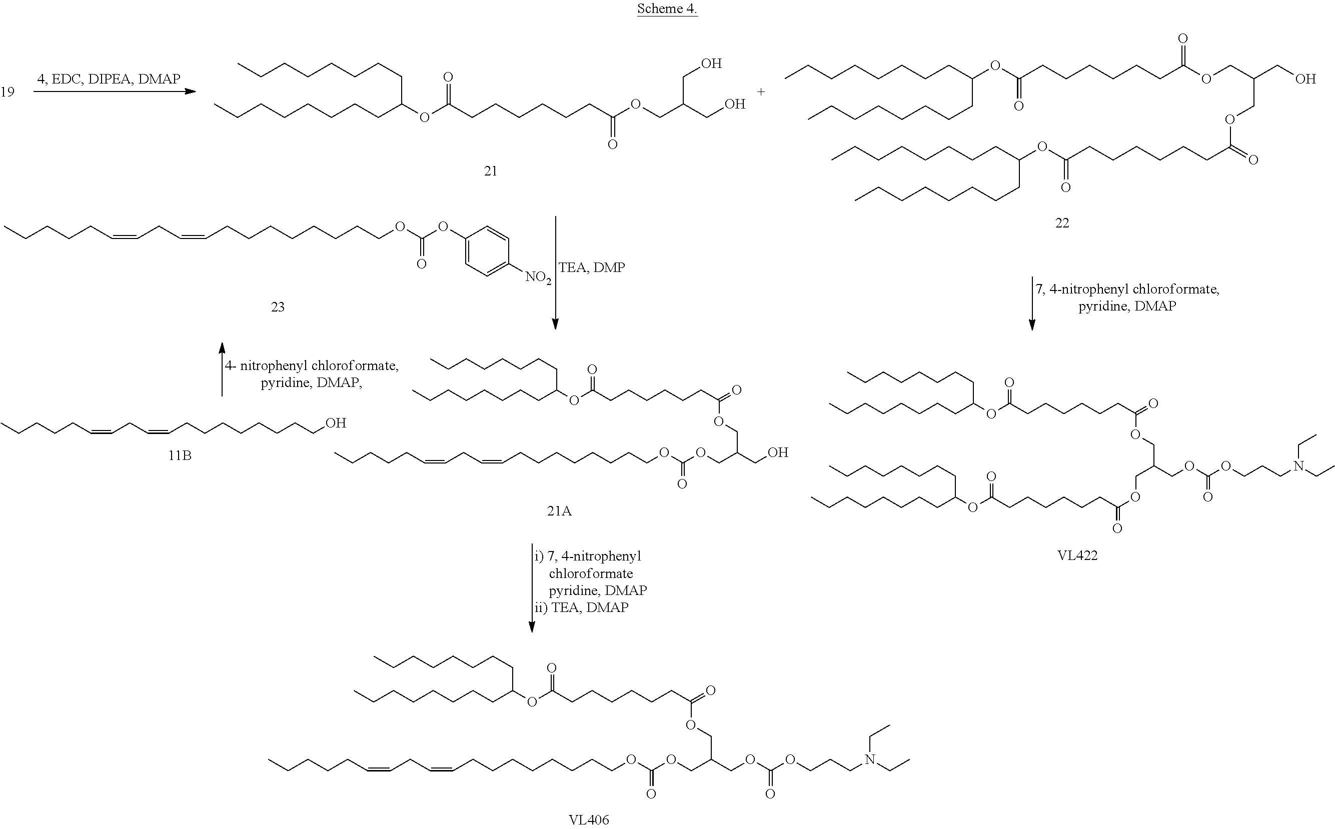

To a solution of compound 11B (5 g, 18.76 mmol) in CH₂Cl₂ (50 mL) were added 4-nitrophenyl chloroformate (11.37 mg, 56.29 mmol) followed by pyridine (4.53 mL, 56.29 mmol) and DMAP (687 mg, 5.62 mmol) and the reaction was stirred for 16 h. Reaction mixture was then filtered and the white coloured solid was discarded. The filtrate was evaporated and triturated with pet-ether to yield 23 (6 g, 74%) as a gummy liquid. ¹H NMR (400 MHz, CDCl₃): δ ppm 8.27 (d, J=7.2 Hz, 2H), 7.37 (d, J=7.2 Hz, 2H), 5.50-5.30 (m, 4H), 4.28 (t, J=6.4 Hz, 2H), 2.77 (t, J=6.4 Hz, 2H), 2.10-2.00 (m, 4H), 1.85-1.70 (m, 2H), 1.50-1.20 (m, 18H), 0.89 (t, J=7.2 Hz, 3H).

To a solution of 19 (3.0 g, 7.28 mmol) in CH₂Cl₂ (30 mL) was added compound 4 (772 mg, 7.28 mmol) followed by EDC·HCl (2.0 g, 10.9 mmol), DMAP (173 mg, 1.45 mmol) and DIPEA (3.8 mL, 21.8 mmol). Reaction mixture was stirred at RT for 5 h. The reaction mixture was diluted with water and the product was extracted with CH₂Cl₂ (2×50 mL). The organic layer was separated and evaporated to get the crude. The crude mass was purified by silica gel (60-120 mesh) column chromatography by eluting with EtOAc in pet-ether to obtain pure compounds 21 and 22. ¹H NMR (21, 400 MHz, DMSO-d₆): δ ppm 4.90-4.80 (m, 1H), 4.46 (t, J=5.2 Hz, 2H), 4.00 (d, J=6.4 Hz, 2H), 3.50-3.30 (m, 4H), 2.30-2.20 (m, 4H), 1.90-1.80 (m, 1H), 1.60-1.40 (m, 8H), 1.40-1.20 (m, 29H), 0.85 (t, J=6.4 Hz, 6H). ¹H NMR of 22 (400 MHz, CDCl₃): δ ppm 4.90-4.80 (m, 2H), 4.20-4.10 (m, 4H), 3.70-3.60 (m, 2H), 2.50-2.20 (m, 10H), 1.80-1.20 (m, 70H), 1.10-1.00 (m, 2H), 1.00-0.90 (m, 12H).

Compound 21A. To a solution of compound 21 (1.0 g, 1.99 mmol) in CH₂Cl₂ (10 mL) were added compound 23 (517.4 mg, 1.19 mmol) followed by TEA (2.8 mL, 19.98 mmol) and DMAP (244 mg, 1.98 mmol) and the reaction mixture was stirred at RT for 18 h. After completion, the reaction mixture was diluted with water (20 mL) and the product was extracted with CH₂Cl₂ (2×50 mL), washed with brine (20 mL) and dried over Na₂SO₄. The crude product was purified on silica column by eluting with 50% EtOAc in pet-ether to obtain 21A (220 mg, 14%) as a gummy liquid.

To a solution of compound 7 (99.15 mg, 0.756 mmol) in CH₂Cl₂ (3 mL) were added 4-nitrophenyl chloroformate (229 mg, 1.13 mmol) followed by pyridine (0.1 mL, 1.13 mmol) and DMAP (7 mg, 0.05 mmol) and the reaction was stirred for 4 h. 21A was added (150 mg, 0.18 mmol) and the reaction mixture was allowed to stir at 45° C. for 18 h. Reaction mixture was diluted with water (10 mL); the product was extracted with CH₂Cl₂ (2×50 mL); the organic layer was separated and evaporated to get the crude mass. The crude mass was purified by silica gel column eluting with 50% EtOAc in pet-ether to get VL406 (122 mg, 68%) as a gummy liquid. ¹H NMR (400 MHz, CDCl₃): δ ppm 5.45-5.30 (m, 4H), 4.90-4.80 (m, 1H), 4.25-4.10 (m, 10H), 2.77 (t, J=6.4 Hz, 2H), 2.60-2.40 (m, 6H), 2.35-2.25 (m, 4H), 2.10-2.00 (m, 4H), 1.90-1.80 (m, 2H), 1.70-1.40 (m, 8H), 1.40-1.20 (m, 47H), 1.01 (t, J=7.2 Hz, 6H), 0.95-0.80 (m, 9H). m/z: [M+H]⁺ Calcd for 950.7; found 950.6.

To a solution of compound 7 (1.7 g, 13.2 mmol) in CH₂Cl₂ (20 mL) were added 4-nitrophenyl chloroformate (898 mg, 4.46 mmol) followed by pyridine (0.4 mL, 2.23 mmol) and DMAP (54 mg, 0.44 mmol); the reaction was stirred for 30 min. 22 was added and (2.0 g, 2.2 mmol); the reaction mixture was allowed to stir at RT for 16 h. Reaction mixture was diluted with water (10 mL) and extracted with CH₂Cl₂ (2×50 mL) and then the organic layer was separated and evaporated to a crude mass. The crude mass was purified by silica gel column eluting with 50% EtOAc in pet-ether to get VL422 (770 mg, 33%) as a gummy liquid. ¹H NMR (400 MHz, CDCl₃): δ ppm 4.90-4.80 (m, 2H), 4.20-4.10 (m, 8H), 2.60-2.50 (m, 5H), 2.50-2.40 (m, 1H), 2.35-2.25 (m, 8H), 1.90-1.75 (m, 2H), 1.70-1.55 (m, 8H), 1.55-1.45 (m, 8H), 1.40-1.20 (m, 58H), 1.01 (t, J=7.2 Hz, 6H), 0.87 (t, J=7.2 Hz, 12H). Mass: [M+H]⁺ calcd. for 1052.8; found 1052.6.

### Example 5. Synthesis of VL447, VL448, VL449 and VL451

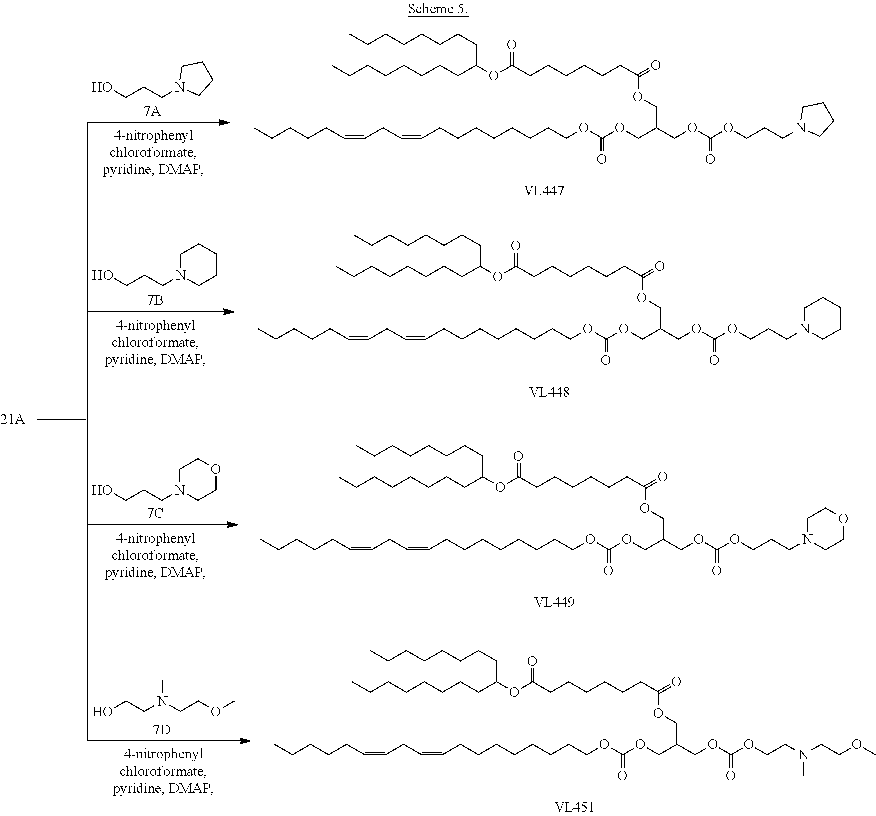

To a solution of compound 7A (195.2 mg, 1.512 mmol) in CH₂Cl₂ (6 mL) were added 4-nitrophenyl chloroformate (458 mg, 2.268 mmol) followed by pyridine (0.18 mL, 2.268 mmol) and DMAP (14 mg, 0.1134 mmol) and the reaction was stirred for 4 h. Compound 21A (300 mg, 0.378 mmol) followed by TEA (0.51 mg, 3.78 mmol) and DMAP (13.91 mg, 0.1134 mmol); the reaction mixture was allowed to stir at 45° C. for 18 h. Reaction mixture was diluted with water (10 mL) and the product was extracted with CH₂Cl₂ (2×50 mL); the organic layer was separated and evaporated to a crude mass. The crude mass was purified by silica gel column eluting with 50% EtOAc in pet-ether to obtain VL447 (41 mg, 11%) as a gummy liquid. ¹H NMR (400 MHz, CDCl₃): δ ppm 5.45-5.30 (m, 4H), 4.90-4.80 (m, 1H), 4.30-4.10 (m, 10H), 2.77 (t, J=6.4 Hz, 2H), 2.60-2.40 (m, 7H), 2.40-2.25 (m, 4H), 2.10-2.00 (m, 4H), 1.95-1.85 (m, 2H), 1.80-1.70 (m, 4H), 1.70-1.40 (m, 8H), 1.40-1.20 (m, 46H), 0.95-0.85 (m, 9H). m/z: [M+H]⁺ Calcd for 948.74; found 948.78.

Compound VL448 was afforded (102 mg, 56%) as a gummy liquid using identical procedure as VL447. ¹H NMR (400 MHz, DMSO-d₆): δ ppm 5.40-5.30 (m, 4H), 4.85-4.75 (m, 1H), 4.20-4.00 (m, 10H), 2.80-2.70 (m, 2H), 2.40-2.20 (m, 11H), 2.10-1.95 (m, 4H), 1.80-1.70 (m, 2H), 1.65-1.40 (m, 14H), 1.40-1.10 (m, 46H), 0.90-0.80 (m, 9H). m/z: [M+H]⁺ Calcd for 962.76; found 962.9.

Compound VL449 was afforded (110 mg, 60%) as a gummy liquid using identical procedure as VL447. ¹H NMR (400 MHz, DMSO-d₆): δ ppm 5.40-5.30 (m, 4H), 4.85-4.75 (m, 1H), 4.20-4.00 (m, 10H), 3.60-3.50 (m, 4H), 2.80-2.70 (m, 2H), 2.40-2.20 (m, 11H), 2.10-1.95 (m, 4H), 1.80-1.70 (m, 2H), 1.65-1.40 (m, 10H), 1.40-1.10 (m, 44H), 0.90-0.80 (m, 9H). m/z: [M+H]⁺ Calcd for 964.74; found 962.9.

Compound VL451 was afforded (118 mg, 39%) as a gummy liquid using identical procedure as VL447. ¹H NMR (400 MHz, DMSO-d₆): δ ppm 5.40-5.30 (m, 4H), 4.85-4.75 (m, 1H), 4.20-4.00 (m, 10H), 2.80-2.70 (m, 2H), 2.40-2.20 (m, 11H), 2.10-1.95 (m, 4H), 1.80-1.70 (m, 2H), 1.65-1.40 (m, 14H), 1.40-1.10 (m, 46H), 0.90-0.80 (m, 9H). m/z: [M+H]⁺ Calcd for 952.74; found 952.6.

### Example 6. Synthesis of VL410, VL411, VL421 and VL435

Synthesis of VL410. To a solution of 4E (3 g, 6.97 mmol) in DCM (30 mL) were added 4-nitrophenyl chloroformate (4.17 g, 20.91 mmol), pyridine (1.68 mL, 20.91 mmol) and DMAP (255 mg, 2.09 mmol) and the reaction was stirred for 16 h. The reaction mixture was evaporated completely to get 24 (3.8 g, 92%) as a gummy liquid. ¹H NMR (400 MHz, CDCl₃): δ ppm 8.33 (d, J=7.2 Hz, 2H), 7.50 (d, J=7.2 Hz, 2H), 2.75-2.65 (m, 2H), 2.18 (s, 3H), 2.14 (s, 6H), 1.90-1.80 (m, 2H), 1.50-1.00 (m, 21H), 1.00-0.80 (m, 15H).

To a stirred solution of 5 (500 mg, 1.28 mmol) in CH₂Cl₂ (10 mL) were added 24 (536 mg, 0.90 mmol) followed by pyridine (0.13 mL, 1.67 mmol) and DMAP (48 mg, 0.3860 mmol) and the reaction was stirred at RT for 16 h. The reaction mixture was diluted with water (20 mL) and extracted with DCM (2×50 mL), washed with brine solution (20 mL) and dried on Na₂SO₄. The crude was purified on silica column by eluting with 25% EtOAc in pet ether to get 25 (320 mg, 97%) as a gummy liquid. ¹H NMR (400 MHz, DMSO-d₆): δ ppm 4.79 (d, J=5.2 Hz, 1H), 4.40-4.20 (m, 2H), 4.20-3.90 (m, 4H), 3.55-3.45 (m, 2H), 2.60-2.50 (m, 2H), 2.40-2.20 (m, 5H), 2.10-1.90 (m, 8H), 1.90-1.70 (m, 2H), 1.70-1.00 (m, 45H), 1.00-0.89 (t, J=7.2 Hz, 12H).

To a 7 (248 mg, 1.89 mmol) in CH₂Cl₂ (3 mL) were added 4-nitrophenyl chloroformate (814 mg, 2.83 mmol) followed by pyridine (0.2 mL, 2.83 mmol) and DMAP (17 mg, 0.05 mmol) and the reaction was stirred for 4 h. 25 was added (400 mg, 0.4732 mmol) and the reaction mixture was allowed to stir at 45° C. for 18 h. Reaction mixture was diluted with water (10 mL) and extracted with CH₂Cl₂ (2×50 mL) and then the organic layer was separated and evaporated to get the crude. Crude material was purified by silica gel column eluting with 50% EtOAc in pet-ether to yield VL-410 (122 mg, 26%) as a gummy liquid. ¹H NMR (400 MHz, CDCl₃): δ ppm 4.33 (d, J=6.0 Hz, 2H), 4.26 (d, J=6.0 Hz, 2H), 4.25-4.15 (m, 4H), 4.05 (t, J=5.6 Hz, 2H), 2.65-2.45 (m, 8H), 2.40-2.25 (m, 4H), 2.08 (s, 3H), 2.05 (s, 3H), 2.02 (s, 3H), 1.80-1.40 (m, 10H), 1.50-1.20 (m, 32H), 1.20-1.00 (m, 12H), 0.90-0.80 (m, 18H). HRMS m/z: [M+H]⁺ Calcd for 1002.75; found 1002.83.

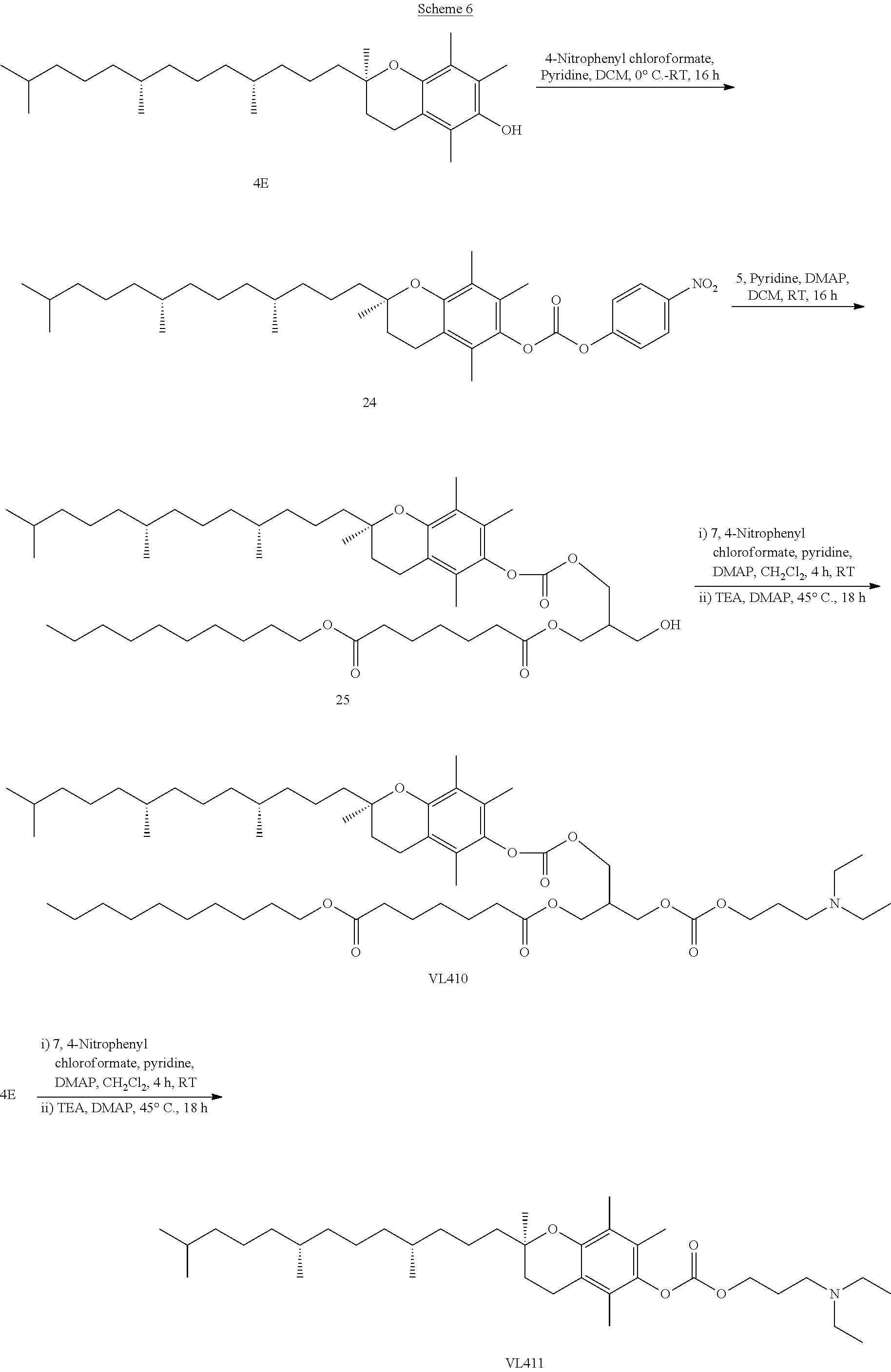

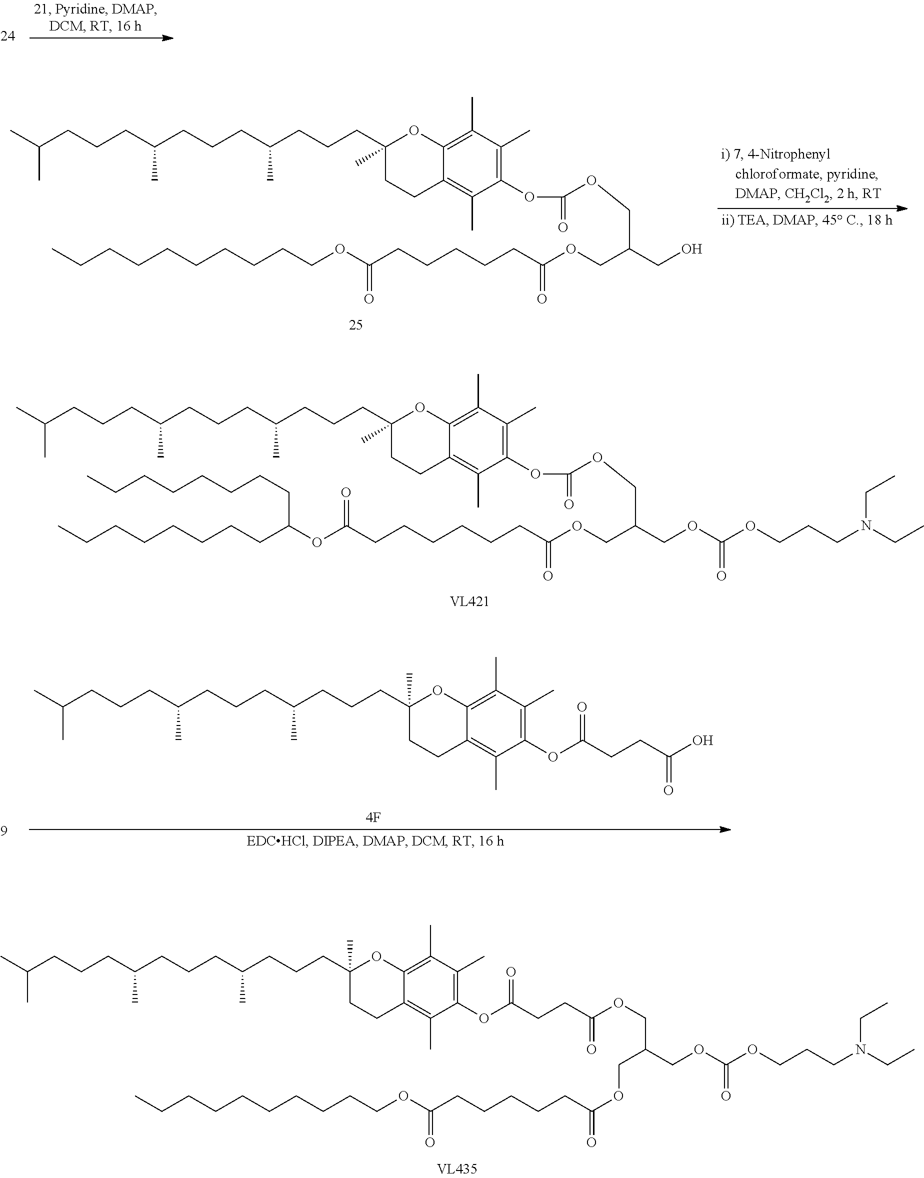

Synthesis of VL411. To a solution of compound 4E (1.3 g, 3.02 mmol) in CH₂Cl₂ (15 mL) were added 4-nitrophenyl chloroformate (1.21 g, 6.04 mmol) followed by pyridine (0.5 mL, 6.04 mmol) and DMAP (110 mg, 0.9 mmol) and the reaction was continued stirring for 1 h. After 1 h added compound 7 (2.37 g, 18.1 mmol) and the reaction mixture was allowed to stir at RT for 17 h. Reaction mixture was diluted with water (50 mL); the product was extracted with CH₂Cl₂ (2×50 mL) and the organic layer was separated and evaporated to get a crude mass. The crude mass was purified by silica gel column eluting with 50% EtOAc and pet-ether to obtain VL411 (420 mg, 24%) as a gummy liquid. ¹H NMR (400 MHz, CDCl₃): δ ppm 4.30 (t, J=6.4 Hz, 2H), 2.70-2.50 (m, 8H), 2.08 (s, 3H), 2.07 (s, 3H), 2.03 (s, 3H), 1.95-1.70 (m, 4H), 1.62-0.95 (m, 30H), 0.90-0.80 (m, 12H).

Synthesis of VL421. To a stirred solution of compound 21 (1.0 g, 1.83 mmol) in CH₂Cl₂ (10 mL) were added compound 24 (1.09 g, 1.83 mmol) followed by pyridine (0.37 mL, 3.54 mmol) and DMAP (67 mg, 0.55 mmol) and the reaction was stirred at RT for 1 h. Reaction mixture was diluted with water (20 mL) and extracted with DCM (2×50 mL), washed with brine solution (20 mL) and dried on Na₂SO₄. The crude was purified on silica column by eluting with 30% EtOAc in pet-ether to get 25 (480 mg, 30%) as a gummy liquid.

To a solution of compound 7 (219 mg, 0.1.6u7 mmol) in CH₂Cl₂ (4 mL) were added 4-nitrophenyl chloroformate (506 mg, 2.50 mmol) followed by pyridine (0.26 mL, 2.50 mmol) and DMAP (16 mg, 0.12 mmol) and the reaction was stirred for 4 h. After 4 h added compound 25 (400 mg, 0.41 mmol) and the reaction mixture was allowed to stir at 45° C. for 16 h. Reaction mixture was diluted with water (10 mL); product was extracted with CH₂Cl₂ (2×50 mL) and then the organic layer was separated and evaporated to get a crude mass. The crude mass was purified by silica gel column eluting with 55% EtOAc in pet-ether to yield VL-421 (110 mg, 24%) as a gummy liquid. ¹H NMR (400 MHz, CDCl₃): δ ppm 4.40-4.20 (m, 8H), 2.65-2.40 (m, 9H), 2.40-2.25 (m, 4H), 2.08 (s, 3H), 2.06 (s, 3H), 2.02 (s, 3H), 1.40-1.20 (m, 4H), 1.70-1.50 (m, 10H), 1.50-1.00 (m, 56H), 0.90-0.80 (m, 18H). Mass m/z: [M+H]⁺ Calcd for 1114.88; found 1114.93.

Synthesis of VL435. To a solution of compound 9 (220 mg, 0.403 mmol) in CH₂Cl₂ (2 mL) was added compound 4F (214 mg, 0.403 mmol) followed by EDC·HCl (115 mg, 0.604 mmol), DMAP (14 mg, 0.12 mmol) and DIPEA (0.2 mL, 1.21 mmol). Reaction mixture was stirred at RT for 16 h. After complete disappearance of compound 9 by TLC, the reaction mixture was diluted with water and the product was extracted into CH₂Cl₂ (2×20 mL). The organic layer was separated and evaporated to get a crude mass. The crude mass was purified by silica gel (60-120 mesh) column chromatography by eluting with 50% EtOAc in pet-ether followed by prep HPLC purification afforded VL435 as a formate salt. Obtained product was washed with 10% NaHCO₃ solution and then lyophilized to yield pure VL435 (60 mg, 14%). ¹H NMR (400 MHz, DMSO-d₆): δ ppm 4.20-4.12 (m, 8H), 4.00-3.90 (m, 2H), 2.95-2.85 (m, 2H), 2.70-2.60 (m, 2H), 2.45-2.20 (m, 8H), 2.00 (s, 3H), 1.90 (s, 3H), 1.88 (s, 3H), 1.80-1.60 (m, 5H), 1.60-1.00 (m, 50H), 1.00-0.80 (m, 21H). Mass m/z: [M+H]⁺ Calcd for 1058.78; found 1058.6.

### Example 7. PEG-Lipid Synthesis

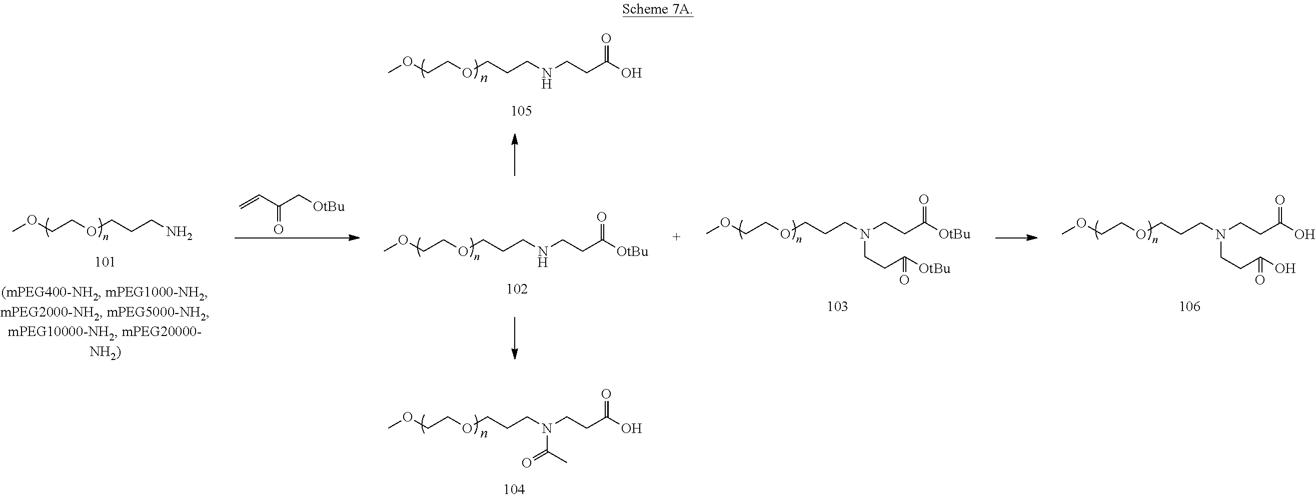

Compound 102 is prepared from desired mPEG-amine 101 by Michael addition of the PEG-amine to tert-butyl acrylate. The mono adduct 102 and diadduct 103 are separated by column chromatography. Acid treatment of 102 affords the acid 105. Acetylation of 102 with acetic anhydride followed by acid treatment affords the carboxylic acid 104. Treatment of 103 with acid affords the dicarboxylic acid 106.

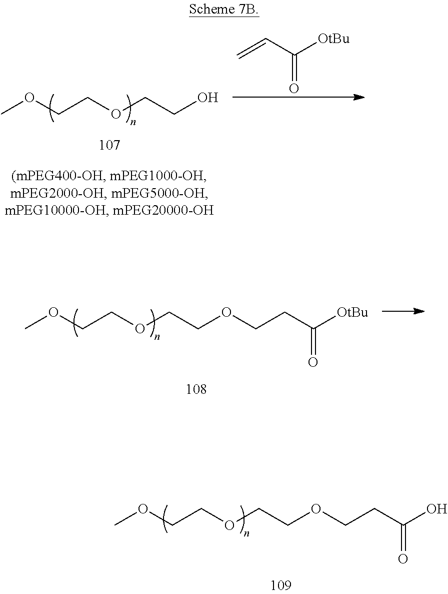

Compound 108 is prepared from desired mPEG-OH 107 by Michael addition of the mPEG-OH to tert-butyl acrylate. Treatment of compound 108 with acid affords the carboxylic acid 109.

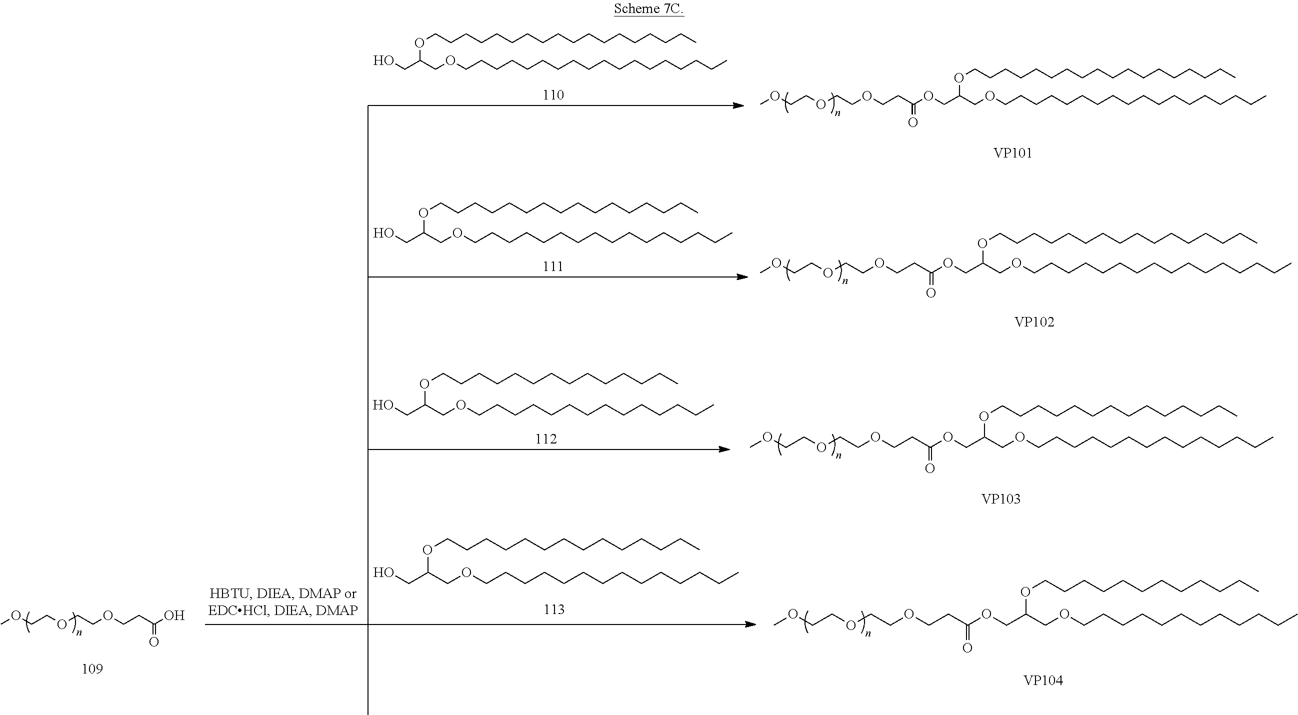

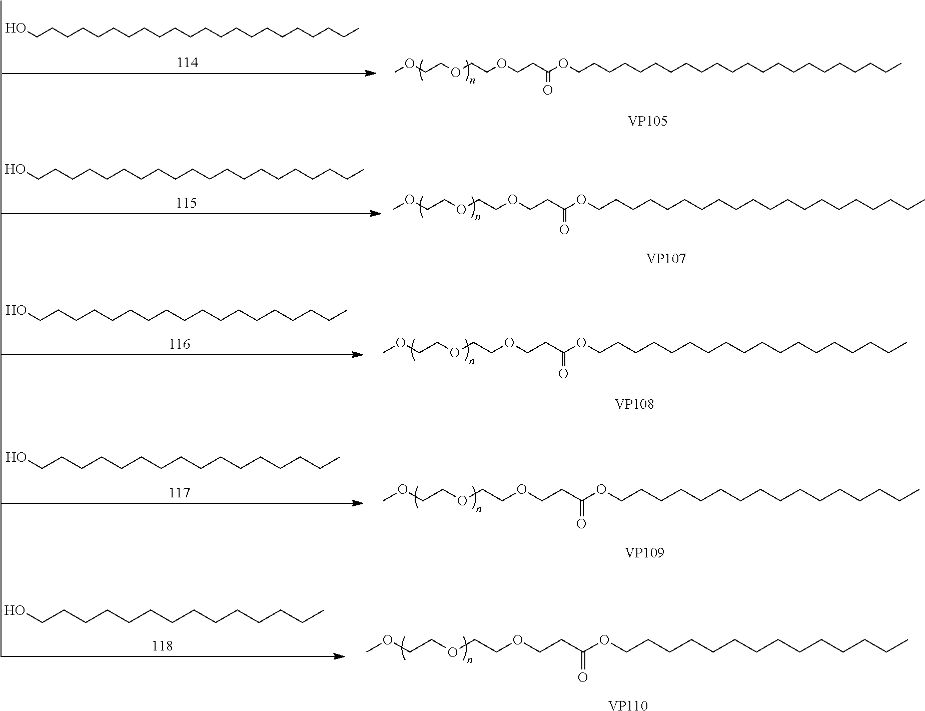

Activation of the carboxylic acid 109 using peptide coupling agent HBTU in the presence of DIEAN and DAMP followed by addition of the alcohol 110 affords the PEG-lipid VP101. Similarly, addition of the alcohols 111-118 to the activated carboxylic acid afford PEG-lipids VP101-VP110.

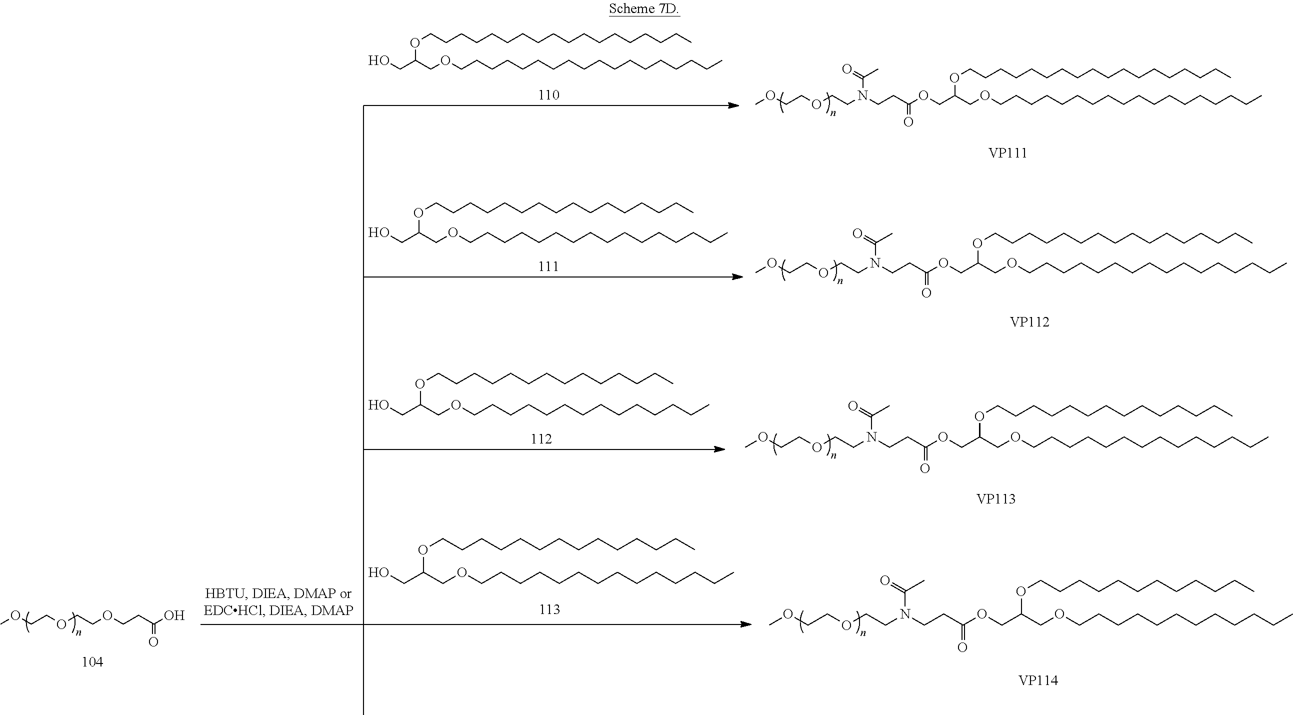

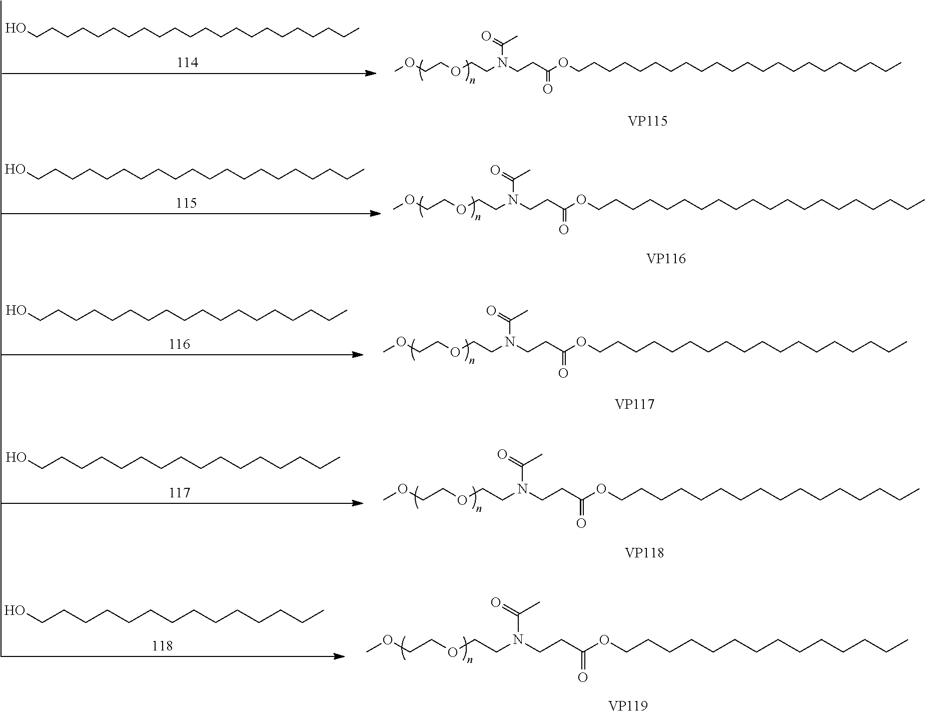

Activation of the carboxylic acid 104 using peptide coupling agent HBTU in the presence of DIEAN and DAMP followed by addition of the alcohol 110 affords the PEG-lipid VP111. Similarly, addition of the alcohols 111-118 to the activated carboxylic acid afford PEG-lipids VP112-VP119.

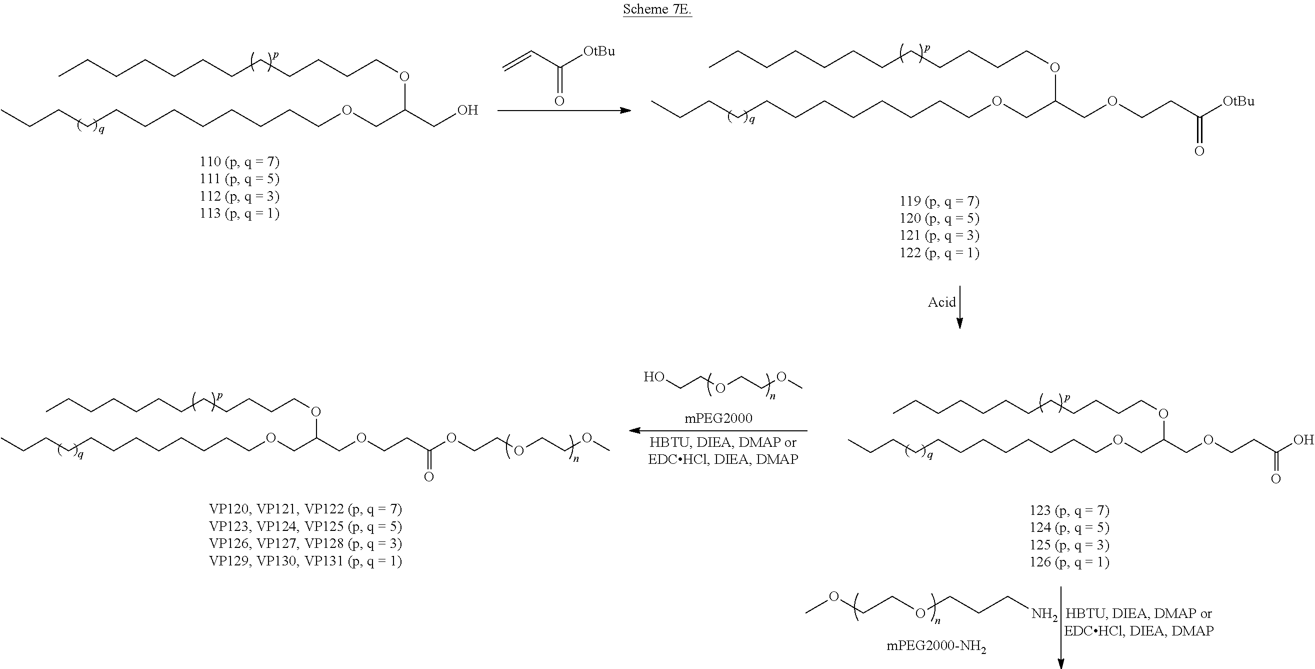

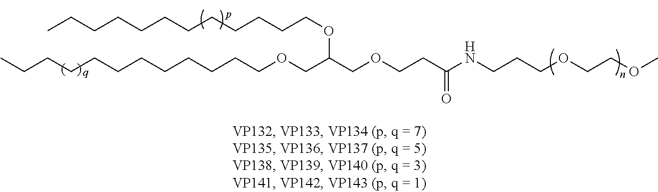

Michael addition of the alcohol 110 to tert-butyl acrylate affords the ester 119. Treatment of the ester 119 with acid affords 123. Activation of the carboxylic acid 123 with peptide coupling agent followed by addition of mPEG2000 affords VP120, the racemic compounds. Similarly, starting with chirally pure 110 affords the enantiomers VP121 and VP122. The PEG-lipids VP123-VP131 are similarly prepared starting from corresponding racemic or chirally pure alcohols 111-113.

Activation of the carboxylic acid 123 with peptide coupling agent followed by addition of mPEG2000-NH₂ affords VP132, the racemic compounds. Similarly, starting with chirally pure 110 affords the enantiomers VP133 and VP134. The PEG-lipids VP135-VP143 are similarly prepared starting from corresponding racemic or chirally pure alcohols 111-113.

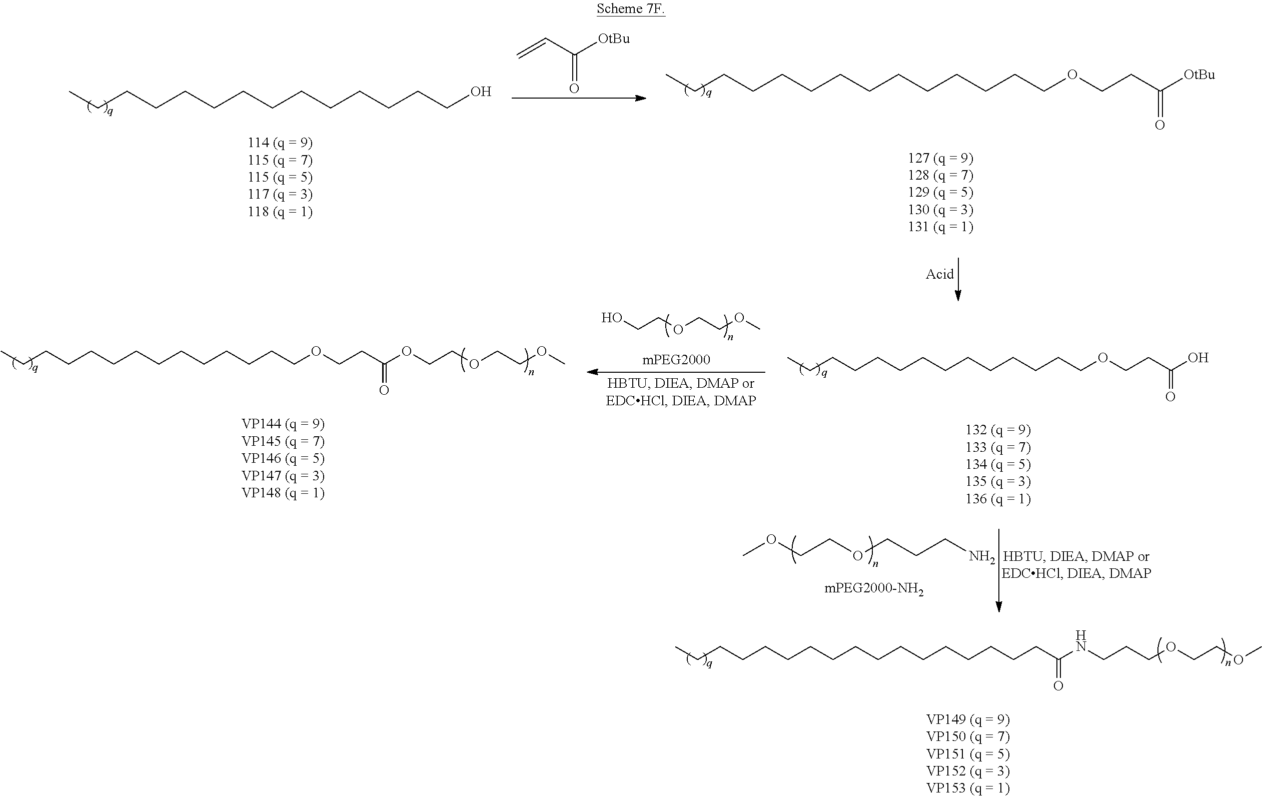

Michael addition of the alcohol 114 to tert-butyl acrylate affords the ester 127. Treatment of the ester 127 with acid affords the carboxylic acid 132. Activation of the carboxylic acid 132 with peptide coupling agent followed by addition of mPEG2000 affords VP144. The PEG-lipids VP145-VP148 are similarly prepared starting from corresponding alcohols 115-118.

Activation of the carboxylic acid 132 with peptide coupling agent followed by addition of mPEG2000-NH₂ affords VP149. The PEG-lipids VP150-VP153 are similarly prepared starting from corresponding racemic or chirally pure alcohols 115-118.

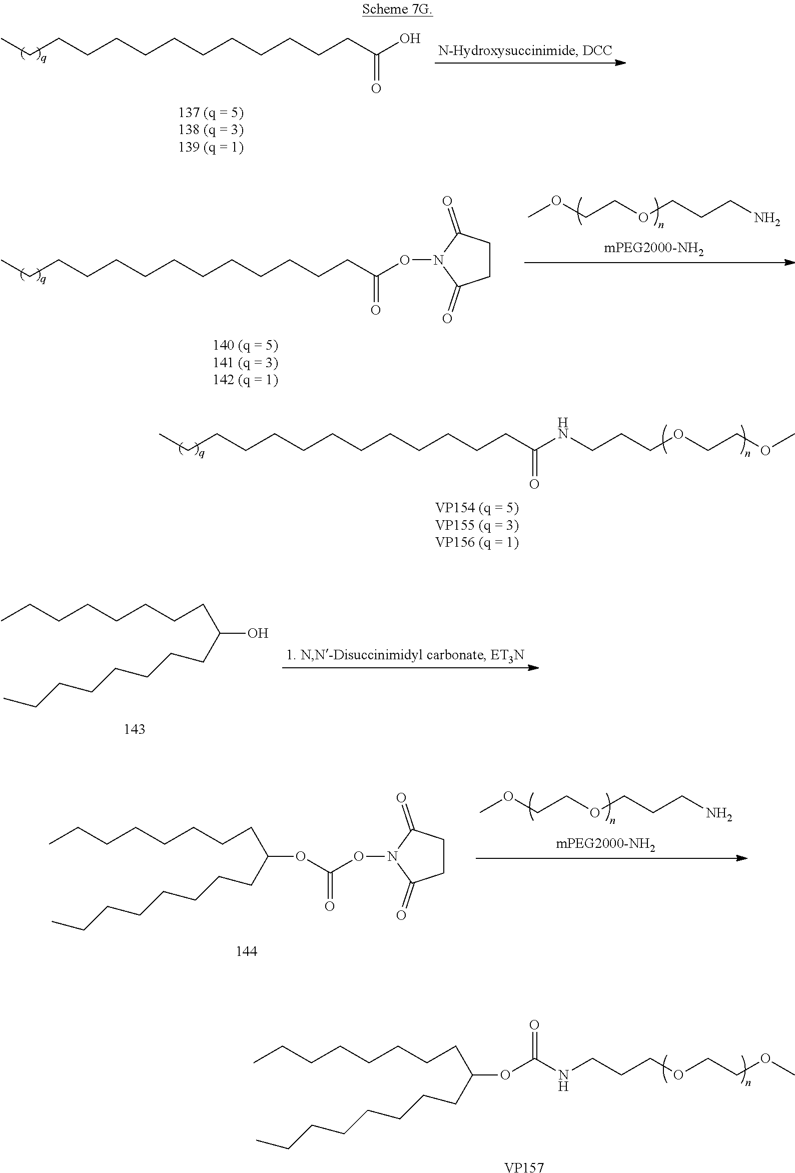

To a stirred solution of the acid 137 (1 g, 3.52 mmol) in THE (10 mL) were added N-hydroxysuccinimide (567 mg, 4.92 mmol) followed by DCC (870 mg, 4.22 mmol) at 0° C. and the reaction mixture was stirred at room temperature for overnight. After completion, solid is filtered and the filtrate was evaporated under reduced pressure to get the crude material. This was recrystallized in isopropyl alcohol, filtered the solid and dried to obtain compound 140 (1 g, 74% yield) as an off white solid. ¹H NMR (400 MHz, CDCl₃): δ 2.83 (s, 4H), 2.61 (t, J=7.2 Hz, 2H), 1.76-1.72 (m, 2H), 1.40-1.38 (m, 2H), 1.29-1.25 (m, 26H), 0.89 (t, J=6.4 Hz, 3H).

To a solution of compound 140 (113 mg, 0.296 mmol) in CH₂Cl₂ (5 mL) were added mPEG2000-NH₂ (500 mg, 0.24 mmol) followed by DIPEA (0.1 mL, 0.73 mmol) at 0° C. and the reaction mixture was stirred at room temperature overnight. The reaction mixture after diluting with dichloromethane (10 mL) was stirred with ice cold water for 15 min. The organic layer was separated, washed with ice cold water, dried over anhydrous Na₂SO₄ and concentrated under reduced pressure to get the crude product. Silica gel column chromatographic purification (10% MeOH in DCM as an eluent) afforded compound VP154 (160 mg) as an off white solid. ¹H NMR (400 MHz, DMSO-d₆): δ 7.70 (t, 1H), 3.68-3.31 (m, 195H), 3.23 (s, 3H), 3.06 (t, J=6.0 Hz, 2H), 2.03 (t, J=7.6 Hz, 2H), 1.61-1.44 (m, 4H), 1.27-1.23 (m, 28H), 0.87 (t, J=6.8 Hz, 3H). HRMS m/z: [M+H]⁺ calculated: 2293.3; found: 2294.26.

To a stirred solution of compound 138 (1 g, 3.90 mmol) in THE (10 mL) were added N-hydroxysuccinimide (628 mg, 5.4 mmol) followed by DCC (964 mg, 4.68 mmol) at 0° C. and the reaction mixture was stirred at RT for overnight. Reaction mixture was filtered and the filtrate was evaporated under reduced pressure to get the crude material. This was recrystallized in isopropyl alcohol, filtered the solid and dried to get 141 (1 g, 72% yield) as a white solid. ¹H NMR (400 MHz, CDCl₃): δ 2.83 (s, 4H), 2.61 (t, J=7.6 Hz, 2H), 1.76-1.70 (m, 2H), 1.42-1.25 (m, 24H), 0.89 (t, J=6.8 Hz, 3H).

To a solution of compound 141 (113 mg, 0.29 mmol) in CH₂Cl₂ (5 mL) were added mPEG2000-NH₂ (500 mg, 0.24 mmol) followed by DIPEA (0.1 mL, 0.73 mmol) at 0° C. and the reaction mixture was stirred at room temperature overnight. The progress of the reaction was monitored by TLC. After completion of the reaction, the mixture was diluted with CH₂Cl₂ (10 mL) and stirred with ice cold water for 15 min. The organic layer was separated, washed with ice cold water, dried over anhydrous Na₂SO₄ and concentrated under reduced pressure to get the crude product. Silica gel column chromatographic purification using 10% MeOH in CH₂Cl₂ as an eluent afforded VP155 as an off white solid (0.44 g, 78.5%). ¹H NMR (400 MHz, DMSO-d₆): δ 7.70 (t, 1H), 3.50-3.36 (m, 185H), 3.23 (s, 3H), 3.06 (t, J=6.8 Hz, 1H), 2.03-1.99 (m, 2H), 1.61-1.46 (m, 4H), 1.27-1.23 (m, 24H), 0.87 (t, J=6.8 Hz, 3H). HRMS m/z: [M+H]⁺ calculated: 2266.8; found: 2266.89.

To a stirred solution of compound 139 (1.6 g, 7.0 mmol) in THE (16 mL) were added N-hydroxysuccinimide (1.12 g, 9.80 mmol) followed by DCC (1.7 g, 8.40 mmol) at 0° C. and the reaction mixture was stirred at RT for overnight. Reaction mixture filtered and the filtrate was evaporated under reduced pressure to get the crude material. The crude was taken in isopropyl alcohol (10 mL) and heated at 80° C. for 45 min and then cooled to 0° C. and the precipitated solid was filtered and dried to get compound 142 (1.6 g, 74% yield) as a white solid. ¹H NMR (400 MHz, CDCl₃): δ 2.83 (s, 4H), 2.61 (t, J=7.2 Hz, 2H), 1.76-1.72 (m, 2H), 1.40-1.25 (m, 20H), 0.89 (t, J=6.4 Hz, 3H).

To a solution of compound 142 (96 mg, 0.296 mmol) in dichloromethane (5 mL) were added mPEG2000-NH₂ (500 mg, 0.24 mmol) followed by DIPEA (0.1 mL, 0.73 mmol) at 0° C. and the reaction mixture was stirred at room temperature overnight. The reaction mixture was diluted with dichloromethane (10 mL) and stirred with ice cold water for 15 min. The organic layer was separated, washed with ice cold water, dried over anhydrous Na₂SO₄ and concentrated under reduced pressure to get the crude product. Silica gel column chromatographic purification using 10% MeOH in DCM as an eluent afforded compound VP156 as an off white solid (0.18 g, 21%). ¹H NMR (400 MHz, DMSO-d₆): δ 7.70 (t, 1H), 3.68-3.34 (m, 189H), 3.23 (s, 3H), 3.06 (q, 2H), 2.03 (t, J=7.2 Hz, 2H), 1.61-1.59 (m, 2H), 1.47-1.46 (m, 2H), 1.46-1.23 (m, 20H), 0.87 (t, J=6.4 Hz, 3H). HRMS m/z: [M+H]⁺ ca calculated: 2238.9; found: 2238.89.

To a stirred solution of compound 143 (1.0 g, 3.89 mmol) in CH₂Cl₂ (10 mL) at 0° C. were added N,N′-disuccinimidylcarbonate (1.5 g, 5.84 mmol) followed by triethylamine (1.6 mL, 11.69 mmol) and the reaction mixture was stirred at RT for overnight. To the above reaction mixture, N,N′-disuccinimidylcarbonate (1.5 g, 5.84 mmol) followed by triethylamine (1.6 mL, 11.69 mmol) were added additionally at RT and heated to 40° C. for 3 days. The reaction mixture was diluted with ice-cold water (20 mL) and separated the organic layer. The organic layer was washed with brine (10 mL), dried over anhydrous Na₂SO₄ and concentrated under reduced pressure. The crude product was purified by flash column chromatography (100-200 mesh Silica gel; eluted with 20% diethyl ether in pet-ether) to afford compound 144 (0.75 g, 1.88 mmol, 48% yield) as a pale-yellow gum. ¹H NMR (400 MHz, CDCl₃): δ 4.82-4.78 (m, 1H), 2.82 (s, 4H), 1.71-1.57 (m, 4H), 1.43-1.22 (m, 24H), 0.91-0.87 (m, 6H).

To a stirred solution of compound mPEG200-NH₂ (950 mg, 0.46 mmol) in 10 mL of CH₂Cl₂ at 0° C. were added pyridine (0.1 mL, 1.40 mmol) followed by compound 144 (279 mg, 0.70 mmol) and stirred at RT for 16 h. The reaction was concentrated completely under reduced pressure mixture. The residue was diluted with CH₂Cl₂ (10 mL) and stirred with ice cold water for 20 min. The organic layer was separated, washed with ice cold water (10 mL), dried over anhydrous Na₂SO₄ and concentrated under reduced pressure to get the crude. This was purified by silica gel column using 10% MeOH in CH₂Cl₂ as an eluent to yield VP157 as an off white solid (0.76 g, 76%). ¹H NMR (400 MHz, DMSO-d₆): δ 6.92 (br t, J=5.6 Hz, 1H), 4.61-4.57 (m, 1H), 3.69-3.67 (m, 1H), 3.59-3.49 (m, 188H), 3.48-3.41 (m, 3H), 3.38-3.333 (m, 2H), 3.24 (s, 3H), 3.02-2.97 (m, 2H), 1.62-1.58 (m, 2H), 1.48-1.39 (m, 4H), 1.31-1.30 (m, 24H), 0.86-0.83 (m, 6H). HRMS m/z: [M+H]⁺ calculated: 2222.1; found: 2222.4.

### Example 8. Guide RNA (gRNA) and mRNA for LNP Evaluation

The guide RNAs (gRNA) shown in Table 3 were synthesized under solid phase oligonucleotide synthesis and deprotection conditions using controlled pore glass support and commercially available phosphoramidite monomers and oligonucleotide synthesis reagents (Methods in Molecular Biology, 1993, 20, 81-114; ACS Chem. Biol. 2015, 10, 1181-1187, incorporated herein by reference in its entirety). The deprotected guide RNAs were purified by HPLC and the integrity of each guide RNA was confirmed by mass spectrometric analysis. The observed mass of each guide RNA was conformed to calculated mass.

TABLE 3
Single guide RNA (gRNA) used in the studies described in Examples 9-23

| | | Protospacer | |
|---|---|---|---|
| Target* | gRNA | (5′-3′) | gRNA sequence (5′-3′)# |
| PCSK9 | GA055 | CAGGTTC | csasgsGUUCCAUGGGAUGCUCUgUUUUAGa |
| | | CATGGG | gcuagaaauagcaaGUUaAaAuAaggcuaGUccGUU |
| | | ATGCTCT | AucAAcuugaaaaagugGcaccgagucggugcusususu |
| PCSK9 | GA010 | GGCTGA | gsgscsUGAUGAGGCCGCACAUGGUUUUAG |
| | | TGAGGC | AgcuagaaauagcAAGUUAAAAUAAGGCUAG |
| | | CGCACA | UCCGUUAUCAacuugaaaaaguggcaccgagucggug |
| | | TG | cusususu |
| PCSK9 | GA255 | CCCATAC | cscscsAUACCUUGGAGCAACGGgUUUUAGa |
| | | CTTGGA | gcuaGaaauagcaaGUUaAaAuAaggCUaGUCcGU |
| | | GCAACG | UAucAAcuuGaaaaaguGgcaccgAgUCggugcusus |
| | | G | usu |
| PCSK9 | GA256 | CCCATAC | cscscsAUACCUUGGAGCAACGGgUUUUAGa |
| | | CTTGGA | gcuagaaauagcaaGUUaAaAuAaggcuaGUccGUU |
| | | GCAACG | AucAAcuugaaaaagugGcaccgagucggugcusususu |
| | | G | |
| PCSK9 | GA257 | CCCATAC | cscscsAUACCUUGGAGCAACGGgUUUUAGa |
| | | CTTGGA | gcuaGaaauagcaaGUUaAaAuAaggcuaGUccGUU |
| | | GCAACG | AucAAcuuGaaaaagugGcaccgagucggugcusususu |
| | | G | |
| PCSK9 | GA292 | CCCATAC | cscscsAUACUUGGAGCAACGGGUUUUAGA |
| | | CTTGGA | GCUAGAAAUAGCAAGUUAAAAUAAGGC |
| | | GCAACG | UAGUCCGUUAUCAACUUGAAAAAGUGGC |
| | | G | ACCGAGUCGGUGCUsususu |
| PCSK9 | GA097 | CCCGCA | cscscsGCACCUUGGCGCAGCGGgUUUUAGa |
| | | CCTTGGC | gcuagaaauagcaaGUUaAaAuAaggcuaGUccGUU |
| | | GCAGCG | AucAAcuugaaaaagugGcaccgagucggugcusususu |
| | | G | |
| ANGPTL3 | GA258 | GAGATA | gsasgsAUACCUGAGUAACUUUCgUUUUAGa |
| | | CCTGAGT | gcuaGaaauagcaaGUUaAaAuAaggCUaGUCcGU |
| | | AACTTTC | UAucAAcuuGaaaaaguGgcaccgAgUCggugcusus |
| | | | usu |
| ANGPTL3 | GA259 | GAGATA | gsasgsAUACCUGAGUAACUUUCgUUUUAGa |
| | | CCTGAGT | gcuagaaauagcaaGUUaAaAuAaggcuaGUccGUU |
| | | AACTTTC | AucAAcuugaaaaagugGcaccgagucggugcusususu |
| ANGPTL3 | GA260 | GAGATA | gsasgsAUACCUGAGUAACUUUCgUUUUAGa |
| | | CCTGAGT | gcuaGaaauagcaaGUUaAaAuAaggcuaGUccGUU |
| | | AACTTTC | AucAAcuuGaaaaagugGcaccgagucggugcusususu |
| ANGPTL3 | GA067 | AAGATA | asasgsAUACCUGAAUAACUCUCGUUUUAG |
| | | CCTGAAT | AgcuagaaauagcAAGUUAAAAUAAGGCUAG |
| | | AACTCTC | UCCGUUAUCAacuugaaaaaguggcaccgagucggug |
| | | | cusususu |
| ANGPTL3 | GA347 | AAGATA | asasgsAUACCUGAAUAACUCUCgUUUUAGa |
| | | CCTGAAT | guaGaaauagcaaGUUaAaAuAaggcuaGUccGUU |
| | | AACTCTC | AucAAcuuGaaaaagugGcaccgagucggugcusususu |

*The gRNAs were designed to target mouse, rat, monkey and human PCSK9 and ANGPTL3 genes.
#uppercase and lowercase letters in the guide RNA sequence indicate nucleotides carrying 2′-ribo (2′-OH) and 2′-O-methyl (2′-OMe) ribosugar moiety, respectively, and the letter ‘s’ indicates phosphorothioate (PS) linkage.

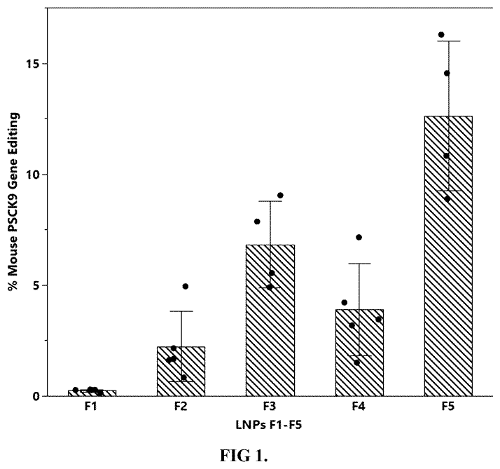

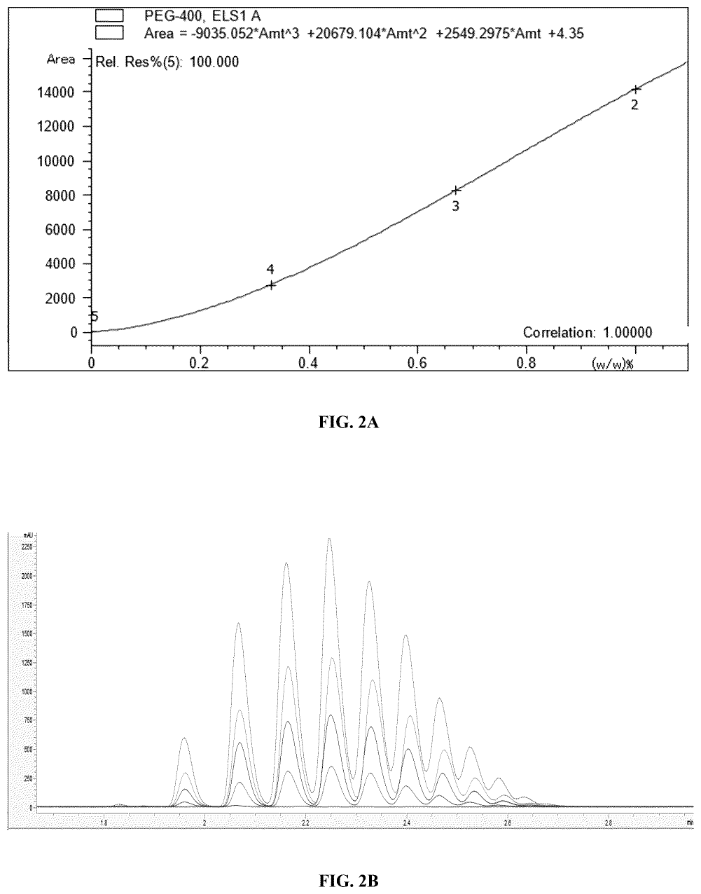

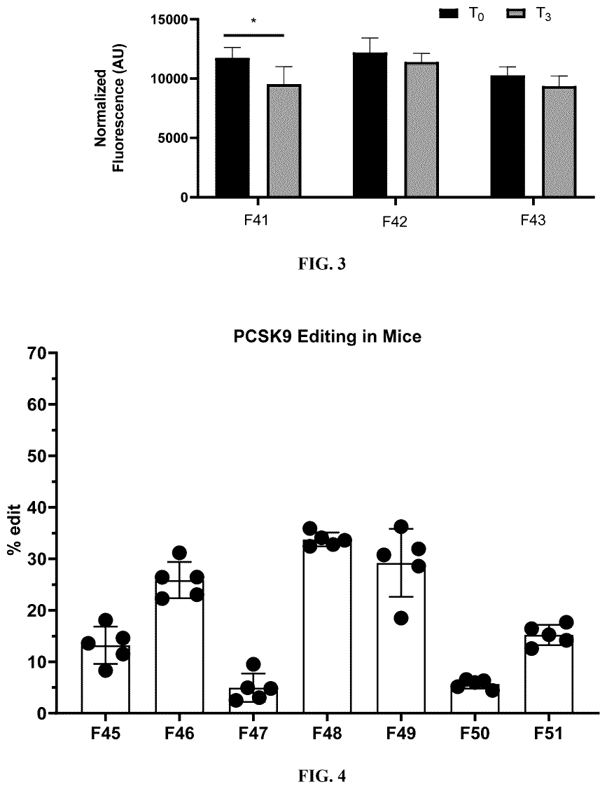

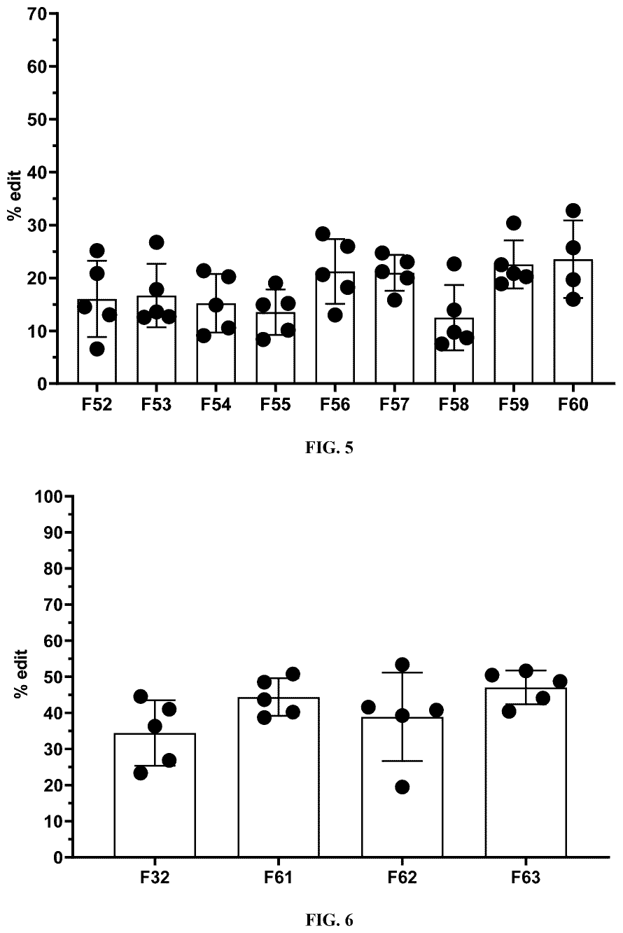

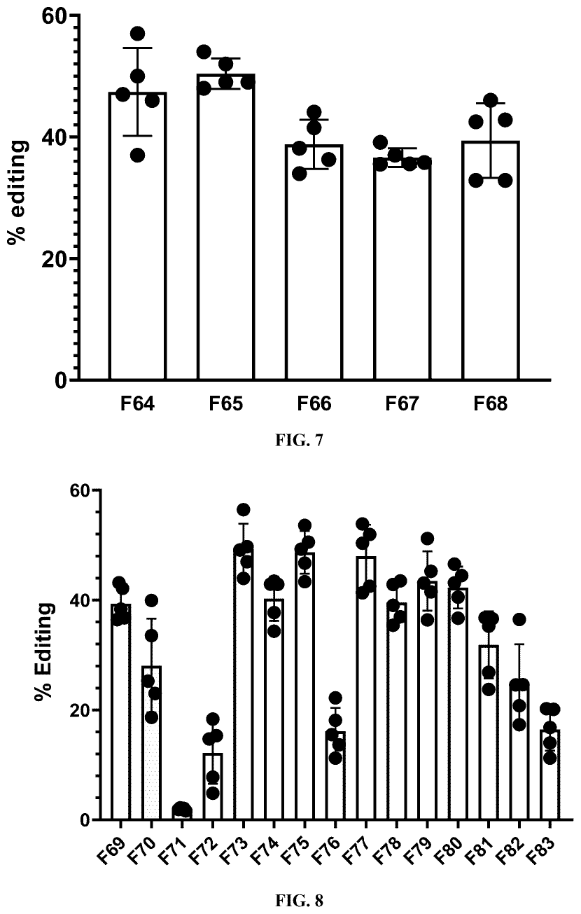

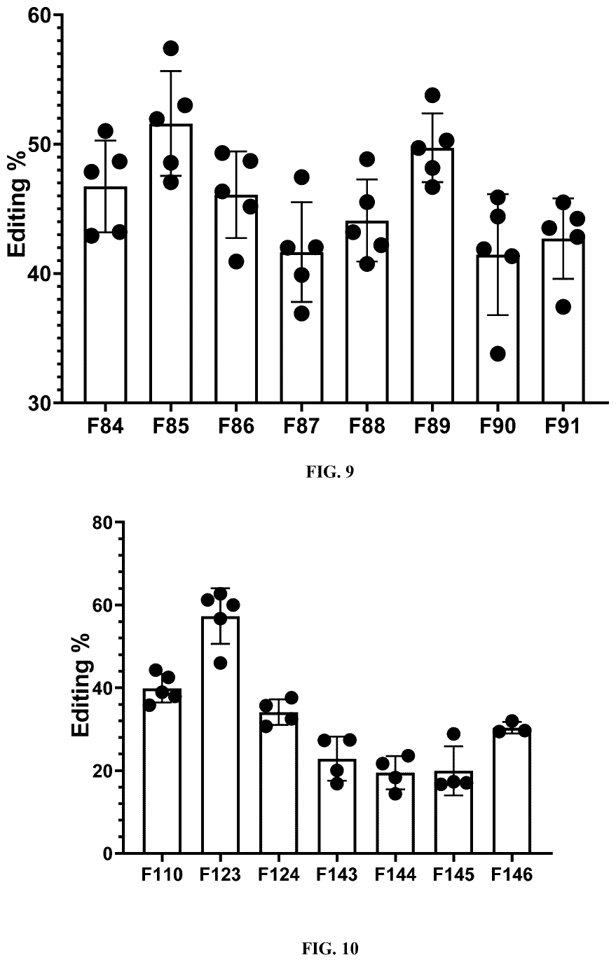

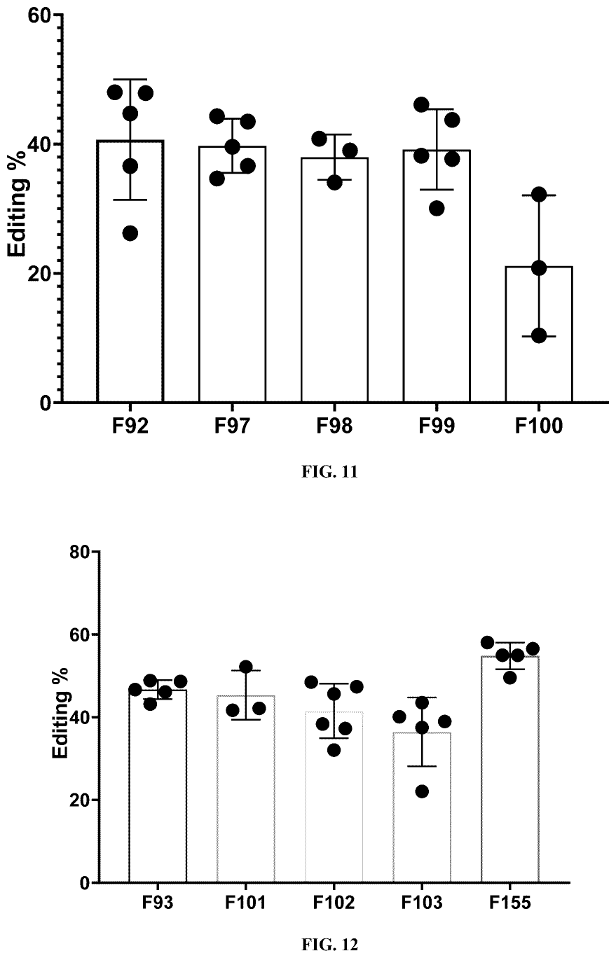

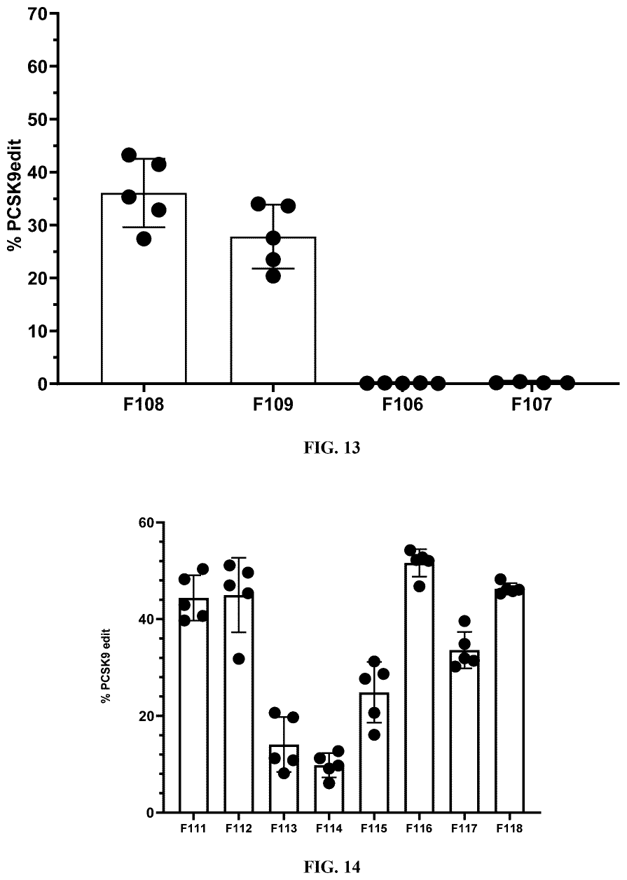

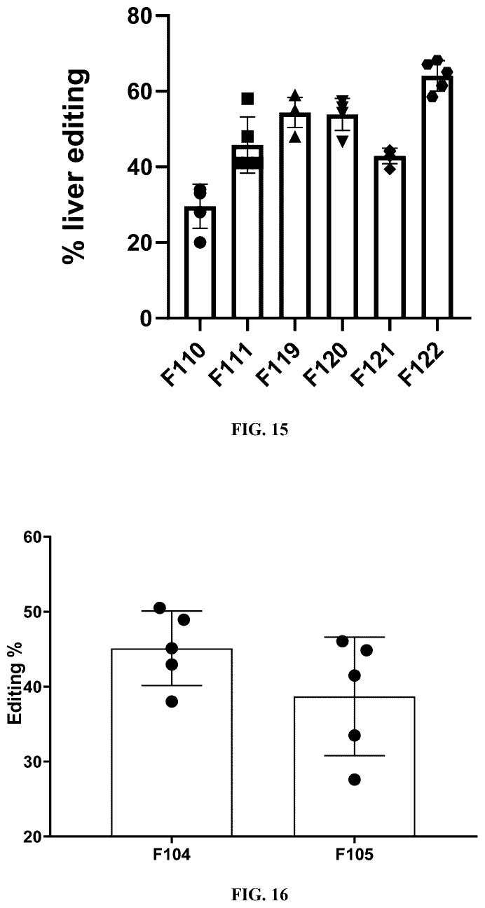

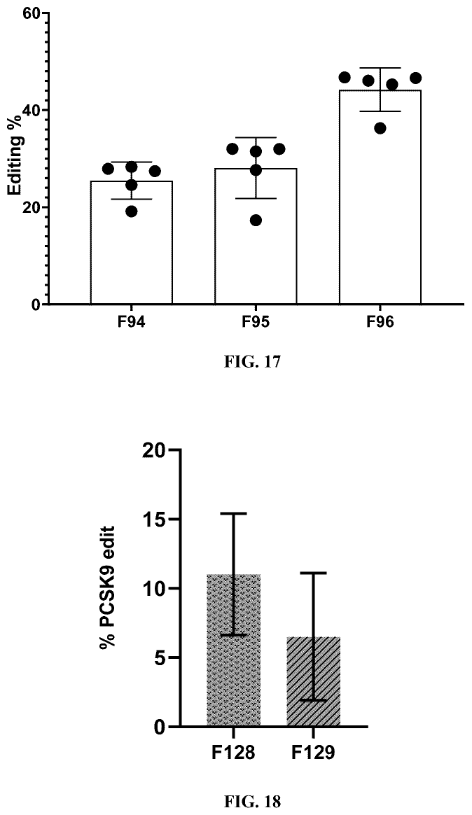

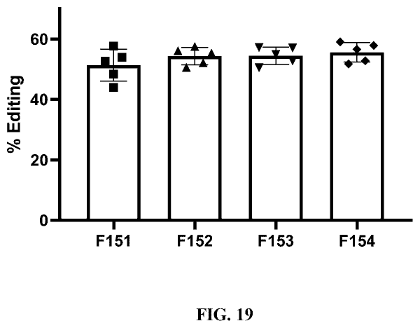

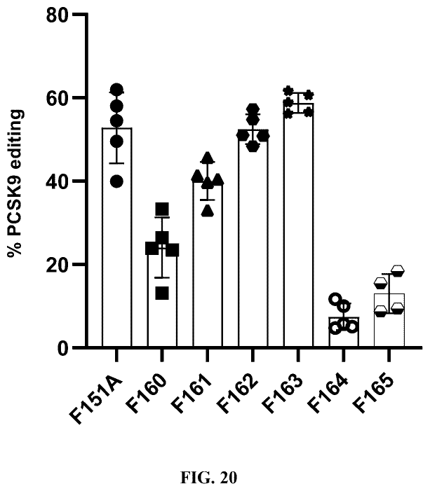

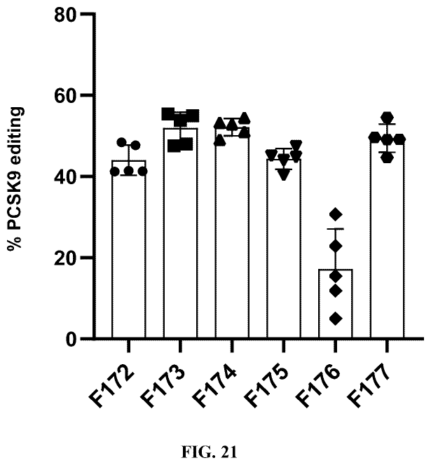

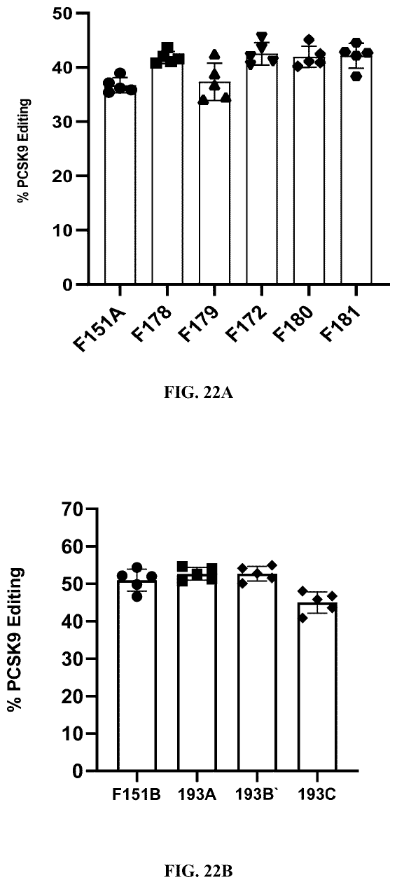

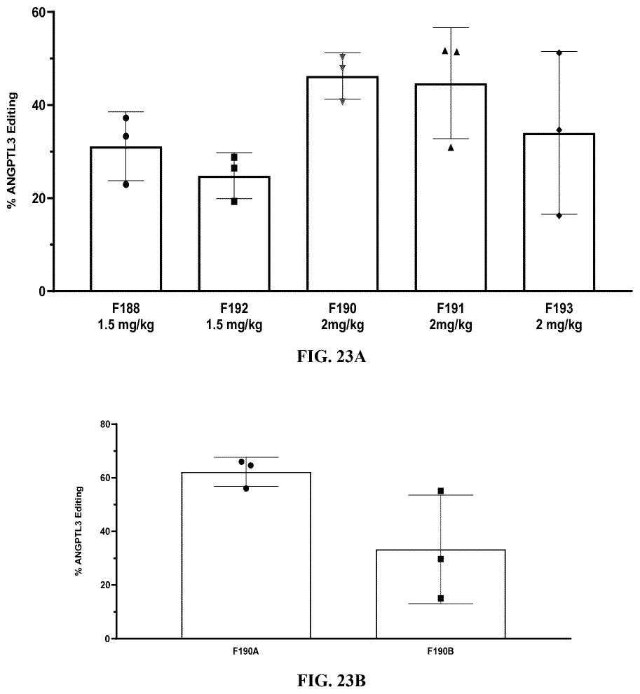

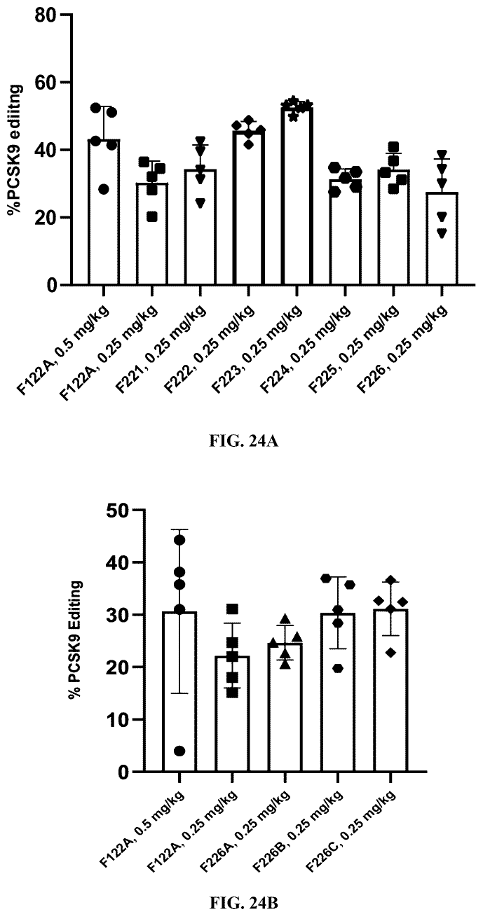

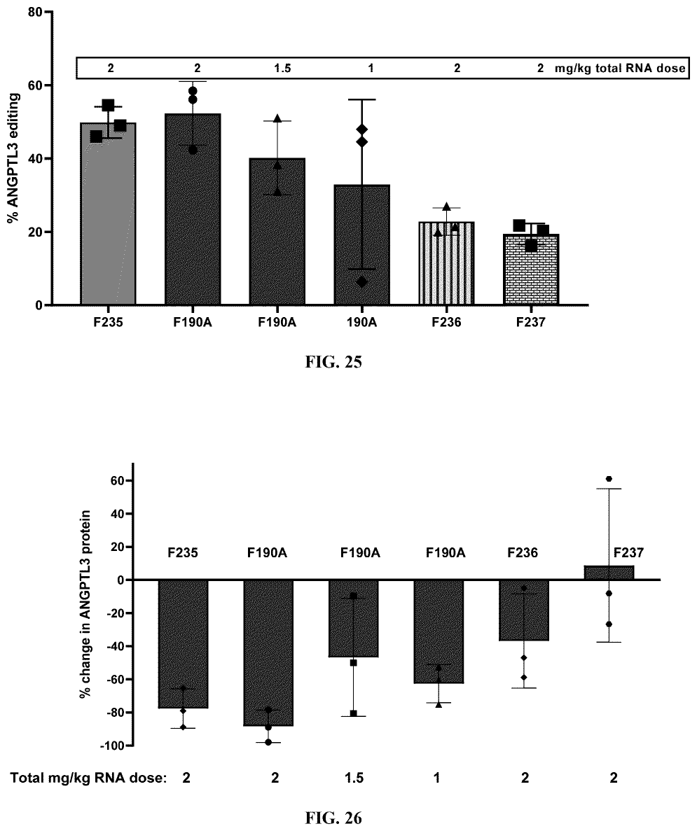

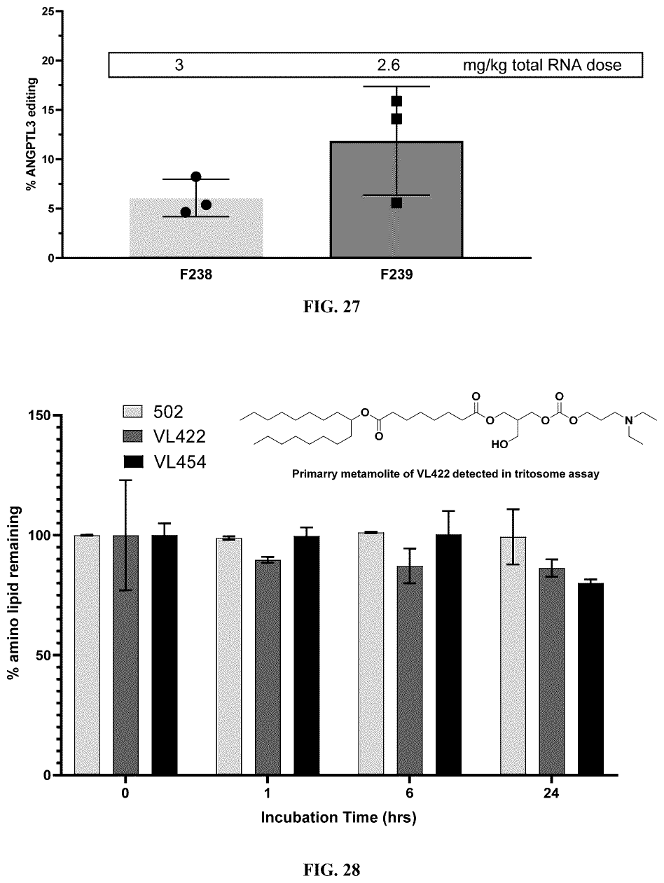

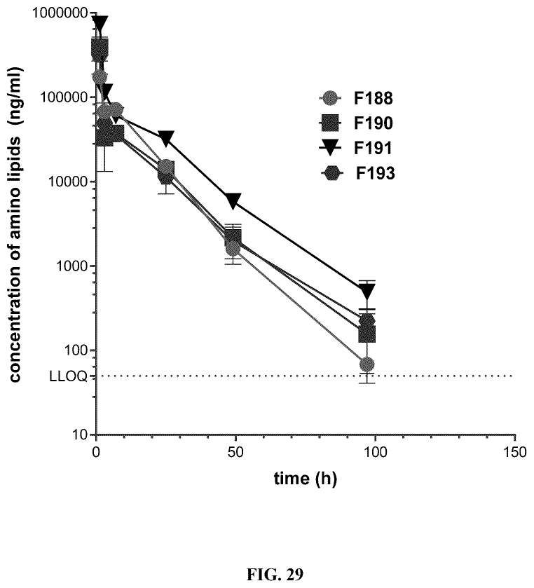

The term “protospacer,” or “target sequence” and their grammatical equivalents as used herein can refer to a DNA sequence of a target gene. In the native state, a protospacer is adjacent to a PAM (protospacer adjacent motif). The term “spacer” can be the RNA version of the protospacer that binds to the complementary strand of the protospacer. A spacer can be within a guide RNA (gRNA). The site of cleavage by an RNA-guided nuclease is within a protospacer sequence. Please see FIG. 1A for an illustration.

mRNA Encoding SpCas9, CBE, and ABE Proteins

mRNA for SpCas9, CBE, and ABE were produced by different methods well known in the art. One of such methods used herein was in vitro transcription (IVT) using T7 polymerase or additional RNA polymerase variants. Typically, IVT of mRNA uses a linearized DNA template that comprises a T7 polymerase promoter, mRNA coding sequence (CDS), 3′ and 5′ untranslated regions (UTRs), poly A tail, and additional replication and transcription regulatory elements. Prior to IVT, the DNA template was in the form of a plasmid, PCR product, or additional double-stranded DNA construct. A typical IVT reaction includes T7 polymerase, DNA template, RNase inhibitor, cap analog, inorganic pyrophosphatase, and naturally occurring ribonucleotides (rNTPs) such as GTP, ATP, CTP, UTP, or substitutions of natural rNTPs with modified rNTPs such as pseudouridine, N1-methylpseudouridine, 5-methyl cytidine, 5-methoxyuridine, N6-methyl adenosine, and N4-acetylcytidine. The cap analog was a dinucleotide or trinucleotide cap structure with the first initiating nucleotide containing standard 2′-hydroxyl group, 2′—O-methyl group, or additional 2′chemical modification. Cap analog also was added after the IVT reaction using a vaccinia capping enzyme. After IVT, in some cases DNase is added to the transcription mixture to remove DNA template; alternatively, residual DNA was removed by ion exchange column chromatography. Purification and concentration of mRNA were performed with ion exchange chromatography, affinity chromatography, precipitation, ion-pairing reverse-phase chromatography, enzymatic reactions, size exclusion chromatography, and/or tangential flow filtration. Similar IVT and purification process were used to produce mRNA encoding SpCas9, CBE, and ABE; in all cases the DNA template, reaction conditions, and purification parameters were optimized for the specific gene of interest. In some examples, capped and polyadenylated SpCas9 mRNA MS002 was obtained from commercially sources (TriLink, for e.g.). The SpCas9 mRNA MS002 and adenosine base editor (ABE) mRNAs MA002 and MA004 prepared in Verve's laboratory.

The adenosine nucleobase editor mRNA MA002 comprises a fusion protein comprising a polypeptide encoded by the polynucleotide sequence provided below

ATGAGCGAGGTCGAGTTCTCTCACGAATATTGGATGAGACACGCTCTCAC
CCTGGCTAAGAGAGCCAGGGACGAAAGAGAGGTGCCAGTTGGCGCTGTCC
TGGTGTTGAACAATCGCGTCATCGGAGAAGGATGGAATCGCGCCATTGGC
CTGCACGATCCAACCGCACATGCCGAAATTATGGCTCTGCGGCAAGGCGG
CCTCGTGATGCAAAATTACAGACTGATCGATGCTACCCTCTACGTCACCT
TCGAGCCCTGTGTCATGTGTGCTGGGGCAATGATTCACTCCCGGATTGGC
CGCGTGGTGTTTGGAGTGCGGAATGCCAAGACTGGCGCCGCTGGATCTCT
GATGGACGTCCTGCACcatCCTGGGATGAACCACCGGGTCGAGATCACAG
AGGGAATTCTGGCTGACGAGTGCGCTGCCCTGCTGTGCaggTTCTTTAGA
ATGCCtAGAaggGTGTTCAACGCCCAGAAAAAAGCTCAGAGCAGCACCGA
TTCCGGCGGAAGCAGCGGAGGATCTTCTGGAAGCGAAACCCCAGGCACCA
GCGAGTCTGCCACACCAGAATCATCTGGCGGTAGCTCCGGCGGCAGCGAC
AAGAAGTATTCTATCGGACTGGCCATCGGCACCAACTCTGTTGGATGGGC
CGTGATCACCGACGAGTACAAGGTGCCCAGCAAGAAATTCAAGGTGCTGG
GCAACACCGACAGGCACAGCATCAAGAAGAACCTGATCGGCGCACTGCTG
TTCGACTCTGGCGAAACAGCCGAGGCCACCAGACTGAAGAGAACAGCCCG
CAGACGGTACACCAGAAGAAAGAACCGGATCTGCTACCTCCAAGAGATCT
TCAGCAACGAGATGGCCAAGGTGGACGACAGCTTCTTCCACAGACTGGAA
GAGTCCTTCCTGGTGGAAGAGGACAAGAAGCACGAGAGACACCCCATCTT
CGGCAACATCGTGGACGAGGTGGC

1-147. (canceled)

148. An amino lipid having a structure of Formula (I*), or a pharmaceutically acceptable salt or solvate thereof, 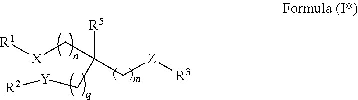
wherein
R¹ and R² are —C₀-C₁₀ alkylene-L-R⁶, wherein the alkylene is unsubstituted, and wherein R¹ and R² are the same;
each of X and Y is independently —OC(═O)— or —OC(═O)—;
Z is —OC(═O)—, —OC(═O)—, —OC(═O)O—, or a bond;
each of L is independently —C(═O)O—, —C(═O)—, or —OC(═O)—;
R³ is —C₀-C₁₀ alkylene-NR⁷R⁸, wherein the alkylene is unsubstituted;
R⁵ is hydrogen;
R⁶ is unsubstituted C₃-C₃₀ alkyl;
each of R⁷ and R⁸ is independently C₁-C₁₆ alkyl or C₁-C₁₆ heteroalkyl, wherein the heteroalkyl is optionally substituted with one or more substituents selected from oxo and C₁-C₆ alkyl;
each of n, q, and m is 1.

149. The amino acid of claim 148, or the pharmaceutically acceptable salt or solvate thereof, wherein R¹ and R² are —C₄-C₈ alkylene-L-R⁶.

150. The amino acid of claim 148, or the pharmaceutically acceptable salt or solvate thereof, wherein R⁶ is C₁₀ alkyl, C₁₁ alkyl, C₁₂ alkyl, C₁₃ alkyl, C₁₄ alkyl, C₁₅ alkyl, C₁₆ alkyl, C₁₇ alkyl, or C₁₈ alkyl.

151. The amino acid of claim 148, or the pharmaceutically acceptable salt or solvate thereof, wherein Z is —OC(═O)O— or a bond.

152. The amino acid of claim 148, or the pharmaceutically acceptable salt or solvate thereof, wherein R³ is —NR⁷R⁸.

153. The amino acid of claim 148, or the pharmaceutically acceptable salt or solvate thereof, wherein R³ is —C₁-C₄ alkylene-NR⁷R⁸.

154. The amino acid of claim 148, or the pharmaceutically acceptable salt or solvate thereof, wherein each of R⁷ and R⁸ is unsubstituted C₁-C₆ alkyl.

155. The amino acid of claim 148, or the pharmaceutically acceptable salt or solvate thereof, each of R⁷ and R⁸ is independently C₁-C₁₆ alkyl or C₁-C₁₆ heteroalkyl, wherein the alkyl is unsubstituted, and wherein the heteroalkyl is optionally substituted with one or more substituents selected from oxo and C₁-C₆ alkyl, and wherein the heteroalkyl has one or more skeletal oxygen atom, nitrogen atom, or both.

156. The amino acid of claim 148, or the pharmaceutically acceptable salt or solvate thereof, wherein the amino acid has a structure of

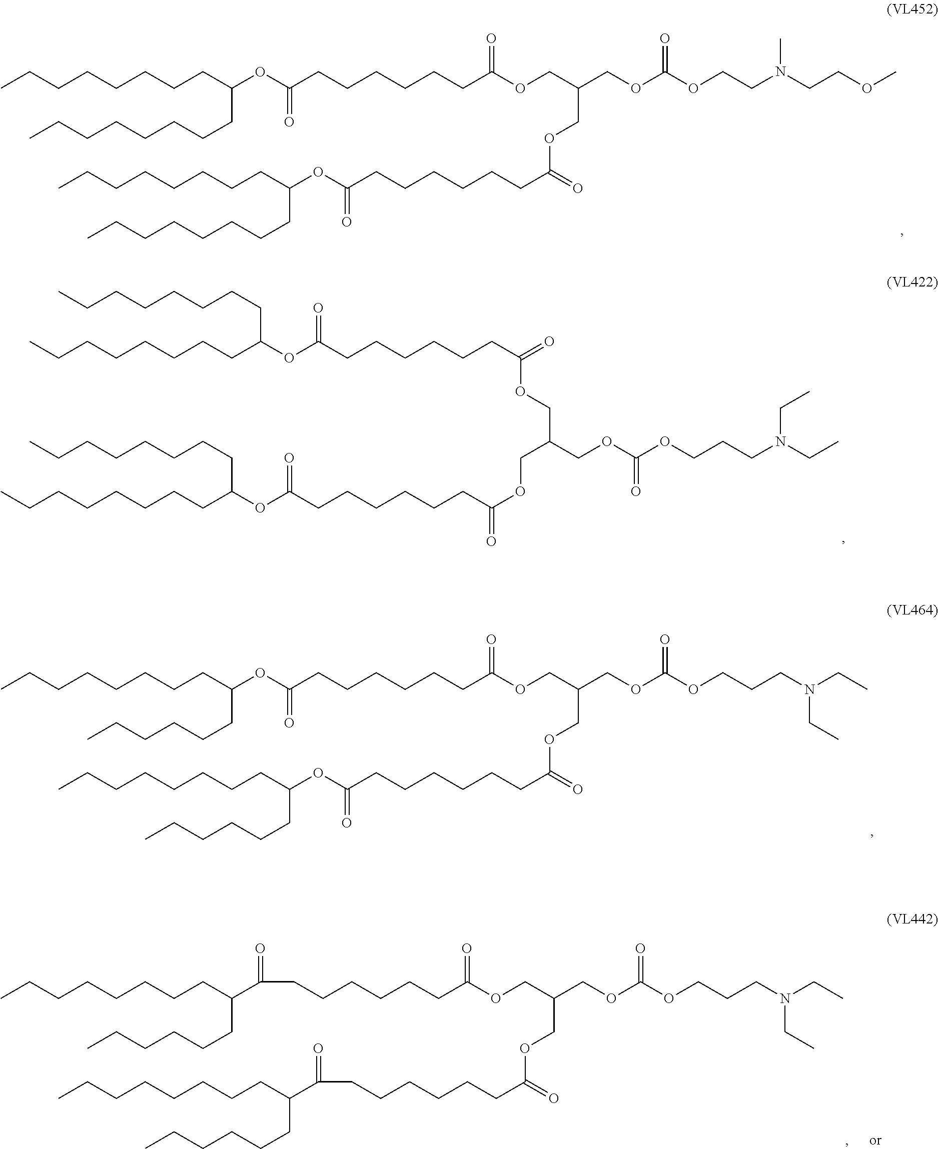

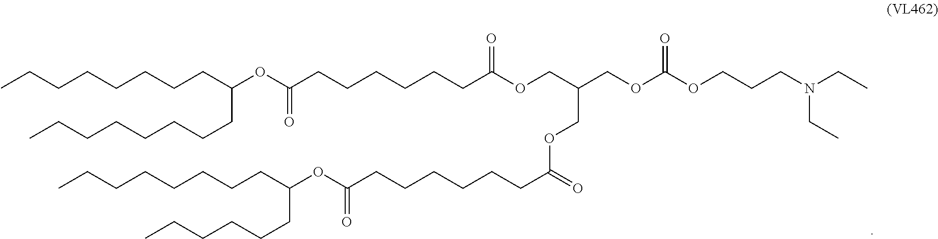 [SMILES: CCCCCCCCC(CCCCCC)OC(=O)CCCCCCC(=O)OCC(COC(=O)CCCCCCC(=O)OC(CCCCCCCC)CCCCCCCC)COC(=O)OCCCN(CC)CC]

157. A nanoparticle composition that comprises an amino lipid of claim 148, or a pharmaceutically acceptable salt or solvate thereof.

158. The nanoparticle composition of claim 157, wherein the amino lipid or the pharmaceutically acceptable salt or solvate thereof, comprises from 20 mol % to 80 mol % of a total lipid content present in the nanoparticle composition.

159. The nanoparticle composition of 157, wherein the nanoparticle composition comprises one or more nucleic acid molecular entities.

160. The nanoparticle composition of 157, wherein the nanoparticle composition comprises a neutral lipid.

161. The nanoparticle composition of claim 160, wherein the neutral lipid is selected from 1,2-distearoyl-sn-glycero-3-phosphocholine (DSPC), 1,2-dipalmitoyl-sn-glycero-3-phosphocholine (DPPC), 1,2-dimyristoyl-sn-glycero-phosphocholine (DMPC), 2-oleoyl-1-palmitoyl-sn-glycero-3-phosphocholine (POPC), 1,2-dioleoyl-sn-glycero-3-phosphoethanolamine (DOPE), and sphingomyelin.

162. The nanoparticle composition of claim 160, wherein the neutral lipid comprises from about 1 mol % to about 20 mol % of a total lipid content present in the nanoparticle composition.

163. The nanoparticle composition of claim 157, wherein the nanoparticle composition comprises a structural lipid.

164. The nanoparticle composition of claim 163, wherein the structural lipid is sterol or a derivative thereof.

165. The nanoparticle composition of claim 164, wherein the sterol or the derivative thereof is cholesterol or cholesterol derivative.

166. The nanoparticle composition of claim 165, wherein the structural lipid comprises from about 15 mol % to about 65 mol % of a total lipid content present in the nanoparticle composition.

167. The nanoparticle composition of claim 157, wherein the nanoparticle composition comprises a PEG-lipid.

168. A nanoparticle composition comprising:
(a) one or more nucleic acid molecular entities;
(b) an amino lipid of claim 148, or a salt thereof,
wherein the amino lipid or a salt thereof, comprises from 20 mol % to 80 mol % of a total lipid content present in the nanoparticle composition;
(c) a neutral lipid, comprising from 2 mol % to 25 mol % of the total lipid content present in the nanoparticle composition;
(d) a structural lipid, comprising from 30 mol % to 60 mol % of the total lipid content present in the nanoparticle composition;
(e) a PEG-lipid, comprising from 0.1 mol % to 6 mol % of the total lipid content present in the nanoparticle composition.


SEQUENCE LISTING (21 sequences)

SEQ ID NO: 1 - DNA, 20 bp, synthetic construct
  source 1..20: mol_type = other DNA
caggttccatgggatgctct

SEQ ID NO: 2 - DNA, 20 bp, synthetic construct
  source 1..20: mol_type = other DNA
ggctgatgaggccgcacatg

SEQ ID NO: 3 - DNA, 20 bp, synthetic construct
  source 1..20: mol_type = other DNA
cccataccttggagcaacgg

SEQ ID NO: 4 - DNA, 20 bp, synthetic construct
  source 1..20: mol_type = other DNA
cccgcaccttggcgcagcgg

SEQ ID NO: 5 - DNA, 20 bp, synthetic construct
  source 1..20: mol_type = other DNA
gagatacctgagtaactttc

SEQ ID NO: 6 - DNA, 20 bp, synthetic construct
  source 1..20: mol_type = other DNA
aagatacctgaataactctc

SEQ ID NO: 7 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 55: mod_base = OTHER; note = 2'-O-methyl nucleotide
  modified_base 56: mod_base = OTHER; note = 2'-O-methyl nucleotide
  modified_base 57: mod_base = OTHER; note = 2'-O-methyl nucleotide
  modified_base 60: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
caggttccatgggatgctctgttttagagctagaaatagcaagttaaaataaggctagtccgttatcaacttgaaaaagtggcaccgagtcggtgctttt

SEQ ID NO: 8 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
ggctgatgaggccgcacatggttttagagctagaaatagcaagttaaaataaggctagtccgttatcaacttgaaaaagtggcaccgagtcggtgctttt

SEQ ID NO: 9 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 57: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 89: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
cccataccttggagcaacgggttttagagctagaaatagcaagttaaaataaggctagtccgttatcaacttgaaaaagtggcaccgagtcggtgctttt

SEQ ID NO: 10 - RNA, 100 nt, synthetic construct; identical to SEQ ID NO: 9
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 55: mod_base = OTHER; note = 2'-O-methyl nucleotide
  modified_base 56: mod_base = OTHER; note = 2'-O-methyl nucleotide
  modified_base 57: mod_base = OTHER; note = 2'-O-methyl nucleotide
  modified_base 60: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide

SEQ ID NO: 11 - RNA, 100 nt, synthetic construct; identical to SEQ ID NO: 9
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 55: mod_base = OTHER; note = 2'-O-methyl nucleotide
  modified_base 56: mod_base = OTHER; note = 2'-O-methyl nucleotide
  modified_base 57: mod_base = OTHER; note = 2'-O-methyl nucleotide
  modified_base 60: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide

SEQ ID NO: 12 - RNA, 99 nt, synthetic construct
  source 1..99: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 96: mod_base = OTHER; note = nucleotide with phosphorothioate linkage
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide
cccatacttggagcaacgggttttagagctagaaatagcaagttaaaataaggctagtccgttatcaacttgaaaaagtggcaccgagtcggtgctttt

SEQ ID NO: 13 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 55: mod_base = OTHER; note = 2'-O-methyl nucleotide
  modified_base 56: mod_base = OTHER; note = 2'-O-methyl nucleotide
  modified_base 57: mod_base = OTHER; note = 2'-O-methyl nucleotide
  modified_base 60: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
cccgcaccttggcgcagcgggttttagagctagaaatagcaagttaaaataaggctagtccgttatcaacttgaaaaagtggcaccgagtcggtgctttt

SEQ ID NO: 14 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 57: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 89: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
gagatacctgagtaactttcgttttagagctagaaatagcaagttaaaataaggctagtccgttatcaacttgaaaaagtggcaccgagtcggtgctttt

SEQ ID NO: 15 - RNA, 100 nt, synthetic construct; identical to SEQ ID NO: 14
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 55: mod_base = OTHER; note = 2'-O-methyl nucleotide
  modified_base 56: mod_base = OTHER; note = 2'-O-methyl nucleotide
  modified_base 57: mod_base = OTHER; note = 2'-O-methyl nucleotide
  modified_base 60: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide

SEQ ID NO: 16 - RNA, 100 nt, synthetic construct; identical to SEQ ID NO: 14
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 55: mod_base = OTHER; note = 2'-O-methyl nucleotide
  modified_base 56: mod_base = OTHER; note = 2'-O-methyl nucleotide
  modified_base 57: mod_base = OTHER; note = 2'-O-methyl nucleotide
  modified_base 60: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide

SEQ ID NO: 17 - RNA, 100 nt, synthetic construct
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 33: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 69: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 73: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 82: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide
aagatacctgaataactctcgttttagagctagaaatagcaagttaaaataaggctagtccgttatcaacttgaaaaagtggcaccgagtcggtgctttt

SEQ ID NO: 18 - RNA, 100 nt, synthetic construct; identical to SEQ ID NO: 17
  source 1..100: mol_type = other RNA
  modified_base 1: mod_base = OTHER; note = 2'-O-methyl nucleotide with phosphorothioate linkage
  modified_base 2: mod_base = OTHER; note = 2'-O-methyl nucleotide with phosphorothioate linkage
  modified_base 3: mod_base = OTHER; note = 2'-O-methyl nucleotide with phosphorothioate linkage
  modified_base 21: mod_base = OTHER; note = 2'-O-methyl nucleotide
  modified_base 28: mod_base = OTHER; note = 2'-O-methyl nucleotide
  modified_base 29: mod_base = OTHER; note = 2'-O-methyl nucleotide
  modified_base 30: mod_base = OTHER; note = 2'-O-methyl nucleotide
  modified_base 31: mod_base = OTHER; note = 2'-O-methyl nucleotide
  modified_base 32: mod_base = OTHER; note = 2'-O-methyl nucleotide
  modified_base 34: mod_base = OTHER; note = 2'-O-methyl nucleotide
  modified_base 35: mod_base = OTHER; note = 2'-O-methyl nucleotide
  modified_base 36: mod_base = OTHER; note = 2'-O-methyl nucleotide
  modified_base 37: mod_base = OTHER; note = 2'-O-methyl nucleotide
  modified_base 38: mod_base = OTHER; note = 2'-O-methyl nucleotide
  modified_base 39: mod_base = OTHER; note = 2'-O-methyl nucleotide
  modified_base 40: mod_base = OTHER; note = 2'-O-methyl nucleotide
  modified_base 41: mod_base = OTHER; note = 2'-O-methyl nucleotide
  modified_base 42: mod_base = OTHER; note = 2'-O-methyl nucleotide
  modified_base 46: mod_base = OTHER; note = 2'-O-methyl nucleotide
  modified_base 48: mod_base = OTHER; note = 2'-O-methyl nucleotide
  modified_base 50: mod_base = OTHER; note = 2'-O-methyl nucleotide
  modified_base 52: mod_base = OTHER; note = 2'-O-methyl nucleotide
  modified_base 53: mod_base = OTHER; note = 2'-O-methyl nucleotide
  modified_base 54: mod_base = OTHER; note = 2'-O-methyl nucleotide
  modified_base 55: mod_base = OTHER; note = 2'-O-methyl nucleotide
  modified_base 56: mod_base = OTHER; note = 2'-O-methyl nucleotide
  modified_base 57: mod_base = OTHER; note = 2'-O-methyl nucleotide
  modified_base 60: mod_base = OTHER; note = 2'-O-methyl nucleotide
  modified_base 61: mod_base = OTHER; note = 2'-O-methyl nucleotide
  modified_base 66: mod_base = OTHER; note = 2'-O-methyl nucleotide
  modified_base 67: mod_base = OTHER; note = 2'-O-methyl nucleotide
  modified_base 70: mod_base = OTHER; note = 2'-O-methyl nucleotide
  modified_base 71: mod_base = OTHER; note = 2'-O-methyl nucleotide
  modified_base 72: mod_base = OTHER; note = 2'-O-methyl nucleotide
  modified_base 74: mod_base = OTHER; note = 2'-O-methyl nucleotide
  modified_base 75: mod_base = OTHER; note = 2'-O-methyl nucleotide
  modified_base 76: mod_base = OTHER; note = 2'-O-methyl nucleotide
  modified_base 77: mod_base = OTHER; note = 2'-O-methyl nucleotide
  modified_base 78: mod_base = OTHER; note = 2'-O-methyl nucleotide
  modified_base 79: mod_base = OTHER; note = 2'-O-methyl nucleotide
  modified_base 80: mod_base = OTHER; note = 2'-O-methyl nucleotide
  modified_base 81: mod_base = OTHER; note = 2'-O-methyl nucleotide
  modified_base 83: mod_base = OTHER; note = 2'-O-methyl nucleotide
  modified_base 84: mod_base = OTHER; note = 2'-O-methyl nucleotide
  modified_base 85: mod_base = OTHER; note = 2'-O-methyl nucleotide
  modified_base 86: mod_base = OTHER; note = 2'-O-methyl nucleotide
  modified_base 87: mod_base = OTHER; note = 2'-O-methyl nucleotide
  modified_base 88: mod_base = OTHER; note = 2'-O-methyl nucleotide
  modified_base 89: mod_base = OTHER; note = 2'-O-methyl nucleotide
  modified_base 90: mod_base = OTHER; note = 2'-O-methyl nucleotide
  modified_base 91: mod_base = OTHER; note = 2'-O-methyl nucleotide
  modified_base 92: mod_base = OTHER; note = 2'-O-methyl nucleotide
  modified_base 93: mod_base = OTHER; note = 2'-O-methyl nucleotide
  modified_base 94: mod_base = OTHER; note = 2'-O-methyl nucleotide
  modified_base 95: mod_base = OTHER; note = 2'-O-methyl nucleotide
  modified_base 96: mod_base = OTHER; note = 2'-O-methyl nucleotide
  modified_base 97: mod_base = OTHER; note = 2'-O-methyl nucleotide with phosphorothioate linkage
  modified_base 98: mod_base = OTHER; note = 2'-O-methyl nucleotide with phosphorothioate linkage
  modified_base 99: mod_base = OTHER; note = 2'-O-methyl nucleotide with phosphorothioate linkage
  modified_base 100: mod_base = OTHER; note = 2'-O-methyl nucleotide

SEQ ID NO: 19 - DNA, 9528 bp, synthetic construct
  source 1..9528: mol_type = other DNA
  modified_base 367: mod_base = OTHER; note = 2'-O-methyl modified nucleotide
  modified_base 368: mod_base = OTHER; note = 2'-O-methyl modified nucleotide
  modified_base 369: mod_base = OTHER; note = 2'-O-methyl modified nucleotide
  modified_base 439: mod_base = OTHER; note = 2'-O-methyl modified nucleotide
  modified_base 440: mod_base = OTHER; note = 2'-O-methyl modified nucleotide
  modified_base 441: mod_base = OTHER; note = 2'-O-methyl modified nucleotide
  modified_base 456: mod_base = OTHER; note = 2'-O-methyl modified nucleotide
  modified_base 460: mod_base = OTHER; note = 2'-O-methyl modified nucleotide
  modified_base 461: mod_base = OTHER; note = 2'-O-methyl modified nucleotide
  modified_base 462: mod_base = OTHER; note = 2'-O-methyl modified nucleotide
  modified_base 5131: mod_base = OTHER; note = 2'-O-methyl modified nucleotide
  modified_base 5132: mod_base = OTHER; note = 2'-O-methyl modified nucleotide
  modified_base 5133: mod_base = OTHER; note = 2'-O-methyl modified nucleotide
  modified_base 5203: mod_base = OTHER; note = 2'-O-methyl modified nucleotide
  modified_base 5204: mod_base = OTHER; note = 2'-O-methyl modified nucleotide
  modified_base 5205: mod_base = OTHER; note = 2'-O-methyl modified nucleotide
  modified_base 5220: mod_base = OTHER; note = 2'-O-methyl modified nucleotide
  modified_base 5224: mod_base = OTHER; note = 2'-O-methyl modified nucleotide
  modified_base 5225: mod_base = OTHER; note = 2'-O-methyl modified nucleotide
  modified_base 5226: mod_base = OTHER; note = 2'-O-methyl modified nucleotide
atgagcgaggtcgagttctctcacgaatattggatgagacacgctctcaccctggctaagagagccagggacgaaagagaggtgccagttggcgctgtcctggtgttgaacaatcgcgtcatcggagaaggatggaatcgcgccattggcctgcacgatccaaccgcacatgccgaaattatggctctgcggcaaggcggcctcgtgatgcaaaattacagactgatcgatgctaccctctacgtcaccttcgagccctgtgtcatgtgtgctggggcaatgattcactcccggattggccgcgtggtgtttggagtgcggaatgccaagactggcgccgctggatctctgatggacgtcctgcaccatcctgggatgaaccaccgggtcgagatcacagagggaattctggctgacgagtgcgctgccctgctgtgcaggttctttagaatgcctagaagggtgttcaacgcccagaaaaaagctcagagcagcaccgattccggcggaagcagcggaggatcttctggaagcgaaaccccaggcaccagcgagtctgccacaccagaatcatctggcggtagctccggcggcagcgacaagaagtattctatcggactggccatcggcaccaactctgttggatgggccgtgatcaccgacgagtacaaggtgcccagcaagaaattcaaggtgctgggcaacaccgacaggcacagcatcaagaagaacctgatcggcgcactgctgttcgactctggcgaaacagccgaggccaccagactgaagagaacagcccgcagacggtacaccagaagaaagaaccggatctgctacctccaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaatctgatcgcccagctgcctggcgagaagaagaatggcctgttcggcaacctgattgccctgagcctgggcctgacacctaacttcaagagcaacttcgacctggccgaggacgccaaactgcagctgagcaaggacacctacgacgacgacctggacaatctgctggcccagatcggcgatcagtacgccgacttgtttctggccgccaagaatctgagcgacgccatcctgctgtccgacatcctgagagtgaacaccgagatcaccaaggcacctctgagcgcctctatgatcaagagatacgacgagcaccaccaggatctgaccctgctgaaggccctcgttagacagcagctgccagagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgagaaagcagagaaccttcgacaacggcagcatccctcaccagatccacctgggagaactgcacgccattctgcggagacaagaggacttttacccattcctgaaggacaaccgggaaaagatcgagaaaatcctgaccttcaggatcccctactacgtgggaccactggccagaggcaatagcagattcgcctggatgaccagaaagagcgaggaaaccatcactccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccatcgtggatctgctgttcaagaccaaccggaaagtgaccgtgaagcagctgaaagaggactacttcaagaaaatcgagtgcttcgacagcgtcgagatctccggcgtggaagatcggttcaatgccagcctgggcacataccacgatctgctgaaaattatcaaggacaaggacttcctggacaacgaagagaacgaggacatccttgaggacatcgtgctgacactgaccctgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaacagaaacttcatgcagctgattcacgacgacagcctcaccttcaaagaggatatccagaaagcccaggtgtccggccagggcgattctctgcatgagcacattgccaacctggccggctctcccgccattaagaaaggcatcctgcagacagtgaaggtggtggacgagcttgtgaaagtgatgggcagacacaagcccgagaacatcgtgatcgaaatggccagagagaaccagaccacacagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatggacgggatatgtacgtggaccaagagctggacatcaacagactgtccgactacgatgtggaccatatcgtgccccagtcttttctgaaggacgactccatcgacaacaaggtcctgaccagatccgacaagaatcggggcaagagcgacaacgtgccctccgaagaggtggtcaagaagatgaagaactactggcgacagctgctgaacgccaagctgattacccagcggaagttcgacaatctgaccaaggccgaaagaggcggcctgagcgaactggataaggccggcttcatcaagagacagctggtggaaacccggcagatcacaaagcacgtggcacagattctggactctcggatgaacactaagtacgacgagaacgacaaactgatccgcgaagtgaaagtcatcaccctgaagtccaagctggtgtccgatttccggaaggatttccagttctacaaagtgcgcgagatcaacaactaccatcacgcccacgacgcctacctgaatgccgttgttggaacagccctgatcaaaaagtaccctaagctggaaagcgagttcgtgtacggcgactacaaggtgtacgacgtgcggaagatgatcgccaagagcgagcaagagattggcaaggcaaccgccaagtacttcttctacagcaacatcatgaactttttcaagacagagatcaccctcgccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtctatcctgcctaagcggaactccgacaagctgatcgccagaaagaaggactgggaccccaagaagtacggcggcttcgattctcctaccgtggcctatagcgtgctggtggtggccaaagtggaaaagggcaagtccaagaaactcaagagcgtgaaagagctgctggggatcaccatcatggaaagaagcagcttcgagaagaatccgatcgatttcctcgaggccaagggctacaaagaagtgaaaaaggacctgatcatcaagctccccaagtactccctgttcgagctggaaaacggccggaagagaatgctggcctctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagtttagcaagagagtgattctggccgacgccaatctggacaaagtgctgtccgcctacaacaagcaccgggacaagcctatcagagagcaggccgagaatatcatccacctgtttaccctgaccaacctgggagcccctgccgccttcaagtactttgacaccaccatcgaccggaagcggtacacctccaccaaagaggtgctggacgccactctgatccaccagtctatcaccggcctgtacgagacacggatcgacctgtctcaactcggaggcgacgaaggcgccgataagagaaccgccgatggctctgagttcgagagccccaagaaaaagcgcaaagtgatgagcgaggtcgagttctctcacgaatattggatgagacacgctctcaccctggctaagagagccagggacgaaagagaggtgccagttggcgctgtcctggtgttgaacaatcgcgtcatcggagaaggatggaatcgcgccattggcctgcacgatccaaccgcacatgccgaaattatggctctgcggcaaggcggcctcgtgatgcaaaattacagactgatcgatgctaccctctacgtcaccttcgagccctgtgtcatgtgtgctggggcaatgattcactcccggattggccgcgtggtgtttggagtgcggaatgccaagactggcgccgctggatctctgatggacgtcctgcaccatcctgggatgaaccaccgggtcgagatcacagagggaattctggctgacgagtgcgctgccctgctgtgcaggttctttagaatgcctagaagggtgttcaacgcccagaaaaaagctcagagcagcaccgattccggcggaagcagcggaggatcttctggaagcgaaaccccaggcaccagcgagtctgccacaccagaatcatctggcggtagctccggcggcagcgacaagaagtattctatcggactggccatcggcaccaactctgttggatgggccgtgatcaccgacgagtacaaggtgcccagcaagaaattcaaggtgctgggcaacaccgacaggcacagcatcaagaagaacctgatcggcgcactgctgttcgactctggcgaaacagccgaggccaccagactgaagagaacagcccgcagacggtacaccagaagaaagaaccggatctgctacctccaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctatctggccctggctcacatgatcaagttccggggccacttcctgatcgagggcgacctgaatcctgacaacagcgacgtggacaagctgttcatccagctggtgcagacctacaaccagctgttcgaggaaaaccccatcaacgccagcggagtggatgccaaggccatcctgtctgccagactgagcaagagcagacggctggaaaatctgatcgcccagctgcctggcgagaagaagaatggcctgttcggcaacctgattgccctgagcctgggcctgacacctaacttcaagagcaacttcgacctggccgaggacgccaaactgcagctgagcaaggacacctacgacgacgacctggacaatctgctggcccagatcggcgatcagtacgccgacttgtttctggccgccaagaatctgagcgacgccatcctgctgtccgacatcctgagagtgaacaccgagatcaccaaggcacctctgagcgcctctatgatcaagagatacgacgagcaccaccaggatctgaccctgctgaaggccctcgttagacagcagctgccagagaagtacaaagagattttcttcgaccagagcaagaacggctacgccggctacattgatggcggagccagccaagaggaattctacaagttcatcaagcccatcctcgagaagatggacggcaccgaggaactgctggtcaagctgaacagagaggacctgctgagaaagcagagaaccttcgacaacggcagcatccctcaccagatccacctgggagaactgcacgccattctgcggagacaagaggacttttacccattcctgaaggacaaccgggaaaagatcgagaaaatcctgaccttcaggatcccctactacgtgggaccactggccagaggcaatagcagattcgcctggatgaccagaaagagcgaggaaaccatcactccctggaacttcgaggaagtggtggacaagggcgccagcgctcagtccttcatcgagcggatgaccaacttcgataagaacctgcctaacgagaaggtgctgcccaagcacagcctgctgtacgagtacttcaccgtgtacaacgagctgaccaaagtgaaatacgtgaccgagggaatgagaaagcccgcctttctgagcggcgagcagaaaaaggccatcgtggatctgctgttcaagaccaaccggaaagtgaccgtgaagcagctgaaagaggactacttcaagaaaatcgagtgcttcgacagcgtcgagatctccggcgtggaagatcggttcaatgccagcctgggcacataccacgatctgctgaaaattatcaaggacaaggacttcctggacaacgaagagaacgaggacatccttgaggacatcgtgctgacactgaccctgtttgaggacagagagatgatcgaggaacggctgaaaacatacgcccacctgttcgacgacaaagtgatgaagcaactgaagcggcggagatacaccggctggggcagactgtctcggaagctgatcaacggcatccgggataagcagtccggcaagaccatcctggactttctgaagtccgacggcttcgccaacagaaacttcatgcagctgattcacgacgacagcctcaccttcaaagaggatatccagaaagcccaggtgtccggccagggcgattctctgcatgagcacattgccaacctggccggctctcccgccattaagaaaggcatcctgcagacagtgaaggtggtggacgagcttgtgaaagtgatgggcagacacaagcccgagaacatcgtgatcgaaatggccagagagaaccagaccacacagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatggacgggatatgtacgtggaccaagagctggacatcaacagactgtccgactacgatgtggaccatatcgtgccccagtcttttctgaaggacgactccatcgacaacaaggtcctgaccagatccgacaagaatcggggcaagagcgacaacgtgccctccgaagaggtggtcaagaagatgaagaactactggcgacagctgctgaacgccaagctgattacccagcggaagttcgacaatctgaccaaggccgaaagaggcggcctgagcgaactggataaggccggcttcatcaagagacagctggtggaaacccggcagatcacaaagcacgtggcacagattctggactctcggatgaacactaagtacgacgagaacgacaaactgatccgcgaagtgaaagtcatcaccctgaagtccaagctggtgtccgatttccggaaggatttccagttctacaaagtgcgcgagatcaacaactaccatcacgcccacgacgcctacctgaatgccgttgttggaacagccctgatcaaaaagtaccctaagctggaaagcgagttcgtgtacggcgactacaaggtgtacgacgtgcggaagatgatcgccaagagcgagcaagagattggcaaggcaaccgccaagtacttcttctacagcaacatcatgaactttttcaagacagagatcaccctcgccaacggcgagatcagaaagcggcctctgatcgagacaaacggcgaaaccggcgagattgtgtgggataagggcagagactttgccacagtgcggaaagtgctgagcatgccccaagtgaatatcgtgaagaaaaccgaggtgcagacaggcggcttcagcaaagagtctatcctgcctaagcggaactccgacaagctgatcgccagaaagaaggactgggaccccaagaagtacggcggcttcgattctcctaccgtggcctatagcgtgctggtggtggccaaagtggaaaagggcaagtccaagaaactcaagagcgtgaaagagctgctggggatcaccatcatggaaagaagcagcttcgagaagaatccgatcgatttcctcgaggccaagggctacaaagaagtgaaaaaggacctgatcatcaagctccccaagtactccctgttcgagctggaaaacggccggaagagaatgctggcctctgctggcgaactgcagaagggaaacgaactggccctgcctagcaaatatgtgaacttcctgtacctggccagccactatgagaagctgaagggcagccccgaggacaatgagcaaaagcagctgtttgtggaacagcacaagcactacctggacgagatcatcgagcagatcagcgagtttagcaagagagtgattctggccgacgccaatctggacaaagtgctgtccgcctacaacaagcaccgggacaagcctatcagagagcaggccgagaatatcatccacctgtttaccctgaccaacctgggagcccctgccgccttcaagtactttgacaccaccatcgaccggaagcggtacacctccaccaaagaggtgctggacgccactctgatccaccagtctatcaccggcctgtacgagacacggatcgacctgtctcaactcggaggcgacgaaggcgccgataagagaaccgccgatggctctgagttcgagagccccaagaaaaagcgcaaagtg

SEQ ID NO: 20 - RNA, 4929 nt, synthetic construct
  source 1..4929: mol_type = other RNA
  modified_base 7: mod_base = OTHER; note = N1-methylpseudouridine
  modified_base 25: mod_base = OTHER; note = N1-methylpseudouridine
  modified_base 35: mod_base = OTHER; note = N1-methylpseudouridine
  modified_base 37: mod_base = OTHER; note = N1-methylpseudouridine
  modified_base 49: mod_base = OTHER; note = N1-methylpseudouridine
  modified_base 58: mod_base = OTHER; note = N1-methylpseudouridine
  modified_base 63: mod_base = OTHER; note = N1-methylpseudouridine
  modified_base 64: mod_base = OTHER; note = N1-methylpseudouridine
  modified_base 75: mod_base = OTHER; note = N1-methylpseudouridine
  modified_base 78: mod_base = OTHER; note = N1-methylpseudouridine
  modified_base 82: mod_base = OTHER; note = N1-methylpseudouridine
  modified_base 94: mod_base = OTHER; note = N1-methylpseudouridine
  modified_base 100: mod_base = OTHER; note = N1-methylpseudouridine
  modified_base 130: mod_base = OTHER; note = N1-methylpseudouridine
  modified_base 136: mod_base = OTHER; note = N1-methylpseudouridine
  modified_base 145: mod_base = OTHER; note = N1-methylpseudouridine
  modified_base 148: mod_base = OTHER; note = N1-methylpseudouridine
  modified_base 151: mod_base = OTHER; note = N1-methylpseudouridine
  modified_base 154: mod_base = OTHER; note = N1-methylpseudouridine
  modified_base 166: mod_base = OTHER; note = N1-methylpseudouridine
  modified_base 169: mod_base = OTHER; note = N1-methylpseudouridine
  modified_base 180: mod_base = OTHER; note = N1-methylpseudouridine
  modified_base 193: mod_base = OTHER; note = N1-methylpseudouridine
  modified_base 199: mod_base = OTHER; note = N1-methylpseudouridine
  modified_base 226: mod_base = OTHER; note = N1-methylpseudouridine
  modified_base 229: mod_base = OTHER; note = N1-methylpseudouridine
  modified_base 235: mod_base = OTHER; note = N1-methylpseudouridine
  modified_base 250: mod_base = OTHER; note = N1-methylpseudouridine
  modified_base 253: mod_base = OTHER; note = N1-methylpseudouridine
  modified_base 256: mod_base = OTHER; note = N1-methylpseudouridine
  modified_base 264: mod_base = OTHER; note = N1-methylpseudouridine
  modified_base 271: mod_base = OTHER; note = N1-methylpseudouridine
  modified_base 274: mod_base = OTHER; note = N1-methylpseudouridine
  modified_base 286: mod_base = OTHER; note = N1-methylpseudouridine
  modified_base 288: mod_base = OTHER; note = N1-methylpseudouridine
  modified_base 292: mod_base = OTHER; note = N1-methylpseudouridine
  modified_base 297: mod_base = OTHER; note = N1-methylpseudouridine
  modified_base 298: mod_base = OTHER; note = N1-methylpseudouridine
  modified_base 306: mod_base = OTHER; note = N1-methylpseudouridine
  modified_base 310: mod_base = OTHER; note = N1-methylpseudouridine
  modified_base 313: mod_base = OTHER; note = N1-methylpseudouridine
  modified_base 315: mod_base = OTHER; note = N1-methylpseudouridine
  modified_base 328: mod_base = OTHER; note = N1-methylpseudouridine
  modified_base 331: mod_base = OTHER; note = N1-methylpseudouridine
  modified_base 343: mod_base = OTHER; note = N1-methylpseudouridine
  modified_base 352: mod_base = OTHER; note = N1-methylpseudouridine
  modified_base 355: mod_base = OTHER; note = N1-methylpseudouridine
  modified_base 357: mod_base = OTHER; note = N1-methylpseudouridine
  modified_base 358: mod_base = OTHER; note = N1-methylpseudouridine
  modified_base 364: mod_base = OTHER; note = N1-methylpseudouridine
  modified_base 397: mod_base = OTHER; note = N1-methylpseudouridine
  modified_base 400: mod_base = OTHER; note = N1-methylpseudouridine
  modified_base 406: mod_base = OTHER; note = N1-methylpseudouridine
  modified_base 409: mod_base = OTHER; note = N1-methylpseudouridine
  modified_base 424: mod_base = OTHER; note = N1-methylpseudouridine
  modified_base 436: mod_base = OTHER; note = N1-methylpseudouridine
  modified_base 442: mod_base = OTHER; note = N1-methylpseudouridine
  modified_base 454: mod_base = OTHER; note = N1-methylpseudouridine
  modified_base 457: mod_base = OTHER; note = N1-methylpseudouridine
  modified_base 468: mod_base = OTHER; note = N1-methylpseudouridine
  modified_base 478: mod_base = OTHER; note = N1-methylpseudouridine
  modified_base 481: mod_base = OTHER; note = N1-methylpseudouridine
  modified_base 483: mod_base = OTHER; note = N1-methylpseudouridine
  modified_base 489: mod_base = OTHER; note = N1-methylpseudouridine
  modified_base 490: mod_base = OTHER; note = N1-methylpseudouridine
  modified_base 492: mod_base = OTHER; note = N1-methylpseudouridine
  modified_base 493: mod_base = OTHER; note = N1-methylpseudouridine
  modified_base 499: mod_base = OTHER; note = N1-methylpseudouridine
  modified_base 511: mod_base = OTHER; note = N1-methylpseudouridine
  modified_base 513: mod_base = OTHER; note = N1-methylpseudouridine
  modified_base 514: mod_base = OTHER; note = N1-methylpseudouridine
  modified_base 654: mod_base = OTHER; note = N1-methylpseudouridine
  modified_base 661: mod_base = OTHER; note = N1-methylpseudouridine
  modified_base 667: mod_base = OTHER; note = N1-methylpseudouridine
  modified_base 673: mod_base = OTHER; note = N1-methylpseudouridine
  modified_base 688: mod_base = OTHER; note = N1-methylpseudouridine
  modified_base 693: mod_base = OTHER; note = N1-methylpseudouridine
  modified_base 700: mod_base = OTHER; note = N1-methylpseudouridine
  modified_base 703: mod_base = OTHER; note = N1-methylpseudouridine
  modified_base 714: mod_base = OTHER; note = N1-methylpseudouridine
  modified_base 721: mod_base = OTHER; note = N1-methylpseudouridine
  modified_base 735: mod_base = OTHER; note = N1-methylpseudouridine
  modified_base 736: mod_base = OTHER; note = N1-methylpseudouridine
  modified_base 742: mod_base = OTHER; note = N1-methylpseudouridine
  modified_base 745: mod_base = OTHER; note = N1-methylpseudouridine
  modified_base 769: mod_base = OTHER; note = N1-methylpseudouridine
  modified_base 781: mod_base = OTHER; note = N1-methylpseudouridine
  modified_base 784: mod_base = OTHER; note = N1-methylpseudouridine
  modified_base 793: mod_base = OTHER; note = N1-methylpseudouridine
  modified_base 796: mod_base = OTHER; note = N1-methylpseudouridine
  modified_base 798: mod_base = OTHER; note = N1-methylpseudouridine
  modified_base 799: mod_base = OTHER; note = N1-methylpseudouridine
  modified_base 832: mod_base = OTHER; note = N1-methylpseudouridine
  modified_base 855: mod_base = OTHER; note = N1-methylpseudouridine
  modified_base 877: mod_base = OTHER; note = N1-methylpseudouridine
  modified_base 879: mod_base = OTHER; note = N1-methylpseudouridine
  modified_base 882: mod_base = OTHER; note = N1-methylpseudouridine
  modified_base 886: mod_base = OTHER; note = N1-methylpseudouridine
  modified_base 895: mod_base = OTHER; note = N1-methylpseudouridine
  modified_base 897: mod_base = OTHER; note = N1-methylpseudouridine
  modified_base 898: mod_base = OTHER; note = N1-methylpseudouridine
  modified_base 910: mod_base = OTHER; note = N1-methylpseudouridine
  modified_base 919: mod_base = OTHER; note = N1-methylpseudouridine
  modified_base 930: mod_base = OTHER; note = N1-methylpseudouridine
  modified_base 931: mod_base = OTHER; note = N1-methylpseudouridine
  modified_base 933: mod_base = OTHER; note = N1-methylpseudouridine
  modified_base 934: mod_base = OTHER; note = N1-methylpseudouridine
  modified_base 943: mod_base = OTHER; note = N1-methylpseudouridine
  modified_base 954: mod_base = OTHER; note = N1-methylpseudouridine
  modified_base 955: mod_base = OTHER; note = N1-methylpseudouridine
  modified_base 958: mod_base = OTHER; note = N1-methylpseudouridine
  modified_base 961: mod_base = OTHER; note = N1-methylpseudouridine
  modified_base 994: mod_base = OTHER; note = N1-methylpseudouridine
  modified_base 996: mod_base = OTHER; note = N1-methylpseudouridine
  modified_base 997: mod_base = OTHER; note = N1-methylpseudouridine
  modified_base 1006: mod_base = OTHER; note = N1-methylpseudouridine
  modified_base 1009: mod_base = OTHER; note = N1-methylpseudouridine
  modified_base 1018: mod_base = OTHER; note = N1-methylpseudouridine
  modified_base 1023: mod_base = OTHER; note = N1-methylpseudouridine
  modified_base 1035: mod_base = OTHER; note = N1-methylpseudouridine
  modified_base 1045: mod_base = OTHER; note = N1-methylpseudouridine
  modified_base 1047: mod_base = OTHER; note = N1-methylpseudouridine
  modified_base 1054: mod_base = OTHER; note = N1-methylpseudouridine
  modified_base 1066: mod_base = OTHER; note = N1-methylpseudouridine
  modified_base 1069: mod_base = OTHER; note = N1-methylpseudouridine
  modified_base 1093: mod_base = OTHER; note = N1-methylpseudouridine
  modified_base 1099: mod_base = OTHER; note = N1-methylpseudouridine
  modified_base 1102: mod_base = OTHER; note = N1-methylpseudouridine
  modified_base 1104: mod_base = OTHER; note = N1-methylpseudouridine
  modified_base 1108: mod_base = OTHER; note = N1-methylpseudouridine
  modified_base 1114: mod_base = OTHER; note = N1-methylpseudouridine
  modified_base 1123: mod_base = OTHER; note = N1-methylpseudouridine
  modified_base 1126: mod_base = OTHER; note = N1-methylpseudouridine
  modified_base 1131: mod_base = OTHER; note = N1-methylpseudouridine
  modified_base 1132: mod_base = OTHER; note = N1-methylpseudouridine
  modified_base 1143: mod_base = OTHER; note = N1-methylpseudouridine
  modified_base 1144: mod_base = OTHER; note = N1-methylpseudouridine
  modified_base 1147: mod_base = OTHER; note = N1-methylpseudouridine
  modified_base 1150: mod_base = OTHER; note = N1-methylpseudouridine
  modified_base 1162: mod_base = OTHER; note = N1-methylpseudouridine
  modified_base 1183: mod_base = OTHER; note = N1-methylpseudouridine
  modified_base 1192: mod_base = OTHER; note = N1-methylpseudouridine
  modified_base 1194: mod_base = OTHER; note = N1-methylpseudouridine
  modified_base 1195: mod_base = OTHER; note = N1-methylpseudouridine
  modified_base 1198: mod_base = OTHER; note = N1-methylpseudouridine
  modified_base 1204: mod_base = OTHER; note = N1-methylpseudouridine
  modified_base 1207: mod_base = OTHER; note = N1-methylpseudouridine
  modified_base 1215: mod_base = OTHER; note = N1-methylpseudouridine
  modified_base 1225: mod_base = OTHER; note = N1-methylpseudouridine
  modified_base 1227: mod_base = OTHER; note = N1-methylpseudouridine
  modified_base 1228: mod_base = OTHER; note = N1-methylpseudouridine
  modified_base 1243: mod_base = OTHER; note = N1-methylpseudouridine
  modified_base 1258: mod_base = OTHER; note = N1-methylpseudouridine
  modified_base 1273: mod_base = OTHER; note = N1-methylpseudouridine
  modified_base 1276: mod_base = OTHER; note = N1-methylpseudouridine
  modified_base 1288: mod_base = OTHER; note = N1-methylpseudouridine
  modified_base 1306: mod_base = OTHER; note = N1-methylpseudouridine
  modified_base 1315: mod_base = OTHER; note = N1-methylpseudouridine
  modified_base 1318: mod_base = OTHER; note = N1-methylpseudouridine
  modified_base 1327: mod_base = OTHER; note = N1-methylpseudouridine
  modified_base 1351: mod_base = OTHER; note = N1-methylpseudouridine
  modified_base 1353: mod_base = OTHER; note = N1-methylpseudouridine
  modified_base 1354: mod_base = OTHER; note = N1-methylpseudouridine
  modified_base 1363: mod_base = OTHER; note = N1-methylpseudouridine
  modified_base 1366: mod_base = OTHER; note = N1-methylpseudouridine
  modified_base 1372: mod_base = OTHER; note = N1-methylpseudouridine
  modified_base 1378: mod_base = OTHER; note = N1-methylpseudouridine
  modified_base 1384: mod_base = OTHER; note = N1-methylpseudouridine
  modified_base 1395: mod_base = OTHER; note = N1-methylpseudouridine
  modified_base 1396: mod_base = OTHER; note = N1-methylpseudouridine
  modified_base 1407: mod_base = OTHER; note = N1-methylpseudouridine
  modified_base 1408: mod_base = OTHER; note = N1-methylpseudouridine
  modified_base 1414: mod_base = OTHER; note = N1-methylpseudouridine
  modified_base 1432: mod_base = OTHER; note = N1-methylpseudouridine
  modified_base 1438: mod_base = OTHER; note = N1-methylpseudouridine
  modified_base 1452: mod_base = OTHER; note = N1-methylpseudouridine
  modified_base 1465: mod_base = OTHER; note = N1-methylpseudouridine
  modified_base 1474: mod_base = OTHER; note = N1-methylpseudouridine
  modified_base 1477: mod_base = OTHER; note = N1-methylpseudouridine
  modified_base 1486: mod_base = OTHER; note = N1-methylpseudouridine
  modified_base 1497: mod_base = OTHER; note = N1-methylpseudouridine
  modified_base 1507: mod_base = OTHER; note = N1-methylpseudouridine
  modified_base 1509: mod_base = OTHER; note = N1-methylpseudouridine
  modified_base 1510: mod_base = OTHER; note = N1-methylpseudouridine
  modified_base 1513: mod_base = OTHER; note = N1-methylpseudouridine
  modified_base 1528: mod_base = OTHER; note = N1-methylpseudouridine
  modified_base 1540: mod_base = OTHER; note = N1-methylpseudouridine
  modified_base 1543: mod_base = OTHER; note = N1-methylpseudouridine
  modified_base 1546: mod_base = OTHER; note = N1-methylpseudouridine
  modified_base 1555: mod_base = OTHER; note = N1-methylpseudouridine
  modified_base 1558: mod_base = OTHER; note = N1-methylpseudouridine
  modified_base 1564: mod_base = OTHER; note = N1-methylpseudouridine
  modified_base 1576: mod_base = OTHER; note = N1-methylpseudouridine
  modified_base 1591: mod_base = OTHER; note = N1-methylpseudouridine
  modified_base 1603: mod_base = OTHER; note = N1-methylpseudouridine
  modified_base 1606: mod_base = OTHER; note = N1-methylpseudouridine
  modified_base 1614: mod_base = OTHER; note = N1-methylpseudouridine
  modified_base 1636: mod_base = OTHER; note = N1-methylpseudouridine
  modified_base 1642: mod_base = OTHER; note = N1-methylpseudouridine
  modified_base 1645: mod_base = OTHER; note = N1-methylpseudouridine
  modified_base 1654: mod_base = OTHER; note = N1-methylpseudouridine
  modified_base 1657: mod_base = OTHER; note = N1-methylpseudouridine
  modified_base 1669: mod_base = OTHER; note = N1-methylpseudouridine
  modified_base 1680: mod_base = OTHER; note = N1-methylpseudouridine
  modified_base 1690: mod_base = OTHER; note = N1-methylpseudouridine
  modified_base 1692: mod_base = OTHER; note = N1-methylpseudouridine
  modified_base 1693: mod_base = OTHER; note = N1-methylpseudouridine
  modified_base 1695: mod_base = OTHER; note = N1-methylpseudouridine
  modified_base 1696: mod_base = OTHER; note = N1-methylpseudouridine
  modified_base 1716: mod_base = OTHER; note = N1-methylpseudouridine
  modified_base 1725: mod_base = OTHER; note = N1-methylpseudouridine
  modified_base 1729: mod_base = OTHER; note = N1-methylpseudouridine
  modified_base 1755: mod_base = OTHER; note = N1-methylpseudouridine
  modified_base 1756: mod_base = OTHER; note = N1-methylpseudouridine
  modified_base 1758: mod_base = OTHER; note = N1-methylpseudouridine
  modified_base 1764: mod_base = OTHER; note = N1-methylpseudouridine
  modified_base 1765: mod_base = OTHER; note = N1-methylpseudouridine
  modified_base 1768: mod_base = OTHER; note = N1-methylpseudouridine
  modified_base 1777: mod_base = OTHER; note = N1-methylpseudouridine
  modified_base 1780: mod_base = OTHER; note = N1-methylpseudouridine
  modified_base 1789: mod_base = OTHER; note = N1-methylpseudouridine
  modified_base 1807: mod_base = OTHER; note = N1-methylpseudouridine
  modified_base 1810: mod_base = OTHER; note = N1-methylpseudouridine
  modified_base 1813: mod_base = OTHER; note = N1-methylpseudouridine
  modified_base 1819: mod_base = OTHER; note = N1-methylpseudouridine
  modified_base 1834: mod_base = OTHER; note = N1-methylpseudouridine
  modified_base 1837: mod_base = OTHER; note = N1-methylpseudouridine
  modified_base 1854: mod_base = OTHER; note = N1-methylpseudouridine
  modified_base 1855: mod_base = OTHER; note = N1-methylpseudouridine
  modified_base 1870: mod_base = OTHER; note = N1-methylpseudouridine
  modified_base 1882: mod_base = OTHER; note = N1-methylpseudouridine
  modified_base 1888: mod_base = OTHER; note = N1-methylpseudouridine
  modified_base 1897: mod_base = OTHER; note = N1-methylpseudouridine
  modified_base 1906: mod_base = OTHER; note = N1-methylpseudouridine
  modified_base 1909: mod_base = OTHER; note = N1-methylpseudouridine
  modified_base 1926: mod_base = OTHER; note = N1-methylpseudouridine
  modified_base 1927: mod_base = OTHER; note = N1-methylpseudouridine
  modified_base 1929: mod_base = OTHER; note = N1-methylpseudouridine
  modified_base 1935: mod_base = OTHER; note = N1-methylpseudouridine
  modified_base 1936: mod_base = OTHER; note = N1-methylpseudouridine
  modified_base 1939: mod_base = OTHER; note = N1-methylpseudouridine
  modified_base 1960: mod_base = OTHER; note = N1-methylpseudouridine
  modified_base 1969: mod_base = OTHER; note = N1-methylpseudouridine
  modified_base 1972: mod_base = OTHER; note = N1-methylpseudouridine
  modified_base 1977: mod_base = OTHER; note = N1-methylpseudouridine
  modified_base 1978: mod_base = OTHER; note = N1-methylpseudouridine
  modified_base 1984: mod_base = OTHER; note = N1-methylpseudouridine
  modified_base 1989: mod_base = OTHER; note = N1-methylpseudouridine
  modified_base 1992: mod_base = OTHER; note = N1-methylpseudouridine
  modified_base 1996: mod_base = OTHER; note = N1-methylpseudouridine
  modified_base 2005: mod_base = OTHER; note = N1-methylpseudouridine
  modified_base 2025: mod_base = OTHER; note = N1-methylpseudouridine
  modified_base 2026: mod_base = OTHER; note = N1-methylpseudouridine
  modified_base 2031: mod_base = OTHER; note = N1-methylpseudouridine
  modified_base 2035: mod_base = OTHER; note = N1-methylpseudouridine
  modified_base 2059: mod_base = OTHER; note = N1-methylpseudouridine
  modified_base 2067: mod_base = OTHER; note = N1-methylpseudouridine
  modified_base 2073: mod_base = OTHER; note = N1-methylpseudouridine
  modified_base 2074: mod_base = OTHER; note = N1-methylpseudouridine
  modified_base 2083: mod_base = OTHER; note = N1-methylpseudouridine
  modified_base 2086: mod_base = OTHER; note = N1-methylpseudouridine
  modified_base 2112: mod_base = OTHER; note = N1-methylpseudouridine
  modified_base 2113: mod_base = OTHER; note = N1-methylpseudouridine
  modified_base 2116: mod_base = OTHER; note = N1-methylpseudouridine
  modified_base 2125: mod_base = OTHER; note = N1-methylpseudouridine
  modified_base 2133: mod_base = OTHER; note = N1-methylpseudouridine
  modified_base 2134: mod_base = OTHER; note = N1-methylpseudouridine
  modified_base 2146: mod_base = OTHER; note = N1-methylpseudouridine
  modified_base 2161: mod_base = OTHER; note = N1-methylpseudouridine
  modified_base 2164: mod_base = OTHER; note = N1-methylpseudouridine
  modified_base 2179: mod_base = OTHER; note = N1-methylpseudouridine
  modified_base 2182: mod_base = OTHER; note = N1-methylpseudouridine
  modified_base 2184: mod_base = OTHER; note = N1-methylpseudouridine
  modified_base 2190: mod_base = OTHER; note = N1-methylpseudouridine
  modified_base 2193: mod_base = OTHER; note = N1-methylpseudouridine
  modified_base 2194: mod_base = OTHER; note = N1-methylpseudouridine
  modified_base 2200: mod_base = OTHER; note = N1-methylpseudouridine
  modified_base 2202: mod_base = OTHER; note = N1-methylpseudouridine
  modified_base 2212: mod_base = OTHER; note = N1-methylpseudouridine
  modified_base 2221: mod_base = OTHER; note = N1-methylpseudouridine
  modified_base 2226: mod_base = OTHER; note = N1-methylpseudouridine
  modified_base 2230: mod_base = OTHER; note = N1-methylpseudouridine
  modified_base 2242: mod_base = OTHER; note = N1-methylpseudouridine
  modified_base 2256: mod_base = OTHER; note = N1-methylpseudouridine
  modified_base 2257: mod_base = OTHER; note = N1-methylpseudouridine
  modified_base 2260: mod_base = OTHER; note = N1-methylpseudouridine
  modified_base 2284: mod_base = OTHER; note = N1-methylpseudouridine
  modified_base 2287: mod_base = OTHER; note = N1-methylpseudouridine
  modified_base 2293: mod_base = OTHER; note = N1-methylpseudouridine
  modified_base 2296: mod_base = OTHER; note = N1-methylpseudouridine
  modified_base 2298: mod_base = OTHER; note = N1-methylpseudouridine
  modified_base 2299: mod_base = OTHER; note = N1-methylpseudouridine
  modified_base 2317: mod_base = OTHER; note = N1-methylpseudouridine
  modified_base 2323: mod_base = OTHER; note = N1-methylpseudouridine
  modified_base 2332: mod_base = OTHER; note = N1-methylpseudouridine
  modified_base 2343: mod_base = OTHER; note = N1-methylpseudouridine
  modified_base 2346: mod_base = OTHER; note = N1-methylpseudouridine
  modified_base 2347: mod_base = OTHER; note = N1-methylpseudouridine
  modified_base 2356: mod_base = OTHER; note = N1-methylpseudouridine
  modified_base 2361: mod_base = OTHER; note = N1-methylpseudouridine
  modified_base 2364: mod_base = OTHER; note = N1-methylpseudouridine
  modified_base 2365: mod_base = OTHER; note = N1-methylpseudouridine
  modified_base 2374: mod_base = OTHER; note = N1-methylpseudouridine
  modified_base 2380: mod_base = OTHER; note = N1-methylpseudouridine
  modified_base 2389: mod_base = OTHER; note = N1-methylpseudouridine
  modified_base 2400: mod_base = OTHER; note = N1-methylpseudouridine
  modified_base 2401: mod_base = OTHER; note = N1-methylpseudouridine
  modified_base 2413: mod_base = OTHER; note = N1-methylpseudouridine
  modified_base 2421: mod_base = OTHER; note = N1-methylpseudouridine
  modified_base 2431: mod_base = OTHER; note = N1-methylpseudouridine
  modified_base 2434: mod_base = OTHER; note = N1-methylpseudouridine
  modified_base 2440: mod_base = OTHER; note = N1-methylpseudouridine
  modified_base 2443: mod_base = OTHER; note = N1-methylpseudouridine
  modified_base 2457: mod_base = OTHER; note = N1-methylpseudouridine
  modified_base 2458: mod_base = OTHER; note = N1-methylpseudouridine
  modified_base 2461: mod_base = OTHER; note = N1-methylpseudouridine
  modified_base 2485: mod_base = OTHER; note = N1-methylpseudouridine
  modified_base 2488: mod_base = OTHER; note = N1-methylpseudouridine
  modified_base 2497: mod_base = OTHER; note = N1-methylpseudouridine
  modified_base 2500: mod_base = OTHER; note = N1-methylpseudouridine
  modified_base 2503: mod_base = OTHER; note = N1-methylpseudouridine
  modified_base 2509: mod_base = OTHER; note = N1-methylpseudouridine
  modified_base 2515: mod_base = OTHER; note = N1-methylpseudouridine
  modified_base 2517: mod_base = OTHER; note = N1-methylpseudouridine
  modified_base 2518: mod_base = OTHER; note = N1-methylpseudouridine
  modified_base 2533: mod_base = OTHER; note = N1-methylpseudouridine
  modified_base 2536: mod_base = OTHER; note = N1-methylpseudouridine
  modified_base 2548: mod_base = OTHER; note = N1-methylpseudouridine
  modified_base 2556: mod_base = OTHER; note = N1-methylpseudouridine
  modified_base 2566: mod_base = OTHER; note = N1-methylpseudouridine
  modified_base 2568: mod_base = OTHER; note = N1-methylpseudouridine
  modified_base 2569: mod_base = OTHER; note = N1-methylpseudouridine
  modified_base 2581: mod_base = OTHER; note = N1-methylpseudouridine
  modified_base 2584: mod_base = OTHER; note = N1-methylpseudouridine
  modified_base 2593: mod_base = OTHER; note = N1-methylpseudouridine
  modified_base 2607: mod_base = OTHER; note = N1-methylpseudouridine
  modified_base 2616: mod_base = OTHER; note = N1-methylpseudouridine
  modified_base 2626: mod_base = OTHER; note = N1-methylpseudouridine
  modified_base 2638: mod_base = OTHER; note = N1-methylpseudouridine
  modified_base 2641: mod_base = OTHER; note = N1-methylpseudouridine
  modified_base 2650: mod_base = OTHER; note = N1-methylpseudouridine
  modified_base 2677: mod_base = OTHER; note = N1-methylpseudouridine
  modified_base 2680: mod_base = OTHER; note = N1-methylpseudouridine
  modified_base 2685: mod_base = OTHER; note = N1-methylpseudouridine
  modified_base 2686: mod_base = OTHER; note = N1-methylpseudouridine
  modified_base 2689: mod_base = OTHER; note = N1-methylpseudouridine
  modified_base 2703: mod_base = OTHER; note = N1-methylpseudouridine
  modified_base 2704: mod_base = OTHER; note = N1-methylpseudouridine
  modified_base 2718: mod_base = OTHER; note = N1-methylpseudouridine
  modified_base 2719: mod_base = OTHER; note = N1-methylpseudouridine
  modified_base 2722: mod_base = OTHER; note = N1-methylpseudouridine
  modified_base 2728: mod_base = OTHER; note = N1-methylpseudouridine
  modified_base 2731: mod_base = OTHER; note = N1-methylpseudouridine
  modified_base 2746: mod_base = OTHER; note = N1-methylpseudouridine
  modified_base 2751: mod_base = OTHER; note = N1-methylpseudouridine
  modified_base 2752: mod_base = OTHER; note = N1-methylpseudouridine
  modified_base 2764: mod_base = OTHER; note = N1-methylpseudouridine
  modified_base 2779: mod_base = OTHER; note = N1-methylpseudouridine
  modified_base 2800: mod_base = OTHER; note = N1-methylpseudouridine
  modified_base 2812: mod_base = OTHER; note = N1-methylpseudouridine
  modified_base 2821: mod_base = OTHER; note = N1-methylpseudouridine
  modified_base 2839: mod_base = OTHER; note = N1-methylpseudouridine
  modified_base 2851: mod_base = OTHER; note = N1-methylpseudouridine
  modified_base 2854: mod_base = OTHER; note = N1-methylpseudouridine
  modified_base 2863: mod_base = OTHER; note = N1-methylpseudouridine
  modified_base 2869: mod_base = OTHER; note = N1-methylpseudouridine
  modified_base 2872: mod_base = OTHER; note = N1-methylpseudouridine
  modified_base 2881: mod_base = OTHER; note = N1-methylpseudouridine
  modified_base 2884: mod_base = OTHER; note = N1-methylpseudouridine
  modified_base 2890: mod_base = OTHER; note = N1-methylpseudouridine
  modified_base 2893: mod_base = OTHER; note = N1-methylpseudouridine
  modified_base 2917: mod_base = OTHER; note = N1-methylpseudouridine
  modified_base 2920: mod_base = OTHER; note = N1-methylpseudouridine
  modified_base 2923: mod_base = OTHER; note = N1-methylpseudouridine
  modified_base 2929: mod_base = OTHER; note = N1-methylpseudouridine
  modified_base 2983: mod_base = OTHER; note = N1-methylpseudouridine
  modified_base 2992: mod_base = OTHER; note = N1-methylpseudouridine
  modified_base 3004: mod_base = OTHER; note = N1-methylpseudouridine
  modified_base 3013: mod_base = OTHER; note = N1-methylpseudouridine
  modified_base 3025: mod_base = OTHER; note = N1-methylpseudouridine
  modified_base 3028: mod_base = OTHER; note = N1-methylpseudouridine
  modified_base 3043: mod_base = OTHER; note = N1-methylpseudouridine
  modified_base 3058: mod_base = OTHER; note = N1-methylpseudouridine
  modified_base 3073: mod_base = OTHER; note = N1-methylpseudouridine
  modified_base 3075: mod_base = OTHER; note = N1-methylpseudouridine
  modified_base 3079: mod_base = OTHER; note = N1-methylpseudouridine
  modified_base 3081: mod_base = OTHER; note = N1-methylpseudouridine
  modified_base 3084: mod_base = OTHER; note = N1-methylpseudouridine
  modified_base 3088: mod_base = OTHER; note = N1-methylpseudouridine
  modified_base 3106: mod_base = OTHER; note = N1-methylpseudouridine
  modified_base 3108: mod_base = OTHER; note = N1-methylpseudouridine
  modified_base 3112: mod_base = OTHER; note = N1-methylpseudouridine
  modified_base 3124: mod_base = OTHER; note = N1-methylpseudouridine
  modified_base 3130: mod_base = OTHER; note = N1-methylpseudouridine
  modified_base 3139: mod_base = OTHER; note = N1-methylpseudouridine
  modified_base 3147: mod_base = OTHER; note = N1-methylpseudouridine
  modified_base 3154: mod_base = OTHER; note = N1-methylpseudouridine
  modified_base 3163: mod_base = OTHER; note = N1-methylpseudouridine
  modified_base 3166: mod_base = OTHER; note = N1-methylpseudouridine
  modified_base 3177: mod_base = OTHER; note = N1-methylpseudouridine
  modified_base 3178: mod_base = OTHER; note = N1-methylpseudouridine
  modified_base 3181: mod_base = OTHER; note = N1-methylpseudouridine
  modified_base 3196: mod_base = OTHER; note = N1-methylpseudouridine
  modified_base 3208: mod_base = OTHER; note = N1-methylpseudouridine
  modified_base 3211: mod_base = OTHER; note = N1-methylpseudouridine
  modified_base 3250: mod_base = OTHER; note = N1-methylpseudouridine
  modified_base 3265: mod_base = OTHER; note = N1-methylpseudouridine
  modified_base 3268: mod_base = OTHER; note = N1-methylpseudouridine
  modified_base 3277: mod_base = OTHER; note = N1-methylpseudouridine
  modified_base 3285: mod_base = OTHER; note = N1-methylpseudouridine
  modified_base 3288: mod_base = OTHER; note = N1-methylpseudouridine
  modified_base 3298: mod_base = OTHER; note = N1-methylpseudouridine
  modified_base 3301: mod_base = OTHER; note = N1-methylpseudouridine
  modified_base 3313: mod_base = OTHER; note = N1-methylpseudouridine
  modified_base 3316: mod_base = OTHER; note = N1-methylpseudouridine
  modified_base 3330: mod_base = OTHER; note = N1-methylpseudouridine
  modified_base 3331: mod_base = OTHER; note = N1-methylpseudouridine
  modified_base 3340: mod_base = OTHER; note = N1-methylpseudouridine
  modified_base 3364: mod_base = OTHER; note = N1-methylpseudouridine
  modified_base 3373: mod_base = OTHER; note = N1-methylpseudouridine
  modified_base 3387: mod_base = OTHER; note = N1-methylpseudouridine
  modified_base 3388: mod_base = OTHER; note = N1-methylpseudouridine
  modified_base 3391: mod_base = OTHER; note = N1-methylpseudouridine
  modified_base 3403: mod_base = OTHER; note = N1-methylpseudouridine
  modified_base 3406: mod_base = OTHER; note = N1-methylpseudouridine
  modified_base 3421: mod_base = OTHER; note = N1-methylpseudouridine
  modified_base 3433: mod_base = OTHER; note = N1-methylpseudouridine
  modified_base 3442: mod_base = OTHER; note = N1-methylpseudouridine
  modified_base 3445: mod_base = OTHER; note = N1-methylpseudouridine
  modified_base 3457: mod_base = OTHER; note = N1-methylpseudouridine
  modified_base 3468: mod_base = OTHER; note = N1-methylpseudouridine
  modified_base 3487: mod_base = OTHER; note = N1-methylpseudouridine
  modified_base 3490: mod_base = OTHER; note = N1-methylpseudouridine
  modified_base 3499: mod_base = OTHER; note = N1-methylpseudouridine
  modified_base 3505: mod_base = OTHER; note = N1-methylpseudouridine
  modified_base 3508: mod_base = OTHER; note = N1-methylpseudouridine
  modified_base 3514: mod_base = OTHER; note = N1-methylpseudouridine
  modified_base 3526: mod_base = OTHER; note = N1-methylpseudouridine
  modified_base 3529: mod_base = OTHER; note = N1-methylpseudouridine
  modified_base 3537: mod_base = OTHER; note = N1-methylpseudouridine
  modified_base 3538: mod_base = OTHER; note = N1-methylpseudouridine
  modified_base 3549: mod_base = OTHER; note = N1-methylpseudouridine
  modified_base 3550: mod_base = OTHER; note = N1-methylpseudouridine
  modified_base 3555: mod_base = OTHER; note = N1-methylpseudouridine
  modified_base 3556: mod_base = OTHER; note = N1-methylpseudouridine
  modified_base 3558: mod_base = OTHER; note = N1-methylpseudouridine
  modified_base 3565: mod_base = OTHER; note = N1-methylpseudouridine
  modified_base 3574: mod_base = OTHER; note = N1-methylpseudouridine
  modified_base 3582: mod_base = OTHER; note = N1-methylpseudouridine
  modified_base 3603: mod_base = OTHER; note = N1-methylpseudouridine
  modified_base 3607: mod_base = OTHER; note = N1-methylpseudouridine
  modified_base 3616: mod_base = OTHER; note = N1-methylpseudouridine
  modified_base 3619: mod_base = OTHER; note = N1-methylpseudouridine
  modified_base 3631: mod_base = OTHER; note = N1-methylpseudouridine
  modified_base 3634: mod_base = OTHER; note = N1-methylpseudouridine
  modified_base 3642: mod_base = OTHER; note = N1-methylpseudouridine
  modified_base 3652: mod_base = OTHER; note = N1-methylpseudouridine
  modified_base 3663: mod_base = OTHER; note = N1-methylpseudouridine
  modified_base 3664: mod_base = OTHER; note = N1-methylpseudouridine
  modified_base 3667: mod_base = OTHER; note = N1-methylpseudouridine
  modified_base 3669: mod_base = OTHER; note = N1-methylpseudouridine
  modified_base 3678: mod_base = OTHER; note = N1-methylpseudouridine
  modified_base 3685: mod_base = OTHER; note = N1-methylpseudouridine
  modified_base 3687: mod_base = OTHER; note = N1-methylpseudouridine
  modified_base 3694: mod_base = OTHER; note = N1-methylpseudouridine
  modified_base 3703: mod_base = OTHER; note = N1-methylpseudouridine
  modified_base 3706: mod_base = OTHER; note = N1-methylpseudouridine
  modified_base 3727: mod_base = OTHER; note = N1-methylpseudouridine
  modified_base 3747: mod_base = OTHER; note = N1-methylpseudouridine
  modified_base 3750: mod_base = OTHER; note = N1-methylpseudouridine
  modified_base 3751: mod_base = OTHER; note = N1-methylpseudouridine
  modified_base 3753: mod_base = OTHER; note = N1-methylpseudouridine
  modified_base 3754: mod_base = OTHER; note = N1-methylpseudouridine
  modified_base 3756: mod_base = OTHER; note = N1-methylpseudouridine
  modified_base 3766: mod_base = OTHER; note = N1-methylpseudouridine
  modified_base 3769: mod_base = OTHER; note = N1-methylpseudouridine
  modified_base 3774: mod_base = OTHER; note = N1-methylpseudouridine
  modified_base 3775: mod_base = OTHER; note = N1-methylpseudouridine
  modified_base 3777: mod_base = OTHER; note = N1-methylpseudouridine
  modified_base 3778: mod_base = OTHER; note = N1-methylpseudouridine
  modified_base 3790: mod_base = OTHER; note = N1-methylpseudouridine
  modified_base 3796: mod_base = OTHER; note = N1-methylpseudouridine
  modified_base 3811: mod_base = OTHER; note = N1-methylpseudouridine
  modified_base 3826: mod_base = OTHER; note = N1-methylpseudouridine
  modified_base 3829: mod_base = OTHER; note = N1-methylpseudouridine
  modified_base 3856: mod_base = OTHER; note = N1-methylpseudouridine
  modified_base 3859: mod_base = OTHER; note = N1-methylpseudouridine
  modified_base 3861: mod_base = OTHER; note = N1-methylpseudouridine
  modified_base 3879: mod_base = OTHER; note = N1-methylpseudouridine
  modified_base 3880: mod_base = OTHER; note = N1-methylpseudouridine
  modified_base 3889: mod_base = OTHER; note = N1-methylpseudouridine
  modified_base 3898: mod_base = OTHER; note = N1-methylpseudouridine
  modified_base 3901: mod_base = OTHER; note = N1-methylpseudouridine
  modified_base 3907: mod_base = OTHER; note = N1-methylpseudouridine
  modified_base 3916: mod_base = OTHER; note = N1-methylpseudouridine
  modified_base 3922: mod_base = OTHER; note = N1-methylpseudouridine
  modified_base 3925: mod_base = OTHER; note = N1-methylpseudouridine
  modified_base 3940: mod_base = OTHER; note = N1-methylpseudouridine
  modified_base 3954: mod_base = OTHER; note = N1-methylpseudouridine
  modified_base 3955: mod_base = OTHER; note = N1-methylpseudouridine
  modified_base 3970: mod_base = OTHER; note = N1-methylpseudouridine
  modified_base 3973: mod_base = OTHER; note = N1-methylpseudouridine
  modified_base 3997: mod_base = OTHER; note = N1-methylpseudouridine
  modified_base 4000: mod_base = OTHER; note = N1-methylpseudouridine
  modified_base 4017: mod_base = OTHER; note = N1-methylpseudouridine
  modified_base 4032: mod_base = OTHER; note = N1-methylpseudouridine
  modified_base 4041: mod_base = OTHER; note = N1-methylpseudouridine
  modified_base 4042: mod_base = OTHER; note = N1-methylpseudouridine
  modified_base 4057: mod_base = OTHER; note = N1-methylpseudouridine
  modified_base 4062: mod_base = OTHER; note = N1-methylpseudouridine
  modified_base 4069: mod_base = OTHER; note = N1-methylpseudouridine
  modified_base 4072: mod_base = OTHER; note = N1-methylpseudouridine
  modified_base 4075: mod_base = OTHER; note = N1-methylpseudouridine
  modified_base 4078: mod_base = OTHER; note = N1-methylpseudouridine
  modified_base 4087: mod_base = OTHER; note = N1-methylpseudouridine
  modified_base 4111: mod_base = OTHER; note = N1-methylpseudouridine
  modified_base 4120: mod_base = OTHER; note = N1-methylpseudouridine
  modified_base 4129: mod_base = OTHER; note = N1-methylpseudouridine
  modified_base 4132: mod_base = OTHER; note = N1-methylpseudouridine
  modified_base 4138: mod_base = OTHER; note = N1-methylpseudouridine
  modified_base 4144: mod_base = OTHER; note = N1-methylpseudouridine
  modified_base 4147: mod_base = OTHER; note = N1-methylpseudouridine
  modified_base 4161: mod_base = OTHER; note = N1-methylpseudouridine
  modified_base 4162: mod_base = OTHER; note = N1-methylpseudouridine
  modified_base 4177: mod_base = OTHER; note = N1-methylpseudouridine
  modified_base 4182: mod_base = OTHER; note = N1-methylpseudouridine
  modified_base 4183: mod_base = OTHER; note = N1-methylpseudouridine
  modified_base 4186: mod_base = OTHER; note = N1-methylpseudouridine
  modified_base 4200: mod_base = OTHER; note = N1-methylpseudouridine
  modified_base 4210: mod_base = OTHER; note = N1-methylpseudouridine
  modified_base 4222: mod_base = OTHER; note = N1-methylpseudouridine
  modified_base 4225: mod_base = OTHER; note = N1-methylpseudouridine
  modified_base 4228: mod_base = OTHER; note = N1-methylpseudouridine
  modified_base 4234: mod_base = OTHER; note = N1-methylpseudouridine
  modified_base 4242: mod_base = OTHER; note = N1-methylpseudouridine
  modified_base 4249: mod_base = OTHER; note = N1-methylpseudouridine
  modified_base 4251: mod_base = OTHER; note = N1-methylpseudouridine
  modified_base 4252: mod_base = OTHER; note = N1-methylpseudouridine
  modified_base 4258: mod_base = OTHER; note = N1-methylpseudouridine
  modified_base 4279: mod_base = OTHER; note = N1-methylpseudouridine
  modified_base 4282: mod_base = OTHER; note = N1-methylpseudouridine
  modified_base 4300: mod_base = OTHER; note = N1-methylpseudouridine
  modified_base 4318: mod_base = OTHER; note = N1-methylpseudouridine
  modified_base 4324: mod_base = OTHER; note = N1-methylpseudouridine
  modified_base 4335: mod_base = OTHER; note = N1-methylpseudouridine
  modified_base 4339: mod_base = OTHER; note = N1-methylpseudouridine
  modified_base 4344: mod_base = OTHER; note = N1-methylpseudouridine
  modified_base 4345: mod_base = OTHER; note = N1-methylpseudouridine
  modified_base 4348: mod_base = OTHER; note = N1-methylpseudouridine
  modified_base 4350: mod_base = OTHER; note = N1-methylpseudouridine
  modified_base 4354: mod_base = OTHER; note = N1-methylpseudouridine
  modified_base 4365: mod_base = OTHER; note = N1-methylpseudouridine
  modified_base 4375: mod_base = OTHER; note = N1-methylpseudouridine
  modified_base 4411: mod_base = OTHER; note = N1-methylpseudouridine
  modified_base 4413: mod_base = OTHER; note = N1-methylpseudouridine
  modified_base 4414: mod_base = OTHER; note = N1-methylpseudouridine
  modified_base 4417: mod_base = OTHER; note = N1-methylpseudouridine
  modified_base 4434: mod_base = OTHER; note = N1-methylpseudouridine
  modified_base 4438: mod_base = OTHER; note = N1-methylpseudouridine
  modified_base 4447: mod_base = OTHER; note = N1-methylpseudouridine
  modified_base 4450: mod_base = OTHER; note = N1-methylpseudouridine
  modified_base 4459: mod_base = OTHER; note = N1-methylpseudouridine
  modified_base 4467: mod_base = OTHER; note = N1-methylpseudouridine
  modified_base 4468: mod_base = OTHER; note = N1-methylpseudouridine
  modified_base 4480: mod_base = OTHER; note = N1-methylpseudouridine
  modified_base 4483: mod_base = OTHER; note = N1-methylpseudouridine
  modified_base 4486: mod_base = OTHER; note = N1-methylpseudouridine
  modified_base 4501: mod_base = OTHER; note = N1-methylpseudouridine
  modified_base 4510: mod_base = OTHER; note = N1-methylpseudouridine
  modified_base 4513: mod_base = OTHER; note = N1-methylpseudouridine
  modified_base 4521: mod_base = OTHER; note = N1-methylpseudouridine
  modified_base 4546: mod_base = OTHER; note = N1-methylpseudouridine
  modified_base 4567: mod_base = OTHER; note = N1-methylpseudouridine
  modified_base 4570: mod_base = OTHER; note = N1-methylpseudouridine
  modified_base 4576: mod_base = OTHER; note = N1-methylpseudouridine
  modified_base 4578: mod_base = OTHER; note = N1-methylpseudouridine
  modified_base 4579: mod_base = OTHER; note = N1-methylpseudouridine
  modified_base 4585: mod_base = OTHER; note = N1-methylpseudouridine
  modified_base 4594: mod_base = OTHER; note = N1-methylpseudouridine
  modified_base 4611: mod_base = OTHER; note = N1-methylpseudouridine
  modified_base 4612: mod_base = OTHER; note = N1-methylpseudouridine
  modified_base 4617: mod_base = OTHER; note = N1-methylpseudouridine
  modified_base 4620: mod_base = OTHER; note = N1-methylpseudouridine
  modified_base 4621: mod_base = OTHER; note = N1-methylpseudouridine
  modified_base 4633: mod_base = OTHER; note = N1-methylpseudouridine
  modified_base 4647: mod_base = OTHER; note = N1-methylpseudouridine
  modified_base 4666: mod_base = OTHER; note = N1-methylpseudouridine
  modified_base 4669: mod_base = OTHER; note = N1-methylpseudouridine
  modified_base 4681: mod_base = OTHER; note = N1-methylpseudouridine
  modified_base 4684: mod_base = OTHER; note = N1-methylpseudouridine
  modified_base 4696: mod_base = OTHER; note = N1-methylpseudouridine
  modified_base 4705: mod_base = OTHER; note = N1-methylpseudouridine
  modified_base 4707: mod_base = OTHER; note = N1-methylpseudouridine
  modified_base 4720: mod_base = OTHER; note = N1-methylpseudouridine
  modified_base 4726: mod_base = OTHER; note = N1-methylpseudouridine
  modified_base 4735: mod_base = OTHER; note = N1-methylpseudouridine
  modified_base 4782: mod_base = OTHER; note = N1-methylpseudouridine
  modified_base 4783: mod_base = OTHER; note = N1-methylpseudouridine
  modified_base 4810: mod_base = OTHER; note = N1-methylpseudouridine
  modified_base 4812: mod_base = OTHER; note = N1-methylpseudouridine
  modified_base 4823: mod_base = OTHER; note = N1-methylpseudouridine
  modified_base 4824: mod_base = OTHER; note = N1-methylpseudouridine
  modified_base 4827: mod_base = OTHER; note = N1-methylpseudouridine
  modified_base 4828: mod_base = OTHER; note = N1-methylpseudouridine
  modified_base 4833: mod_base = OTHER; note = N1-methylpseudouridine
  modified_base 4837: mod_base = OTHER; note = N1-methylpseudouridine
  modified_base 4838: mod_base = OTHER; note = N1-methylpseudouridine
  modified_base 4840: mod_base = OTHER; note = N1-methylpseudouridine
  modified_base 4848: mod_base = OTHER; note = N1-methylpseudouridine
  modified_base 4849: mod_base = OTHER; note = N1-methylpseudouridine
  modified_base 4853: mod_base = OTHER; note = N1-methylpseudouridine
  modified_base 4854: mod_base = OTHER; note = N1-methylpseudouridine
  modified_base 4856: mod_base = OTHER; note = N1-methylpseudouridine
  modified_base 4862: mod_base = OTHER; note = N1-methylpseudouridine
  modified_base 4867: mod_base = OTHER; note = N1-methylpseudouridine
  modified_base 4868: mod_base = OTHER; note = N1-methylpseudouridine
  modified_base 4870: mod_base = OTHER; note = N1-methylpseudouridine
  modified_base 4871: mod_base = OTHER; note = N1-methylpseudouridine
  modified_base 4873: mod_base = OTHER; note = N1-methylpseudouridine
  modified_base 4875: mod_base = OTHER; note = N1-methylpseudouridine
  modified_base 4879: mod_base = OTHER; note = N1-methylpseudouridine
  modified_base 4880: mod_base = OTHER; note = N1-methylpseudouridine
  modified_base 4886: mod_base = OTHER; note = N1-methylpseudouridine
  modified_base 4888: mod_base = OTHER; note = N1-methylpseudouridine
  modified_base 4892: mod_base = OTHER; note = N1-methylpseudouridine
  modified_base 4894: mod_base = OTHER; note = N1-methylpseudouridine
  modified_base 4895: mod_base = OTHER; note = N1-methylpseudouridine
  modified_base 4898: mod_base = OTHER; note = N1-methylpseudouridine
  modified_base 4900: mod_base = OTHER; note = N1-methylpseudouridine
  modified_base 4901: mod_base = OTHER; note = N1-methylpseudouridine
  modified_base 4902: mod_base = OTHER; note = N1-methylpseudouridine
  modified_base 4906: mod_base = OTHER; note = N1-methylpseudouridine
  modified_base 4913: mod_base = OTHER; note = N1-methylpseudouridine
  modified_base 4917: mod_base = OTHER; note = N1-methylpseudouridine
  modified_base 4924: mod_base = OTHER; note = N1-methylpseudouridine
  modified_base 4926: mod_base = OTHER; note = N1-methylpseudouridine
aggaaataagagagaaaagaagagtaagaagaaatataagagccaccatgagcgaggtggagttcagccacgagtactggatgcggcacgccctgaccctggccaagcgggcccgggacgagcgggaggtgcccgtgggcgccgtgctggtgctgaacaaccgggtgatcggcgagggctggaaccgggccatcggcctgcacgaccccaccgcccacgccgagatcatggccctgcggcagggcggcctggtgatgcagaactaccggctgatcgacgccaccctgtacgtgaccttcgagccctgcgtgatgtgcgccggcgccatgatccacagccggatcggccgggtggtgttcggcgtgcggaacgccaagaccggcgccgccggcagcctgatggacgtgctgcaccaccccggcatgaaccaccgggtggagatcaccgagggcatcctggccgacgagtgcgccgccctgctgtgccggttcttccggatgccccggcgggtgttcaacgcccagaagaaggcccagagcagcaccgacagcggcggcagcagcggcggcagcagcggcagcgagacacccggcaccagcgagagcgccacccccgagagcagcggcggcagcagcggcggcagcgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagacagccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccgcaagagcgaggagacaatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctcaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacacggcagatcaccaagcacgtggcccagatcctggacagccggatgaacaccaagtacgacgagaacgacaagctgatccgggaggtgaaggtgatcaccctcaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagacaaacggcgagacaggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctcaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacacggatcgacctgagccagctgggcggcgacgagggcgccgacaagcggaccgccgacggcagcgagttcgagagccccaagaagaagcggaaggtgtgagcggccgcttaattaagctgccttctgcggggcttgccttctggccatgcccttcttctctcccttgcacctgtacctcttggtctttgaataaagcctgagtaggaagtctaga

SEQ ID NO: 21 - RNA, 4380 nt, synthetic construct
  source 1..4380: mol_type = other RNA
  modified_base 7: mod_base = OTHER; note = N1-methylpseudouridine
  modified_base 25: mod_base = OTHER; note = N1-methylpseudouridine
  modified_base 35: mod_base = OTHER; note = N1-methylpseudouridine
  modified_base 37: mod_base = OTHER; note = N1-methylpseudouridine
  modified_base 49: mod_base = OTHER; note = N1-methylpseudouridine
  modified_base 73: mod_base = OTHER; note = N1-methylpseudouridine
  modified_base 79: mod_base = OTHER; note = N1-methylpseudouridine
  modified_base 88: mod_base = OTHER; note = N1-methylpseudouridine
  modified_base 108: mod_base = OTHER; note = N1-methylpseudouridine
  modified_base 115: mod_base = OTHER; note = N1-methylpseudouridine
  modified_base 121: mod_base = OTHER; note = N1-methylpseudouridine
  modified_base 127: mod_base = OTHER; note = N1-methylpseudouridine
  modified_base 142: mod_base = OTHER; note = N1-methylpseudouridine
  modified_base 147: mod_base = OTHER; note = N1-methylpseudouridine
  modified_base 154: mod_base = OTHER; note = N1-methylpseudouridine
  modified_base 157: mod_base = OTHER; note = N1-methylpseudouridine
  modified_base 168: mod_base = OTHER; note = N1-methylpseudouridine
  modified_base 175: mod_base = OTHER; note = N1-methylpseudouridine
  modified_base 189: mod_base = OTHER; note = N1-methylpseudouridine
  modified_base 190: mod_base = OTHER; note = N1-methylpseudouridine
  modified_base 196: mod_base = OTHER; note = N1-methylpseudouridine
  modified_base 199: mod_base = OTHER; note = N1-methylpseudouridine
  modified_base 223: mod_base = OTHER; note = N1-methylpseudouridine
  modified_base 235: mod_base = OTHER; note = N1-methylpseudouridine
  modified_base 238: mod_base = OTHER; note = N1-methylpseudouridine
  modified_base 247: mod_base = OTHER; note = N1-methylpseudouridine
  modified_base 250: mod_base = OTHER; note = N1-methylpseudouridine
  modified_base 252: mod_base = OTHER; note = N1-methylpseudouridine
  modified_base 253: mod_base = OTHER; note = N1-methylpseudouridine
  modified_base 286: mod_base = OTHER; note = N1-methylpseudouridine
  modified_base 309: mod_base = OTHER; note = N1-methylpseudouridine
  modified_base 331: mod_base = OTHER; note = N1-methylpseudouridine
  modified_base 333: mod_base = OTHER; note = N1-methylpseudouridine
  modified_base 336: mod_base = OTHER; note = N1-methylpseudouridine
  modified_base 340: mod_base = OTHER; note = N1-methylpseudouridine
  modified_base 349: mod_base = OTHER; note = N1-methylpseudouridine
  modified_base 351: mod_base = OTHER; note = N1-methylpseudouridine
  modified_base 352: mod_base = OTHER; note = N1-methylpseudouridine
  modified_base 364: mod_base = OTHER; note = N1-methylpseudouridine
  modified_base 373: mod_base = OTHER; note = N1-methylpseudouridine
  modified_base 384: mod_base = OTHER; note = N1-methylpseudouridine
  modified_base 385: mod_base = OTHER; note = N1-methylpseudouridine
  modified_base 387: mod_base = OTHER; note = N1-methylpseudouridine
  modified_base 388: mod_base = OTHER; note = N1-methylpseudouridine
  modified_base 397: mod_base = OTHER; note = N1-methylpseudouridine
  modified_base 408: mod_base = OTHER; note = N1-methylpseudouridine
  modified_base 409: mod_base = OTHER; note = N1-methylpseudouridine
  modified_base 412: mod_base = OTHER; note = N1-methylpseudouridine
  modified_base 415: mod_base = OTHER; note = N1-methylpseudouridine
  modified_base 448: mod_base = OTHER; note = N1-methylpseudouridine
  modified_base 450: mod_base = OTHER; note = N1-methylpseudouridine
  modified_base 451: mod_base = OTHER; note = N1-methylpseudouridine
  modified_base 460: mod_base = OTHER; note = N1-methylpseudouridine
  modified_base 463: mod_base = OTHER; note = N1-methylpseudouridine
  modified_base 472: mod_base = OTHER; note = N1-methylpseudouridine
  modified_base 477: mod_base = OTHER; note = N1-methylpseudouridine
  modified_base 489: mod_base = OTHER; note = N1-methylpseudouridine
  modified_base 499: mod_base = OTHER; note = N1-methylpseudouridine
  modified_base 501: mod_base = OTHER; note = N1-methylpseudouridine
  modified_base 508: mod_base = OTHER; note = N1-methylpseudouridine
  modified_base 520: mod_base = OTHER; note = N1-methylpseudouridine
  modified_base 523: mod_base = OTHER; note = N1-methylpseudouridine
  modified_base 547: mod_base = OTHER; note = N1-methylpseudouridine
  modified_base 553: mod_base = OTHER; note = N1-methylpseudouridine
  modified_base 556: mod_base = OTHER; note = N1-methylpseudouridine
  modified_base 558: mod_base = OTHER; note = N1-methylpseudouridine
  modified_base 562: mod_base = OTHER; note = N1-methylpseudouridine
  modified_base 568: mod_base = OTHER; note = N1-methylpseudouridine
  modified_base 577: mod_base = OTHER; note = N1-methylpseudouridine
  modified_base 580: mod_base = OTHER; note = N1-methylpseudouridine
  modified_base 585: mod_base = OTHER; note = N1-methylpseudouridine
  modified_base 586: mod_base = OTHER; note = N1-methylpseudouridine
  modified_base 597: mod_base = OTHER; note = N1-methylpseudouridine
  modified_base 598: mod_base = OTHER; note = N1-methylpseudouridine
  modified_base 601: mod_base = OTHER; note = N1-methylpseudouridine
  modified_base 604: mod_base = OTHER; note = N1-methylpseudouridine
  modified_base 616: mod_base = OTHER; note = N1-methylpseudouridine
  modified_base 637: mod_base = OTHER; note = N1-methylpseudouridine
  modified_base 646: mod_base = OTHER; note = N1-methylpseudouridine
  modified_base 648: mod_base = OTHER; note = N1-methylpseudouridine
  modified_base 649: mod_base = OTHER; note = N1-methylpseudouridine
  modified_base 652: mod_base = OTHER; note = N1-methylpseudouridine
  modified_base 658: mod_base = OTHER; note = N1-methylpseudouridine
  modified_base 661: mod_base = OTHER; note = N1-methylpseudouridine
  modified_base 669: mod_base = OTHER; note = N1-methylpseudouridine
  modified_base 679: mod_base = OTHER; note = N1-methylpseudouridine
  modified_base 681: mod_base = OTHER; note = N1-methylpseudouridine
  modified_base 682: mod_base = OTHER; note = N1-methylpseudouridine
  modified_base 697: mod_base = OTHER; note = N1-methylpseudouridine
  modified_base 712: mod_base = OTHER; note = N1-methylpseudouridine
  modified_base 727: mod_base = OTHER; note = N1-methylpseudouridine
  modified_base 730: mod_base = OTHER; note = N1-methylpseudouridine
  modified_base 742: mod_base = OTHER; note = N1-methylpseudouridine
  modified_base 760: mod_base = OTHER; note = N1-methylpseudouridine
  modified_base 769: mod_base = OTHER; note = N1-methylpseudouridine
  modified_base 772: mod_base = OTHER; note = N1-methylpseudouridine
  modified_base 781: mod_base = OTHER; note = N1-methylpseudouridine
  modified_base 805: mod_base = OTHER; note = N1-methylpseudouridine
  modified_base 807: mod_base = OTHER; note = N1-methylpseudouridine
  modified_base 808: mod_base = OTHER; note = N1-methylpseudouridine
  modified_base 817: mod_base = OTHER; note = N1-methylpseudouridine
  modified_base 820: mod_base = OTHER; note = N1-methylpseudouridine
  modified_base 826: mod_base = OTHER; note = N1-methylpseudouridine
  modified_base 832: mod_base = OTHER; note = N1-methylpseudouridine
  modified_base 838: mod_base = OTHER; note = N1-methylpseudouridine
  modified_base 849: mod_base = OTHER; note = N1-methylpseudouridine
  modified_base 850: mod_base = OTHER; note = N1-methylpseudouridine
  modified_base 861: mod_base = OTHER; note = N1-methylpseudouridine
  modified_base 862: mod_base = OTHER; note = N1-methylpseudouridine
  modified_base 868: mod_base = OTHER; note = N1-methylpseudouridine
  modified_base 886: mod_base = OTHER; note = N1-methylpseudouridine
  modified_base 892: mod_base = OTHER; note = N1-methylpseudouridine
  modified_base 906: mod_base = OTHER; note = N1-methylpseudouridine
  modified_base 919: mod_base = OTHER; note = N1-methylpseudouridine
  modified_base 928: mod_base = OTHER; note = N1-methylpseudouridine
  modified_base 931: mod_base = OTHER; note = N1-methylpseudouridine
  modified_base 940: mod_base = OTHER; note = N1-methylpseudouridine
  modified_base 951: mod_base = OTHER; note = N1-methylpseudouridine
  modified_base 961: mod_base = OTHER; note = N1-methylpseudouridine
  modified_base 963: mod_base = OTHER; note = N1-methylpseudouridine
  modified_base 964: mod_base = OTHER; note = N1-methylpseudouridine
  modified_base 967: mod_base = OTHER; note = N1-methylpseudouridine
  modified_base 982: mod_base = OTHER; note = N1-methylpseudouridine
  modified_base 994: mod_base = OTHER; note = N1-methylpseudouridine
  modified_base 997: mod_base = OTHER; note = N1-methylpseudouridine
  modified_base 1000: mod_base = OTHER; note = N1-methylpseudouridine
  modified_base 1009: mod_base = OTHER; note = N1-methylpseudouridine
  modified_base 1012: mod_base = OTHER; note = N1-methylpseudouridine
  modified_base 1018: mod_base = OTHER; note = N1-methylpseudouridine
  modified_base 1030: mod_base = OTHER; note = N1-methylpseudouridine
  modified_base 1045: mod_base = OTHER; note = N1-methylpseudouridine
  modified_base 1057: mod_base = OTHER; note = N1-methylpseudouridine
  modified_base 1060: mod_base = OTHER; note = N1-methylpseudouridine
  modified_base 1068: mod_base = OTHER; note = N1-methylpseudouridine
  modified_base 1090: mod_base = OTHER; note = N1-methylpseudouridine
  modified_base 1096: mod_base = OTHER; note = N1-methylpseudouridine
  modified_base 1099: mod_base = OTHER; note = N1-methylpseudouridine
  modified_base 1108: mod_base = OTHER; note = N1-methylpseudouridine
  modified_base 1111: mod_base = OTHER; note = N1-methylpseudouridine
  modified_base 1123: mod_base = OTHER; note = N1-methylpseudouridine
  modified_base 1134: mod_base = OTHER; note = N1-methylpseudouridine
  modified_base 1144: mod_base = OTHER; note = N1-methylpseudouridine
  modified_base 1146: mod_base = OTHER; note = N1-methylpseudouridine
  modified_base 1147: mod_base = OTHER; note = N1-methylpseudouridine
  modified_base 1149: mod_base = OTHER; note = N1-methylpseudouridine
  modified_base 1150: mod_base = OTHER; note = N1-methylpseudouridine
  modified_base 1170: mod_base = OTHER; note = N1-methylpseudouridine
  modified_base 1179: mod_base = OTHER; note = N1-methylpseudouridine
  modified_base 1183: mod_base = OTHER; note = N1-methylpseudouridine
  modified_base 1209: mod_base = OTHER; note = N1-methylpseudouridine
  modified_base 1210: mod_base = OTHER; note = N1-methylpseudouridine
  modified_base 1212: mod_base = OTHER; note = N1-methylpseudouridine
  modified_base 1218: mod_base = OTHER; note = N1-methylpseudouridine
  modified_base 1219: mod_base = OTHER; note = N1-methylpseudouridine
  modified_base 1222: mod_base = OTHER; note = N1-methylpseudouridine
  modified_base 1231: mod_base = OTHER; note = N1-methylpseudouridine
  modified_base 1234: mod_base = OTHER; note = N1-methylpseudouridine
  modified_base 1243: mod_base = OTHER; note = N1-methylpseudouridine
  modified_base 1261: mod_base = OTHER; note = N1-methylpseudouridine
  modified_base 1264: mod_base = OTHER; note = N1-methylpseudouridine
  modified_base 1267: mod_base = OTHER; note = N1-methylpseudouridine
  modified_base 1273: mod_base = OTHER; note = N1-methylpseudouridine
  modified_base 1288: mod_base = OTHER; note = N1-methylpseudouridine
  modified_base 1291: mod_base = OTHER; note = N1-methylpseudouridine
  modified_base 1308: mod_base = OTHER; note = N1-methylpseudouridine
  modified_base 1309: mod_base = OTHER; note = N1-methylpseudouridine
  modified_base 1324: mod_base = OTHER; note = N1-methylpseudouridine
  modified_base 1336: mod_base = OTHER; note = N1-methylpseudouridine
  modified_base 1342: mod_base = OTHER; note = N1-methylpseudouridine
  modified_base 1351: mod_base = OTHER; note = N1-methylpseudouridine
  modified_base 1360: mod_base = OTHER; note = N1-methylpseudouridine
  modified_base 1363: mod_base = OTHER; note = N1-methylpseudouridine
  modified_base 1380: mod_base = OTHER; note = N1-methylpseudouridine
  modified_base 1381: mod_base = OTHER; note = N1-methylpseudouridine
  modified_base 1383: mod_base = OTHER; note = N1-methylpseudouridine
  modified_base 1389: mod_base = OTHER; note = N1-methylpseudouridine
  modified_base 1390: mod_base = OTHER; note = N1-methylpseudouridine
  modified_base 1393: mod_base = OTHER; note = N1-methylpseudouridine
  modified_base 1414: mod_base = OTHER; note = N1-methylpseudouridine
  modified_base 1423: mod_base = OTHER; note = N1-methylpseudouridine
  modified_base 1426: mod_base = OTHER; note = N1-methylpseudouridine
  modified_base 1431: mod_base = OTHER; note = N1-methylpseudouridine
  modified_base 1432: mod_base = OTHER; note = N1-methylpseudouridine
  modified_base 1438: mod_base = OTHER; note = N1-methylpseudouridine
  modified_base 1443: mod_base = OTHER; note = N1-methylpseudouridine
  modified_base 1446: mod_base = OTHER; note = N1-methylpseudouridine
  modified_base 1450: mod_base = OTHER; note = N1-methylpseudouridine
  modified_base 1459: mod_base = OTHER; note = N1-methylpseudouridine
  modified_base 1479: mod_base = OTHER; note = N1-methylpseudouridine
  modified_base 1480: mod_base = OTHER; note = N1-methylpseudouridine
  modified_base 1485: mod_base = OTHER; note = N1-methylpseudouridine
  modified_base 1489: mod_base = OTHER; note = N1-methylpseudouridine
  modified_base 1513: mod_base = OTHER; note = N1-methylpseudouridine
  modified_base 1521: mod_base = OTHER; note = N1-methylpseudouridine
  modified_base 1527: mod_base = OTHER; note = N1-methylpseudouridine
  modified_base 1528: mod_base = OTHER; note = N1-methylpseudouridine
  modified_base 1537: mod_base = OTHER; note = N1-methylpseudouridine
  modified_base 1540: mod_base = OTHER; note = N1-methylpseudouridine
  modified_base 1566: mod_base = OTHER; note = N1-methylpseudouridine
  modified_base 1567: mod_base = OTHER; note = N1-methylpseudouridine
  modified_base 1570: mod_base = OTHER; note = N1-methylpseudouridine
  modified_base 1579: mod_base = OTHER; note = N1-methylpseudouridine
  modified_base 1587: mod_base = OTHER; note = N1-methylpseudouridine
  modified_base 1588: mod_base = OTHER; note = N1-methylpseudouridine
  modified_base 1600: mod_base = OTHER; note = N1-methylpseudouridine
  modified_base 1615: mod_base = OTHER; note = N1-methylpseudouridine
  modified_base 1618: mod_base = OTHER; note = N1-methylpseudouridine
  modified_base 1633: mod_base = OTHER; note = N1-methylpseudouridine
  modified_base 1636: mod_base = OTHER; note = N1-methylpseudouridine
  modified_base 1638: mod_base = OTHER; note = N1-methylpseudouridine
  modified_base 1644: mod_base = OTHER; note = N1-methylpseudouridine
  modified_base 1647: mod_base = OTHER; note = N1-methylpseudouridine
  modified_base 1648: mod_base = OTHER; note = N1-methylpseudouridine
  modified_base 1654: mod_base = OTHER; note = N1-methylpseudouridine
  modified_base 1656: mod_base = OTHER; note = N1-methylpseudouridine
  modified_base 1666: mod_base = OTHER; note = N1-methylpseudouridine
  modified_base 1675: mod_base = OTHER; note = N1-methylpseudouridine
  modified_base 1680: mod_base = OTHER; note = N1-methylpseudouridine
  modified_base 1684: mod_base = OTHER; note = N1-methylpseudouridine
  modified_base 1696: mod_base = OTHER; note = N1-methylpseudouridine
  modified_base 1710: mod_base = OTHER; note = N1-methylpseudouridine
  modified_base 1711: mod_base = OTHER; note = N1-methylpseudouridine
  modified_base 1714: mod_base = OTHER; note = N1-methylpseudouridine
  modified_base 1738: mod_base = OTHER; note = N1-methylpseudouridine
  modified_base 1741: mod_base = OTHER; note = N1-methylpseudouridine
  modified_base 1747: mod_base = OTHER; note = N1-methylpseudouridine
  modified_base 1750: mod_base = OTHER; note = N1-methylpseudouridine
  modified_base 1752: mod_base = OTHER; note = N1-methylpseudouridine
  modified_base 1753: mod_base = OTHER; note = N1-methylpseudouridine
  modified_base 1771: mod_base = OTHER; note = N1-methylpseudouridine
  modified_base 1777: mod_base = OTHER; note = N1-methylpseudouridine
  modified_base 1786: mod_base = OTHER; note = N1-methylpseudouridine
  modified_base 1797: mod_base = OTHER; note = N1-methylpseudouridine
  modified_base 1800: mod_base = OTHER; note = N1-methylpseudouridine
  modified_base 1801: mod_base = OTHER; note = N1-methylpseudouridine
  modified_base 1810: mod_base = OTHER; note = N1-methylpseudouridine
  modified_base 1815: mod_base = OTHER; note = N1-methylpseudouridine
  modified_base 1818: mod_base = OTHER; note = N1-methylpseudouridine
  modified_base 1819: mod_base = OTHER; note = N1-methylpseudouridine
  modified_base 1828: mod_base = OTHER; note = N1-methylpseudouridine
  modified_base 1834: mod_base = OTHER; note = N1-methylpseudouridine
  modified_base 1843: mod_base = OTHER; note = N1-methylpseudouridine
  modified_base 1854: mod_base = OTHER; note = N1-methylpseudouridine
  modified_base 1855: mod_base = OTHER; note = N1-methylpseudouridine
  modified_base 1867: mod_base = OTHER; note = N1-methylpseudouridine
  modified_base 1875: mod_base = OTHER; note = N1-methylpseudouridine
  modified_base 1885: mod_base = OTHER; note = N1-methylpseudouridine
  modified_base 1888: mod_base = OTHER; note = N1-methylpseudouridine
  modified_base 1894: mod_base = OTHER; note = N1-methylpseudouridine
  modified_base 1897: mod_base = OTHER; note = N1-methylpseudouridine
  modified_base 1911: mod_base = OTHER; note = N1-methylpseudouridine
  modified_base 1912: mod_base = OTHER; note = N1-methylpseudouridine
  modified_base 1915: mod_base = OTHER; note = N1-methylpseudouridine
  modified_base 1939: mod_base = OTHER; note = N1-methylpseudouridine
  modified_base 1942: mod_base = OTHER; note = N1-methylpseudouridine
  modified_base 1951: mod_base = OTHER; note = N1-methylpseudouridine
  modified_base 1954: mod_base = OTHER; note = N1-methylpseudouridine
  modified_base 1957: mod_base = OTHER; note = N1-methylpseudouridine
  modified_base 1963: mod_base = OTHER; note = N1-methylpseudouridine
  modified_base 1969: mod_base = OTHER; note = N1-methylpseudouridine
  modified_base 1971: mod_base = OTHER; note = N1-methylpseudouridine
  modified_base 1972: mod_base = OTHER; note = N1-methylpseudouridine
  modified_base 1987: mod_base = OTHER; note = N1-methylpseudouridine
  modified_base 1990: mod_base = OTHER; note = N1-methylpseudouridine
  modified_base 2002: mod_base = OTHER; note = N1-methylpseudouridine
  modified_base 2010: mod_base = OTHER; note = N1-methylpseudouridine
  modified_base 2020: mod_base = OTHER; note = N1-methylpseudouridine
  modified_base 2022: mod_base = OTHER; note = N1-methylpseudouridine
  modified_base 2023: mod_base = OTHER; note = N1-methylpseudouridine
  modified_base 2035: mod_base = OTHER; note = N1-methylpseudouridine
  modified_base 2038: mod_base = OTHER; note = N1-methylpseudouridine
  modified_base 2047: mod_base = OTHER; note = N1-methylpseudouridine
  modified_base 2061: mod_base = OTHER; note = N1-methylpseudouridine
  modified_base 2070: mod_base = OTHER; note = N1-methylpseudouridine
  modified_base 2080: mod_base = OTHER; note = N1-methylpseudouridine
  modified_base 2092: mod_base = OTHER; note = N1-methylpseudouridine
  modified_base 2095: mod_base = OTHER; note = N1-methylpseudouridine
  modified_base 2104: mod_base = OTHER; note = N1-methylpseudouridine
  modified_base 2131: mod_base = OTHER; note = N1-methylpseudouridine
  modified_base 2134: mod_base = OTHER; note = N1-methylpseudouridine
  modified_base 2139: mod_base = OTHER; note = N1-methylpseudouridine
  modified_base 2140: mod_base = OTHER; note = N1-methylpseudouridine
  modified_base 2143: mod_base = OTHER; note = N1-methylpseudouridine
  modified_base 2157: mod_base = OTHER; note = N1-methylpseudouridine
  modified_base 2158: mod_base = OTHER; note = N1-methylpseudouridine
  modified_base 2172: mod_base = OTHER; note = N1-methylpseudouridine
  modified_base 2173: mod_base = OTHER; note = N1-methylpseudouridine
  modified_base 2176: mod_base = OTHER; note = N1-methylpseudouridine
  modified_base 2182: mod_base = OTHER; note = N1-methylpseudouridine
  modified_base 2185: mod_base = OTHER; note = N1-methylpseudouridine
  modified_base 2200: mod_base = OTHER; note = N1-methylpseudouridine
  modified_base 2205: mod_base = OTHER; note = N1-methylpseudouridine
  modified_base 2206: mod_base = OTHER; note = N1-methylpseudouridine
  modified_base 2218: mod_base = OTHER; note = N1-methylpseudouridine
  modified_base 2233: mod_base = OTHER; note = N1-methylpseudouridine
  modified_base 2254: mod_base = OTHER; note = N1-methylpseudouridine
  modified_base 2266: mod_base = OTHER; note = N1-methylpseudouridine
  modified_base 2275: mod_base = OTHER; note = N1-methylpseudouridine
  modified_base 2293: mod_base = OTHER; note = N1-methylpseudouridine
  modified_base 2305: mod_base = OTHER; note = N1-methylpseudouridine
  modified_base 2308: mod_base = OTHER; note = N1-methylpseudouridine
  modified_base 2317: mod_base = OTHER; note = N1-methylpseudouridine
  modified_base 2323: mod_base = OTHER; note = N1-methylpseudouridine
  modified_base 2326: mod_base = OTHER; note = N1-methylpseudouridine
  modified_base 2335: mod_base = OTHER; note = N1-methylpseudouridine
  modified_base 2338: mod_base = OTHER; note = N1-methylpseudouridine
  modified_base 2344: mod_base = OTHER; note = N1-methylpseudouridine
  modified_base 2347: mod_base = OTHER; note = N1-methylpseudouridine
  modified_base 2371: mod_base = OTHER; note = N1-methylpseudouridine
  modified_base 2374: mod_base = OTHER; note = N1-methylpseudouridine
  modified_base 2377: mod_base = OTHER; note = N1-methylpseudouridine
  modified_base 2383: mod_base = OTHER; note = N1-methylpseudouridine
  modified_base 2437: mod_base = OTHER; note = N1-methylpseudouridine
  modified_base 2446: mod_base = OTHER; note = N1-methylpseudouridine
  modified_base 2458: mod_base = OTHER; note = N1-methylpseudouridine
  modified_base 2467: mod_base = OTHER; note = N1-methylpseudouridine
  modified_base 2479: mod_base = OTHER; note = N1-methylpseudouridine
  modified_base 2482: mod_base = OTHER; note = N1-methylpseudouridine
  modified_base 2497: mod_base = OTHER; note = N1-methylpseudouridine
  modified_base 2512: mod_base = OTHER; note = N1-methylpseudouridine
  modified_base 2527: mod_base = OTHER; note = N1-methylpseudouridine
  modified_base 2529: mod_base = OTHER; note = N1-methylpseudouridine
  modified_base 2533: mod_base = OTHER; note = N1-methylpseudouridine
  modified_base 2535: mod_base = OTHER; note = N1-methylpseudouridine
  modified_base 2538: mod_base = OTHER; note = N1-methylpseudouridine
  modified_base 2542: mod_base = OTHER; note = N1-methylpseudouridine
  modified_base 2560: mod_base = OTHER; note = N1-methylpseudouridine
  modified_base 2562: mod_base = OTHER; note = N1-methylpseudouridine
  modified_base 2566: mod_base = OTHER; note = N1-methylpseudouridine
  modified_base 2578: mod_base = OTHER; note = N1-methylpseudouridine
  modified_base 2584: mod_base = OTHER; note = N1-methylpseudouridine
  modified_base 2593: mod_base = OTHER; note = N1-methylpseudouridine
  modified_base 2601: mod_base = OTHER; note = N1-methylpseudouridine
  modified_base 2608: mod_base = OTHER; note = N1-methylpseudouridine
  modified_base 2617: mod_base = OTHER; note = N1-methylpseudouridine
  modified_base 2620: mod_base = OTHER; note = N1-methylpseudouridine
  modified_base 2631: mod_base = OTHER; note = N1-methylpseudouridine
  modified_base 2632: mod_base = OTHER; note = N1-methylpseudouridine
  modified_base 2635: mod_base = OTHER; note = N1-methylpseudouridine
  modified_base 2650: mod_base = OTHER; note = N1-methylpseudouridine
  modified_base 2662: mod_base = OTHER; note = N1-methylpseudouridine
  modified_base 2665: mod_base = OTHER; note = N1-methylpseudouridine
  modified_base 2704: mod_base = OTHER; note = N1-methylpseudouridine
  modified_base 2719: mod_base = OTHER; note = N1-methylpseudouridine
  modified_base 2722: mod_base = OTHER; note = N1-methylpseudouridine
  modified_base 2731: mod_base = OTHER; note = N1-methylpseudouridine
  modified_base 2739: mod_base = OTHER; note = N1-methylpseudouridine
  modified_base 2742: mod_base = OTHER; note = N1-methylpseudouridine
  modified_base 2752: mod_base = OTHER; note = N1-methylpseudouridine
  modified_base 2755: mod_base = OTHER; note = N1-methylpseudouridine
  modified_base 2767: mod_base = OTHER; note = N1-methylpseudouridine
  modified_base 2770: mod_base = OTHER; note = N1-methylpseudouridine
  modified_base 2784: mod_base = OTHER; note = N1-methylpseudouridine
  modified_base 2785: mod_base = OTHER; note = N1-methylpseudouridine
  modified_base 2794: mod_base = OTHER; note = N1-methylpseudouridine
  modified_base 2818: mod_base = OTHER; note = N1-methylpseudouridine
  modified_base 2827: mod_base = OTHER; note = N1-methylpseudouridine
  modified_base 2841: mod_base = OTHER; note = N1-methylpseudouridine
  modified_base 2842: mod_base = OTHER; note = N1-methylpseudouridine
  modified_base 2845: mod_base = OTHER; note = N1-methylpseudouridine
  modified_base 2857: mod_base = OTHER; note = N1-methylpseudouridine
  modified_base 2860: mod_base = OTHER; note = N1-methylpseudouridine
  modified_base 2875: mod_base = OTHER; note = N1-methylpseudouridine
  modified_base 2887: mod_base = OTHER; note = N1-methylpseudouridine
  modified_base 2896: mod_base = OTHER; note = N1-methylpseudouridine
  modified_base 2899: mod_base = OTHER; note = N1-methylpseudouridine
  modified_base 2911: mod_base = OTHER; note = N1-methylpseudouridine
  modified_base 2922: mod_base = OTHER; note = N1-methylpseudouridine
  modified_base 2941: mod_base = OTHER; note = N1-methylpseudouridine
  modified_base 2944: mod_base = OTHER; note = N1-methylpseudouridine
  modified_base 2953: mod_base = OTHER; note = N1-methylpseudouridine
  modified_base 2959: mod_base = OTHER; note = N1-methylpseudouridine
  modified_base 2962: mod_base = OTHER; note = N1-methylpseudouridine
  modified_base 2968: mod_base = OTHER; note = N1-methylpseudouridine
  modified_base 2980: mod_base = OTHER; note = N1-methylpseudouridine
  modified_base 2983: mod_base = OTHER; note = N1-methylpseudouridine
  modified_base 2991: mod_base = OTHER; note = N1-methylpseudouridine
  modified_base 2992: mod_base = OTHER; note = N1-methylpseudouridine
  modified_base 3003: mod_base = OTHER; note = N1-methylpseudouridine
  modified_base 3004: mod_base = OTHER; note = N1-methylpseudouridine
  modified_base 3009: mod_base = OTHER; note = N1-methylpseudouridine
  modified_base 3010: mod_base = OTHER; note = N1-methylpseudouridine
  modified_base 3012: mod_base = OTHER; note = N1-methylpseudouridine
  modified_base 3019: mod_base = OTHER; note = N1-methylpseudouridine
  modified_base 3028: mod_base = OTHER; note = N1-methylpseudouridine
  modified_base 3036: mod_base = OTHER; note = N1-methylpseudouridine
  modified_base 3057: mod_base = OTHER; note = N1-methylpseudouridine
  modified_base 3061: mod_base = OTHER; note = N1-methylpseudouridine
  modified_base 3070: mod_base = OTHER; note = N1-methylpseudouridine
  modified_base 3073: mod_base = OTHER; note = N1-methylpseudouridine
  modified_base 3085: mod_base = OTHER; note = N1-methylpseudouridine
  modified_base 3088: mod_base = OTHER; note = N1-methylpseudouridine
  modified_base 3096: mod_base = OTHER; note = N1-methylpseudouridine
  modified_base 3106: mod_base = OTHER; note = N1-methylpseudouridine
  modified_base 3117: mod_base = OTHER; note = N1-methylpseudouridine
  modified_base 3118: mod_base = OTHER; note = N1-methylpseudouridine
  modified_base 3121: mod_base = OTHER; note = N1-methylpseudouridine
  modified_base 3123: mod_base = OTHER; note = N1-methylpseudouridine
  modified_base 3132: mod_base = OTHER; note = N1-methylpseudouridine
  modified_base 3139: mod_base = OTHER; note = N1-methylpseudouridine
  modified_base 3141: mod_base = OTHER; note = N1-methylpseudouridine
  modified_base 3148: mod_base = OTHER; note = N1-methylpseudouridine
  modified_base 3157: mod_base = OTHER; note = N1-methylpseudouridine
  modified_base 3160: mod_base = OTHER; note = N1-methylpseudouridine
  modified_base 3181: mod_base = OTHER; note = N1-methylpseudouridine
  modified_base 3201: mod_base = OTHER; note = N1-methylpseudouridine
  modified_base 3204: mod_base = OTHER; note = N1-methylpseudouridine
  modified_base 3205: mod_base = OTHER; note = N1-methylpseudouridine
  modified_base 3207: mod_base = OTHER; note = N1-methylpseudouridine
  modified_base 3208: mod_base = OTHER; note = N1-methylpseudouridine
  modified_base 3210: mod_base = OTHER; note = N1-methylpseudouridine
  modified_base 3220: mod_base = OTHER; note = N1-methylpseudouridine
  modified_base 3223: mod_base = OTHER; note = N1-methylpseudouridine
  modified_base 3228: mod_base = OTHER; note = N1-methylpseudouridine
  modified_base 3229: mod_base = OTHER; note = N1-methylpseudouridine
  modified_base 3231: mod_base = OTHER; note = N1-methylpseudouridine
  modified_base 3232: mod_base = OTHER; note = N1-methylpseudouridine
  modified_base 3244: mod_base = OTHER; note = N1-methylpseudouridine
  modified_base 3250: mod_base = OTHER; note = N1-methylpseudouridine
  modified_base 3265: mod_base = OTHER; note = N1-methylpseudouridine
  modified_base 3280: mod_base = OTHER; note = N1-methylpseudouridine
  modified_base 3283: mod_base = OTHER; note = N1-methylpseudouridine
  modified_base 3310: mod_base = OTHER; note = N1-methylpseudouridine
  modified_base 3313: mod_base = OTHER; note = N1-methylpseudouridine
  modified_base 3315: mod_base = OTHER; note = N1-methylpseudouridine
  modified_base 3333: mod_base = OTHER; note = N1-methylpseudouridine
  modified_base 3334: mod_base = OTHER; note = N1-methylpseudouridine
  modified_base 3343: mod_base = OTHER; note = N1-methylpseudouridine
  modified_base 3352: mod_base = OTHER; note = N1-methylpseudouridine
  modified_base 3355: mod_base = OTHER; note = N1-methylpseudouridine
  modified_base 3361: mod_base = OTHER; note = N1-methylpseudouridine
  modified_base 3370: mod_base = OTHER; note = N1-methylpseudouridine
  modified_base 3376: mod_base = OTHER; note = N1-methylpseudouridine
  modified_base 3379: mod_base = OTHER; note = N1-methylpseudouridine
  modified_base 3394: mod_base = OTHER; note = N1-methylpseudouridine
  modified_base 3408: mod_base = OTHER; note = N1-methylpseudouridine
  modified_base 3409: mod_base = OTHER; note = N1-methylpseudouridine
  modified_base 3424: mod_base = OTHER; note = N1-methylpseudouridine
  modified_base 3427: mod_base = OTHER; note = N1-methylpseudouridine
  modified_base 3451: mod_base = OTHER; note = N1-methylpseudouridine
  modified_base 3454: mod_base = OTHER; note = N1-methylpseudouridine
  modified_base 3471: mod_base = OTHER; note = N1-methylpseudouridine
  modified_base 3486: mod_base = OTHER; note = N1-methylpseudouridine
  modified_base 3495: mod_base = OTHER; note = N1-methylpseudouridine
  modified_base 3496: mod_base = OTHER; note = N1-methylpseudouridine
  modified_base 3511: mod_base = OTHER; note = N1-methylpseudouridine
  modified_base 3516: mod_base = OTHER; note = N1-methylpseudouridine
  modified_base 3523: mod_base = OTHER; note = N1-methylpseudouridine
  modified_base 3526: mod_base = OTHER; note = N1-methylpseudouridine
  modified_base 3529: mod_base = OTHER; note = N1-methylpseudouridine
  modified_base 3532: mod_base = OTHER; note = N1-methylpseudouridine
  modified_base 3541: mod_base = OTHER; note = N1-methylpseudouridine
  modified_base 3565: mod_base = OTHER; note = N1-methylpseudouridine
  modified_base 3574: mod_base = OTHER; note = N1-methylpseudouridine
  modified_base 3583: mod_base = OTHER; note = N1-methylpseudouridine
  modified_base 3586: mod_base = OTHER; note = N1-methylpseudouridine
  modified_base 3592: mod_base = OTHER; note = N1-methylpseudouridine
  modified_base 3598: mod_base = OTHER; note = N1-methylpseudouridine
  modified_base 3601: mod_base = OTHER; note = N1-methylpseudouridine
  modified_base 3615: mod_base = OTHER; note = N1-methylpseudouridine
  modified_base 3616: mod_base = OTHER; note = N1-methylpseudouridine
  modified_base 3631: mod_base = OTHER; note = N1-methylpseudouridine
  modified_base 3636: mod_base = OTHER; note = N1-methylpseudouridine
  modified_base 3637: mod_base = OTHER; note = N1-methylpseudouridine
  modified_base 3640: mod_base = OTHER; note = N1-methylpseudouridine
  modified_base 3654: mod_base = OTHER; note = N1-methylpseudouridine
  modified_base 3664: mod_base = OTHER; note = N1-methylpseudouridine
  modified_base 3676: mod_base = OTHER; note = N1-methylpseudouridine
  modified_base 3679: mod_base = OTHER; note = N1-methylpseudouridine
  modified_base 3682: mod_base = OTHER; note = N1-methylpseudouridine
  modified_base 3688: mod_base = OTHER; note = N1-methylpseudouridine
  modified_base 3696: mod_base = OTHER; note = N1-methylpseudouridine
  modified_base 3703: mod_base = OTHER; note = N1-methylpseudouridine
  modified_base 3705: mod_base = OTHER; note = N1-methylpseudouridine
  modified_base 3706: mod_base = OTHER; note = N1-methylpseudouridine
  modified_base 3712: mod_base = OTHER; note = N1-methylpseudouridine
  modified_base 3733: mod_base = OTHER; note = N1-methylpseudouridine
  modified_base 3736: mod_base = OTHER; note = N1-methylpseudouridine
  modified_base 3754: mod_base = OTHER; note = N1-methylpseudouridine
  modified_base 3772: mod_base = OTHER; note = N1-methylpseudouridine
  modified_base 3778: mod_base = OTHER; note = N1-methylpseudouridine
  modified_base 3789: mod_base = OTHER; note = N1-methylpseudouridine
  modified_base 3793: mod_base = OTHER; note = N1-methylpseudouridine
  modified_base 3798: mod_base = OTHER; note = N1-methylpseudouridine
  modified_base 3799: mod_base = OTHER; note = N1-methylpseudouridine
  modified_base 3802: mod_base = OTHER; note = N1-methylpseudouridine
  modified_base 3804: mod_base = OTHER; note = N1-methylpseudouridine
  modified_base 3808: mod_base = OTHER; note = N1-methylpseudouridine
  modified_base 3819: mod_base = OTHER; note = N1-methylpseudouridine
  modified_base 3829: mod_base = OTHER; note = N1-methylpseudouridine
  modified_base 3865: mod_base = OTHER; note = N1-methylpseudouridine
  modified_base 3867: mod_base = OTHER; note = N1-methylpseudouridine
  modified_base 3868: mod_base = OTHER; note = N1-methylpseudouridine
  modified_base 3871: mod_base = OTHER; note = N1-methylpseudouridine
  modified_base 3888: mod_base = OTHER; note = N1-methylpseudouridine
  modified_base 3892: mod_base = OTHER; note = N1-methylpseudouridine
  modified_base 3901: mod_base = OTHER; note = N1-methylpseudouridine
  modified_base 3904: mod_base = OTHER; note = N1-methylpseudouridine
  modified_base 3913: mod_base = OTHER; note = N1-methylpseudouridine
  modified_base 3921: mod_base = OTHER; note = N1-methylpseudouridine
  modified_base 3922: mod_base = OTHER; note = N1-methylpseudouridine
  modified_base 3934: mod_base = OTHER; note = N1-methylpseudouridine
  modified_base 3937: mod_base = OTHER; note = N1-methylpseudouridine
  modified_base 3940: mod_base = OTHER; note = N1-methylpseudouridine
  modified_base 3955: mod_base = OTHER; note = N1-methylpseudouridine
  modified_base 3964: mod_base = OTHER; note = N1-methylpseudouridine
  modified_base 3967: mod_base = OTHER; note = N1-methylpseudouridine
  modified_base 3975: mod_base = OTHER; note = N1-methylpseudouridine
  modified_base 4000: mod_base = OTHER; note = N1-methylpseudouridine
  modified_base 4021: mod_base = OTHER; note = N1-methylpseudouridine
  modified_base 4024: mod_base = OTHER; note = N1-methylpseudouridine
  modified_base 4030: mod_base = OTHER; note = N1-methylpseudouridine
  modified_base 4032: mod_base = OTHER; note = N1-methylpseudouridine
  modified_base 4033: mod_base = OTHER; note = N1-methylpseudouridine
  modified_base 4039: mod_base = OTHER; note = N1-methylpseudouridine
  modified_base 4048: mod_base = OTHER; note = N1-methylpseudouridine
  modified_base 4065: mod_base = OTHER; note = N1-methylpseudouridine
  modified_base 4066: mod_base = OTHER; note = N1-methylpseudouridine
  modified_base 4071: mod_base = OTHER; note = N1-methylpseudouridine
  modified_base 4074: mod_base = OTHER; note = N1-methylpseudouridine
  modified_base 4075: mod_base = OTHER; note = N1-methylpseudouridine
  modified_base 4087: mod_base = OTHER; note = N1-methylpseudouridine
  modified_base 4101: mod_base = OTHER; note = N1-methylpseudouridine
  modified_base 4120: mod_base = OTHER; note = N1-methylpseudouridine
  modified_base 4123: mod_base = OTHER; note = N1-methylpseudouridine
  modified_base 4135: mod_base = OTHER; note = N1-methylpseudouridine
  modified_base 4138: mod_base = OTHER; note = N1-methylpseudouridine
  modified_base 4150: mod_base = OTHER; note = N1-methylpseudouridine
  modified_base 4159: mod_base = OTHER; note = N1-methylpseudouridine
  modified_base 4161: mod_base = OTHER; note = N1-methylpseudouridine
  modified_base 4174: mod_base = OTHER; note = N1-methylpseudouridine
  modified_base 4180: mod_base = OTHER; note = N1-methylpseudouridine
  modified_base 4189: mod_base = OTHER; note = N1-methylpseudouridine
  modified_base 4257: mod_base = OTHER; note = N1-methylpseudouridine
  modified_base 4260: mod_base = OTHER; note = N1-methylpseudouridine
  modified_base 4263: mod_base = OTHER; note = N1-methylpseudouridine
  modified_base 4274: mod_base = OTHER; note = N1-methylpseudouridine
  modified_base 4275: mod_base = OTHER; note = N1-methylpseudouridine
  modified_base 4278: mod_base = OTHER; note = N1-methylpseudouridine
  modified_base 4279: mod_base = OTHER; note = N1-methylpseudouridine
  modified_base 4284: mod_base = OTHER; note = N1-methylpseudouridine
  modified_base 4288: mod_base = OTHER; note = N1-methylpseudouridine
  modified_base 4289: mod_base = OTHER; note = N1-methylpseudouridine
  modified_base 4291: mod_base = OTHER; note = N1-methylpseudouridine
  modified_base 4299: mod_base = OTHER; note = N1-methylpseudouridine
  modified_base 4300: mod_base = OTHER; note = N1-methylpseudouridine
  modified_base 4304: mod_base = OTHER; note = N1-methylpseudouridine
  modified_base 4305: mod_base = OTHER; note = N1-methylpseudouridine
  modified_base 4307: mod_base = OTHER; note = N1-methylpseudouridine
  modified_base 4313: mod_base = OTHER; note = N1-methylpseudouridine
  modified_base 4318: mod_base = OTHER; note = N1-methylpseudouridine
  modified_base 4319: mod_base = OTHER; note = N1-methylpseudouridine
  modified_base 4321: mod_base = OTHER; note = N1-methylpseudouridine
  modified_base 4322: mod_base = OTHER; note = N1-methylpseudouridine
  modified_base 4324: mod_base = OTHER; note = N1-methylpseudouridine
  modified_base 4326: mod_base = OTHER; note = N1-methylpseudouridine
  modified_base 4330: mod_base = OTHER; note = N1-methylpseudouridine
  modified_base 4331: mod_base = OTHER; note = N1-methylpseudouridine
  modified_base 4337: mod_base = OTHER; note = N1-methylpseudouridine
  modified_base 4339: mod_base = OTHER; note = N1-methylpseudouridine
  modified_base 4343: mod_base = OTHER; note = N1-methylpseudouridine
  modified_base 4345: mod_base = OTHER; note = N1-methylpseudouridine
  modified_base 4346: mod_base = OTHER; note = N1-methylpseudouridine
  modified_base 4349: mod_base = OTHER; note = N1-methylpseudouridine
  modified_base 4351: mod_base = OTHER; note = N1-methylpseudouridine
  modified_base 4352: mod_base = OTHER; note = N1-methylpseudouridine
  modified_base 4353: mod_base = OTHER; note = N1-methylpseudouridine
  modified_base 4357: mod_base = OTHER; note = N1-methylpseudouridine
  modified_base 4364: mod_base = OTHER; note = N1-methylpseudouridine
  modified_base 4368: mod_base = OTHER; note = N1-methylpseudouridine
  modified_base 4375: mod_base = OTHER; note = N1-methylpseudouridine
  modified_base 4377: mod_base = OTHER; note = N1-methylpseudouridine
aggaaataagagagaaaagaagagtaagaagaaatataagagccaccatggcccccaagaagaagcggaaggtgggcatccacggcgtgcccgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagacggccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccgcaagagcgaggagacgatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctcaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacgcggcagatcaccaagcacgtggcccagatcctggacagccggatgaacaccaagtacgacgagaacgacaagctgatccgggaggtgaaggtgatcaccctcaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagacgaacggcgagacgggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctcaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacgcggatcgacctgagccagctgggcggcgacagcggcggcaagcggcccgccgccaccaagaaggccggccaggccaagaagaagaagtaatgataggcggccgcttaattaagctgccttctgcggggcttgccttctggccatgcccttcttctctcccttgcacctgtacctcttggtctttgaataaagcctgagtaggaagtctaga